(19) United States
(12) Patent Application Publication
(10) Pub. No.: US 2024/0392353 A1
(43) Pub. Date: Nov. 28, 2024

(54) SPATIAL TRANSCRIPTOMICS FOR ANTIGEN-RECEPTORS

(71) Applicant: 10x Genomics, Inc., Pleasanton, CA (US)

(72) Inventors: Camilla Engblom, Solna (SE); Kim Thrane, Stockholm (SE); Jeffrey Mold, Pleasanton, CA (US); Jonas Frisen, Stockholm (SE); Joakim Lundeberg, Stockholm (SE); Qirong Lin, Solna (SE)

(21) Appl. No.: 18/800,754

(22) Filed: Aug. 12, 2024

Related U.S. Application Data
(63) Continuation of application No. 18/497,415, filed on Oct. 30, 2023, now Pat. No. 12,098,417.
(63) Continuation of application No. 18/315,289, filed on May 10, 2023, now Pat. No. 11,845,979.
Application No. 18/315,289 is a continuation of application No. 17/552,135, filed on Dec. 15, 2021, now Pat. No. 11,692,218.
Application No. 17/552,135 is a continuation of application No. PCT/US2021/035242, filed on Jun. 1, 2021.
(60) Provisional application No. 63/033,568, filed on Jun. 2, 2020.

(51) Int. Cl. C12Q 1/6837; C12Q 1/6869; C12Q 1/6881; H04L 9/40

(52) U.S. Cl. CPC C12Q 1/6837; C12Q 1/6869; C12Q 1/6881; H04L 63/30

(57) ABSTRACT

Provided herein are methods, compositions, and kits for the detection of immune cell clonotypes and immune cell analytes within a biological sample.

### CROSS-REFERENCE TO RELATED APPLICATIONS

This application is a continuation of U.S. patent application Ser. No. 18/497,415, filed on Oct. 30, 2024, which is a continuation of U.S. patent application Ser. No. 18/315,289, now U.S. Pat. No. 11,845,979, filed May 10, 2023, which is application is a continuation of U.S. patent application Ser. No. 17/552,135, now U.S. Pat. No. 11,692,218, filed Dec. 15, 2021, which is a continuation of International Patent Application No. PCT/US2021/035242 with an international filing date of Jun. 1, 2021, which claims priority to U.S. Provisional Patent Application Ser. No. 63/033,568, filed on Jun. 2, 2020, the contents of each of which are incorporated herein by reference in its entirety.

### SEQUENCE LISTING

This application contains a Sequence Listing that has been submitted electronically as an XML file named “47706-0208004_SL_ST26.XML.” The XML file, created on Aug. 12, 2024, is 591,820 bytes in size. The material in the XML file is hereby incorporated by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Understanding spatial heterogeneity in the context of immune cell clonotypes (e.g., T-cell receptor, B-cell receptor) within an intact biological sample, or a portion thereof, can give insight into which cells or cell-types specific T-cell or B-cell clonotypes may be interacting. Single-cell methods can identify clonotype populations, but fail to link the spatial organization of immune cell clonotypes within a biological sample.

### SUMMARY

A fundamental understanding of spatial heterogeneity with respect to T-cell receptor (TCR) and B-cell receptor (BCR) clonotypes within a biological sample is needed to understand which cells a TCR or BCR may interacting with, the identity of TCR and/or BCR clonotypes in a given biological sample, or the identity of TCR and/or BCR clonotypes that are autoreactive in different autoimmune disorders. Numerous single-cell sequencing approaches can identify TCR and BCR clonotypes from a biological sample, however, at present methods are needed to link TCR and BCR sequences to spatial locations within a biological sample. Additionally, identifying the clonal regions, that is, regions defined by the places where variable (V), diverse (D), and joining (J) segments join to form the complementarity determining regions, including CDR1, CDR2, and CDR3, which provide specificity to the TCRs and/or BCRs, is important in understanding the TCR and BCR biological interactions. By coupling clonal information to spatial information it is possible to understand which T-cell and B-cell clonotypes may be specifically interacting with given cell types within a biological sample.

Provided herein are methods for determining the presence and/or abundance of an immune cell clonotype at a location in a biological sample. Some embodiments of any of the methods described herein include capturing transcripts to identify an immune cell clonotype. Some embodiments of any of the methods herein include generating a nucleic acid library from captured transcripts. Some embodiments of any of the methods described herein include enriching analytes of interest in the nucleic acid library, including analytes to identify an immune cell clonotype.

Provided herein are methods for determining the presence and/or abundance of an immune cell receptor at a location in a biological sample. Some embodiments of any of the methods described herein include capturing analytes to identify an immune cell receptor. Some embodiments of any of the methods described herein include generating a nucleic acid library from captured analytes. Some embodiments of any of the methods described here include enriching analytes of interest in the nucleic acid library, including analytes to identify an immune cell receptor.

Thus provided herein are methods for determining the presence and/or abundance of an immune cell clonotype at a location in a biological sample, the method including: (a) contacting a biological sample with an array including a plurality of capture probes, where a capture probe of the plurality of capture probes includes (i) a spatial barcode and (ii) a capture domain that binds to a nucleic acid encoding an immune cell receptor of the immune cell clonotype; and (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell clonotype at a location in the biological sample.

In some embodiments, the immune cell clonotype is a T cell clonotype. In some embodiments, the T cell clonotype is a T cell receptor alpha chain. In some embodiments, the capture domain binds to a nucleic acid sequence encoding a constant region of the T cell receptor alpha chain. In some embodiments, step (b) includes determining a sequence encoding CDR3 of the T cell receptor alpha chain. In some embodiments, step (b) includes determining a sequence encoding one or both of CDR1 and CDR2 of the T cell receptor alpha chain. In some embodiments, step (b) includes determining a sequence encoding a full-length variable domain of the T cell receptor alpha chain.

In some embodiments, the immune cell receptor is a T cell receptor beta chain. In some embodiments, the capture domain binds specifically to a nucleic acid sequence encoding a constant region of the T cell receptor beta chain. In some embodiments, step (b) includes determining a sequence encoding CDR3 of the T cell receptor beta chain. In some embodiments, step (b) includes determining a sequence encoding one or both of CDR1 and CDR2 of the T cell receptor beta chain. In some embodiments, step (b) includes determining a sequence encoding a full-length variable domain of the T cell receptor beta chain.

In some embodiments, the immune cell clonotype is a B cell clonotype. In some embodiments, the B cell clonotype is an immunoglobulin kappa light chain. In some embodiments, the capture domain binds specifically to a nucleic acid sequence encoding a constant region of the immunoglobulin kappa light chain. In some embodiments, step (b) includes determining a sequence encoding CDR3 of the immunoglobulin kappa light chain. In some embodiments, step (b) includes determining a sequence encoding one or both of CDR1 and CDR2 of the immunoglobulin kappa light chain. In some embodiments, step (b) includes determining a sequence encoding a full-length variable domain of the immunoglobulin kappa light chain. In some embodiments, the B cell clonotype is an immunoglobulin lambda light chain. In some embodiments, the capture domain binds specifically to a nucleic acid sequence encoding a constant region of the immunoglobulin lambda light chain. In some embodiments, step (b) includes determining a sequence encoding CDR3 of the immunoglobulin lambda light chain. In some embodiments, step (b) includes determining a sequence encoding one or both of CDR1 and CDR2 of the immunoglobulin lambda light chain. In some embodiments, step (b) includes determining a sequence encoding a full-length variable domain of the immunoglobulin lambda light chain. In some embodiments, the B cell clonotype is an immunoglobulin heavy chain. In some embodiments, the capture domain binds specifically to a nucleic acid sequence encoding a constant region of the immunoglobulin heavy chain. In some embodiments, step (b) includes determining a sequence encoding CDR3 of the immunoglobulin heavy chain. In some embodiments, step (b) includes determining a sequence encoding one or both of CDR1 and CDR2 of the immunoglobulin heavy chain. In some embodiments, step (b) includes determining a sequence encoding a full-length variable domain of the immunoglobulin heavy chain.

In some embodiments, the capture domain binds a poly(A) sequence of a nucleic acid encoding an immune cell clonotype. In some embodiments, the capture domain binds to a nucleic acid sequence encoding a T cell clonotype. In some embodiments, the T cell clonotype is a T cell receptor alpha chain, a T cell receptor beta chain, and combinations thereof. In some embodiments, step (b) includes determining: a sequence encoding CDR3 of the T cell receptor alpha chain, a sequence encoding CDR3 of the T cell receptor beta chain, and combinations thereof. In some embodiments, step (b) includes determining: a sequence encoding one or both of CDR1 and CDR2 of the T cell receptor alpha chain, a sequence encoding one or both of CDR1 and CDR2 of the T cell receptor beta chain, and combinations thereof. In some embodiments, step (b) includes determining: a sequence encoding a full-length variable domain of the T cell receptor alpha chain, a sequence encoding a full-length variable domain of the T cell receptor beta chain, and combinations thereof.

In some embodiments, the capture domain binds to a nucleic acid encoding a B cell clonotype. In some embodiments, the B cell clonotype is an immunoglobulin kappa light chain, an immunoglobulin lambda light chain, an immunoglobulin heavy chain, and combinations thereof. In some embodiments, step (b) includes determining: a sequence encoding CDR3 of the immunoglobulin kappa light chain, a sequence encoding CDR3 of immunoglobulin lambda light chain, a sequence encoding CDR3 of the immunoglobulin heavy chain, and combinations thereof. In some embodiments, step (b) includes determining: a sequence encoding one or both of CDR1 and CDR2 of the immunoglobulin kappa light chain, a sequence encoding one or both of CDR1 and CDR2 of the immunoglobulin lambda light chain, a sequence encoding one or both of CDR1 and CDR2 of the immunoglobulin heavy chain, and combinations thereof. In some embodiments, step (b) includes determining: a sequence encoding a full-length variable domain of the immunoglobulin kappa light chain, a sequence encoding a full-length variable domain of the immunoglobulin lambda light chain, a sequence encoding a full-length variable domain of the immunoglobulin heavy chain, and combinations thereof.

In some embodiments, step (b) includes the capture probe using the nucleic acid encoding the immune cell receptor as a template, thereby generating an extended capture probe. In some embodiments, step (b) includes extending a 3′ end of the capture probe.

In some embodiments, step (b) includes generating a second strand of nucleic acid that includes (i) a sequence that is complementary to all or a portion of the spatial barcode, and (ii) a sequence that corresponds to all or a portion of the sequence of the nucleic acid encoding the immune cell receptor.

In some embodiments, the capture probe includes a cleavage domain, a functional domain, a unique molecular identifier, or any combination thereof. In some embodiments, the capture probe includes a functional domain.

In some embodiments, step (b) includes generating a second strand of nucleic acid that includes (i) a sequence that is complementary to all or a portion of the functional domain, (ii) a sequence that is complementary to all or a portion of the spatial barcode, and (iii) a sequence that corresponds to all or a portion of the sequence of the nucleic acid encoding the immune cell receptor.

In some embodiments, the method includes enriching the nucleic acid encoding the immune cell receptor of the immune cell clonotype.

In some embodiments, enriching includes hybridizing a plurality of hybridization probes to the nucleic acid encoding the immune cell receptor of the immune cell clonotype, where a hybridization probe includes (i) a sequence complementary to a portion of the nucleic acid encoding the immune cell receptor and (ii) a binding moiety that interacts with a capture moiety.

In some embodiments, the binding moiety includes biotin and the capture moiety includes streptavidin.

In some embodiments, enriching the nucleic acid encoding the immune cell receptor of the immune cell clonotype includes one or more blocking probes. In some embodiments, the one or more blocking probes includes a sequence having at least 80% identity to SEQ ID NO: 639. In some embodiments, the one or more blocking probes includes a sequence having at least 80% identity to SEQ ID NO: 640.

In some embodiments, the method includes amplifying the nucleic acid encoding the immune cell receptor of the immune cell clonotype, or a complement thereof, using (i) a first primer including all or a portion of the functional domain, where the functional domain is 5′ to the spatial barcode, and (ii) a second primer including a sequence that is substantially complementary to a portion of a sequence encoding a variable region of the immune cell receptor.

In some embodiments, the method includes amplifying the nucleic acid encoding the immune cell receptor of the immune cell clonotype, or a complement thereof, using (i) the first primer including all or a portion of the functional domain, where the functional domain is 5′ to the spatial barcode, and (ii) a third primer including a sequence that is substantially complementary to a portion of the nucleic acid sequence encoding a variable region of the immune cell receptor, where the third primer is 5′ to the second primer.

In some embodiments, the biological sample includes a tissue sample. In some embodiments, the tissue sample is a tissue section. In some embodiments, the tissue section is a fixed tissue section. In some embodiments, the fixed tissue section is a formalin-fixed paraffin-embedded tissue section. In some embodiments, the tissue section includes a tumor region.

In some embodiments, the nucleic acid encoding the immune cell receptor includes RNA. In some embodiments, the RNA is mRNA. In some embodiments, the nucleic acid encoding the immune cell receptor includes DNA. In some embodiments, the DNA is genomic DNA.

In some embodiments, the method includes imaging the biological sample.

In some embodiments, the determining in step (b) includes sequencing (i) all or a portion of the sequence of the spatial barcode or a complement thereof, and (ii) all or a portion of the sequence of the nucleic acid encoding the immune cell receptor or a complement thereof.

In some embodiments, step (b) includes determining the presence of the immune cell clonotype at a location in the biological sample. In some embodiments, step (b) includes determining the abundance of the immune cell clonotype at a location in the biological sample. In some embodiments, step (b) includes determining the presence and abundance of the immune cell clonotype at a location in the biological sample. In some embodiments, step (b) includes determining the presence of two or more immune cell clonotypes at a location in the biological sample. In some embodiments, step (b) includes determining the abundance of two or more immune cell clonotypes at a location in the biological sample. In some embodiments, step (b) includes determining the presence and abundance of two or more immune cell clonotypes at a location in the biological sample. In some embodiments, the method includes comparing the two or more immune cell clonotypes. In some embodiments, the two or more immune cell clonotypes are each a B cell clonotype.

In some embodiments, the two or more immune cell clonotypes are each a T cell clonotype. In some embodiments, the two or more immune cell clonotypes comprise at least one T cell clonotype and at least one B cell clonotype.

Also provided herein are methods for determining the presence and/or abundance of an immune cell receptor at a location in a biological sample, the method including: (a) contacting a biological sample with an array including a plurality of capture probes, where a capture probe of the plurality of capture probes includes (i) a spatial barcode and (ii) a capture domain that specifically binds to a nucleic acid encoding an immune cell receptor; and (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell receptor at a location in the biological sample.

In some embodiments, the immune cell receptor is a T cell receptor alpha chain. In some embodiments, the capture domain binds specifically to a nucleic acid sequence encoding a constant region of the T cell receptor alpha chain. In some embodiments, step (b) includes determining a sequence encoding CDR3 of the T cell receptor alpha chain. In some embodiments, step (b) includes determining a sequence encoding one or both of CDR1 and CDR2 of the T cell receptor alpha chain. In some embodiments, step (b) includes determining a sequence encoding a full-length variable domain of the T cell receptor alpha chain. In some embodiments, the immune cell receptor is a T cell receptor beta chain. In some embodiments, the capture domain binds specifically to a nucleic acid sequence encoding a constant region of the T cell receptor beta chain. In some embodiments, step (b) includes determining a sequence encoding CDR3 of the T cell receptor beta chain. In some embodiments, step (b) includes determining a sequence encoding one or both of CDR1 and CDR2 of the T cell receptor beta chain. In some embodiments, step (b) includes determining a full-length variable domain of the T cell receptor beta chain. In some embodiments, the immune cell receptor is an immunoglobulin kappa light chain. In some embodiments, the capture domain binds specifically to a nucleic acid sequence encoding a constant region of the immunoglobulin kappa light chain. In some embodiments, step (b) includes determining a sequence encoding CDR3 of the immunoglobulin kappa light chain. In some embodiments, step (b) includes determining a sequence encoding one or both of CDR1 and CDR2 of the immunoglobulin kappa light chain. In some embodiments, step (b) includes determining a sequence encoding a full-length variable domain of the immunoglobulin kappa light chain. In some embodiments, the immune cell receptor is an immunoglobulin lambda light chain. In some embodiments, the capture domain binds specifically to a nucleic acid sequence encoding a constant region of the immunoglobulin lambda light chain. In some embodiments, step (b) includes determining a sequence encoding CDR3 of the immunoglobulin lambda light chain. In some embodiments, step (b) includes determining a sequence encoding one or both of CDR1 and CDR2 of the immunoglobulin lambda light chain. In some embodiments, step (b) includes determining a sequence encoding a full-length variable domain of the immunoglobulin lambda light chain. In some embodiments, the immune cell receptor is an immunoglobulin heavy chain. In some embodiments, the capture domain binds specifically to a nucleic acid sequence encoding a constant region of the immunoglobulin heavy chain. In some embodiments, step (b) includes determining a sequence encoding CDR3 of the immunoglobulin heavy chain. In some embodiments, step (b) includes determining a sequence encoding one or both of CDR1 and CDR2 of the immunoglobulin heavy chain. In some embodiments, step (b) includes determining a sequence encoding a full-length variable domain of the immunoglobulin heavy chain.

In some embodiments, the capture domain binds a poly(A) sequence of a nucleic acid encoding an immune cell receptor. In some embodiments, the immune cell receptor is a T cell receptor alpha chain, a T cell receptor beta chain, and combinations thereof. In some embodiments, step (b) includes determining: a sequence encoding CDR3 of the T cell receptor alpha chain, a sequence encoding CDR3 of the T cell receptor beta chain, and combinations thereof. In some embodiments, step (b) includes determining: a sequence encoding one or both of CDR1 and CDR2 of the T cell receptor alpha chain, a sequence encoding one or both of CDR1 and CDR2 of the T cell receptor beta chain, and combinations thereof. In some embodiments, step (b) includes determining: a sequence encoding a full-length variable domain of the T cell receptor alpha chain, a sequence encoding a full-length variable domain of the T cell receptor beta chain, and combinations thereof.

In some embodiments, the immune cell receptor is a B cell receptor an immunoglobulin kappa light chain, an immunoglobulin lambda light chain, an immunoglobulin heavy chain, and combinations thereof. In some embodiments, step (b) includes determining: a sequence encoding CDR3 of the immunoglobulin kappa light chain, a sequence encoding CDR3 of immunoglobulin lambda light chain, a sequence encoding CDR3 of the immunoglobulin heavy chain, and combinations thereof. In some embodiments, step (b) includes determining: a sequence encoding one or both of CDR1 and CDR2 of the immunoglobulin kappa light chain, a sequence encoding one or both of CDR1 and CDR2 of the immunoglobulin lambda light chain, a sequence encoding one or both of CDR1 and CDR2 of the immunoglobulin heavy chain, and combinations thereof. In some embodiments, step (b) includes determining: a sequence encoding a full-length variable domain of the immunoglobulin kappa light chain, a sequence encoding a full-length variable domain of the immunoglobulin lambda light chain, a sequence encoding a full-length variable domain of the immunoglobulin heavy chain, and combinations thereof.

In some embodiments, step (b) includes extending an end of the capture probe using the nucleic acid encoding the immune cell receptor as a template, thereby generating an extended capture probe. In some embodiments, step (b) includes extending a 3′ end of the capture probe.

In some embodiments, step (b) includes generating a second strand of nucleic acid that includes (i) a sequence that is complementary to all or a portion of the spatial barcode, and (ii) a sequence that corresponds to all or a portion of the sequence of the nucleic acid encoding the immune cell receptor.

In some embodiments, the capture probe includes a cleavage domain, a functional domain, a unique molecular identifier, or any combination thereof. In some embodiments, the capture probe includes a functional domain.

In some embodiments, step (b) includes generating a second strand of nucleic acid that includes (i) a sequence that is complementary to all or a portion of the functional domain, (ii) a sequence that is complementary to all or a portion of the spatial barcode, and (iii) a sequence that corresponds to all or a portion of the sequence of the nucleic acid encoding the immune cell receptor.

In some embodiments, the method includes enriching the nucleic acid encoding the immune cell receptor. In some embodiments, enriching includes hybridizing a plurality of hybridization probes to the nucleic acid encoding the immune cell receptor, where a hybridization probe includes (i) a sequence complementary to a portion of the nucleic acid encoding the immune cell receptor and (ii) a binding moiety that interacts with a capture moiety. In some embodiments, the binding moiety includes biotin and the capture moiety includes streptavidin. In some embodiments, enriching the nucleic acid encoding the immune cell receptor of the immune cell receptor includes one or more blocking probes. In some embodiments, the one or more blocking probes includes a sequence having at least 80% identity to SEQ ID NO: 639. In some embodiments, the one or more blocking probes includes a sequence having at least 80% identity to SEQ ID NO: 640.

In some embodiments, the method includes amplifying the nucleic acid encoding an immune cell receptor, or a complement thereof, using (i) a first primer including all or a portion of the functional domain, where the functional domain is 5′ to the spatial barcode in the second strand of nucleic acid, and (ii) a second primer including a sequence that is substantially complementary to a portion of a sequence encoding a variable region of the immune cell receptor.

In some embodiments, the method includes amplifying the nucleic acid encoding the immune cell receptor, or a complement thereof, using (i) the first primer including all or a portion of the functional domain, where the functional domain is 5′ to the spatial barcode, and (ii) a third primer including a sequence that is substantially complementary to a portion of the nucleic acid sequence encoding a variable region of the immune cell receptor, where the third primer is 5′ to the second primer.

In some embodiments, the biological sample includes a tissue sample. In some embodiments, the tissue sample is a tissue section. In some embodiments, the tissue section is a fixed tissue section. In some embodiments, the fixed tissue section is a formalin-fixed paraffin-embedded tissue section. In some embodiments, the tissue section includes a tumor region.

In some embodiments, the nucleic acid encoding the immune cell receptor includes RNA. In some embodiments, the RNA is mRNA. In some embodiments, the nucleic acid encoding the immune cell receptor includes DNA. In some embodiments, the DNA is genomic DNA.

In some embodiments, the method includes, prior to step (b), contacting the biological sample with ribosomal RNA depletion probes and mitochondrial RNA depletion probes.

In some embodiments, the method includes imaging the biological sample.

In some embodiments, the determining in step (b) includes sequencing (i) all or a portion of the sequence of the spatial barcode or a complement thereof, and (ii) all or a portion of the sequence of the nucleic acid encoding the immune cell receptor or a complement thereof.

In some embodiments, step (b) includes determining the presence of the immune cell receptor at a location in the biological sample. In some embodiments, step (b) includes determining the abundance of the immune cell receptor at a location in the biological sample. In some embodiments, step (b) includes determining the presence and abundance of the immune cell receptor at a location in the biological sample. In some embodiments, step (b) includes determining the presence of two or more immune cell receptors at a location in the biological sample. In some embodiments, step (b) includes determining the abundance of two or more immune cell receptors at a location in the biological sample. In some embodiments, step (b) includes determining the presence and abundance of two or more immune cell receptors at a location in the biological sample. In some embodiments, the method includes comparing the two or more immune cell receptors. In some embodiments, the two or more immune cell clonotypes are each an immune cell receptor of a B cell. In some embodiments, the two or more immune cell clonotypes are each an immune cell receptor of a T cell. In some embodiments, the two or more immune cell clonotypes comprise at least one immune cell receptor of a T cell and at least one immune cell receptor from a B cell.

Also provided herein are arrays including a plurality of capture probes, where a capture probe of the plurality of capture probes includes (i) a spatial barcode and (ii) a capture domain that binds to a nucleic acid encoding an immune cell receptor of an immune cell clonotype.

In some embodiments, the immune cell clonotype is a T cell clonotype. In some embodiments, the immune cell receptor is a T cell receptor alpha chain. In some embodiments, the capture domain binds specifically to a nucleic acid sequence encoding a constant region of the T cell receptor alpha chain. In some embodiments, the immune cell receptor is a T cell receptor beta chain. In some embodiments, the capture domain binds specifically to a nucleic acid sequence encoding a constant region of the T cell receptor beta chain.

In some embodiments, the immune cell clonotype is a B cell clonotype. In some embodiments, the immune cell receptor is an immunoglobulin kappa light chain. In some embodiments, the capture domain binds specifically to a nucleic acid sequence encoding a constant region of the immunoglobulin kappa light chain. In some embodiments, the immune cell receptor is an immunoglobulin lambda light chain. In some embodiments, the capture domain binds specifically to a nucleic acid sequence encoding a constant region of the immunoglobulin lambda light chain. In some embodiments, the immune cell receptor is an immunoglobulin heavy chain. In some embodiments, the capture domain binds specifically to a nucleic acid sequence encoding a constant region of the immunoglobulin heavy chain.

In some embodiments, the capture probe includes a cleavage domain, a functional domain, a unique molecular identifier, or any combination thereof.

Also provided herein are kits any one of the arrays described herein; one or more hybridization probes, where a hybridization probe includes (i) a sequence substantially complementary to a nucleic acid encoding an immune cell receptor and (ii) a binding moiety that interacts with a capturing moiety; and one or more blocking probes.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 shows an exemplary workflow for spatial transcriptomics for antigen-receptors.

FIG. 3A shows an exemplary capture probe with a capture sequence complementary to a constant region of an analyte.

FIG. 3B shows an exemplary capture probe with a poly(dT) capture domain.

FIG. 4 shows an exemplary enrichment strategy with a Read1 primer and a primer(s) complementary to a variable region of an analyte.

FIG. 5 shows an exemplary sequencing strategy with a ligated sequencing handle (P5) and a custom sequencing primer complementary to a portion of the constant region of an analyte.

FIG. 6 shows a graph showing the number of unique clonotypes detected from lymph node (LN) and spleen (SP) tissues.

FIG. 7A shows an exemplary graph of the number of unique T-cell receptor A (TRA) and T-cell receptor B (TRB) clonotypes detected with a targeted capture probe compared with a poly(dT) capture probe.

FIG. 7B shows an exemplary graph of the number of unique IG heavy chain clonotypes (e.g., A, G, M and E clonotypes) detected with a targeted capture probe compared with a poly(dT) capture probe.

FIG. 8 shows an exemplary graph of the number of unique IG clonotypes detected in tonsil tissue on a spatial array (Vis) compared with single cell (SS2) analysis, with or without an enrichment strategy.

FIG. 9 shows an exemplary graph of the number of unique IG clonotypes detected in lymph node (LN) tissue on a spatial array with and without an enrichment strategy. Lymph node and spleen (SP) non-enrichment samples serve as controls.

FIG. 10A shows H&E stained tonsil tissue.

FIG. 10B shows a gene expression library generated from the tonsil tissue in FIG. 10A.

FIG. 11 shows single-cell clustering analysis of the T-cell receptor and B-cell receptor clonotypes present in a breast tumor sample.

FIG. 12 shows an exemplary capture probe with a poly(dT) capture domain (top) followed by reverse transcription to generate cDNA of an analyte.

FIG. 13 shows cDNA libraries for either B-cell receptors (BCR), T-cell receptors (TCR), or other analytes and pools of BCR and TCR with enrichment hybridization probes.

FIG. 14 shows hybridization of the BCR and TCR specific enrichment hybridization probes to their respective targets in the cDNA library.

FIG. 15 shows hybridization of blocking oligonucleotides targeting various domains present in the cDNA library.

FIG. 16A shows replicate tonsil sections (top and bottom) and detection of BCR and TCR clonotype count (left) and BCR and TCR unique molecular identifier count (right).

FIG. 16B shows a graph showing the total number of unique clonotypes found in the replicate tonsil sections from FIG. 16A.

FIG. 16C shows a graph showing the clonotype count split by IGH isotype found in the replicate tonsil sections from FIG. 16A.

FIG. 17A shows H&E stained tonsil tissue (left), CD20 spatial expression (middle), and CD138 expression (control).

FIG. 17B shows spatial expression in tonsil tissue of the heavy chain IGH constant gene (top) including IGHM, IGHG1, IGHA1, and IGHD and the light chain (bottom) including IGKC and IGLC2.

FIG. 17C shows T-cell specific spatial expression for CD3E, TRBC1, TRABC2, and TRAC in tonsil tissue.

FIG. 18A shows a detected IG clone expression (IGKC) restricted to about one B-cell follicle of in tonsil tissue in replicate experiments.

FIG. 18B shows a detected IG clone expression (IGLC) restricted to a B-cell follicle in tonsil tissue in replicate experiments.

FIG. 18C shows detected IG clone expression (IGLV3-1, IGLJ2, IGLC2/IGLC3) with expression not restricted to B-cell follicles in tonsil tissue in replicate experiments.

FIG. 18D shows detected IG clone expression (IGHM) in single B-cell follicles in tonsil tissue in replicate experiments.

FIG. 18E shows detected IG clone expression (IGHA) expression outside B-cell follicles in tonsil tissue in replicate experiments.

FIG. 18F shows a representative T-cell clone expression (TRB) distributed outside of B-cell follicles in tonsil tissue in replicate experiments.

FIG. 18G shows a representative T-cell clone expression (TRA) distributed outside of B-cell follicles in tonsil tissue in replicate experiments.

FIG. 19A shows clonotype distribution in replicate breast tumor samples (Tumor D1,Tumor D2) and clonotype count (left) and UMI count (right).

FIG. 19B is a graph showing total clonotype count of the replicate breast tumor samples shown in FIG. 19A.

FIG. 19C is a graph showing clonotype count split by IGH isotype of the replicate breast tumor samples shown in FIG. 19A.

FIG. 20 shows the distribution of a representative IGH clonotypes of the replicate breast tumor samples shown in FIG. 19A.

FIG. 21A shows spatial patterning of paired IG receptors (IGHG2 and IGK) (left) in breast tumor tissue and single-cell RNA-seq (right).

FIG. 21B shows spatial patterning of paired IG receptors (IGHG1 and IGK) (left) in breast tumor tissue and single-cell RNA-seq (right).

FIG. 21C shows spatial patterning of paired IG receptors (IGHG2 and IGK) (left) in breast tumor tissue and single-cell RNA-seq (right).

FIG. 22 shows an exemplary nested PCR strategy for additional TCR enrichment.

### DETAILED DESCRIPTION

A fundamental understanding of spatial heterogeneity with respect to T-cell receptor (TCR) and B-cell receptor (BCR) clonotypes within a biological sample is needed to understand which cells a TCR or BCR may be interacting with, the identity of TCR and/or BCR clonotypes in a given biological sample, or the identity of TCR and/or BCR clonotypes that are autoreactive in different autoimmune disorders. Numerous single-cell sequencing approaches can identify TCR and BCR clonotypes from a biological sample, however, at present methods are need to link TCR and BCR sequences to spatial locations within a biological sample. Additionally, identifying the clonal regions, that is, regions defined by the places where variable (V), diverse (D), and joining (J) segments join to from the complementarity determining regions, including CDR1, CDR2, and CDR3, which provide specificity to the TCRs and/or BCRs, is needed to help determine biological interactions. By coupling clonal information to spatial information it is possible to understand which T-cell and B-cell clonotypes may be specifically interacting with given cell types within a biological sample.

Provided herein are methods for determining the presence and/or abundance of an immune cell clonotype at a location in a biological sample. Some embodiments of any of the methods described herein include capturing transcripts to identify an immune cell clonotype. Some embodiments of any of the methods herein include generating a nucleic acid library from captured transcripts. Some embodiments of any of the methods described herein include enriching analytes of interest in the nucleic acid library, including analytes to identify an immune cell clonotype.

Provided herein are methods for determining the presence and/or abundance of an immune cell receptor at a location in a biological sample. Some embodiments of any of the methods described herein include capturing analytes to identify an immune cell receptor. Some embodiments of any of the methods described herein include generating a nucleic acid library from captured analytes. Some embodiments of any of the methods described here include enriching analytes of interest in the nucleic acid library, including analytes to identify an immune cell receptor.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics® Support Documentation website, and can be used herein in any combination. Further non-limiting aspects of spatial analysis methodologies and compositions are described herein.

Some general terminology that may be used in this disclosure can be found in Section (I) (b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

In some embodiments, the analyte is an immune cell receptor. In some embodiments, the immune cell receptor is a B cell receptor. In some embodiments, the B cell receptor is an immunoglobulin kappa light chain. In some embodiments, the variable region of the analyte includes a CDR3 region of the immunoglobulin kappa light chain. In some embodiments, the variable region of the analyte includes one or both of CDR1 and CDR2 of the immunoglobulin kappa light chain. In some embodiments, the variable region of the analyte includes a full-length variable domain of the immunoglobulin kappa light chain.

In some embodiments, the B cell receptor is an immunoglobulin lambda light chain. In some embodiments, the variable region of the analyte includes a CDR3 of the immunoglobulin lambda light chain. In some embodiments, the variable region of the analyte includes one or both of CDR1 and CDR2 of the immunoglobulin lambda light chain. In some embodiments, the variable region of the analyte includes a full-length variable domain of the immunoglobulin lambda light chain.

In some embodiments, the B cell receptor is an immunoglobulin heavy chain. In some embodiments, the variable region of the analyte includes a CDR3 of the immunoglobulin heavy chain. In some embodiments, the variable region of the analyte includes one or both of CDR1 and CDR2 of the immunoglobulin heavy chain. In some embodiments, the variable region of the analyte includes a full-length variable domain of the immunoglobulin heavy chain.

In some embodiments, the immune cell receptor is a T cell receptor. In some embodiments, the T cell receptor is a T cell receptor alpha chain. In some embodiments, the variable region of the analyte includes a CDR3 of the T cell receptor alpha chain. In some embodiments, the variable region of the analyte includes one or both of CDR1 and CDR2 of the T cell receptor alpha chain. In some embodiments, the variable region of the analyte includes a full-length variable domain of the T cell receptor alpha chain.

In some embodiments, the T cell receptor is a T cell receptor beta chain. In some embodiments, the variable region of the analyte includes a CDR3 of the T cell receptor beta chain. In some embodiments, the variable region of the analyte includes one or both of CDR1 and CDR2 of the T cell receptor beta chain. In some embodiments, the variable region of the analyte further includes a full-length variable domain of the T cell receptor beta chain.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II) (b) (viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

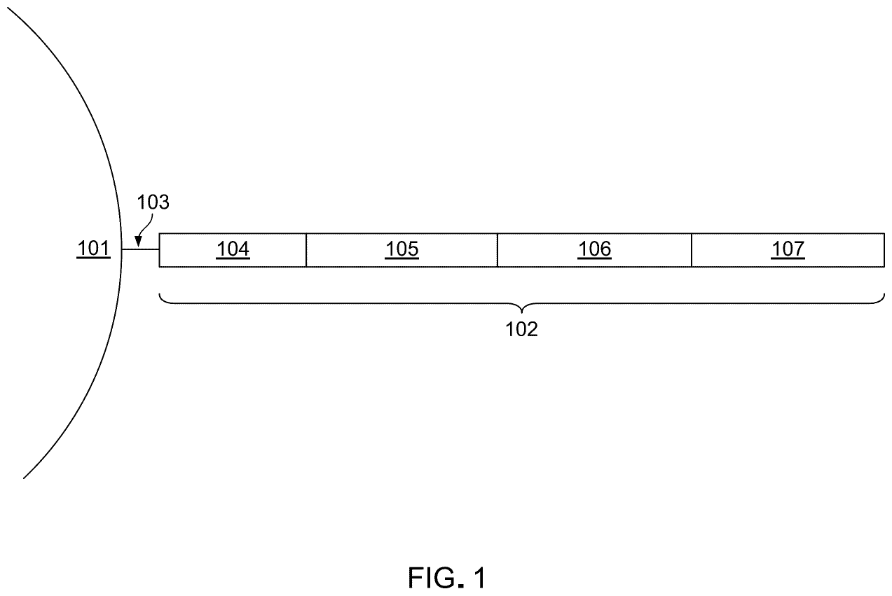

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein. The functional sequences can generally be selected for compatibility with any of a variety of different sequencing systems, e.g., Ion Torrent Proton or PGM, ILLUMINA® (sequencing technology) sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, ILLUMINA® (sequencing technology) sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II) (h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21;45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(c)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Spatial Transcriptomics for Antigen Receptors

A fundamental understanding of spatial heterogeneity with respect to T-cell receptor (TCR) and B-cell receptor (BCR) clonotypes within a biological sample is needed to understand multiple facets of their functionality, including, for example, which cells a particular TCR or BCR may be interacting with within the biological sample, the identity of TCR and/or BCR clonotypes in a given biological sample, and/or the identity of TCR and/or BCR clonotypes that are autoreactive in different autoimmune disorders. Numerous single-cell sequencing approaches can identify TCR and BCR clonotypes from a biological sample, however, at present methods are needed to link TCR and BCR sequences to spatial locations within a biological sample. Additionally, identifying the clonal regions, that is, regions defined by the places where variable (V), diverse (D), and joining (J) segments join to form the complementarity determining regions, including CDR1, CDR2, and CDR3, which provide specificity to the TCRs and/or BCRs, would greatly benefit the scientific arts. By coupling clonal information to spatial information it is possible to understand which T-cell and B-cell clonotypes may be specifically interacting with given cell types within a biological sample.

However, capturing analytes encoding immune cell receptors can provide unique challenges. For example, spatially capturing the TCR and BCR gene components with sufficient efficiency to profile the majority of clonotypes in a given tissue is difficult. Capturing analytes encoding immune cell receptors with conventional short-read sequencing methods can result in a loss of sequenced regions that are more than about 1 kb away from the point where sequencing starts. Linking separate TCR or BCR gene components that together form a complete receptor using sequencing data from spots containing multiple different cells are challenges addressed by the methods described herein.

Methods described herein are utilized to analyze the various sequences of TCRs and BCRs from immune cells, for example, various clonotypes. In some embodiments, the methods are used to analyze the sequence of a TCR alpha chain, a TCR beta chain, a TCR delta chain, a TCR gamma chain, or any fragment thereof (e.g., variable regions including V(D)J or VJ regions, constant regions, transmembrane regions, fragments thereof, combinations thereof, and combinations of fragments thereof). In some embodiments, the methods described herein can be used to analyze the sequence of a B cell receptor heavy chain, B cell receptor light chain, or any fragment thereof (e.g., variable regions including V(D)J or VJ regions, constant regions, transmembrane regions, fragments thereof, combinations thereof, and combinations of fragments thereof).

### Analytes

The analyte sequences present in the nucleic acid library (e.g., nucleic acid library generated from single-cells or from a biological sample on an array) can be captured from a biological sample (e.g., any of the biological samples described herein). In some embodiments, the biological sample is a tissue sample. In some embodiments, the tissue sample is a tissue section. In some embodiments, the tissue section is a fixed tissue section. In some embodiments, the fixed tissue section is formalin-fixed paraffin-embedded tissue section. In some embodiments, the tissue section is a fresh, frozen tissue section.

The analytes to be detected can be any of the analytes described herein. Analytes can include a nucleic acid molecule with a nucleic acid sequence encoding at least a portion of a V(D)J sequence of an immune cell receptor (e.g., a TCR or BCR). In some embodiments, the analyte is RNA. In some embodiments, the RNA is mRNA. In some embodiments, the analyte is DNA. In some embodiments, the DNA is genomic DNA. In some embodiments, the analytes are analytes encoding immune cell receptors. In some embodiments, analytes encoding immune cell receptors identify clonotype populations from a biological sample.

In some embodiments, analytes include a constant region, such as a constant region present in analytes encoding immune cell receptors. In some embodiments, analytes include a variable region, such as analytes encoding immune cell receptors. In some embodiments, analytes encoding immune cell receptors identify clonotype populations present in a biological sample.

In some embodiments, the analyte is an immune cell receptor. In some embodiments, the immune cell receptor is a B cell receptor. In some embodiments, the B cell receptor is an immunoglobulin kappa light chain. In some embodiments, the variable region of the analyte includes a CDR3 region of the immunoglobulin kappa light chain. In some embodiments, the variable region of the analyte includes one or both of CDR1 and CDR2 of the immunoglobulin kappa light chain. In some embodiments, the variable region of the analyte includes a full-length variable domain of the immunoglobulin kappa light chain.

In some embodiments, the B cell receptor is an immunoglobulin lambda light chain. In some embodiments, the variable region of the analyte includes a CDR3 of the immunoglobulin lambda light chain. In some embodiments, the variable region of the analyte includes one or both of CDR1 and CDR2 of the immunoglobulin lambda light chain. In some embodiments, the variable region of the analyte includes a full-length variable domain of the immunoglobulin lambda light chain.

In some embodiments, the B cell receptor is an immunoglobulin heavy chain. In some embodiments, the variable region of the analyte includes a CDR3 of the immunoglobulin heavy chain. In some embodiments, the variable region of the analyte includes one or both of CDR1 and CDR2 of the immunoglobulin heavy chain. In some embodiments, the variable region of the analyte includes a full-length variable domain of the immunoglobulin heavy chain.

In some embodiments, the immune cell receptor is a T cell receptor. In some embodiments, the T cell receptor is a T cell receptor alpha chain. In some embodiments, the variable region of the analyte includes a CDR3 of the T cell receptor alpha chain. In some embodiments, the variable region of the analyte includes one or both of CDR1 and CDR2 of the T cell receptor alpha chain. In some embodiments, the variable region of the analyte includes a full-length variable domain of the T cell receptor alpha chain.

In some embodiments, the T cell receptor is a T cell receptor beta chain. In some embodiments, the variable region of the analyte includes a CDR3 of the T cell receptor beta chain. In some embodiments, the variable region of the analyte includes one or both of CDR1 and CDR2 of the T cell receptor beta chain. In some embodiments, the variable region of the analyte further includes a full-length variable domain of the T cell receptor beta chain.

### Capturing Analytes Encoding Immune Cell Receptors

Provided herein are methods for determining the presence and/or abundance of an immune cell clonotype at a location in a biological sample, the method including (a) contacting a biological sample with an array including a plurality of capture probes, wherein a capture probe of the plurality of capture probes includes (i) a spatial barcode and (ii) a capture domain that specifically binds to a nucleic acid encoding an immune cell receptor of the immune cell clonotype, and, (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell clonotype at a location in the biological sample.

Also provided herein are methods for determining the presence and/or abundance of an immune cell receptor at a location in a biological sample, the method including (a) contacting a biological sample with an array including a plurality of capture probes, wherein a capture probe of the plurality of capture probes includes (i) a spatial barcode and (ii) a capture domain that specifically binds to a nucleic acid encoding an immune cell receptor; and (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell receptor at a location in the biological sample.

Also provided herein are methods for determining the presence and/or abundance of an immune cell clonotype at a location in a biological sample, the method including (a) contacting a biological sample with an array including a plurality of capture probes, wherein a capture probe of the plurality of capture probes includes (i) a spatial barcode and (ii) a capture domain that binds to a nucleic acid encoding an immune cell receptor of the immune cell clonotype; (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell clonotype at a location in the biological sample.

Also provided herein are methods for determining the presence and/or abundance of an immune cell receptor at a location in a biological sample, the method including (a) contacting a biological sample with an array including a plurality of capture probes, wherein a capture probe of the plurality of capture probes includes (i) a spatial barcode and (ii) a capture domain that binds to a nucleic acid encoding an immune cell receptor and (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell receptor at a location in the biological sample.

In some embodiments of determining the presence and/or abundance of an immune cell clonotype or an immune cell receptor at a location in a biological sample, step (b) includes extending an end of the capture probe using the nucleic acid encoding the immune cell receptor as a template, thereby generating an extended capture probe. In some embodiments, extending an end of the capture probe includes using a reverse transcriptase (e.g., any of the reverse transcriptases described herein). In some embodiments, step (b) includes extending a 3′ end of the capture probe. In some embodiments, step (b) includes generating a second strand of nucleic acid that includes (i) a sequence that is complementary to all or a portion of the spatial barcode, and (ii) a sequence that corresponds to all or a portion of the sequence of the nucleic acid encoding the immune cell receptor.

In some embodiments of determining the presence and/or abundance of an immune cell clonotype or an immune cell receptor at a location in a biological sample, the capture probe includes a cleavage domain, a functional domain, a unique molecular identifier, or any combination thereof. In some embodiments, the capture probe includes a functional domain. In some embodiments, the capture domain includes a poly(T) sequence. In some embodiments, the capture domain binds specifically to a nucleic acid sequence encoding a constant region of the T cell receptor alpha chain. In some embodiments, the capture domain binds specifically to a nucleic acid sequence encoding a constant region of the T cell receptor beta chain. In some embodiments, the capture domain binds specifically to a nucleic acid sequence encoding a constant region of the immunoglobulin kappa light chain. In some embodiments, the capture domain binds specifically to a nucleic acid sequence encoding a constant region of the immunoglobulin lambda light chain. In some embodiments, the capture probe binds specifically to a nucleic acid sequence encoding a constant region of the immunoglobulin heavy chain.

### Variable Region Primer Enrichment

As demonstrated in the Examples, analytes encoding immune cell receptors were captured and identified with capture domains designed to specifically bind a constant region of a particular immune cell receptor from a biological sample. However, such a strategy does not capture analytes other than analytes encoding immune cell receptors. An additional and alternative approach can include using one or more variable region (V-region) specific primer sets to amplify analytes encoding immune cell receptors (e.g., TCRs and/or BCRs) from nucleic acid libraries generated from poly(T) captured total cDNA libraries, thus allowing sequencing into CDR regions (e.g., CDR3 region) from the 5′ end of an amplicon. An advantage of this approach would be the simultaneous detection of lymphocyte clonality alongside global spatial gene expression. An additional consideration is capturing full IGH complexity (e.g., IGH isotypes, e.g., IGHA1-2, IGHG1-4, IGHM, IGHD, and IGHE) without paired end sequencing reads through the CDR3 region. Additional receptor diversity is added to the BCR throughout development and may be difficult to distinguish from sequencing errors with only a single CDR3 read. Additionally, some analytes encoding immune cell receptors are known to be in low abundance (See e.g., Tu, A.A., et al., TCR sequencing paired with massively parallel 3′ RNAseq reveals clonotypic T cell signatures, Nature Immunology, 20, 1692-1699 (2019); Singh M., et al., High-throughput long-read single cell sequencing reveals the clonal and transcriptional landscape of lymphocytes. Nature Communications, 10, 3120 (2019), both of which are incorporated herein by reference in their entireties). Thus, for example, variable region primer enrichment can provide an alternate method to enrich for analytes encoding immune cell receptors from arrays with capture probes including a poly(T) capture domain, followed by one or more amplification reactions (e.g., PCR).

In some embodiments of any of the spatial methods described herein, step (b) further includes generating a second strand of nucleic acid that includes (i) a sequence that is complementary to all or a portion of the functional domain, (ii) a sequence that is complementary to all or a portion of the spatial barcode, and (iii) a sequence that corresponds to all or a portion of the sequence of the nucleic acid encoding the immune cell receptor. In some embodiments, step (b) further includes amplifying the second strand of the nucleic acid using (i) a first primer including all or a portion of the functional domain, wherein the functional domain is 5′ to the spatial barcode in the second strand of nucleic acid, and (ii) a second primer including a sequence that is substantially complementary to a portion of a sequence encoding a variable region of the immune cell receptor.

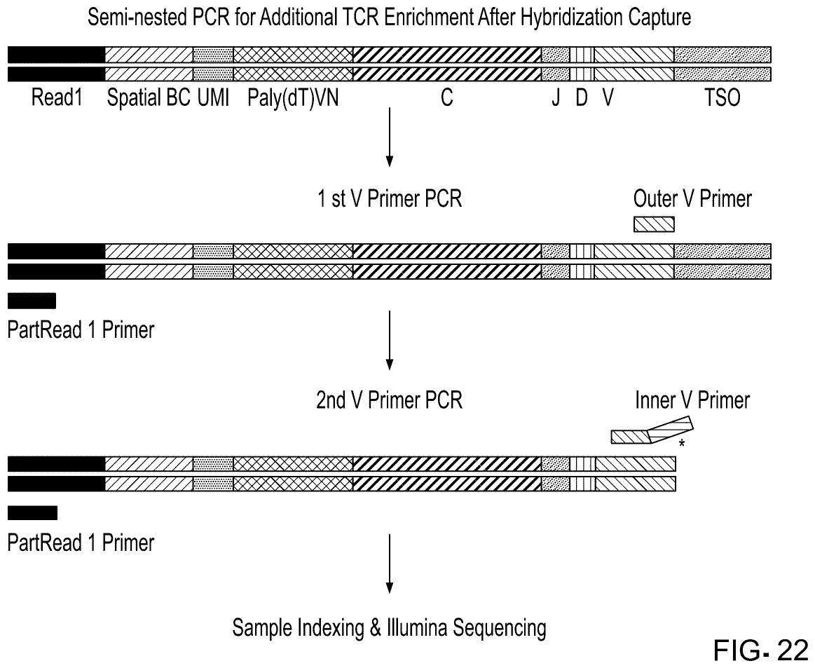

In some embodiments, more than one second primer including a sequence substantially complementary to a portion of the sequence encoding the variable region of the immune cell receptor is used. For example, a nested PCR strategy can be used where a first amplification product is generated with a variable region primer and a primer substantially complementary to the functional domain 5′ to the spatial barcode, followed by a second, a third, or a fourth round of amplification using a second, a third, or a fourth variable region primer internal to the first region variable region primer (e.g., 5′ to the first variable region primer) (for example, see FIG. 22). It will be understood to a person of ordinary skill in the art that additional rounds of amplification require an internal (e.g., 5′) located variable region primer in subsequent amplification rounds.

### Hybridization Probes and Blocking Probes

In some embodiments, targeted enrichment of cDNAs of interest are enriched from cDNA derived libraries generated from captured analytes (e.g., immune cell analytes). For example, a pool of hybridization probes to an analyte of interest, or a complement thereof, can be designed. In some embodiments, about 10 to about 500 hybridization probes, about 25 to about 450 hybridization probes, about 50 to about 400 hybridization probes, about 75 to about 350 hybridization probes, or about 100 to 300 hybridization probes can be designed for hybridizing to an analyte of interest, or a complement thereof. In some embodiments, the hybridization probes can include an additional moiety, such as a binding moiety, (e.g., biotin) capable of binding another moiety, such as a capture moiety, (e.g., streptavidin). Thus, in some embodiments, one or more hybridization probes (e.g., including an additional moiety, such as biotin) hybridize to the analyte of interest, or complement thereof, in the cDNA library and the total cDNA library is processed on streptavidin beads, for example. The biotin moieties of the hybridization probes specifically bind the streptavidin molecules, thereby enriching for the analytes of interest, or complements thereof. Hybridization probes can be designed to be complementary to any analyte or its complementary sequence, including, for example, analytes encoding immune cell analytes.

In some embodiments, enriching analytes of interest includes the use of blocking probes. Blocking probes can be added to the cDNA library before, after, or concurrently with hybridization probes. In some embodiments, blocking probes reduce background (e.g., non-specific binding events) when enriching for targets within the cDNA library. In some embodiments, blocking probes can be about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150 nucleotides long. In some embodiments, blocking probes are designed specifically to domains present in one or more members of the cDNA library. In some embodiments, one blocking probe is added to the cDNA library. In some embodiments, two or more blocking probes (e.g., different blocking probes). In some embodiments, 3, 4, 5 or more different blocking probes are added to the cDNA library (e.g., blocking probes having a different sequence). In some embodiments, the blocking probe comprises SEQ ID NO: 639. In some embodiments, the blocking probe comprises SEQ ID NO: 640. In some embodiments, the blocking probe comprises at least 80%, at least 85%, at least 90%, at least 95%, at least 96%, at least 97%, at least 98%, or at least 99% identity to SEQ ID NO: 639. In some embodiments, the blocking probe comprises at least 80%, at least 85%, at least 90%, at least 95%, at least 96%, at least 97%, at least 98%, or at least 99% identity to SEQ ID NO: 640.

### Identifying Immune Cell Receptors

In some embodiments of determining the presence and/or abundance of an immune cell clonotype at a location in a biological sample, determining in step (b) includes sequencing (i) all or a portion of the sequence of the spatial barcode or a complement thereof, and (ii) all or a portion of the sequence of the nucleic acid encoding the immune cell receptor or a complement thereof. Any of the sequencing methods described herein can be used. In some embodiments, step (b) includes determining the presence of the immune cell clonotype at a location in the biological sample. In some embodiments, step (b) includes determining the abundance of the immune cell clonotype at a location in the biological sample. In some embodiments, step (b) includes determining the presence and abundance of the immune cell clonotype at a location in the biological sample. In some embodiments, step (b) includes determining the presence of two or more immune cell clonotypes at a location in the biological sample. In some embodiments, step (b) includes determining the abundance of two or more immune cell clonotypes at a location in the biological sample. In some embodiments, step (b) includes determining the presence and abundance of two or more immune cell clonotypes at a location in the biological sample. In some embodiments, the method includes comparing the two or more immune cell clonotypes. In some embodiments, the two or more immune cell clonotypes are each a B cell clonotype. In some embodiments, the two or more immune cell clonotypes are each a T cell clonotype. In some embodiments, the two or more immune cell clonotypes include at least one T cell clonotype and at least one B cell clonotype.

In some embodiments of determining the presence and/or abundance of an immune cell receptor at a location in a biological sample, the determining in step (b) includes sequencing (i) all or a portion of the sequence of the spatial barcode or a complement thereof, and (ii) all or a portion of the sequence of the nucleic acid encoding the immune cell receptor or a complement thereof. In some embodiments, step (b) includes determining the presence of the immune cell receptor at a location in the biological sample. In some embodiments, step (b) includes determining the abundance of the immune cell receptor at a location in the biological sample. In some embodiments, step (b) includes determining the presence and abundance of the immune cell receptor at a location in the biological sample. In some embodiments, step (b) includes determining the presence of two or more immune cell receptors at a location in the biological sample. In some embodiments, step (b) includes determining the abundance of two or more immune cell receptors at a location in the biological sample. In some embodiments, step (b) includes determining the presence and abundance of two or more immune cell receptors at a location in the biological sample. In some embodiments, the method includes comparing the two or more immune cell receptors. In some embodiments, the two or more immune cell clonotypes are each an immune cell receptor of a B cell. In some embodiments, two or more immune cell clonotypes are each an immune cell receptor of a T cell. In some embodiments, two or more immune cell clonotypes include at least one immune cell receptor of a T cell and at least one immune cell receptor from a B cell.

In some embodiments of determining the presence and/or abundance of an immune cell clonotype or an immune cell receptor at a location in a biological sample, includes prior to step (b), contacting the biological sample with ribosomal RNA depletion probes and/or mitochondrial RNA depletion probes. In some embodiments, the biological sample is imaged. In some embodiments, the biological sample is stained.

### Arrays and Kits

Provided herein are arrays including a plurality of capture probes, where a capture probe of the plurality of capture probes includes (i) a spatial barcode and (ii) a capture domain that specifically binds to a nucleic acid encoding an immune cell receptor of an immune cell clonotype. In some arrays, the immune cell clonotype is a T cell clonotype. In some arrays, the immune cell receptor is a T cell receptor alpha chain. In some arrays, the capture domain binds specifically to a nucleic acid sequence encoding a constant region of the T cell receptor alpha chain. In some arrays, the immune cell receptor is a T cell receptor beta chain. In some arrays, the capture domain binds specifically to a nucleic acid sequence encoding a constant region of the T cell receptor beta chain. In some arrays, the immune cell clonotype is a B cell clonotype. In some arrays, the immune cell receptor is an immunoglobulin kappa light chain. In some arrays, the capture domain binds specifically to a nucleic acid sequence encoding a constant region of the immunoglobulin kappa light chain. In some arrays, the immune cell receptor is an immunoglobulin lambda light chain. In some arrays, the capture domain binds specifically to a nucleic acid sequence encoding a constant region of the immunoglobulin lambda light chain. In some arrays, the immune cell receptor is an immunoglobulin heavy chain. In some arrays, the capture domain binds specifically to a nucleic acid sequence encoding a constant region of the immunoglobulin heavy chain. In some arrays, the capture probe includes a cleavage domain, a functional domain, a unique molecular identifier, or any combination thereof.

Also provided herein are kits including an array (e.g., any of the arrays described herein) and one or more hybridization probes, wherein a hybridization probe includes (i) a sequence substantially complementary to a nucleic acid encoding an immune cell receptor and (ii) a binding moiety that interacts with a capturing moiety and one or more blocking probes.

Also provided herein are kits, including an array of any of the arrays described herein and one or both of ribosomal RNA depletion probes and mitochondrial RNA depletion probes.

Targeted RNA depletion allows for depletion or removal of one or more species of undesirable RNA molecules (e.g., ribosomal RNA and/or mitochondrial RNA), thereby reducing the pool and concentration of undesirable RNA molecules in the sample which could interfere with desired target detection (e.g., detection of mRNA). To achieve depletion, one or more probes are designed that hybridize to one or more undesirable RNA molecules. For example, in one embodiment, probes can be administered to a biological sample that selectively hybridize to ribosomal RNA (rRNA), thereby reducing the pool and concentration of rRNA in the sample. In one embodiment, probes can be administered to a biological sample that selectively hybridize to mitochondria RNA (mtRNA), thereby reducing the pool and concentration of mtRNA in the sample. Subsequent application of capture probes to the sample can result in improved capture of other types of RNA due to a reduction in undesirable RNA (e.g., down-selected RNA) present in the sample.

Upon depletion of the undesirable RNA, the sample will contain an enriched population of the RNA target of interest (e.g., an mRNA target). In some embodiments, the undesirable RNA comprises less than 20%, 19%, 18%, 17%, 16% 15%, 14%, 13%, 12%, 11% 10%, 9%, 8%, 7%, 6%, 5%, 4%, 3%, 2%, or 1%, or any range therein, of the total RNA in the sample after depletion of the undesirable RNA (i.e., less than 20%, 19%, 18%, 17%, 16% 15%, 14%, 13%, 12%, 11% 10%, 9%, 8%, 7%, 6%, 5%, 4%, 3%, 2%, or 1%, or any range therein compared to a sample that undergoes no depletion step). Consequently, the enriched population of the RNA target of interest may comprise at least 99%, 98%, 97%, 96%, 95%, 94%, 93%, 92%, 91%, 90%, 89%, 88%, 87%, 86%, 85%, 84%, 83%, 82%, 81%, or 80%, or any range therein, of the total RNA in the sample.

As used herein, the term “undesirable RNA molecule”, or “undesirable RNA”, refers to an undesired RNA that is the target for depletion from the biological sample. In some embodiments, examples of the undesirable RNA include, but are not limited to, messenger RNA (mRNA), ribosomal RNA (rRNA), mitochondrial RNA (mtRNA), transfer RNA (tRNA), microRNA (miRNA), and viral RNA. In some embodiments, the undesirable RNA can be a transcript (e.g., present in a tissue section). The undesirable RNA can be small (e.g., less than 200 nucleic acid bases in length) or large (e.g., RNA greater than 200 nucleic acid bases in length).

In some embodiments, the undesirable RNA molecule includes 5.8S ribosomal RNA (rRNA), 5S rRNA, transfer RNA (IRNA), microRNA (miRNA), a small nucleolar RNA (snoRNAs), Piwi-interacting RNA (piRNA), tRNA-derived small RNA (tsRNA), and small rDNA-derived RNA (srRNA), or mitochondrial RNA (mtRNA). In some embodiments, the undesirable RNA molecule includes an RNA molecule that is added (e.g., transfected) into a sample (e.g., a small interfering RNA (siRNA)). The undesirable RNA can be double-stranded RNA or single-stranded RNA. In embodiments where the undesirable RNA is double-stranded it is processed as a single-stranded RNA prior to depletion. In some embodiments, the undesirable RNA can be circular RNA. In some embodiments, the undesirable RNA can be a bacterial rRNA (e.g., 16s rRNA or 23s rRNA). In some embodiments, the undesirable RNA is from E. coli.

In some embodiments, the undesirable RNA molecule is rRNA. In some embodiments, the rRNA is eukaryotic rRNA. In some embodiments, the rRNA is cytoplasmic rRNA. In some embodiments, the rRNA is mitochondrial rRNA. Cytoplasmic rRNAs include, for example, 28S, 5.8S, 5S and 18S rRNAs. Mitochondrial rRNAs include, for example, 12S and 16S rRNAs. The rRNA may also be prokaryotic rRNA, which includes, for example, 5S, 16S, and 23S rRNA. The sequences for rRNAs are well known to those skilled in the art and can be readily found in sequence databases such as GenBank or may be found in the literature. For example, the sequence for the human 18S rRNA can be found in GenBank as Accession No. M10098 and the human 28S rRNA as Accession No. M11167.

In some embodiments, the undesirable RNA molecule is mitochondrial RNA. Mitochondrial RNAs include, for example, 12S rRNA (encoded by MT-RNR1), and 16S rRNA (encoded by MT-RNR2), RNAs encoding electron transport chain proteins (e.g., NADH dehydrogenase, coenzyme Q-cytochrome c reductase/cytochrome b, cytochrome c oxidase, ATP synthase, or humanin), and tRNAs (encoded by MT-TA, MT-TR, MT-TN, MT-TD, MT-TC, MT-TE, MT-TQ, MT-TG, MT-TH, MT-TI, MT-TL1, MT-TL2, MT-TK, MT-TM, MT-TF, MT-TP, MT-TS1, MT-TS2, MT-TT, MT-TW, MT-TY, or MT-TV).

In some embodiments, the one or more undesirable RNA depletion probes is a DNA probe. In some embodiments, the DNA probe includes a single-stranded DNA oligonucleotide having a sequence partially or completely complementary to an undesirable RNA and specifically hybridizes to the undesirable RNA. In some embodiments, the one or more undesirable RNA depletion probes are at least 70%, at least 75%, at least 80%, at least 85%, at least 90%, at least 91%, at least 92%, at least 93%, at least 94%, at least 95%, at least 96%, at least 97%, at least 98%, or at least 99% complementary to one or more undesirable RNA molecules. In some embodiments, the one or more undesirable RNA depletion probes is 100% (i.e., completely) complementary to one or more undesirable RNA molecules.

In some embodiments, probes used herein have been described in Morlan et al., PLoS One. 2012; 7(8):e42882, which is incorporated by reference in its entirety. In some embodiments, probes used herein have been described in U.S. Appl. Publ. No. 2011/0111409, which is incorporated by reference in its entirety. In some embodiments, probes used herein have been described in Adiconis et al., Nat Methods. 2013 July; 10 (7): 623-9, which is incorporated by reference in its entirety.

The DNA probe can be produced by techniques known in the art. For example, in some embodiments, a DNA probe is produced by chemical synthesis, by in vitro expression from recombinant nucleic acid molecules, or by in vivo expression from recombinant nucleic acid molecules. The undesirable RNA depletion probe may also be produced by amplification of the undesirable RNA, e.g., RT-PCR, asymmetric PCR, or rolling circle amplification.

### EXAMPLES

### Example 1: Analyte Capture and Enrichment Strategies

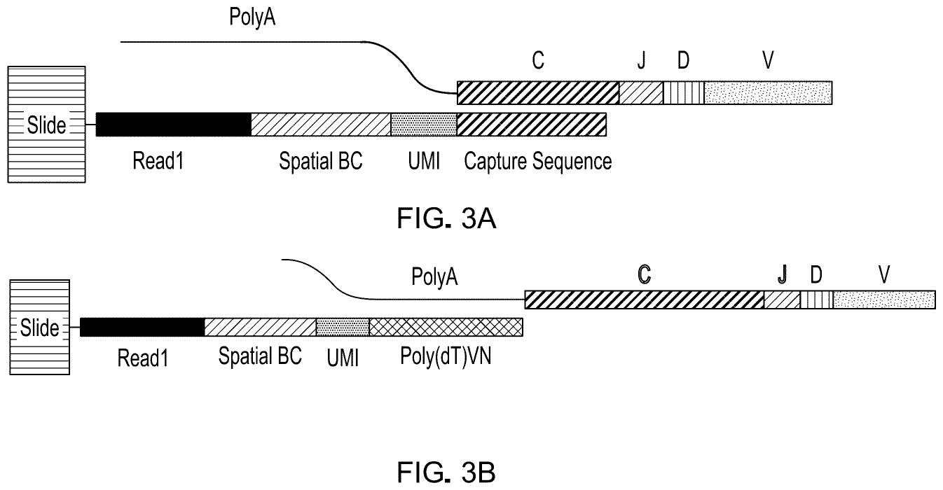

FIGS. 3A and 3B show two different capture probes with different exemplary capture strategies to capture analytes encoding immune cell receptors in a biological sample. FIG. 3A shows “targeted” capture where the capture domain is substantially complementary to the constant region of the analyte encoding an immune cell receptor to be detected. Targeted capture increases the likelihood that the portion of interest in the variable domain, CDR3, is retained during library preparation. Alternatively, FIG. 3B shows poly(A) capture with a poly(T) capture domain. A poly(T) capture domain can capture other analytes, including analytes encoding immune cell receptors within the biological sample.

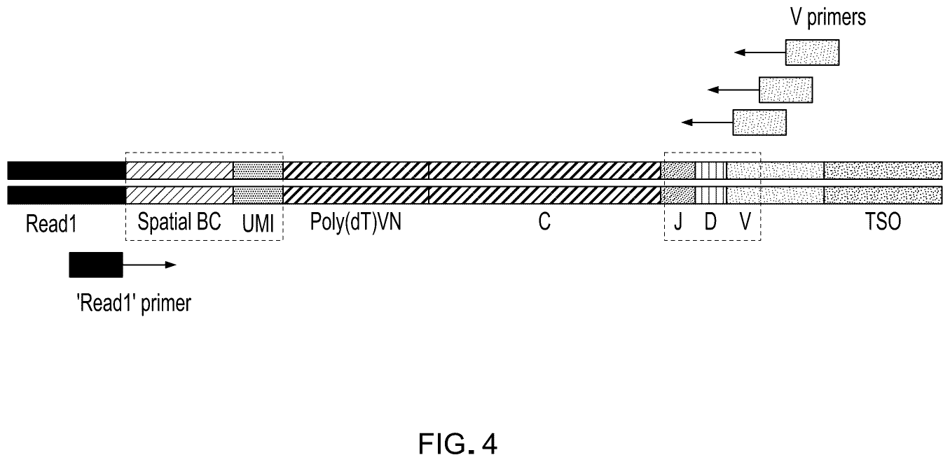

FIG. 4 shows an exemplary analyte enrichment strategy following analyte capture on the array. The portion of the immune cell analyte of interest includes the sequence of the V(D)J region. The CDR sequences are also important because these sequences define the immune cell receptor's binding specificity. As described herein, a poly(T) capture probe captures an analyte encoding an immune cell receptor, an extended capture probe is generated by a reverse transcription reaction, and a second strand is generated. The resulting nucleic acid library can be enriched by the exemplary scheme shown in FIG. 4, where an amplification reaction including a Read 1 primer complementary to the Read 1 sequence of the capture probe and a primer complementary to a portion of the variable region of the immune cell analyte, can enrich the library via PCR. The enriched library can be further enriched by nested primers complementary to a portion of the variable region internal (e.g., 5′) to the initial variable region primer for practicing nested PCR.

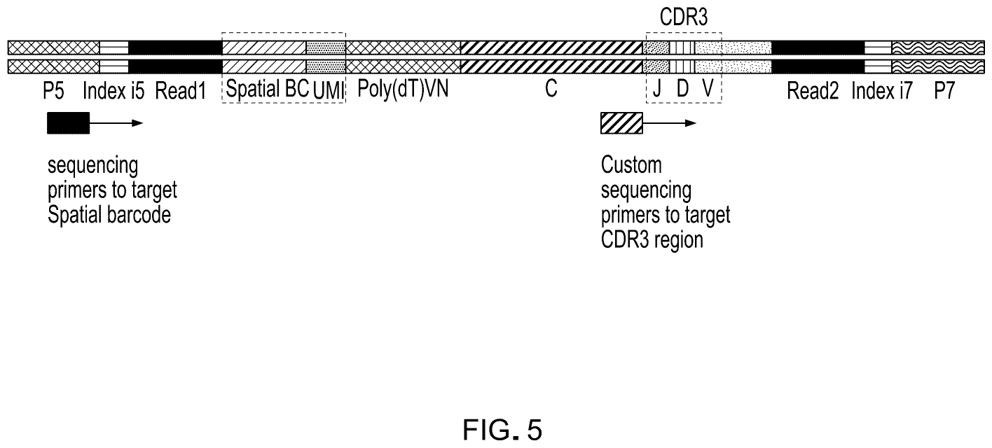

FIG. 5 shows a sequencing strategy with a primer specific complementary to the sequencing flow cell attachment sequence (e.g., P5) and a custom sequencing primer complementary to a portion of the constant region of the analyte. This sequencing strategy targets the constant region to obtain the sequence of the CDR regions, including CDR3, while concurrently or sequentially sequencing the spatial barcode (BC) and/or unique molecular identifier (UMI) of the capture probe. By capturing the sequence of a spatial barcode, UMI and a V(D)J region the receptor is not only determined, but its spatial location and abundance within a cell or tissue is also identified.

### Example 2—Capture of Analytes Encoding Immune Cell Receptors

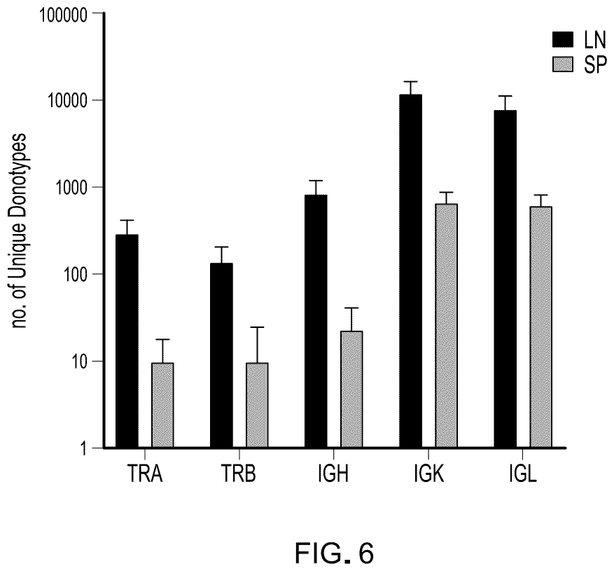

FIG. 6 shows the number of unique clonotypes detected for TRA, TRB, IGH, IGK, and IGL on an array for both lymph node tissue (LN, black) and spleen tissue (SP, gray). It is contemplated that the lack of detected clonotypes found may be the result of inefficient or decreased TRAC/TRBC/IGH transcript capture or decreased sequencing of the variable region (e.g., CDR3 region) due to its distance from the sequencing domain (e.g. Read 1 sequencing domain). A greater abundance of unique clonotypes were detected for IGK and IGL, which may be due in part to the shorter constant regions present in these clonotypes relative to the constant regions present in TRAC, TRAB, and IGH transcripts.

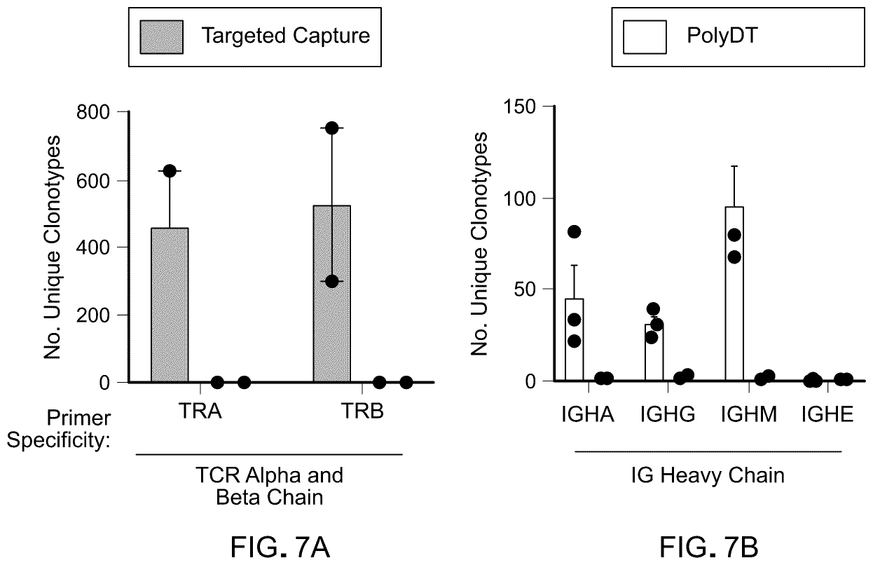

FIGS. 7A-B show the number of unique clonotypes detected for TRA and TRB (FIG. 7A) and the number of unique clonotypes detected for IGA, IGHG, IGHM, and IGHE (FIG. 7B). The data show that targeted capture (gray bars) yields a higher number of TRA and TRB clonotypes (FIG. 7A) than poly(T) capture as demonstrated by the lack of clonotypes detected. Similarly, targeted capture of IGHA, IGHG, and IGHM yielded a higher number of unique clonotypes detected than poly(T) capture, as demonstrated by the lack of clonotypes detected. Thus the data demonstrate that targeted capture of analytes encoding immune cell receptors is possible for some analytes, but may not be sufficient for other analytes encoding immune cell receptors (e.g., IGHE).

As discussed, undetectable levels of T-cell receptor and B-cell receptor transcripts were captured with poly(T) capture domains as shown in FIGS. 7A-B. Targeted capture, however, requires custom capture domains for each analyte encoding an immune cell receptor and does not allow for the simultaneous capture of analytes other than targeted analytes encoding immune cell receptors.

A strategy to detect whether analytes encoding immune cell receptors were captured was investigated and includes using poly(T) capture sequences in combination with PCR amplification performed on full length cDNA from several different sources, including lymph node tissue and tonsil tissue (Table 1).

TABLE 1

| | |
|---|---|
| Lymph Node (LN) spatial library | n = 6 |
| Tonsil spatial library | n = 2 |
| Tonsil SmartSeq2 (SS2) (single-cell) | n = 1 |
| RNAseq (positive control) | |

The tonsil SS2 sample was derived from the same tonsil as the tonsil spatial libraries and adapted from Picelli et al., Full-length RNA-seq from single cells using Smart-seq2, 9, 171-181, Nature (2014), and used as a positive control and without PCR enrichment.

To begin, 0.5 ng of each library in Table 1 as input material was run in triplicate for each sample and PCR reaction (TRB, IGHG, and IGHM), except for one LN (#9) and the Tonsil SS2 bulk sample, which were run in duplicate and once, respectively. The PCR primers targeted: a) the constant region of either TRB, IGHG, or IGHM (Table 2), and b) the variable segments for TRB (Balazs, A.B., et al., Isolation of unknown rearranged T-cell receptors from single cells WO 2011/008502, which is incorporated herein by reference in its entirety) and IGH (Vázquez, B., et al., High-Quality Library Preparation for NGS-Based Immunoglobulin Germline Gene Inference and Repertoire Expression Analysis, Frontiers Immunol, 10, 660 (2019), which is incorporated herein by reference in its entirety). The constant primers were selected based on their proximity to the CDR3 region and testing of various primers for each target was performed in PCR optimization experiments. Both the forward and reverse primers were tagged with partial P5 and P7 domains that allowed subsequent Truseq indexing for ILLUMINA® (sequencing technology) sequencing. PCR was performed using the KAPA HiFi Hotstart ready mix according to the manufacturer's instructions with 30 amplification cycles.

TABLE 2

| Constant | |
|---|---|
| Primer | Sequence |
| TRB | SEQ ID NO: 1 TCTGATGGCTCAAACACAGC |
| IGHG | SEQ ID NO: 2 GCCAGGGGGAAGACCGATGGG |
| IGHM | SEQ ID NO: 3 CACGCTGCTCGTATCCGA |

TABLE 3

| | |
|---|---|
| Variable | |
| Region Primer | Sequence |

TCRa V inner pool

| | |
|---|---|
| TCRaV17 | SEQ ID NO: 4 CAACAGGGAGAAGAGGATCCTCAGGCC |
| TCRaV1-2 | SEQ ID NO: 5 GGACAAAACATTGACCAGCCCACTGAGAT |
| TCRaV10 | SEQ ID NO: 6 AAAAACCAAGTGGAGCAGAGTCCTCAGTCC |
| TCRaV12-1 | SEQ ID NO: 7 CAACGGAAGGAGGTGGAGCAGGATC |
| TCRaV12-2 | SEQ ID NO: 8 CAACAGAAGGAGGTGGAGCAGAATTCTGG |
| TCRaV12-3 | SEQ ID NO: 9 CAACAGAAGGAGGTGGAGCAGGATCCT |
| TCRaV13-1 | SEQ ID NO: 10 GAGAATGTGGAGCAGCATCCTTCAACC |
| TCRaV13-2 | SEQ ID NO: 11 GAGAGTGTGGGGCTGCATCTTCCTACC |
| TCRaV14D4 | SEQ ID NO: 12 CAGAAGATAACTCAAACCCAACCAGGAATGTTC |
| TCRav16 | SEQ ID NO: 13 CAGAGAGTGACTCAGCCCGAGAAGCTC |
| TCRaV18 | SEQ ID NO: 14 GACTCGGTTACCCAGACAGAAGGCCC |
| TCRaV19 | SEQ ID NO: 15 CAGAAGGTAACTCAAGCGCAGACTGAAATTTCT |
| TCRaV2 | SEQ ID NO: 16 AAGGACCAAGTGTTTCAGCCTTCCACAGTG |
| TCRaV20 | SEQ ID NO: 17 GAAGACCAGGTGACGCAGAGTCCCG |
| TCRaV21 | SEQ ID NO: 18 AAACAGGAGGTGACGCAGATTCCTGC |
| TCRaV22 | SEQ ID NO: 19 ATACAAGTGGAGCAGAGTCCTCCAGACCTGA |
| TCRaV23DV6 | SEQ ID NO: 20 CAACAGAAGGAGAAAAGTGACCAGCAGCA |
| TCRaV24 | SEQ ID NO: 21 ATACTGAACGTGGAACAAAGTCCTCAGTCACTG |
| TCRaV25 | SEQ ID NO: 22 CAACAGGTAATGCAAATTCCTCAGTACCAGC |
| TCRaV26-1 | SEQ ID NO: 23 AAGACCACCCAGCCCCCCTCC |
| TCRaV26-2 | SEQ ID NO: 24 AAGACCACACAGCCAAATTCAATGGAGAGTAAC |
| TCRaV27 | SEQ ID NO: 25 CAGCTGCTGGAGCAGAGCCCTCAGT |
| TCRaV29DV5 | SEQ ID NO: 26 CAACAGAAGAATGATGACCAGCAAGTTAAGCAA |
| TCRaV3 | SEQ ID NO: 27 CAGTCAGTGGCTCAGCCGGAAGATC |
| TCRaV30 | SEQ ID NO: 28 CAACAACCAGTGCAGAGTCCTCAAGCC |
| TCRaV34 | SEQ ID NO: 29 CAAGAACTGGAGCAGAGTCCTCAGTCCTTG |
| TCRaV35 | SEQ ID NO: 30 CAACAGCTGAATCAGAGTCCTCAATCTATGTTTATC |
| TCRaV36DV7 | SEQ ID NO: 31 GAAGACAAGGTGGTACAAAGCCCTCTATCTCTG |
| TCRaV38-2DV8 | SEQ ID NO: 32 CAGACAGTCACTCAGTCTCAACCAGAGATGTCT |
| TCRaV39 | SEQ ID NO: 33 GAGCTGAAAGTGGAACAAAACCCTCTGTTC |
| TCRaV4 | SEQ ID NO: 34 AAGACCACCCAGCCCATCTCCATG |
| TCRaV40 | SEQ ID NO: 35 AATTCAGTCAAGCAGACGGGCCAAATAAC |
| TCRaV41 | SEQ ID NO: 36 GCCAAAAATGAAGTGGAGCAGAGTCCTC |
| TCRaV5 | SEQ ID NO: 37 GAGGATGTGGAGCAGAGTCTTTTCCTGAGTG |
| TCRaV6 | SEQ ID NO: 38 CAAAAGATAGAACAGAATTCCGAGGCCCTG |
| TCRaV7 | SEQ ID NO: 39 GAAAACCAGGTGGAGCACAGCCCTC |
| TCRaV8-1 | SEQ ID NO: 40 CAGTCTGTGAGCCAGCATAACCACCAC |
| TCRaV8-2 | SEQ ID NO: 41 CAGTCGGTGACCCAGCTTGACAGC |
| TCRaV8-3 | SEQ ID NO: 42 CAGTCAGTGACCCAGCCTGACATCCAC |
| TCRaV8-4 | SEQ ID NO: 43 CAGTCGGTGACCCAGCTTGGCAG |
| TCRaV8-6 | SEQ ID NO: 44 CAGTCTGTGACCCAGCTTGACAGCCA |
| TCRaV8-7 | SEQ ID NO: 45 CAGTCGGTGACCCAGCTTGATGGC |
| TCRaV9-1 | SEQ ID NO: 46 GATTCAGTGGTCCAGACAGAAGGCCAAGT |
| TCRaV9-2 | SEQ ID NO: 47 AATTCAGTGACCCAGATGGAAGGGCC |

TCRb V Inner Pool

| | |
|---|---|
| TCRb_JM_V2 | SEQ ID NO: 48 GAACCTGAAGTCACCCAGACTCCCAGC |
| TCRb_JM_V3-1 | SEQ ID NO: 49 GCTGTTTCCCAGACTCCAAAATACCTGGTC |
| TCRb_JM_V4-1 | SEQ ID NO: 50 GAAGTTACCCAGACACCAAAACACCTGGTC |
| TCRb_JM_V5-1 | SEQ ID NO: 51 GGAGTCACTCAAACTCCAAGATATCTGATCAAAAC |
| TCRb_JM_V6-1 | SEQ ID NO: 52 GGTGTCACTCAGACCCCAAAATTCCAG |
| TCRb_JM_V7-1 | SEQ ID NO: 53 GGAGTCTCCCAGTCCCTGAGACACAAGG |
| TCRb_JM_V4-2 | SEQ ID NO: 54 GGAGTTACGCAGACACCAAGACACCTGG |
| TCRb_JM_V6-2 | SEQ ID NO: 55 GGTGTCACTCAGACCCCAAAATTCCG |
| TCRb_JM_V7-2 | SEQ ID NO: 56 GGAGTCTCCCAGTCCCCCAGTAACAAG |
| TCRb_JM_V6-4 | SEQ ID NO: 57 GGGATCACCCAGGCACCAACATCTC |
| TCRb_JM_V7-3 | SEQ ID NO: 58 GGAGTCTCCCAGACCCCCAGTAACAAG |
| TCRb_JM_V5-3 | SEQ ID NO: 59 GGAGTCACCCAAAGTCCCACACACCT |
| TCRb_JM_V9 | SEQ ID NO: 60 GGAGTCACACAAACCCCAAAGCACCT |
| TCRb_JM_V10-1 | SEQ ID NO: 61 GAAATCACCCAGAGCCCAAGACACAAGA |
| TCRb_JM_V11-1 | SEQ ID NO: 62 GAAGTTGCCCAGTCCCCCAGATATAAGATTA |
| TCRb_JM_V10-2 | SEQ ID NO: 63 GGAATCACCCAGAGCCCAAGATACAAGAT |
| TCRb_JM_V11-2 | SEQ ID NO: 64 GGAGTTGCCCAGTCTCCCAGATATAAGATTATAGAG |
| TCRb_JM_V7-4 | SEQ ID NO: 65 GGAGTCTCCCAGTCCCCAAGGTACAAAG |
| TCRb_JM_V7-5 | SEQ ID NO: 66 GGAGTCTCCCAGTCCCCAAGGTACGA |
| TCRb_JM_V6-7 | SEQ ID NO: 67 GGTGTCACTCAGACCCCAAAATTCCAC |
| TCRb_JM_V7-6 | SEQ ID NO: 68 GGAGTCTCCCAGTCTCCCAGGTACAAAGTC |
| TCRb_JM_V6-8 | SEQ ID NO: 69 GGTGTCACTCAGACCCCAAAATTCCACAT |
| TCRb_JM_V7-8 | SEQ ID NO: 70 GGAGTCTCCCAGTCCCCTAGGTACAAAGTC |
| TCRb_JM_V5-8 | SEQ ID NO: 71 GGAGTCACACAAAGTCCCACACACCTGA |
| TCRb_JM_V7-9 | SEQ ID NO: 72 GGAGTCTCCCAGAACCCCAGACACAAG |
| TCRb_JM_V13 | SEQ ID NO: 73 GGAGTCATCCAGTCCCCAAGACATCTGAT |
| TCRb_JM_V12-3 | SEQ ID NO: 74 GGAGTTATCCAGTCACCCCGCCATG |
| TCRb_JM_V12-4 | SEQ ID NO: 75 GGAGTTATCCAGTCACCCCGGCAC |
| TCRb_JM_V12-5 | SEQ ID NO: 76 AGAGTCACCCAGACACCAAGGCACAAG |
| TCRb_JM_V14 | SEQ ID NO: 77 GGAGTTACTCAGTTCCCCAGCCACAGC |
| TCRb_JM_V15 | SEQ ID NO: 78 ATGGTCATCCAGAACCCAAGATACCAGGTT |
| TCRb_JM_V17 | SEQ ID NO: 79 GAGCCTGGAGTCAGCCAGACCCC |
| TCRb_JM_V18 | SEQ ID NO: 80 GGCGTCATGCAGAACCCAAGACAC |
| TCRb_JM_V19 | SEQ ID NO: 81 GGAATCACTCAGTCCCCAAAGTACCTGTTCA |
| TCRb_JM_V20-1 | SEQ ID NO: 82 GCTGTCGTCTCTCAACATCCGAGCTG |
| TCRb_JM_V22 | SEQ ID NO: 83 ATTCCAGCTCACTGGGGCTGGATG |
| TCRb_JM_V23-1 | SEQ ID NO: 84 AAAGTCACACAGACTCCAGGACATTTGGTCA |
| TCRb_JM_V24-1 | SEQ ID NO: 85 GATGTTACCCAGACCCCAAGGAATAGGATC |
| TCRb_JM_V25-1 | SEQ ID NO: 86 GACATCTACCAGACCCCAAGATACCTTGTTATAGG |
| TCRb_JM_V26 | SEQ ID NO: 87 GTAGTTACACAATTCCCAAGACACAGAATCATTGG |
| TCRb_JM_V27 | SEQ ID NO: 88 CAAGTGACCCAGAACCCAAGATACCTCATC |

IGH V pool

| | |
|---|---|
| IGH_MTPX_1 | SEQ ID NO: 89 GGTGGCAGCAGTCACAGATGCCTACTC |
| IGH_MTPX_2 | SEQ ID NO: 90 GGTGGCAGCAGCCACAGGTGCCCACTC |
| IGH_MTPX_3 | SEQ ID NO: 91 GGTGGCAGCAGCTACAGGTGTCCAGTC |
| IGH_MTPX_4 | SEQ ID NO: 92 GGTGGSAGCAGCAACARGWGCCCACTC |
| IGH_MTPX_5 | SEQ ID NO: 93 GCTGGCTGTAGCTCCAGGTGCTCACTC |
| IGH_MTPX_6 | SEQ ID NO: 94 CCTGCTGCTGACCAYCCCTTCMTGGGTCTTGTC |
| IGH_MTPX_7 | SEQ ID NO: 95 CCTGCTACTGACTGTCCCGTCCTGGGTCTTATC |
| IGH_MTPX_8 | SEQ ID NO: 96 GGGTTTTCCTCGTTGCTCTTTTAAGAGGTGTCCAGTG |
| IGH_MTPX_9 | SEQ ID NO: 97 GGGTTTTCCTTGTTGCTATTTTAAAAGGTGTCCARTG |
| IGH_MTPX_10 | SEQ ID NO: 98 GGATTTTCCTTGCTGCTATTTTAAAAGGTGTCCAGTG |
| IGH_MTPX_11 | SEQ ID NO: 99 GGGTTTTCCTTKTKGCTATWTTAGAAGGTGTCCAGTG |
| IGH_MTPX_12 | SEQ ID NO: 100 GGTGGCRGCTCCCAGATGGGTCCTGTC |
| IGH_MTPX_13 | SEQ ID NO: 101 CTGGCTGTTCTCCAAGGAGTCTGTG |
| IGH_MTPX_14 | SEQ ID NO: 102 GGCCTCCCATGGGGTGTCCTGTC |
| IGH_MTPX_15 | SEQ ID NO: 103 GGTGGCAGCAGCAACAGGTGCCCACT |
| IGH_MTPX_16 | SEQ ID NO: 104 ATGGAACTGGGGCTCCGCTGGGTTTTCC |
| IGH_MTPX_17 | SEQ ID NO: 105 ATGGACTGCACCTGGAGGATCCTCCTC |
| IGH_MTPX_18 | SEQ ID NO: 106 TGGCTGAGCTGGGTTTYCCTTGTTGC |
| IGH_MTPX_19 | SEQ ID NO: 107 GGAGTTKGGGCTGMGCTGGGTTTTCC |
| IGH_MTPX_20 | SEQ ID NO: 108 GCACCTGTGGTTTTTCCTCCTGCTGGTG |
| IGH_MTPX_21 | SEQ ID NO: 109 CACCTGTGGTTCTTCCTCCTSCTGG |
| IGH_MTPX_22 | SEQ ID NO: 110 CCAGGATGGGGTCAACCGCCATCCTC |
| IGH_MTPX_23 | SEQ ID NO: 111 CAGAGGACTCACCATGGAGTTTGGGCTGAG |
| IGH_MTPX_24 | SEQ ID NO: 112 GGACTCACCATGGAGTTGGGACTGAGC |
| IGH_MTPX_25 | SEQ ID NO: 113 GGGCTGAGCTGGCTTTTTCTTGTGGC |
| TSO Sequence | SEQ ID NO: 114 AAGCAGTGGTATCAACGCAGAGTACATGGG |
| TSO Sequence | SEQ ID NO: 115 TCTGCGTTGATACCACT |
| Portion | |

CDR3

| | |
|---|---|
| TRAV1-2 | SEQ ID NO: 116 gaaggagctccagatgaaagactctgcctc |
| TRAV2 | SEQ ID NO: 117 gttctcttcatcgctgctcatcctccaggt |
| TRAV3 | SEQ ID NO: 118 cttgtgagcgactccgctttgtacttctgt |
| TRAV4 | SEQ ID NO: 119 ttatccctgccgacagaaagtccagcactc |
| TRAV5 | SEQ ID NO: 120 aaggataaacatctgtctctgcgcattgcag |
| TRAV6 | SEQ ID NO: 121 ttgtttcatatcacagcctcccagcctgca |
| TRAV7 | SEQ ID NO: 122 tacattacagccgtgcagcctgaagattcag |
| TRAV8-1 | SEQ ID NO: 123 aatctgaggaaaccctctgtgcagtggagt |
| TRAV8-2 | SEQ ID NO: 124 gaaacctccttccacctgacgaaaccctca |
| TRAV8-3 | SEQ ID NO: 125 caatctgaggaaaccctctgtgcattggag |
| TRAV8-4 | SEQ ID NO: 126 cacctgacgaaaccctcagcccatatgagc |
| TRAV8-6 | SEQ ID NO: 127 ggaaaccctcagtccatataagcgacacgg |
| TRAV8-7 | SEQ ID NO: 128 gaggaaaccatcaacccatgtgagtgatgc |
| TRAV9-1 | SEQ ID NO: 129 ggaaggaacaaaggttttgaagccatgtaccg |
| TRAV9-2 | SEQ ID NO: 130 tccacttggagaaaggctcagttcaagtgt |
| TRAV10 | SEQ ID NO: 131 gcagacacaaagcaaagctctctgcacatc |
| TRAV12-1 | SEQ ID NO: 132 gccagccagtatatttccctgctcatcaga |
| TRAV12-2 | SEQ ID NO: 133 gccagccagtatgtttctctgctcatcaga |
| TRAV12-3 | SEQ ID NO: 134 ggtttacagcacaggtogataaatccagca |
| TRAV13-1 | SEQ ID NO: 135 gccaaacatttctccctgcacatcacagag |
| TRAV13-2 | SEQ ID NO: 136 tctgcaaattgcagctactcaacctggaga |
| TRAV14D4 | SEQ ID NO: 137 gccaaccttgtcatctccgcttcacaactg |
| TRAV16 | SEQ ID NO: 138 gaccttaacaaaggcgagacatctttccacc |
| TRAV17 | SEQ ID NO: 139 gtcacgcttgacacttccaagaaaagcagt |
| TRAV18 | SEQ ID NO: 140 cctatcaagagtgacagttccttccacctg |
| TRAV19 | SEQ ID NO: 141 ggaacttccagaaatccaccagttccttca |
| TRAV20 | SEQ ID NO: 142 agaaggaaagctttctgcacatcacagcc |
| TRAV21 | SEQ ID NO: 143 caagtggaagacttaatgcctcgctggata |
| TRAV22 | SEQ ID NO: 144 gactgtcgctacggaacgctacagcttatt |
| TRAV23DV6 | SEQ ID NO: 145 tgccaagcagttctcatcgcatatcatgga |
| TRAV24 | SEQ ID NO: 146 gccactcttaataccaaggagggttacagc |
| TRAV25 | SEQ ID NO: 147 cacatcacagccacccagactacagatgta |
| TRAV26-1 | SEQ ID NO: 148 tcatcacagaagacagaaagtccagcacct |
| TRAV26-2 | SEQ ID NO: 149 agaaagtccagtaccttgatcctgcaccgt |
| TRAV27 | SEQ ID NO: 150 gttctctccacatcactgcagcccagactg |
| TRAV29DV5 | SEQ ID NO: 151 aaagtgccaagcacctctctctgcacattg |
| TRAV30 | SEQ ID NO: 152 ctgtaccttacggcctcccagctcagttac |
| TRAV34 | SEQ ID NO: 154 gacctcaaatggaagactgactgctcagtt |
| TRAV35 | SEQ ID NO: 153 gccaagttggatgagaaaaagcagcaaagt |
| TRAV36DV7 | SEQ ID NO: 155 tttcagcatcctgaacatcacagccaccca |
| TRAV38-2DV8 | SEQ ID NO: 156 ccttcagtctcaagatctcagactcacagc |
| TRAV39 | SEQ ID NO: 157 aatggcctcacttgataccaaagcccgtc |
| TRAV40 | SEQ ID NO: 158 ctcccccattgtgaaatattcagtccaggt |
| TRAV41 | SEQ ID NO: 159 catacaggaaaagcacagctccctgcacat |
| TRAV11 | SEQ ID NO: 160 atatcgcagcctctcatctgggagattcagc |
| TRAV1-1 | SEQ ID NO: 161 caggagctccagatgaaagactctgcctctt |
| TRAV8-5 | SEQ ID NO: 162 acttccttccacttgaggaaaccctcagtcca |

Inner TRAV Primers

| | |
|---|---|
| TRAV-Handle 1 | SEQ ID NO: 163 |
| | gtgactggagttcagacgtgtgctcttccgatctgaaggagctccagatgaaagactctgcctc |
| TRAV-Handle 2 | SEQ ID NO: 164 |
| | gtgactggagttcagacgtgtgctcttccgatctgttctcttcatcgctgctcatcctccaggt |
| TRAV-Handle 3 | SEQ ID NO: 165 |
| | gtgactggagttcagacgtgtgctcttccgatctcttgtgagcgactccgctttgtacttctgt |
| TRAV-Handle 4 | SEQ ID NO: 166 |
| | gtgactggagttcagacgtgtgctcttccgatctttatccctgccgacagaaagtccagcactc |
| TRAV-Handle 5 | SEQ ID NO: 167 |
| | gtgactggagttcagacgtgtgctcttccgatctaaggataaacatctgtctctgcgcattgcag |
| TRAV-Handle 6 | SEQ ID NO: 168 |
| | gtgactggagttcagacgtgtgctcttccgatctttgtttcatatcacagcctcccagcctgca |
| TRAV-Handle 7 | SEQ ID NO: 169 |
| | gtgactggagttcagacgtgtgctcttccgatcttacattacagccgtgcagcctgaagattcag |
| TRAV-Handle 8 | SEQ ID NO: 170 |
| | gtgactggagttcagacgtgtgctcttccgatctaatctgaggaaaccctctgtgcagtggagt |
| TRAV-Handle 9 | SEQ ID NO: 171 |
| | gtgactggagttcagacgtgtgctcttccgatctgaaacctccttccacctgacgaaaccctca |
| TRAV-Handle 10 | SEQ ID NO: 172 |
| | gtgactggagttcagacgtgtgctcttccgatctcaatctgaggaaaccctctgtgcattggag |
| TRAV-Handle 11 | SEQ ID NO: 173 |
| | gtgactggagttcagacgtgtgctcttccgatctcacctgacgaaaccctcagcccatatgagc |
| TRAV-Handle 12 | SEQ ID NO: 174 |
| | gtgactggagttcagacgtgtgctcttccgatctggaaaccctcagtccatataagcgacacgg |
| TRAV-Handle 13 | SEQ ID NO: 175 |
| | gtgactggagttcagacgtgtgctcttccgatctgaggaaaccatcaacccatgtgagtgatgc |
| TRAV-Handle 14 | SEQ ID NO: 176 |
| | gtgactggagttcagacgtgtgctcttccgatctggaaggaacaaaggttttgaagccatgtaccg |
| TRAV-Handle 15 | SEQ ID NO: 177 |
| | gtgactggagttcagacgtgtgctcttccgatcttccacttggagaaaggctcagttcaagtgt |
| TRAV-Handle 16 | SEQ ID NO: 178 |
| | gtgactggagttcagacgtgtgctcttccgatctgcagacacaaagcaaagctctctgcacatc |
| TRAV-Handle 17 | SEQ ID NO: 179 |
| | gtgactggagttcagacgtgtgctcttccgatctgccagccagtatatttccctgctcatcaga |
| TRAV-Handle 18 | SEQ ID NO: 180 |
| | gtgactggagttcagacgtgtgctcttccgatctgccagccagtatgtttctctgctcatcaga |
| TRAV-Handle 19 | SEQ ID NO: 181 |
| | gtgactggagttcagacgtgtgctcttccgatctggtttacagcacaggtcgataaatccagca |
| TRAV-Handle 20 | SEQ ID NO: 182 |
| | gtgactggagttcagacgtgtgctcttccgatctgccaaacatttctccctgcacatcacagag |
| TRAV-Handle 21 | SEQ ID NO: 183 |
| | gtgactggagttcagacgtgtgctcttccgatcttctgcaaattgcagctactcaacctggaga |
| TRAV-Handle 22 | SEQ ID NO: 184 |
| | gtgactggagttcagacgtgtgctcttccgatctgccaaccttgtcatctccgcttcacaactg |
| TRAV-Handle 23 | SEQ ID NO: 185 |
| | gtgactggagttcagacgtgtgctcttccgatctgaccttaacaaaggcgagacatctttccacc |
| TRAV-Handle 24 | SEQ ID NO: 186 |
| | gtgactggagttcagacgtgtgctcttccgatctgtcacgcttgacacttccaagaaaagcagt |
| TRAV-Handle 25 | SEQ ID NO: 187 |
| | gtgactggagttcagacgtgtgctcttccgatctcctatcaagagtgacagttccttccacctg |
| TRAV-Handle 26 | SEQ ID NO: 188 |
| | gtgactggagttcagacgtgtgctcttccgatctggaacttccagaaatccaccagttccttca |
| TRAV-Handle 27 | SEQ ID NO: 189 |
| | gtgactggagttcagacgtgtgctcttccgatctagaaggaaagctttctgcacatcacagcc |
| TRAV-Handle 28 | SEQ ID NO: 190 |
| | gtgactggagttcagacgtgtgctcttccgatctcaagtggaagacttaatgcctcgctggata |
| TRAV-Handle 29 | SEQ ID NO: 191 |
| | gtgactggagttcagacgtgtgctcttccgatctgactgtcgctacggaacgctacagcttatt |
| TRAV-Handle 30 | SEQ ID NO: 192 |
| | gtgactggagttcagacgtgtgctcttccgatcttgccaagcagttctcatcgcatatcatgga |
| TRAV-Handle 31 | SEQ ID NO: 193 |
| | gtgactggagttcagacgtgtgctcttccgatctgccactcttaataccaaggagggttacagc |
| TRAV-Handle 32 | SEQ ID NO: 194 |
| | gtgactggagttcagacgtgtgctcttccgatctcacatcacagccacccagactacagatgta |
| TRAV-Handle 33 | SEQ ID NO: 195 |
| | gtgactggagttcagacgtgtgctcttccgatottcatcacagaagacagaaagtccagcacct |
| TRAV-Handle 34 | SEQ ID NO: 196 |
| | gtgactggagttcagacgtgtgctcttccgatctagaaagtccagtaccttgatcctgcaccgt |
| TRAV-Handle 35 | SEQ ID NO: 197 |
| | gtgactggagttcagacgtgtgctcttccgatctgttctctccacatcactgcagcccagactg |
| TRAV-Handle 36 | SEQ ID NO: 198 |
| | gtgactggagttcagacgtgtgctcttccgatctaaagtgccaagcacctctctctgcacattg |
| TRAV-Handle 37 | SEQ ID NO: 199 |
| | gtgactggagttcagacgtgtgctcttccgatctctgtaccttacggcctcccagctcagttac |
| TRAV-Handle 38 | SEQ ID NO: 200 |
| | gtgactggagttcagacgtgtgctcttccgatctgccaagttggatgagaaaaagcagcaaagt |
| TRAV-Handle 39 | SEQ ID NO: 201 |
| | gtgactggagttcagacgtgtgctcttccgatctgacctcaaatggaagactgactgctcagtt |
| TRAV-Handle 40 | SEQ ID NO: 202 |
| | gtgactggagttcagacgtgtgctcttccgatcttttcagcatcctgaacatcacagccaccca |
| TRAV-Handle 41 | SEQ ID NO: 203 |
| | gtgactggagttcagacgtgtgctcttccgatctccttcagtctcaagatctcagactcacagc |
| TRAV-Handle 42 | SEQ ID NO: 204 |
| | gtgactggagttcagacgtgtgctcttccgatctaatggcctcacttgataccaaagcccgtc |
| TRAV-Handle 43 | SEQ ID NO: 205 |
| | gtgactggagttcagacgtgtgctcttccgatctctcccccattgtgaaatattcagtccaggt |
| TRAV-Handle 44 | SEQ ID NO: 206 |
| | gtgactggagttcagacgtgtgctcttccgatctcatacaggaaaagcacagctccctgcacat |
| TRAV-Handle 45 | SEQ ID NO: 207 |
| | gtgactggagttcagacgtgtgctcttccgatctatatcgcagcctctcatctgggagattcagc |
| TRAV-Handle 46 | SEQ ID NO: 208 |
| | gtgactggagttcagacgtgtgctcttccgatctcaggagctccagatgaaagactctgcctctt |
| TRAV-Handle 47 | SEQ ID NO: 209 |
| | gtgactggagttcagacgtgtgctcttccgatctacttccttccacttgaggaaaccctcagtcca |
| 5′ Sequence | SEQ ID NO: 210 gtgactggagttcagacgtgtgctcttccgatct |
| Handle LN2 | |

Outer TRAV Primers

| | |
|---|---|
| TRAV10*01_outer | SEQ ID NO: 211 aaaaaccaagtggagcagagtcctcagtccctg |
| TRAV21*01_outer | SEQ ID NO: 212 aaacaggaggtgacgcagattcctgcagctc |
| TRAV2*01_outer | SEQ ID NO: 213 aaggaccaagtgtttcagccttccacagtggc |
| TRAV8-6*02_outer | SEQ ID NO: 214 acccagcttgacagccaagtccctgtct |
| TRAV8-7*02_outer | SEQ ID NO: 215 acccagcttgatggccacatcactgtctct |
| TRAV8-4*01_outer | SEQ ID NO: 216 acccagcttggcagccacgtctctg |
| TRAV19*01_outer | SEQ ID NO: 217 actcaagcgcagactgaaatttctgtggtgg |
| TRAV12-3*01_outer | SEQ ID NO: 218 agaaggaggtggagcaggatcctggacca |
| TRAV6*01_outer | SEQ ID NO: 219 agaattccgaggctctgaacattcaggagggtaa |
| TRAV16*01_outer | SEQ ID NO: 220 agagagtgactcagcccgagaagctcctct |
| TRAV8-3*01_outer | SEQ ID NO: 221 agagcccagtcagtgacccagcctgac |
| TRAV8-5*01_outer | SEQ ID NO: 222 agagcccagtcagtgacccagcctgac |
| TRAV27*01_outer | SEQ ID NO: 223 agctgctggagcagagccctcagtttc |
| TRAV17*01_outer | SEQ ID NO: 224 agtcaacagggagaagaggatcctcaggccttg |
| TRAV18*01_outer | SEQ ID NO: 225 agtggagactcggttacccagacagaaggcc |
| TRAV22*01_outer | SEQ ID NO: 226 agtggagcagagtcctccagacctgattctc |
| TRAV13-2*01_outer | SEQ ID NO: 227 agtgtggggctgcatcttcctaccctga |
| TRAV24*01_outer | SEQ ID NO: 228 atactgaacgtggaacaaagtcctcagtcactgcatg |
| TRAV9-2*01_outer | SEQ ID NO: 229 attcagtgacccagatggaagggccagtga |
| TRAV26-1*01_outer | SEQ ID NO: 230 attgatgctaagaccacccagcccacctc |
| TRAV12-2*01_outer | SEQ ID NO: 231 cagaaggaggtggagcagaattctggacccc |
| TRAV40*01_outer | SEQ ID NO: 232 cagcaattcagtcaagcagacgggccaa |
| TRAV30*01_outer | SEQ ID NO: 233 ccaacaaccagtgcagagtcctcaagccg |
| TRAV12-1*01_outer | SEQ ID NO: 234 cggaaggaggtggagcaggatcctgga |
| TRAV11-1*01_outer | SEQ ID NO: 235 ctacatacgccggagcagagtccttcattcctgag |
| TRAV14/DV4*02_outer | SEQ ID NO: 236 ctcaaacccaaccaggaatgttcgtgcagga |
| TRAV4*01_outer | SEQ ID NO: 237 cttgctaagaccacccagcccatctccatggactc |
| TRAV7*01_outer | SEQ ID NO: 238 gaaaaccaggtggagcacagccctcattttctg |
| TRAV36/DV7*01_outer | SEQ ID NO: 239 gaagacaaggtggtacaaagccctctatctctggt |
| TRAV20*01_outer | SEQ ID NO: 240 gaagaccaggtgacgcagagtcccgag |
| TRAV23/DV6*01_outer | SEQ ID NO: 241 gaccagcagcaggtgaaacaaagtcctcaat |
| TRAV41*01_outer | SEQ ID NO: 242 gagcagagtcctcagaacctgactgccc |
| TRAV29/DV5*01_outer | SEQ ID NO: 243 gatgaccagcaagttaagcaaaattcaccatccct |
| TRAV34*01_outer | SEQ ID NO: 244 gccaagaactggagcagagtcctcagtcc |
| TRAV8-2*01_outer | SEQ ID NO: 245 gcccagtcggtgacccagcttgacag |
| TRAV8-1*01_outer | SEQ ID NO: 246 gcccagtctgtgagccagcataaccaccac |
| TRAV26-2*01_outer | SEQ ID NO: 247 gcctgttcacttgccttgtaaccactccac |
| TRAV3*01_outer | SEQ ID NO: 248 gctcagtcagtggctcagccggaagatcagg |
| TRAV1-2*01_outer | SEQ ID NO: 249 ggacaaaacattgaccagcccactgagatgacagc |
| TRAV1-1*01_outer | SEQ ID NO: 250 ggacaaagccttgagcagccctctgaagtgac |
| TRAV25*01_outer | SEQ ID NO: 251 ggacaacaggtaatgcaaattcctcagtaccagcatg |
| TRAV13-1*01_outer | SEQ ID NO: 252 ggagagaatgtggagcagcatccttcaaccctg |
| TRAV5*01_outer | SEQ ID NO: 253 ggagaggatgtggagcagagtcttttcctgagtgtc |
| TRAV9-1*01_outer | SEQ ID NO: 254 ggagattcagtggtccagacagaaggccaagtg |
| TRAV38-2/DV8*01_ | SEQ ID NO: 255 gtctcaaccagagatgtctgtgcaggagg |
| outer | |
| TRAV39*01_outer | SEQ ID NO: 256 gtggaacaaaaccctctgttcctgagcatgc |
| TRAV35*01_outer | SEQ ID NO: 257 gtggtcaacagctgaatcagagtcctcaatcta |
| TRAV11*01_outer | SEQ ID NO: 258 gttccggcaggatccggggagaagact |

CDR3

| | |
|---|---|
| TRBV10-1 | SEQ ID NO: 259 gcctcctcccagacatctgtatatttctgcg |
| TRBV10-2 | SEQ ID NO: 260 aatttccccctcactctggagtcagctacc |
| TRBV10-3 | SEQ ID NO: 261 gatttcctcctcactctggagtccgctacc |
| TRBV11-1 | SEQ ID NO: 262 aggctcaaaggagtagactccactctcaaga |
| TRBV11-2 | SEQ ID NO: 263 caagatccagcctgcaaagcttgaggact |
| TRBV11-3 | SEQ ID NO: 264 tagactccactctcaagatccagcctgcag |
| TRBV12-1 | SEQ ID NO: 265 tggaacccagggacttgggcctatatttct |
| TRBV12-2 | SEQ ID NO: 266 tcattctctactctgaagatccagcctgcag |
| TRBV12-3 | SEQ ID NO: 267 cattctccactctgaagatccagccctcag |
| TRBV12-4 | SEQ ID NO: 268 catcattctccactctgaagatccagccctc |
| TRBV12-5 | SEQ ID NO: 269 cagcagagatgcctgatgcaactttagcca |
| TRBV13 | SEQ ID NO: 270 gaactgaacatgagctccttggagctggg |
| TRBV14 | SEQ ID NO: 271 ggaggattctggagtttatttctgtgccagc |
| TRBV15 | SEQ ID NO: 272 ttctgctttcttgacatccgctcaccaggc |
| TRBV16 | SEQ ID NO: 273 gagatccaggctacgaagcttgaggattcag |
| TRBV17 | SEQ ID NO: 274 aacgtcttccacgctgaagatccatccc |
| TRBV18 | SEQ ID NO: 275 aggatccagcaggtagtgcgaggagattcg |
| TRBV19 | SEQ ID NO: 276 acccgacagctttctatctctgtgccagta |
| TRBV20-1 | SEQ ID NO: 277 gtgcccatcctgaagacagcagcttctaca |
| TRBV2 | SEQ ID NO: 278 cacaaagctggaggactcagccatgtac |
| TRBV21-1 | SEQ ID NO: 279 tcaggggacacagcactgtatttctgtgcc |
| TRBV22-1 | SEQ ID NO: 280 cacaccagccaaacagctttgtacttctgt |
| TRBV23-1 | SEQ ID NO: 281 aatcctgtcctcagaaccgggagacacg |
| TRBV24-1 | SEQ ID NO: 282 ccaaccagacagctctttacttctgtgccac |
| TRBV25-1 | SEQ ID NO: 283 cacatacctctcagtacctctgtgccagca |
| TRBV26 | SEQ ID NO: 284 ccaaccagacatctgtgtatctctatgccagc |
| TRBV27 | SEQ ID NO: 285 accagacctctctgtacttctgtgccagca |
| TRBV28 | SEQ ID NO: 286 aaccagacatctatgtacctctgtgccagc |
| TRBV29-1 | SEQ ID NO: 287 acatgagccctgaagacagcagcatatatctc |
| TRBV3-1 | SEQ ID NO: 288 agcttggtgactctgctgtgtatttctgtg |
| TRBV3-2 | SEQ ID NO: 289 cttggtgactctgctgtgtatttctgtgcc |
| TRBV4-1 | SEQ ID NO: 290 cagccagaagactcagccctgtatctctg |
| TRBV4-2 | SEQ ID NO: 291 gccagaagactcggccctgtatctctgt |
| TRBV4-3 | SEQ ID NO: 292 tattccttcacctacacaccctgcagccag |
| TRBV5-1 | SEQ ID NO: 293 agatgaatgtgagcaccttggagctgg |
| TRBV5-2 | SEQ ID NO: 294 tactgagtcaaacacggagctaggggact |
| TRBV5-3 | SEQ ID NO: 295 gttgctctgagatgaatgtgagtgccttgg |
| TRBV5-4 | SEQ ID NO: 296 atagctctgagctgaatgtgaacgccttgg |
| TRBV5-5 | SEQ ID NO: 297 gagctgaatgtgaacgccttgttgctgg |
| TRBV5-6 | SEQ ID NO: 298 aactatagctctgagctgaatgtgaacgcct |
| TRBV5-7 | SEQ ID NO: 299 agctgaatgtgaacgccttgttgctaggg |
| TRBV5-8 | SEQ ID NO: 300 ctgaatgtgaacgccttggagctggagga |
| TRBV6-1 | SEQ ID NO: 301 gctccctcccagacatctgtgtacttct |
| TRBV6-2 | SEQ ID NO: 302 gctgctccctcccaaacatctgtgtact |
| TRBV6-3 | SEQ ID NO: 303 gctccctcccaaacatctgtgtacttctgt |
| TRBV6-4 | SEQ ID NO: 304 aacacagatgatttccccctcacgttggc |
| TRBV6-5 | SEQ ID NO: 305 gctgctccctcccagacatctgtgtactt |
| TRBV6-6 | SEQ ID NO: 306 agttggctgctccctcccagacatctg |
| TRBV6-7 | SEQ ID NO: 307 tcagctgctccctctcagacttctgtttac |
| TRBV6-8 | SEQ ID NO: 308 taaacacagaggatttcccactcaggctggt |
| TRBV6-9 | SEQ ID NO: 309 agtcagctgctccctcccagacatctgtata |
| TRBV7-1 | SEQ ID NO: 310 cagcagggggacttggctgtgtatctc |
| TRBV7-2 | SEQ ID NO: 311 gcaggaggactcggccgtgtatctc |
| TRBV7-3 | SEQ ID NO: 312 tctactctgaagatccagcgcacagagcg |
| TRBV7-4 | SEQ ID NO: 313 cacagagcagggggactcagctgtgtat |
| TRBV7-5 | SEQ ID NO: 314 atctttctccacctgaagatccagcgcaca |
| TRBV7-6 | SEQ ID NO: 315 ttctctgcagagaggcctgagggatccat |
| TRBV7-8 | SEQ ID NO: 316 ctgagggatccgtctccactctgaagatcc |
| TRBV7-9 | SEQ ID NO: 317 ggcctaagggatctttctccaccttggaga |
| TRBV8-1 | SEQ ID NO: 318 ttccctcaaccctggagtctactagcacca |
| TRBV8-2 | SEQ ID NO: 319 ttgagcatttccccaatcctggcatccac |
| TRBV9 | SEQ ID NO: 320 gggactcagctttgtatttctgtgccagca |

Inner TRBV Primers

| | |
|---|---|
| TRBV-Handle 1 | SEQ ID NO: 321 |
| | gtgactggagttcagacgtgtgctcttccgatctgcctcctcccagacatctgtatatttctgcg |
| TRBV-Handle 2 | SEQ ID NO: 322 |
| | gtgactggagttcagacgtgtgctcttccgatctaatttccccctcactctggagtcagctacc |
| TRBV-Handle 3 | SEQ ID NO: 323 |
| | gtgactggagttcagacgtgtgctcttccgatctgatttcctcctcactctggagtccgctacc |
| TRBV-Handle 4 | SEQ ID NO: 324 |
| | gtgactggagttcagacgtgtgctcttccgatctaggctcaaaggagtagactccactctcaaga |
| TRBV-Handle 5 | SEQ ID NO: 325 |
| | gtgactggagttcagacgtgtgctcttccgatctcaagatccagcctgcaaagcttgaggact |
| TRBV-Handle 6 | SEQ ID NO: 326 |
| | gtgactggagttcagacgtgtgctcttccgatcttagactccactctcaagatccagcctgcag |
| TRBV-Handle 7 | SEQ ID NO: 327 |
| | gtgactggagttcagacgtgtgctcttccgatcttggaacccagggacttgggcctatatttct |
| TRBV-Handle 8 | SEQ ID NO: 328 |
| | gtgactggagttcagacgtgtgctcttccgatcttcattctctactctgaagatccagcctgcag |
| TRBV-Handle 9 | SEQ ID NO: 329 |
| | gtgactggagttcagacgtgtgctcttccgatctcattctccactctgaagatccagccctcag |
| TRBV-Handle 10 | SEQ ID NO: 330 |
| | gtgactggagttcagacgtgtgctcttccgatctcatcattctccactctgaagatccagccctc |
| TRBV-Handle 11 | SEQ ID NO: 331 |
| | gtgactggagttcagacgtgtgctcttccgatctcagcagagatgcctgatgcaactttagcca |
| TRBV-Handle 12 | SEQ ID NO: 332 |
| | gtgactggagttcagacgtgtgctcttccgatctgaactgaacatgagctccttggagctggg |
| TRBV-Handle 13 | SEQ ID NO: 333 |
| | gtgactggagttcagacgtgtgctcttccgatctggaggattctggagtttatttctgtgccagc |
| TRBV-Handle 14 | SEQ ID NO: 334 |
| | gtgactggagttcagacgtgtgctcttccgatctttctgctttcttgacatccgctcaccaggc |
| TRBV-Handle 15 | SEQ ID NO: 335 |
| | gtgactggagttcagacgtgtgctcttccgatctgagatccaggctacgaagcttgaggattcag |
| TRBV-Handle 16 | SEQ ID NO: 336 |
| | gtgactggagttcagacgtgtgctcttccgatctaacgtcttccacgctgaagatccatccc |
| TRBV-Handle 17 | SEQ ID NO: 337 |
| | gtgactggagttcagacgtgtgctcttccgatctaggatccagcaggtagtgcgaggagattcg |
| TRBV-Handle 18 | SEQ ID NO: 338 |
| | gtgactggagttcagacgtgtgctcttccgatctacccgacagctttctatctctgtgccagta |
| TRBV-Handle 19 | SEQ ID NO: 339 |
| | gtgactggagttcagacgtgtgctcttccgatctgtgcccatcctgaagacagcagcttctaca |
| TRBV-Handle 20 | SEQ ID NO: 340 |
| | gtgactggagttcagacgtgtgctcttccgatctcacaaagctggaggactcagccatgtac |
| TRBV-Handle 21 | SEQ ID NO: 341 |
| | gtgactggagttcagacgtgtgctcttccgatcttcaggggacacagcactgtatttctgtgcc |
| TRBV-Handle 22 | SEQ ID NO: 342 |
| | gtgactggagttcagacgtgtgctcttccgatctcacaccagccaaacagctttgtacttctgt |
| TRBV-Handle 23 | SEQ ID NO: 343 |
| | gtgactggagttcagacgtgtgctcttccgatctaatcctgtcctcagaaccgggagacacg |
| TRBV-Handle 24 | SEQ ID NO: 344 |
| | gtgactggagttcagacgtgtgctcttccgatctccaaccagacagctctttacttctgtgccac |
| TRBV-Handle 25 | SEQ ID NO: 345 |
| | gtgactggagttcagacgtgtgctcttccgatctcacatacctctcagtacctctgtgccagca |
| TRBV-Handle 26 | SEQ ID NO: 346 |
| | gtgactggagttcagacgtgtgctcttccgatctccaaccagacatctgtgtatctctatgccagc |
| TRBV-Handle 27 | SEQ ID NO: 347 |
| | gtgactggagttcagacgtgtgctcttccgatctaccagacctctctgtacttctgtgccagca |
| TRBV-Handle 28 | SEQ ID NO: 348 |
| | gtgactggagttcagacgtgtgctcttccgatctaaccagacatctatgtacctctgtgccagc |
| TRBV-Handle 29 | SEQ ID NO: 349 |
| | gtgactggagttcagacgtgtgctcttccgatctacatgagccctgaagacagcagcatatatctc |
| TRBV-Handle 30 | SEQ ID NO: 350 |
| | gtgactggagttcagacgtgtgctcttccgatctagcttggtgactctgctgtgtatttctgtg |
| TRBV-Handle 31 | SEQ ID NO: 351 |
| | gtgactggagttcagacgtgtgctcttccgatctcttggtgactctgctgtgtatttctgtgcc |
| TRBV-Handle 32 | SEQ ID NO: 352 |
| | gtgactggagttcagacgtgtgctcttccgatctcagccagaagactcagccctgtatctctg |
| TRBV-Handle 33 | SEQ ID NO: 353 |
| | gtgactggagttcagacgtgtgctcttccgatctgccagaagactcggccctgtatctctgt |
| TRBV-Handle 34 | SEQ ID NO: 354 |
| | gtgactggagttcagacgtgtgctcttccgatcttattccttcacctacacaccctgcagccag |
| TRBV-Handle 35 | SEQ ID NO: 355 |
| | gtgactggagttcagacgtgtgctcttccgatctagatgaatgtgagcaccttggagctgg |
| TRBV-Handle 36 | SEQ ID NO: 356 |
| | gtgactggagttcagacgtgtgctcttccgatcttactgagtcaaacacggagctaggggact |
| TRBV-Handle 37 | SEQ ID NO: 357 |
| | gtgactggagttcagacgtgtgctcttccgatctgttgctctgagatgaatgtgagtgccttgg |
| TRBV-Handle 38 | SEQ ID NO: 358 |
| | gtgactggagttcagacgtgtgctcttccgatctatagctctgagctgaatgtgaacgccttgg |
| TRBV-Handle 39 | SEQ ID NO: 359 |
| | gtgactggagttcagacgtgtgctcttccgatctgagctgaatgtgaacgccttgttgctgg |
| TRBV-Handle 40 | SEQ ID NO: 360 |
| | gtgactggagttcagacgtgtgctcttccgatctaactatagctctgagctgaatgtgaacgcct |
| TRBV-Handle 41 | SEQ ID NO: 361 |
| | gtgactggagttcagacgtgtgctcttccgatctagctgaatgtgaacgccttgttgctaggg |
| TRBV-Handle 42 | SEQ ID NO: 362 |
| | gtgactggagttcagacgtgtgctcttccgatctctgaatgtgaacgccttggagctggagga |
| TRBV-Handle 43 | SEQ ID NO: 363 |
| | gtgactggagttcagacgtgtgctcttccgatctgctccctcccagacatctgtgtacttct |
| TRBV-Handle 44 | SEQ ID NO: 364 |
| | gtgactggagttcagacgtgtgctcttccgatctgctgctccctcccaaacatctgtgtact |
| TRBV-Handle 45 | SEQ ID NO: 365 |
| | gtgactggagttcagacgtgtgctcttccgatctgctccctcccaaacatctgtgtacttctgt |
| TRBV-Handle 46 | SEQ ID NO: 366 |
| | gtgactggagttcagacgtgtgctcttccgatctaacacagatgatttccccctcacgttggc |
| TRBV-Handle 47 | SEQ ID NO: 367 |
| | gtgactggagttcagacgtgtgctcttccgatctgctgctccctcccagacatctgtgtactt |
| TRBV-Handle 48 | SEQ ID NO: 368 |
| | gtgactggagttcagacgtgtgctcttccgatctagttggctgctccctcccagacatctg |
| TRBV-Handle 49 | SEQ ID NO: 369 |
| | gtgactggagttcagacgtgtgctcttccgatcttcagctgctccctctcagacttctgtttac |
| TRBV-Handle 50 | SEQ ID NO: 370 |
| | gtgactggagttcagacgtgtgctcttccgatcttaaacacagaggatttcccactcaggctggt |
| TRBV-Handle 51 | SEQ ID NO: 371 |
| | gtgactggagttcagacgtgtgctcttccgatctagtcagctgctccctcccagacatctgtata |
| TRBV-Handle 52 | SEQ ID NO: 372 |
| | gtgactggagttcagacgtgtgctcttccgatctcagcagggggacttggctgtgtatctc |
| TRBV-Handle 53 | SEQ ID NO: 373 |
| | gtgactggagttcagacgtgtgctcttccgatctgcaggaggactcggccgtgtatctc |
| TRBV-Handle 54 | SEQ ID NO: 374 |
| | gtgactggagttcagacgtgtgctcttccgatcttctactctgaagatccagcgcacagagcg |
| TRBV-Handle 55 | SEQ ID NO: 375 |
| | gtgactggagttcagacgtgtgctcttccgatctcacagagcagggggactcagctgtgtat |
| TRBV-Handle 56 | SEQ ID NO: 376 |
| | gtgactggagttcagacgtgtgctcttccgatctatctttctccacctgaagatccagcgcaca |
| TRBV-Handle 57 | SEQ ID NO: 377 |
| | gtgactggagttcagacgtgtgctcttccgatctttctctgcagagaggcctgagggatccat |
| TRBV-Handle 58 | SEQ ID NO: 378 |
| | gtgactggagttcagacgtgtgctcttccgatctctgagggatccgtctccactctgaagatcc |
| TRBV-Handle 59 | SEQ ID NO: 379 |
| | gtgactggagttcagacgtgtgctcttccgatctggcctaagggatotttctccaccttggaga |
| TRBV-Handle 60 | SEQ ID NO: 380 |
| | gtgactggagttcagacgtgtgctcttccgatotttccctcaaccctggagtctactagcacca |
| TRBV-Handle 61 | SEQ ID NO: 381 |
| | gtgactggagttcagacgtgtgctcttccgatctttgagcatttccccaatcctggcatccac |
| TRBV-Handle 62 | SEQ ID NO: 382 |
| | gtgactggagttcagacgtgtgctcttccgatctgggactcagctttgtatttctgtgccagca |

Outer TRBV Primers

| | |
|---|---|
| TRBV10-1_outer | SEQ ID NO: 383 gctgaaatcacccagagcccaagacacaag |
| TRBV10-2_outer | SEQ ID NO: 384 cacagagacaggaaggcaggtgaccttga |
| TRBV10-3_outer | SEQ ID NO: 385 gatgctggaatcacccagagcccaagacac |
| TRBV11-1_outer | SEQ ID NO: 386 gccaggctgtggctttttggtgtgatccta |
| TRBV11-2_outer | SEQ ID NO: 387 ggcagagtgtggctttttggtgcaatcct |
| TRBV11-3_outer | SEQ ID NO: 388 ggctttttggtgcaatcctatttctggccac |
| TRBV12-1_outer | SEQ ID NO: 389 gatgctggtgttatccagtcacccaggcac |
| TRBV12-2_outer | SEQ ID NO: 390 gtcacccaagcatgaggtgacagaaatggg |
| TRBV12-3_outer | SEQ ID NO: 391 atgctggagttatccagtcaccccgcc |
| TRBV12-4_outer | SEQ ID NO: 392 gagttatccagtcaccccggcacgaggt |
| TRBV12-5_outer | SEQ ID NO: 393 gctagagtcacccagacaccaaggcaca |
| TRBV13_outer | SEQ ID NO: 394 gctgctggagtcatccagtccccaaga |
| TRBV14_outer | SEQ ID NO: 395 gttactcagttccccagccacagcgtaat |
| TRBV15_outer | SEQ ID NO: 396 gttacccagtttggaaagccagtgaccct |
| TRBV16_outer | SEQ ID NO: 397 gaagtcgcccagactccaaaacatcttgtc |
| TRBV17_outer | SEQ ID NO: 398 cagacacaaggtcaccaacatgggacagg |
| TRBV18_outer | SEQ ID NO: 399 gtcatgtttactggtatcggcagctccca |
| TRBV19_outer | SEQ ID NO: 400 atgccatgtactggtaccgacaggaccca |
| TRBV20-1_outer | SEQ ID NO: 401 gtcgtctctcaacatccgagctgggttat |
| TRBV2_outer | SEQ ID NO: 402 gaacctgaagtcacccagactcccagcca |
| TRBV21-1_outer | SEQ ID NO: 403 cacggacaccaaggtcacccagagacct |
| TRBV22-1_outer | SEQ ID NO: 404 agctcactggggctggatgggatgtgac |
| TRBV23-1_outer | SEQ ID NO: 405 gccaaagtcacacagactccaggacattt |
| TRBV24-1_outer | SEQ ID NO: 406 gtatcgacaagacccaggactgggcctac |
| TRBV25-1_outer | SEQ ID NO: 407 gctgacatctaccagaccccaagatacct |
| TRBV26_outer | SEQ ID NO: 408 gtatcgacaggacccaggacttggactga |
| TRBV27_outer | SEQ ID NO: 409 agcccaagtgacccagaacccaagatac |
| TRBV28_outer | SEQ ID NO: 410 ctcgtagatgtgaaagtaacccagagctcga |
| TRBV29-1_outer | SEQ ID NO: 411 gatatctgtcaacgtggaacctccctgacg |
| TRBV3-1_outer | SEQ ID NO: 412 ggtcacacagatgggaaacgacaagtcca |
| TRBV3-2_outer | SEQ ID NO: 413 ccgtttcccagactccaaaatacctggtc |
| TRBV4-1_outer | SEQ ID NO: 414 gaagttacccagacaccaaaacacctggtc |
| TRBV4-2_outer | SEQ ID NO: 415 gagttacgcagacaccaagacacctggtc |
| TRBV4-3_outer | SEQ ID NO: 416 ggagttacgcagacaccaagacacctgg |
| TRBV5-1_outer | SEQ ID NO: 417 gtgacactgagctgctcccctatctctgg |
| TRBV5-2_outer | SEQ ID NO: 418 gaatcacccaagctccaagacacctgatc |
| TRBV5-3_outer | SEQ ID NO: 419 ctggagtcacccaaagtcccacacacc |
| TRBV5-4_outer | SEQ ID NO: 420 gactggagtcacccaaagtcccacacac |
| TRBV5-5_outer | SEQ ID NO: 421 gtcccacacacctgatcaaaacgagagga |
| TRBV5-6_outer | SEQ ID NO: 422 tagtggacgctggagtcacccaaagtcc |
| TRBV5-7_outer | SEQ ID NO: 423 ctgatcaaaacgagaggacagcacgtgac |
| TRBV5-8_outer | SEQ ID NO: 424 gagtcacacaaagtcccacacacctgatc |
| TRBV6-1_outer | SEQ ID NO: 425 gtgaatgctggtgtcactcagaccccaaa |
| TRBV6-2_outer | SEQ ID NO: 426 gaatgctggtgtcactcagaccccaaaat |
| TRBV6-3_outer | SEQ ID NO: 427 gctggtgtcactcagaccccaaaattccg |
| TRBV6-4_outer | SEQ ID NO: 428 gatcacccaggcaccaacatctcagatcc |
| TRBV6-5_outer | SEQ ID NO: 429 gctggtgtcactcagaccccaaaattcca |
| TRBV6-6_outer | SEQ ID NO: 430 gctggtgtcactcagaccccaaaattccg |
| TRBV6-7_outer | SEQ ID NO: 431 gaatgctggtgtcactcagaccccaaaat |
| TRBV6-8_outer | SEQ ID NO: 432 gctggtgtcactcagaccccaaaattcca |
| TRBV6-9_outer | SEQ ID NO: 433 gaatgctggtgtcactcagaccccaaaat |
| TRBV7-1_outer | SEQ ID NO: 434 gtgctggagtctcccagtccctgagaca |
| TRBV7-2_outer | SEQ ID NO: 435 gtcccccagtaacaaggtcacagagaagg |
| TRBV7-3_outer | SEQ ID NO: 436 gacccccagtaacaaggtcacagagaagg |
| TRBV7-4_outer | SEQ ID NO: 437 cagtccccaaggtacaaagtcgcaaagag |
| TRBV7-5_outer | SEQ ID NO: 438 gtctcccagtccccaaggtacgaagtc |
| TRBV7-6_outer | SEQ ID NO: 439 cacaggtgctggagtctcccagtctc |
| TRBV7-8_outer | SEQ ID NO: 440 gtgctggagtctcccagtcccctagg |
| TRBV7-9_outer | SEQ ID NO: 441 ctggagtctcccagaaccccagacaca |
| TRBV8-1_outer | SEQ ID NO: 442 gaggcagggatcagccagataccaagat |
| TRBV8-2_outer | SEQ ID NO: 443 gatgctgggatcacccagatgccaaga |
| TRBV9_outer | SEQ ID NO: 444 tggagtcacacaaaccccaaagcacctg |

Hybridization Probe Pool
Ig1 SEQ ID NO: 445
GAACTGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGT
Ig2 SEQ ID NO: 446
GGAAGGTGGATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGA
Ig3 SEQ ID NO: 447
CTCGCCCGTCACAAAGAGCTTCAACAGGGGAGAGTGTTAGAGGGAGAAGTGCCCCCACCTGCTCC
TCAGTTCCAGCCTGACCCCCTCCCATCCTTTGGCCTCTGACCCTTTTTCCACAGG
Ig4 SEQ ID NO: 448
GGACCTACCCCTATTGCGGTCCTCCAGCTCATCTTTCACCTCACCCCCCTCCTCCTCCTTGGCTTTA
ATTATGCTAATGTTGGAGGAGAATGAATAAATAAAGTGAATCTTTGCACCTGT
Ig5 SEQ ID NO: 449
GTCAGCCCAAGGCCAACCCCACTGTCACTCTGTTCCCGCCCTCCTCTGAGGAGCTCCAAGCCAACA
AGGCCACACTAGTGTGTCTGATCAGTGACTTCTACCCGGGAGCTGTGACAGTGG
Ig6 SEQ ID NO: 450
GACGCCCGAGCAGTGGAAGTCCCACAGAAGCTACAGCTGCCAGGTCACGCATGAAGGGAGCACC
GTGGAGAAGACAGTGGCCCCTACAGAATGTTCATAGGTTCCCAACTCTAACCCCAC
Ig7 SEQ ID NO: 451
CCACGGGAGCCTGGAGCTGCAGGATCCCAGGGGAGGGGTCTCTCTCCCCATCCCAAGTCATCCAG
CCCTTCTCCCTGCACTCATGAAACCCCAATAAATATCCTCATTGACAACCAGAAA
Ig8 SEQ ID NO: 452
GTCAGCCCAAGGCTGCCCCCTCGGTCACTCTGTTCCCGCCCTCCTCTGAGGAGCTTCAAGCCAACA
AGGCCACACTGGTGTGTCTCATAAGTGACTTCTACCCGGGAGCCGTGACAGTGG
Ig9 SEQ ID NO: 453
CGCCTGAGCAGTGGAAGTCCCACAGAAGCTACAGCTGCCAGGTCACGCATGAAGGGAGCACCGTG
GAGAAGACAGTGGCCCCTACAGAATGTTCATAGGTTCTCAACCCTCACCCCCCAC
Ig10 SEQ ID NO: 454
CACGGGAGACTAGAGCTGCAGGATCCCAGGGGAGGGGTCTCTCCTCCCACCCCAAGGCATCAAGC
CCTTCTCCCTGCACTCAATAAACCCTCAATAAATATTCTCATTGTCAATCAGAAA
Ig11 SEQ ID NO: 455
GTCAGCCCAAGGCTGCCCCCTCGGTCACTCTGTTCCCACCCTCCTCTGAGGAGCTTCAAGCCAACA
AGGCCACACTGGTGTGTCTCATAAGTGACTTCTACCCGGGAGCCGTGACAGTGG
Ig12 SEQ ID NO: 456
CGCCTGAGCAGTGGAAGTCCCACAAAAGCTACAGCTGCCAGGTCACGCATGAAGGGAGCACCGTG
GAGAAGACAGTGGCCCCTACAGAATGTTCATAGGTTCTCATCCCTCACCCCCCAC
Ig13 SEQ ID NO: 457
CACGGGAGACTAGAGCTGCAGGATCCCAGGGGAGGGGTCTCTCCTCCCACCCCAAGGCATCAAGC
CCTTCTCCCTGCACTCAATAAACCCTCAATAAATATTCTCATTGTCAATCAGAAA
Ig14 SEQ ID NO: 458
GTCAGCCCAAGGCTGCCCCCTCGGTCACTCTGTTCCCACCCTCCTCTGAGGAGCTTCAAGCCAACA
AGGCCACACTGGTGTGTCTCGTAAGTGACTTCAACCCGGGAGCCGTGACAGTGG
Ig15 SEQ ID NO: 459
CCAGCAGCTACCTGAGCCTGACGCCCGAGCAGTGGAAGTCCCACAGAAGCTACAGCTGCCGGGTC
ACGCATGAAGGGAGCACCGTGGAGAAGACAGTGGCCCCTGCAGAATGCTCTTAGG
Ig16 SEQ ID NO: 460
CCCCCGACCCTCACCCCACCCACAGGGGCCTGGAGCTGCAGGTTCCCAGGGGAGGGGTCTCTGCC
CCCATCCCAAGTCATCCAGCCCTTCTCAATAAATATCCTCATCGTCAACGAGAAA
Ig17 SEQ ID NO: 461
GCATCCCCGACCAGCCCCAAGGTCTTCCCGCTGAGCCTCGACAGCACCCCCCAAGATGGGAACGT
GGTCGTCGCATGCCTGGTCCAGGGCTTCTTCCCCCAGGAGCCACTCAGTGTGACC
Ig18 SEQ ID NO: 462
TGGAGCGAAAGCGGACAGAACGTGACCGCCAGAAACTTCCCACCTAGCCAGGATGCCTCCGGGGA
CCTGTACACCACGAGCAGCCAGCTGACCCTGCCGGCCACACAGTGCCCAGACGGC
Ig19 SEQ ID NO: 463
AAGTCCGTGACATGCCACGTGAAGCACTACACGAATTCCAGCCAGGATGTGACTGTGCCCTGCCG
AGTTCCCCCACCTCCCCCATGCTGCCACCCCCGACTGTCGCTGCACCGACCGGCC
Ig20 SEQ ID NO: 464
CTCGAGGACCTGCTCTTAGGTTCAGAAGCGAACCTCACGTGCACACTGACCGGCCTGAGAGATGC
CTCTGGTGCCACCTTCACCTGGACGCCCTCAAGTGGGAAGAGCGCTGTTCAAGGA
Ig21 SEQ ID NO: 465
CCACCTGAGCGTGACCTCTGTGGCTGCTACAGCGTGTCCAGTGTCCTGCCTGGCTGTGCCCAGCCA
TGGAACCATGGGGAGACCTTCACCTGCACTGCTGCCCACCCCGAGTTGAAGACC
Ig22 SEQ ID NO: 466
CCACTAACCGCCAACATCACAAAATCCGGAAACACATTCCGGCCCGAGGTCCACCTGCTGCCGCC
GCCGTCGGAGGAGCTGGCCCTGAACGAGCTGGTGACGCTGACGTGCCTGGCACGT
Ig23 SEQ ID NO: 467
GGCTTCAGCCCCAAGGATGTGCTGGTTCGCTGGCTGCAGGGGTCACAGGAGCTGCCCCGCGAGAA
GTACCTGACTTGGGCATCCCGGCAGGAGCCCAGCCAGGGCACCACCACCTACGCT
Ig24 SEQ ID NO: 468
GTAACCAGCATACTGCGCGTGGCAGCTGAGGACTGGAAGAAGGGGGAGACCTTCTCCTGCATGGT
GGGCCACGAGGCCCTGCCGCTGGCCTTCACACAGAAGACCATCGACCGCATGGCG
Ig25 SEQ ID NO: 469
GGCTCTTGCTGTGTTGCAGATTGGCAGATGCCGCCTCCCTATGTGGTGCTGGACTTGCCGCAGGAG
ACCCTGGAGGAGGAGACCCCCGGCGCCAACCTGTGGCCCACCACCATCACCTTC
Ig26 SEQ ID NO: 470
CTCACCCTCTTCCTGCTGAGCCTGTTCTATAGCACAGCACTGACCGTGACCAGCGTCCGGGGCCCA
TCTGGCAAGAGGGAGGGCCCCCAGTACTGAGCGGGAGCCGGCAAGGCACAGGGA
Ig27 SEQ ID NO: 471
GGAAGTGTGGAGGAACCTCTTGGAGAAGCCAGCTATGCTTGCCAGAACTCAGCCCTTTCAGACAT
CACCGACCCGCCCTTACTCACGTGGCTTCCAGGTGCAATAAAGTGGCCCCAAGGA
Ig28 SEQ ID NO: 472
GCCTCCACACAGAGCCCATCCGTCTTCCCCTTGACCCGCTGCTGCAAAAACATTCCCTCCAATGCC
ACCTCCGTGACTCTGGGCTGCCTGGCCACGGGCTACTTCCCGGAGCCGGTGATG
Ig29 SEQ ID NO: 473
GTGACCTGGGACACAGGCTCCCTCAACGGGACAACTATGACCTTACCAGCCACCACCCTCACGCTC
TCTGGTCACTATGCCACCATCAGCTTGCTGACCGTCTCGGGTGCGTGGGCCAAG
Ig30 SEQ ID NO: 474
CAGATGTTCACCTGCCGTGTGGCACACACTCCATCGTCCACAGACTGGGTCGACAACAAAACCTTC
AGCGTCTGCTCCAGGGACTTCACCCCGCCCACCGTGAAGATCTTACAGTCGTCC
Ig31 SEQ ID NO: 475
TGCGACGGCGGCGGGCACTTCCCCCCGACCATCCAGCTCCTGTGCCTCGTCTCTGGGTACACCCCA
GGGACTATCAACATCACCTGGCTGGAGGACGGGCAGGTCATGGACGTGGACTTG
Ig32 SEQ ID NO: 476
TCCACCGCCTCTACCACGCAGGAGGGTGAGCTGGCCTCCACACAAAGCGAGCTCACCCTCAGCCA
GAAGCACTGGCTGTCAGACCGCACCTACACCTGCCAGGTCACCTATCAAGGTCAC
Ig33 SEQ ID NO: 477
ACCTTTGAGGACAGCACCAAGAAGTGTGCAGATTCCAACCCGAGAGGGGTGAGCGCCTACCTAAG
CCGGCCCAGCCCGTTCGACCTGTTCATCCGCAAGTCGCCCACGATCACCTGTCTG
Ig34 SEQ ID NO: 478
TCCCGGGCCAGTGGGAAGCCTGTGAACCACTCCACCAGAAAGGAGGAGAAGCAGCGCAATGGCA
CGTTAACCGTCACGTCCACCCTGCCGGTGGGCACCCGAGACTGGATCGAGGGGGAG
Ig35 SEQ ID NO: 479
ACCTACCAGTGCAGGGTGACCCACCCCCACCTGCCCAGGGCCCTCATGCGGTCCACGACCAAGAC
CAGCGGCCCGCGTGCTGCCCCGGAAGTCTATGCGTTTGCGACGCCGGAGTGGCCG
Ig36 SEQ ID NO: 480
GGGAGCCGGGACAAGCGCACCCTCGCCTGCCTGATCCAGAACTTCATGCCTGAGGACATCTCGGT
GCAGTGGCTGCACAACGAGGTGCAGCTCCCGGACGCCCGGCACAGCACGACGCAG
Ig37 SEQ ID NO: 481
CCCCGCAAGACCAAGGGCTCCGGCTTCTTCGTCTTCAGCCGCCTGGAGGTGACCAGGGCCGAATG
GGAGCAGAAAGATGAGTTCATCTGCCGTGCAGTCCATGAGGCAGCAAGCCCCTCA
Ig38 SEQ ID NO: 482
CAGACCGTCCAGCGAGCGGTGTCTGTAAATCCCGAGCTGGACGTGTGCGTGGAGGAGGCCGAGGG
CGAGGCGCCGTGGACGTGGACCGGCCTCTGCATCTTCGCCGCACTCTTCCTGCTC
Ig39 SEQ ID NO: 483
AGCGTGAGCTACAGCGCCGCCATCACGCTCCTCATGGTGCAGCGGTTCCTCTCAGCCACGCGGCAG
GGGAGGCCCCAGACCTCCCTCGACTACACCAACGTCCTCCAGCCCCACGCCTAG
Ig40 SEQ ID NO: 484
TCCTGCCTCCCTCCCTCCCAGGGCTCCATCCAGCTGTGCAGTGGGGAGGACTGGCCAGACCTTCTG
TCCACTGTTGCAATGACCCCAGGAAGCTACCCCCAATAAACTGTGCCTGCTCAG
Ig41 SEQ ID NO: 485
GCTTCCACCAAGGGCCCATCGGTCTTCCCCCTGGCGCCCTGCTCCAGGAGCACCTCCGAGAGCACA
GCCGCCCTGGGCTGCCTGGTCAAGGACTACTTCCCCGAACCGGTGACGGTGTCG
Ig42 SEQ ID NO: 486
TGGAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACGAAGACC
Ig43 SEQ ID NO: 487
TACACCTGCAACGTAGATCACAAGCCCAGCAACACCAAGGTGGACAAGAGAGTTGAGTCCAAATA
TGGTCCCCCATGCCCATCATGCCCAGCACCTGAGTTCCTGGGGGGACCATCAGTC
Ig44 SEQ ID NO: 488
TTCCTGTTCCCCCCAAAACCCAAGGACACTCTCATGATCTCCCGGACCCCTGAGGTCACGTGCGTG
GTGGTGGACGTGAGCCAGGAAGACCCCGAGGTCCAGTTCAACTGGTACGTGGAT
Ig45 SEQ ID NO: 489
GGCGTGGAGGTGCATAATGCCAAGACAAAGCCGCGGGAGGAGCAGTTCAACAGCACGTACCGTGT
GGTCAGCGTCCTCACCGTCCTGCACCAGGACTGGCTGAACGGCAAGGAGTACAAG
Ig46 SEQ ID NO: 490
TGCAAGGTCTCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAG
Ig47 SEQ ID NO: 491
AACCAGGTCAGCCTGACCTGCCTGGTCAAAGGCTTCTACCCCAGCGACATCGCCGTGGAGTGGGA
GAGCAATGGGCAGCCGGAGAACAACTACAAGACCACGCCTCCCGTGCTGGACTCC
Ig48 SEQ ID NO: 492
GACGGCTCCTTCTTCCTCTACAGCAGGCTCACCGTGGACAAGAGCAGGTGGCAGGAGGGGAATGT
CTTCTCATGCTCCGTGATGCATGAGGCTCTGCACAACCACTACACACAGAAGAGC
Ig49 SEQ ID NO: 493
CTCTCCCTGTCTCTGGAGCTGCAACTGGAGGAGAGCTGTGCGGAGGCGCAGGACGGGGAGCTGGA
CGGGCTGTGGACGACCATCACCATCTTCATCACACTCTTCCTGCTAAGCGTGTGC
Ig50 SEQ ID NO: 494
TACAGTGCCACCGTCACCTTCTTCAAGGTGAAGTGGATCTTCTCCTCAGTGGTGGACCTGAAGCAG
ACCATCGTCCCCGACTACAGGAACATGATAAGGCAGGGGGCCTAGGGCCACCCT
Ig51 SEQ ID NO: 495
CCCCCTGACCTCACCGCCCTCAACCCCATGGCTCTCTGGCTTCGCAGTCGCCCTCTGAGCCCTGAA
ACGCCCCCCTTCCAGACCCTGTGCATAGCAGGTCTACCCCAGACCTCCGCTGCT
Ig52 SEQ ID NO: 496
TGGTGCATGCAGGGCGCTGAGGGCCAGGTGTCCCCTCAGCAGGACGTCCCTGCCCTCTGGACCACC
AGGTGCTCACACAAAAGGAGGTAACCGGCATCCCAGGCCCCCACTCAGGCAGGA
Ig53 SEQ ID NO: 497
CCTCGCCCTGGAGCCAACCCCGTCCACGCCAGCCTCCTGAACACAGGCATGGTTTCCAGATGGTGA
GTGGGAGCATCAGTCGCCAAGGTAGGGAAGCCACAGCACCATCAGGCCCTGTTG
Ig54 SEQ ID NO: 498
GGGAGGCTTCCGAGAGCTGCGAAGGCTCACTCAGACGGCCTTCCTCCCAGCCCGCAGCCAGCCAG
CCTCCATTCCGGGCACTCCCGTGAACTCCTGACATGAGGAATGAGGTTGTTCTGA
Ig55 SEQ ID NO: 499
TTTCAAGCAAAGAACGCTGCTCTCTGGCTCCTGGGAACAGTCTCGGTGCCAGCACCACCCCTTGGC
TGCCTGCCCACACTGCTGGATTCTCGGGTGGAACTGGACCCGCAGGGACAGCCA
Ig56 SEQ ID NO: 500
GCCCCAGAGTCCGCACTGGGGAGAGAAAGGGCCAGGCCCAGGACACTGCCACCTACCACCCACTC
CAGTCCACCGAGATCACTCGGAGAAGAGCCTGGGCCATGTGGCCGCTGCAGGAGC
Ig57 SEQ ID NO: 501
CCCACAGTGCAAGGGTGAGGATAGCCCAAGGAAGGGCTGGGCATCTGCCCAGACAGGCCTCCCAC
AGAAGGCTGGTGACCAGGTCCCAGGCGGGCAAGACTCAGCCTTGGTGGGGCCTGA
Ig58 SEQ ID NO: 502
GGACAGAGGAGGCCCAGGAGCATCGGGGAGAGAGGTGGAGGGACACCGGGAGAGCCAGGAGCG
TGGACACAGCCAGAACTCATCACAGAGGCTGGCGTCCAGTCCCGGGTCACGTGCAGC
Ig59 SEQ ID NO: 503
AGGAACAAGCAGCCACTCTGGGGGCACCAGGTGGAGAGGCAAGACGACAAAGAGGGTGCCCGTG
TTCTTGCGAAAGCGGGGCTGCTGGCCACGAGTGCTGGACAGAGGCCCCCACGCTCT
Ig60 SEQ ID NO: 504
GCTGCCCCCATCACACCGTTCCGTGACTGTCACGCAGAATCCACAGACAGGAAGGGAGGCTCGAG
CGGGACTGCGGCCAGCGCCTGCCTCGGCCGTCAGGGAGGACTCCCGGGCTCACTC
Ig61 SEQ ID NO: 505
GAAGGAGGTGTCACCATTTCAGCTTTGGCTTTTCTTCTTCTTTTAAATTTTCTAAAGCTCATTAATTG
TCTTTGATGTTTCTTTTGTGATGACAATAAAATATCCTTTTTAAGTCTTGTA
Ig62 SEQ ID NO: 506
AGCCCCCGCTCCCCGGGCTCTCGGGGTCGCGCGAGGATGCTTGGCACGTACCCCGTGTACATACTT
CCCGGGCGCCCAGCATGGAAATAAAGCACCCAGCGCTGCCCTGGGCCCCTGCGA
Ig63 SEQ ID NO: 507
GCCTCCACCAAGGGCCCATCGGTCTTCCCCCTGGCGCCCTGCTCCAGGAGCACCTCCGAGAGCACA
GCGGCCCTGGGCTGCCTGGTCAAGGACTACTTCCCCGAACCGGTGACGGTGTCG
Ig64 SEQ ID NO: 508
TGGAACTCAGGCGCTCTGACCAGCGGCGTGCACACCTTCCCGGCTGTCCTACAGTCCTCAGGACTC
TACTCCCTCAGCAGCGTGGTGACCGTGCCCTCCAGCAACTTCGGCACCCAGACC
Ig65 SEQ ID NO: 509
TACACCTGCAACGTAGATCACAAGCCCAGCAACACCAAGGTGGACAAGACAGTTGAGCGCAAATG
TTGTGTCGAGTGCCCACCGTGCCCAGCACCACCTGTGGCAGGACCGTCAGTCTTC
Ig66 SEQ ID NO: 510
CTCTTCCCCCCAAAACCCAAGGACACCCTCATGATCTCCCGGACCCCTGAGGTCACGTGCGTGGTG
GTGGACGTGAGCCACGAAGACCCCGAGGTCCAGTTCAACTGGTACGTGGACGGC
Ig67 SEQ ID NO: 511
GTGGAGGTGCATAATGCCAAGACAAAGCCACGGGAGGAGCAGTTCAACAGCACGTTCCGTGTGGT
CAGCGTCCTCACCGTCGTGCACCAGGACTGGCTGAACGGCAAGGAGTACAAGTGC
Ig68 SEQ ID NO: 512
AAGGTCTCCAACAAAGGCCTCCCAGCCCCCATCGAGAAAACCATCTCCAAAACCAAAGGGCAGCC
CCGAGAACCACAGGTGTACACCCTGCCCCCATCCCGGGAGGAGATGACCAAGAAC
Ig69 SEQ ID NO: 513
CAGGTCAGCCTGACCTGCCTGGTCAAAGGCTTCTACCCCAGCGACATCTCCGTGGAGTGGGAGAG
CAATGGGCAGCCGGAGAACAACTACAAGACCACACCTCCCATGCTGGACTCCGAC
Ig70 SEQ ID NO: 514
GGCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACACAGAAGAGCCTC
Ig71 SEQ ID NO: 515
TCCCTGTCTCCGGAGCTGCAACTGGAGGAGAGCTGTGCGGAGGCGCAGGACGGGGAGCTGGACGG
GCTGTGGACCACCATCACCATCTTCATCACACTCTTCCTGCTAAGCGTGTGCTAC
Ig72 SEQ ID NO: 516
AGTGCCACCATCACCTTCTTCAAGGTGAAGTGGATCTTCTCCTCAGTGGTGGACCTGAAGCAGACC
ATCGTCCCCGACTACAGGAACATGATCAGGCAGGGGGCCTAGGGCCACCCTCTG
Ig73 SEQ ID NO: 517
CCCCCGACCTCACCGCCCTCAACCCCATGGCTCTCTGGCCTCGCAGTCGCCCTCTGACCCTGACAC
GCCCCCCTTCCAGACCCTGTGCATAGCAGGTCTACCCCAGACCTCCGCTGCTTG
Ig74 SEQ ID NO: 518
GTGCATGCAGGGCGCTGGGGGCCAAGTGTCCCCTCAGCAGGACGTCCCTGCCCTCCGGCCCGCCA
GGTGCTCACACAAAAGGAGGTAGTGACCAGCATCCCAGGCCCCCACTCAGGCAGG
Ig75 SEQ ID NO: 519
ACCTCGCCCTGGAGCCAACCCTGTCCACGCCAGCCTCCTGAACACAGGCGTGGTTTCCAGATGGTG
AGTGGGAGCATCAGTCGCCAAGGTAGGGAAGTCACAGCACCATCAGGCCCTGTT
Ig76 SEQ ID NO: 520
GGGGAGGCTTCCGAGAGCTGCGAAGGCTCACTCAGACGGCCTTCCTCCCAGCCCGCAGCCAGCCA
GCCTCCATTCCAGGCACTCCCGTGAACTCCTGACATGAGGAATGAGGTTGTTCTG
Ig77 SEQ ID NO: 521
ATTTCAAGCAAAGAACGCTGCTCTCTGGCTCCTGGGAACAGTCTCAGTGCCAGCACCACCCCTTGG
CTGCCTGCCCACACTGCTGGATTCTCGGGTGGAACTCGACCCGCAGGGACAGCC
Ig78 SEQ ID NO: 522
AGCCCCAGAGTCCGCACTGGGGAGAGAAGGGGCCAGGCCCAGGACACTGCCACCTACCACCCACT
CCAGTCCACCGAGATCACTCGGAGAAGAGCCTGGGCCATGTGGCCGCTGCAGGAG
Ig79 SEQ ID NO: 523
CCCCACGGTGCAAGGGTGAGGATAGCCCAAGGAAGGGCTGGGCATCTGCCCAGACAGGCCTCCCA
GAGAAGGCTGGTGACCAGGTCCCAGGCGGGCAAGACTCAGCCTTGGTGGGGCCTG
Ig80 SEQ ID NO: 524
AGGACAGAGGAGGCCCAGGAGCATCGGGGAGAGAGGTGGAGGGACACCGGGAGAGCCAGGAGC
GTGGACACAGCCAGAACTCATCACAGAGGCTGGCGTCCAGCCCCGGGTCACGTGCAG
Ig81 SEQ ID NO: 525
CAGGAACAAGCAGCCACTCTGGGGGCACCAGGTGGAGAGGCAAGACGACAAAGAGGGTGCCCGT
GTTCTTGTGAAAGCGGGGCTGCTGGCCACGAGTGCTGGACAGAGGCCCCCACGCTC
Ig82 SEQ ID NO: 526
TGCTGCCCCCATCACGCCGTTCCGTGACTGTCACGCAGAATCCGCAGACAGGGAGACTCGAGCGG
GAGTGCGGCCAGCGCCTGCCTCAGCTGTCAGGGAGGACTCCCGGGCTCACTCGAA
Ig83 SEQ ID NO: 527
GGAGGTGCCACCATTTCAGCTTTGGTAGCTTTTCTTCTTCTTTTAAATTTTCTAAAGCTCATTAATTG
TCTTTGATGTTTCTTTTGTGATGACAATAAAATATCCTTTTTAAGTCTTGTA
Ig84 SEQ ID NO: 528
AGCCCCCGCTCCCCAGGCTCTCGGGGTCGCGCGAGGATGCTTGGCACGTACCCCGTCTACATACTT
CCCGGGCACCCAGCATGGAAATAAAGCACCCAGCGCTGCCCTGGGCCCCTGCGA
Ig85 SEQ ID NO: 529
GCATCCCCGACCAGCCCCAAGGTCTTCCCGCTGAGCCTCTGCAGCACCCAGCCAGATGGGAACGT
GGTCATCGCCTGCCTGGTCCAGGGCTTCTTCCCCCAGGAGCCACTCAGTGTGACC
Ig86 SEQ ID NO: 530
TGGAGCGAAAGCGGACAGGGCGTGACCGCCAGAAACTTCCCACCCAGCCAGGATGCCTCCGGGGA
CCTGTACACCACGAGCAGCCAGCTGACCCTGCCGGCCACACAGTGCCTAGCCGGC
Ig87 SEQ ID NO: 531
AAGTCCGTGACATGCCACGTGAAGCACTACACGAATCCCAGCCAGGATGTGACTGTGCCCTGCCC
AGTTCCCTCAACTCCACCTACCCCATCTCCCTCAACTCCACCTACCCCATCTCCC
Ig88 SEQ ID NO: 532
TCATGCTGCCACCCCCGACTGTCACTGCACCGACCGGCCCTCGAGGACCTGCTCTTAGGTTCAGAA
GCGAACCTCACGTGCACACTGACCGGCCTGAGAGATGCCTCAGGTGTCACCTTC
Ig89 SEQ ID NO: 533
ACCTGGACGCCCTCAAGTGGGAAGAGCGCTGTTCAAGGACCACCTGAGCGTGACCTCTGTGGCTG
CTACAGCGTGTCCAGTGTCCTGCCGGGCTGTGCCGAGCCATGGAACCATGGGAAG
Ig90 SEQ ID NO: 534
GGAGGAGCTGGCCCTGAACGAGCTGGTGACGCTGACGTGCCTGGCACGCGGCTTCAGCCCCAAGG
ATGTGCTGGTTCGCTGGCTGCAGGGGTCACAGGAGCTGCCCCGCGAGAAGTACCT
Ig91 SEQ ID NO: 535
GACTTGGGCATCCCGGCAGGAGCCCAGCCAGGGCACCACCACCTTCGCTGTGACCAGCATACTGC
GCGTGGCAGCCGAGGACTGGAAGAAGGGGGACACCTTCTCCTGCATGGTGGGCCA
Ig92 SEQ ID NO: 536
CGAGGCCCTGCCGCTGGCCTTCACACAGAAGACCATCGACCGCTTGGCGGATTGGCAGATGCCGC
CTCCCTATGTGGTGCTGGACTTGCCGCAGGAGACCCTGGAGGAGGAGACCCCCGG
Ig93 SEQ ID NO: 537
CGCCAACCTGTGGCCCACCACCATCACCTTCCTCACCCTCTTCCTGCTGAGCCTGTTCTATAGCACA
GCACTGACCGTGACCAGCGTCCGGGGCCCATCTGGCAACAGGGAGGGCCCCCA
Ig94 SEQ ID NO: 538
GTACTGAGCAGGAGCCGGCAAGGCACAGGGAGGAAGTGTGGAGGAACCTCTTGGAGAAGCCAGC
TATGCTTGCCAGAACTCAGCCCTTTCAGACATCACCGACCCGCCCTTACTCACATG
Ig95 SEQ ID NO: 539
CTTGGCGGGTAAACCCACCCATGTCAATGTGTCTGTTGTCATGGCGGAGGTGGACGGCACCTGCTA
CTGAGCCGCCCGCCTGTCCCCACCCCTGAATAAACTCCATGCTCCCCCAAGCAG
Ig96 SEQ ID NO: 540
GCCTCCACCAAGGGCCCATCGGTCTTCCCCCTGGCACCCTCCTCCAAGAGCACCTCTGGGGGCACA
GCAGCCCTGGGCTGCCTGGTCAAGGACTACTTCCCCGAACCGGTGACGGTGTCG
Ig97 SEQ ID NO: 541
TGGAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACC
Ig98 SEQ ID NO: 542
TACATCTGCAACGTGAATCACAAGCCCAGCAACACCAAGGTGGACAAGAAAGTTGAGCCCAAATC
TTGTGACAAAACTCACACATGCCCACCGTGCCCAGCACCTGAACTCCTGGGGGGA
Ig99 SEQ ID NO: 543
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGG
Ig100 SEQ ID NO: 544
TACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAG
Ig101 SEQ ID NO: 545
GAGTACAAGTGCAAGGTCTCCAACAAAGCCCTCCCAGCCCCCATCGAGAAAACCATCTCCAAAGC
CAAAGGGCAGCCCCGAGAACCACAGGTGTACACCCTGCCCCCATCCCGGGATGAG
Ig102 SEQ ID NO: 546
CTGACCAAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTG
Ig103 SEQ ID NO: 547
CTGGACTCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACA
Ig104 SEQ ID NO: 548
CAGAAGAGCCTCTCCCTGTCTCCGGAGCTGCAACTGGAGGAGAGCTGTGCGGAGGCGCAGGACGG
GGAGCTGGACGGGCTGTGGACGACCATCACCATCTTCATCACACTCTTCCTGTTA
Ig105 SEQ ID NO: 549
AGCGTGTGCTACAGTGCCACCGTCACCTTCTTCAAGGTGAAGTGGATCTTCTCCTCGGTGGTGGAC
CTGAAGCAGACCATCATCCCCGACTACAGGAACATGATCGGACAGGGGGCCTAG
Ig106 SEQ ID NO: 550
CGCCCTCAACCCCATGACTCTCTGGCCTCGCAGTTGCCCTCTGACCCTGACACACCTGACACGCCC
CCCTTCCAGACCCTGTGCATAGCAGGTCTACCCCAGACCTCCGCTGCTTGGTGC
Ig107 SEQ ID NO: 551
ATGCAGGGCACTGGGGGCCAGGTGTCCCCTCAGCAGGACGTCCTTGCCCTCCGGACCACAAGGTG
CTCACACAAAAGGAGGCAGTGACCGGTATCCCAGGCCCCCACCCAGGCAGGACCT
Ig108 SEQ ID NO: 552
CGCCCTGGAGCCAACCCCGTCCACGCCAGCCTCCTGAACACAGGCGTGGTTTCCAGATGGTGAGTG
GGAGCGTCAGCCGCCAAGGTAGGGAAGCCACAGCACCATCAGGCCCTGTTGGGG
Ig109 SEQ ID NO: 553
AGGCTTCCGAGAGCTGCGAAGGCTCACTCAGACGGCCTTCCTCCCAGCCCGCAGCCAGCCAGCCTC
CATTCCGGGCACTCCCGTGAACTCCTGACATGAGGAATGAGGTTGTTCTGATTT
Ig110 SEQ ID NO: 554
CAAGCAAAGAACGCTGCTCTCTGGCTCCTGGGAACAGTCTCAGTGCCAGCACCACCCCTTGGCTGC
CTGCCCACACTGCTGGATTCTCGGGTGGAACTGGACCCGCAGGGACAGCCAGCC
Ig111 SEQ ID NO: 555
CCAGAGTCCGCACTGGGGAGAGAAGGGGCCAGGCCCAGGACACTGCCACCTCCCACCCACTCCAG
TCCACCGAGATCACTCAGAGAAGAGCCTGGGCCATGTGGCCGCTGCAGGAGCCCC
Ig112 SEQ ID NO: 556
ACAGTGCAAGGGTGAGGATAGCCCAAGGAAGGGCTGGGCATCTGCCCAGACAGGCCTCCCAGAG
AAGGCTGGTGACCAGGTCCCAGGCGGGCAAGACTCAGCCTTGGTGGGGCCTGAGGA
Ig113 SEQ ID NO: 557
CAGAGGAGGCCCAGGAGCATCGGGGAGAGAGGTGGAGGGACACCGGGAGAGCCAGGAGCGTGG
ACACAGCCAGAACTCATCACAGAGGCTGGCGTCCAGCCCCGGGTCACGTGCAGCAGG
Ig114 SEQ ID NO: 558
AACAAGCAGCCACTCTGGGGGCACCAGGTGGAGAGGCAAGACGACAAAGAGGGTGCCCGTGTTC
TTGCGAAAGCAGGGCTGCTGGCCACGAGTGCTGGACAGAGGCCCCCACGCTCTGCT
Ig115 SEQ ID NO: 559
GCCCCCATCACGCCGTTCCGTGACTGTCACGCAGAATCTGCAGACAGGAAGGGAGACTCGAGCGG
GAGTGCGGCCAGCGCCTGCCTCGGCCGTCAGGGAGGACTCCTGGGCTCACTCGAA
Ig116 SEQ ID NO: 560
GGAGGTGCCACCATTTCAGCTTTGGTAGCTTTTCTTCTTCTTTTAAATTTTCTAAAGCTCATTAATTG
TCTTTGATGTTTCTTTTGTGATGACAATAAAATATCCTTTTTAAGTCTTGTA
Ig117 SEQ ID NO: 561
AAGCCCCCGCTCCCCAGGCTCTCGGGGTCGCGCGAGGATGCTTGGCACGTACCCCGTGTACATACT
TCCCAGGCACCCAGCATGGAAATAAAGCACCCAGCGCTTCCCTGGGCCCCTGCG
Ig118 SEQ ID NO: 562
CTTCCACCAAGGGCCCATCGGTCTTCCCCCTGGCGCCCTGCTCCAGGAGCACCTCTGGGGGCACAG
CGGCCCTGGGCTGCCTGGTCAAGGACTACTTCCCAGAACCGGTGACGGTGTCGT
Ig119 SEQ ID NO: 563
GGAACTCAGGCGCCCTGACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAGACCT
Ig120 SEQ ID NO: 564
ACACCTGCAACGTGAATCACAAGCCCAGCAACACCAAGGTGGACAAGAGAGTTGAGCTCAAAACC
CCACTTGGTGACACAACTCACACATGCCCACGGTGCCCAGAGCCCAAATCTTGTG
Ig121 SEQ ID NO: 565
ACACACCTCCCCCGTGCCCACGGTGCCCAGAGCCCAAATCTTGTGACACACCTCCCCCATGCCCAC
GGTGCCCAGAGCCCAAATCTTGTGACACACCTCCCCCGTGCCCAAGGTGCCCAG
Ig122 SEQ ID NO: 566
CACCTGAACTCCTGGGAGGACCGTCAGTCTTCCTCTTCCCCCCAAAACCCAAGGATACCCTTATGA
TTTCCCGGACCCCTGAGGTCACGTGCGTGGTGGTGGACGTGAGCCACGAAGACC
Ig123 SEQ ID NO: 567
CCGAGGTCCAGTTCAAGTGGTACGTGGACGGCGTGGAGGTGCATAATGCCAAGACAAAGCCGCGG
GAGGAGCAGTACAACAGCACGTTCCGTGTGGTCAGCGTCCTCACCGTCCTGCACC
Ig124 SEQ ID NO: 568
AGGACTGGCTGAACGGCAAGGAGTACAAGTGCAAGGTCTCCAACAAAGCCCTCCCAGCCCCCATC
GAGAAAACCATCTCCAAAACCAAAGGACAGCCCCGAGAACCACAGGTGTACACCC
Ig125 SEQ ID NO: 569
TGCCCCCATCCCGGGAGGAGATGACCAAGAACCAGGTCAGCCTGACCTGCCTGGTCAAAGGCTTC
TACCCCAGCGACATCGCCGTGGAGTGGGAGAGCAGCGGGCAGCCGGAGAACAACT
Ig126 SEQ ID NO: 570
ACAACACCACGCCTCCCATGCTGGACTCCGACGGCTCCTTCTTCCTCTACAGCAAGCTCACCGTGG
ACAAGAGCAGGTGGCAGCAGGGGAACATCTTCTCATGCTCCGTGATGCATGAGG
Ig127 SEQ ID NO: 571
CTCTGCACAACCGCTTCACGCAGAAGAGCCTCTCCCTGTCTCCGGAGCTGCAACTGGAGGAGAGCT
GTGCGGAGGCGCAGGACGGGGAGCTGGACGGGCTGTGGACGACCATCACCATCT
Ig128 SEQ ID NO: 572
TCATCACACTCTTCCTGTTAAGCGTGTGCTACAGTGCCACCGTCACCTTCTTCAAGGTGAAGTGGAT
CTTCTCCTCGGTGGTGGACCTGAAGCAGACCATCATCCCCGACTATAGGAACA
Ig129 SEQ ID NO: 573
GACCTCACCGCCCTCAACCCCATGGCTCTCTGTCTTTGCAGTCGCCCTCTGAGCCCTGACACGCCCC
CCTTCCAGACCCTGTGCATAGCAGGTCTACCCCAGACCTCCGCTGCTTGGTGC
Ig130 SEQ ID NO: 574
ATGCAGGGAGCTGGGGACCAGGTGTCCCCTCAGCAGGATGTCCCTGCCCTCCAGACCGCCAGATG
CTCACACAAAAGGAGGCAGTGACCAGCATCCGAGGCCCCCACCCAGGCAGGAGCT
Ig131 SEQ ID NO: 575
GGCCCTGGAGCCAACCCCGTCCACGCCAGCCTCCTGAACACAGGCGTGGTTTCCAGATGGTGAGT
GGGAGCATCAGCCGCCAAGGTAGGGAAGCCACAGCACCATCAGGCCCTGTTGGGG
Ig132 SEQ ID NO: 576
AGGCTTCCGAGAGCTGCGAAGGCTCACTCAGACGGCCTTCCTCCCAGCCCGCAGCCAGCCAGCCTC
CATTCCGGGCACTCCCGTGAACTCCTGACATGAGGAATGAGGTTGTTCTGATTT
Ig133 SEQ ID NO: 577
CAAGCAAAGAACGCTGCTCTCTGGCTCCTGGGAACAGTCTCGGTGCCAGCACCACCCCTTGGCTGC
CTGCCTACACTGCTGGATTCTCGGGTGGAACTGGACCCGCAGGGACAGCCAGCC
Ig134 SEQ ID NO: 578
CCAGAGTCCGCACTGGGGAGAGAAGGGGCCAGGCCCAGGACACTGCCACCTCCCACCCACTCCAG
TCCACCGAGATCACTCAGAGAAGAGCCTGGGCCATGTGGCCACTGCAGGAGCCCC
Ig135 SEQ ID NO: 579
ACAGTGCAAGAGTGAGGATAGCCCAAGGAAGGGCTGGGCATCTGCCCAGACAGGCCTCCCAGAG
AAGGCTGGTGACCAGGTCCCAGGCGGGCAAGACTCAGCCTTGGTGGGGCCTGAGGA
Ig136 SEQ ID NO: 580
CAGAGGAGGCCCAGGAGCATCGGGGAGAGAGGTGGAGGGACACCGGGAGAGCCAGGAGCGTGG
ACACAGCCAGAACTCATCACAGAGGCTGGCGTCCAGCCCCGGGTCACGTGCAGCAGG
Ig137 SEQ ID NO: 581
AACAAGCAGCCACTCTGGGGGCACCAGGTGGAGAGGCAAGATGCCAAAGAGGGTGCCCGTGTTCT
TGCGAAAGCGGGGCTGCTGGCCACGAGTGCTGGACAGAGGCCCCCACGCTCTGCT
Ig138 SEQ ID NO: 582
GCCCCCATCACGCCGTTCCGTGACTGTCACGCAGAATCCGCAGACAGGAAGGGAGGCTCGAGCGG
GACTGCGGCCAGCGCCTGCCTCGGCCGTCAGGGAGGACTCCCGGGCTCACTCGAA
Ig139 SEQ ID NO: 583
GGAGGTGCCACCATTTCAGCTTTGGTAGCTTTTCTTCTTCTTTTAAATTTTCTAAAGCTCATTAATTG
TCTTTGATGTTTCTTTTGTGATGACAATAAAATATCCTTTTTAAGTCTTGTA
Ig140 SEQ ID NO: 584
AGCCCCCGCTCCCCGGGCTCTCGGGGTCGCGCGAGGATGCTTGGCACGTACCCCGTGTACATACTT
CCCGGGCACCCAGCATGGAAATAAAGCACCCAGCGCTGCCCTGGGCCCCTGCGA
Ig141 SEQ ID NO: 585
CACCCACCAAGGCTCCGGATGTGTTCCCCATCATATCAGGGTGCAGACACCCAAAGGATAACAGC
CCTGTGGTCCTGGCATGCTTGATAACTGGGTACCACCCAACGTCCGTGACTGTCA
Ig142 SEQ ID NO: 586
CCTGGTACATGGGGACACAGAGCCAGCCCCAGAGAACCTTCCCTGAGATACAAAGACGGGACAGC
TACTACATGACAAGCAGCCAGCTCTCCACCCCCCTCCAGCAGTGGCGCCAAGGCG
Ig143 SEQ ID NO: 587
AGTACAAATGCGTGGTCCAGCACACCGCCAGCAAGAGTAAGAAGGAGATCTTCCGCTGGCCAGAG
TCTCCAAAGGCACAGGCCTCCTCAGTGCCCACTGCACAACCCCAAGCAGAGGGCA
Ig144 SEQ ID NO: 588
GCCTCGCCAAGGCAACCACAGCCCCAGCCACCACCCGTAACACAGGAAGAGGAGGAGAAGAGAA
GAAGAAGGAGAAGGAGAAAGAGGAACAAGAAGAGAGAGAGACAAAGACACCAGAGT
Ig145 SEQ ID NO: 589
GTCCGAGCCACACCCAGCCTCTTGGCGTCTACCTGCTAACCCCTGCAGTGCAGGACCTGTGGCTCC
GGGACAAAGCCACCTTCACCTGCTTCGTGGTGGGCAGTGACCTGAAGGATGCTC
Ig146 SEQ ID NO: 590
ACCTGACCTGGGAGGTGGCTGGGAAGGTCCCCACAGGGGGCGTGGAGGAAGGGCTGCTGGAGCG
GCACAGCAACGGCTCCCAGAGCCAGCACAGCCGTCTGACCCTGCCCAGGTCCTTGT
Ig147 SEQ ID NO: 591
GGCCTCGTCTGACCCTCCCGAGGCGGCCTCGTGGCTCCTGTGTGAGGTGTCTGGCTTCTCGCCCCCC
AACATCCTCCTGATGTGGCTGGAGGACCAGCGTGAGGTGAACACTTCTGGGTT
Ig148 SEQ ID NO: 592
TGCCCCCGCACGCCCCCCTCCACAGCCCAGGAGCACCACGTTCTGGGCCTGGAGTGTGCTGCGTGT
CCCAGCCCCGCCCAGCCCTCAGCCAGCCACCTACACGTGTGTGGTCAGCCACGA
Ig149 SEQ ID NO: 593
GGACTCCCGGACTCTGCTCAACGCCAGCCGGAGCCTAGAAGTCAGCTACCTGGCCATGACCCCCCT
GATCCCTCAGAGCAAGGATGAGAACAGCGATGACTACACGACCTTTGATGATGT
Ig150 SEQ ID NO: 594
GGGCAGCCTGTGGACCACCCTGTCCACGTTTGTGGCCCTCTTCATCCTCACCCTCCTCTACAGCGGC
ATTGTCACTTTCATCAAGGTGAAGTAGCCCCAGAAGAGCAGGACGCCCTGTAC
Ig151 SEQ ID NO: 595
CTGCAGAGAAGGGAAGCAGCCTCTGTACCTCATCTGTGGCTACCAGAGAGCAGAAAGGACCCACC
CTGGACTCTTCTGTGTGCAGGAAGATGCGCCAGCCCCTGCCCCCGGCTCCCCTCT
Ig152 SEQ ID NO: 596
GTCCGCCACAGAACCCAGTCTTCTAGACCAGGGGGACGGGCACCCATCACTCCGCAGGCGAATCA
GAGCCCCCCTGCCCCGGCCCTAACCCCTGTGCCTCCTTCCCATGCTTCCCCGAGA
Ig153 SEQ ID NO: 597
GCCAGCTACACCCCTGCCCCGGCCCTAACCCCCATGCCTCCTTCCTGTGCTTCCCCCAGAGCCAGCT
AGTCCCACCTGCAGCCCGCTGGCCTCCCCATAAACACACTTTGGTTCATTTCA
Ig154 SEQ ID NO: 598
GGGAGTGCATCCGCCCCAACCCTTTTCCCCCTCGTCTCCTGTGAGAATTCCCCGTCGGATACGAGC
AGCGTGGCCGTTGGCTGCCTCGCACAGGACTTCCTTCCCGACTCCATCACTTTC
Ig155 SEQ ID NO: 599
TCCTGGAAATACAAGAACAACTCTGACATCAGCAGCACCCGGGGCTTCCCATCAGTCCTGAGAGG
GGGCAAGTACGCAGCCACCTCACAGGTGCTGCTGCCTTCCAAGGACGTCATGCAG
Ig156 SEQ ID NO: 600
GGCACAGACGAACACGTGGTGTGCAAAGTCCAGCACCCCAACGGCAACAAAGAAAAGAACGTGC
CTCTTCCAGTGATTGCTGAGCTGCCTCCCAAAGTGAGCGTCTTCGTCCCACCCCGC
Ig157 SEQ ID NO: 601
GACGGCTTCTTCGGCAACCCCCGCAAGTCCAAGCTCATCTGCCAGGCCACGGGTTTCAGTCCCCGG
CAGATTCAGGTGTCCTGGCTGCGCGAGGGGAAGCAGGTGGGGTCTGGCGTCACC
Ig158 SEQ ID NO: 602
ACGGACCAGGTGCAGGCTGAGGCCAAAGAGTCTGGGCCCACGACCTACAAGGTGACCAGCACACT
GACCATCAAAGAGAGCGACTGGCTCGGCCAGAGCATGTTCACCTGCCGCGTGGAT
Ig159 SEQ ID NO: 603
CACAGGGGCCTGACCTTCCAGCAGAATGCGTCCTCCATGTGTGTCCCCGATCAAGACACAGCCATC
CGGGTCTTCGCCATCCCCCCATCCTTTGCCAGCATCTTCCTCACCAAGTCCACC
Ig160 SEQ ID NO: 604
AAGTTGACCTGCCTGGTCACAGACCTGACCACCTATGACAGCGTGACCATCTCCTGGACCCGCCAG
AATGGCGAAGCTGTGAAAACCCACACCAACATCTCCGAGAGCCACCCCAATGCC
Ig161 SEQ ID NO: 605
AGCGCCGTGGGTGAGGCCAGCATCTGCGAGGATGACTGGAATTCCGGGGAGAGGTTCACGTGCAC
CGTGACCCACACAGACCTGCCCTCGCCACTGAAGCAGACCATCTCCCGGCCCAAG
Ig162 SEQ ID NO: 606
GGGGTGGCCCTGCACAGGCCCGATGTCTACTTGCTGCCACCAGCCCGGGAGCAGCTGAACCTGCG
GGAGTCGGCCACCATCACGTGCCTGGTGACGGGCTTCTCTCCCGCGGACGTCTTC
Ig163 SEQ ID NO: 607
GTGCAGTGGATGCAGAGGGGGCAGCCCTTGTCCCCGGAGAAGTATGTGACCAGCGCCCCAATGCC
TGAGCCCCAGGCCCCAGGCCGGTACTTCGCCCACAGCATCCTGACCGTGTCCGAA
Ig164 SEQ ID NO: 608
GAGGAATGGAACACGGGGGAGACCTACACCTGCGTGGTGGCCCATGAGGCCCTGCCCAACAGGGT
CACCGAGAGGACCGTGGACAAGTCCACCGAGGGGGAGGTGAGCGCCGACGAGGAG
Ig165 SEQ ID NO: 609
GGCTTTGAGAACCTGTGGGCCACCGCCTCCACCTTCATCGTCCTCTTCCTCCTGAGCCTCTTCTACA
GTACCACCGTCACCTTGTTCAAGGTGAAATGATCCCAACAGAAGAACATCGGA
Ig166 SEQ ID NO: 610
GACCAGAGAGAGGAACTCAAAGGGGCGCTGCCTCCGGGTCTGGGGTCCTGGCCTGCGTGGCCTGT
TGGCACGTGTTTCTCTTCCCCGCCCGGCCTCCAGTTGTGTGCTCTCACACAGGCT
Ig167 SEQ ID NO: 611
TCCTTCTCGACCGGCAGGGGCTGGCTGGCTTGCAGGCCACGAGGTGGGCTCTACCCCACACTGCTT
TGCTGTGTATACGCTTGTTGCCCTGAAATAAATATGCACATTTTATCCATGAAA
Ig168 SEQ ID NO: 612
TGCTGGCCTGCCCACAGGCTCGGGGCGGCTGGCCGCTCTGTGTGTGCATGCAAACTAACCGTGTCA
ACGGGGTGAGATGTTGCATCTTATAAAATTAGAAATAAAAAGATCCATTCAAAA
Ig169 SEQ ID NO: 613
GCCACCCCCTTGGTCACTCTGTTCCCGCCCTCCTCTGAGGAGCTCCAAGCCAACAAGGCCATGCTG
GTGTGTCTCATAAATGACTTCTACCCAGGAGCCATAGAAGGAAAATGGCACCCT
Ig170 SEQ ID NO: 614
ATGCGGCCAGCAGCTACCTGAGCCTGACGCCCGAGCAGTGGAAGTCCCACAGAAGCTACAGCTGC
CAGGTCACGCACAAAGAAAGTACCATGGAGAAGACAATGGCCCATGCAGAATGTT
Ig171 SEQ ID NO: 615
ACAAGGCCACACTGGTGTGTCTCATGAGTGACTTCTACCCGAGAGCCATGACAGTGGCCTGGAAG
ATAGATGGCATCACCATCACCCAGGGTGTGGAGACCACCACACCCTCCAAACAGA
Ig172 SEQ ID NO: 616
TATGCGGCCAGCAGCTACCTAAGACTGGCACCCGACAGTGGAAGTCCCACAACCTCTACAGCTGC
CAGGTCACGCATGAAAGGAACACTGTGGAGAAGACAGTGGCCCCTGCAGAATGTT
Ig173 SEQ ID NO: 617
GTCAGCCCAAGGCTGCCCCATCGGTCACTCTGTTCCCGCCCTCCTCTGAGGAGCTTCAAGCCAACA
AGGCCACACTGGTGTGCCTGATCAGTGACTTCTACCCGGGAGCTGTGAAAGTGG
Ig174 SEQ ID NO: 618
GCGGCCAGCAGCTAGCTACCTGAGCCTGACGCCTGAGCAGTGGAAGTCCCACAGAAGCTACAGTT
GCCAGGTCACGCATGAAGGGAGCACCGTGGAGAAGACAGTGGCCCCTGCAGAATG
TCR1 SEQ ID NO: 619
AGGACCTGAACAAGGTGTTCCCACCCGAGGTCGCTGTGTTTGAGCCATCAGAAGCAGAGATCTCC
CACACCCAAAAGGCCACACTGGTGTGCCTGGCCACAGGCTTCTTCCCTGACCACG
TCR2 SEQ ID NO: 620
TGGAGCTGAGCTGGTGGGTGAATGGGAAGGAGGTGCACAGTGGGGTCAGCACGGACCCGCAGCC
CCTCAAGGAGCAGCCCGCCCTCAATGACTCCAGATACTGCCTGAGCAGCCGCCTGA
TCR3 SEQ ID NO: 621
GGGTCTCGGCCACCTTCTGGCAGAACCCCCGCAACCACTTCCGCTGTCAAGTCCAGTTCTACGGGC
TCTCGGAGAATGACGAGTGGACCCAGGATAGGGCCAAACCCGTCACCCAGATCG
TCR4 SEQ ID NO: 622
CTCGGTGTCCTACCAGCAAGGGGTCCTGTCTGCCACCATCCTCTATGAGATCCTGCTAGGGAAGGC
CACCCTGTATGCTGTGCTGGTCAGCGCCCTTGTGTTGATGGCCATGGTCAAGAG
TCR5 SEQ ID NO: 623
AAAGGATTTCTGAAGGCAGCCCTGGAAGTGGAGTTAGGAGCTTCTAACCCGTCATGGTTTCAATAC
ACATTCTTCTTTTGCCAGCGCTTCTGAAGAGCTGCTCTCACCTCTCTGCATCCC
TCR6 SEQ ID NO: 624
AATAGATATCCCCCTATGTGCATGCACACCTGCACACTCACGGCTGAAATCTCCCTAACCCAGGGG
GACCTTAGCATGCCTAAGTGACTAAACCAATAAAAATGTTCTGGTCTGGCCTGA
TCR7 SEQ ID NO: 625
AGGACCTGAAAAACGTGTTCCCACCCAAGGTCGCTGTGTTTGAGCCATCAGAAGCAGAGATCTCC
CACACCCAAAAGGCCACACTGGTGTGCCTGGCCACAGGCTTCTACCCCGACCACG
TCR8 SEQ ID NO: 626
TGGAGCTGAGCTGGTGGGTGAATGGGAAGGAGGTGCACAGTGGGGTCAGCACAGACCCGCAGCC
CCTCAAGGAGCAGCCCGCCCTCAATGACTCCAGATACTGCCTGAGCAGCCGCCTGA
TCR9 SEQ ID NO: 627
GGGTCTCGGCCACCTTCTGGCAGAACCCCCGCAACCACTTCCGCTGTCAAGTCCAGTTCTACGGGC
TCTCGGAGAATGACGAGTGGACCCAGGATAGGGCCAAACCTGTCACCCAGATCG
TCR10 SEQ ID NO: 628
ACCTCCGAGTCTTACCAGCAAGGGGTCCTGTCTGCCACCATCCTCTATGAGATCTTGCTAGGGAAG
GCCACCTTGTATGCCGTGCTGGTCAGTGCCCTCGTGCTGATGGCCATGGTCAAG
TCR11 SEQ ID NO: 629
AGAAAGGATTCCAGAGGCTAGCTCCAAAACCATCCCAGGTCATTCTTCATCCTCACCCAGGATTCT
CCTGTACCTGCTCCCAATCTGTGTTCCTAAAAGTGATTCTCACTCTGCTTCTCA
TCR12 SEQ ID NO: 630
TCTCCTACTTACATGAATACTTCTCTCTTTTTTCTGTTTCCCTGAAGATTGAGCTCCCAACCCCCAAG
TACGAAATAGGCTAAACCAATAAAAAATTGTGTGTTGGGCCTGGTTGCATTT
TCR13 SEQ ID NO: 631
ATATCCAGAACCCTGACCCTGCCGTGTACCAGCTGAGAGACTCTAAATCCAGTGACAAGTCTGTCT
GCCTATTCACCGATTTTGATTCTCAAACAAATGTGTCACAAAGTAAGGATTCTG
TCR14 SEQ ID NO: 632
ATGTGTATATCACAGACAAAACTGTGCTAGACATGAGGTCTATGGACTTCAAGAGCAACAGTGCT
GTGGCCTGGAGCAACAAATCTGACTTTGCATGTGCAAACGCCTTCAACAACAGCA
TCR15 SEQ ID NO: 633
TTATTCCAGAAGACACCTTCTTCCCCAGCCCAGAAAGTTCCTGTGATGTCAAGCTGGTCGAGAAAA
GCTTTGAAACAGATACGAACCTAAACTTTCAAAACCTGTCAGTGATTGGGTTCC
TCR16 SEQ ID NO: 634
GAATCCTCCTCCTGAAAGTGGCCGGGTTTAATCTGCTCATGACGCTGCGGCTGTGGTCCAGCTGAG
ATCTGCAAGATTGTAAGACAGCCTGTGCTCCCTCGCTCCTTCCTCTGCATTGCC
TCR17 SEQ ID NO: 635
ACAGAGGGAACTCTCCTACCCCCAAGGAGGTGAAAGCTGCTACCACCTCTGTGCCCCCCCGGCAA
TGCCACCAACTGGATCCTACCCGAATTTATGATTAAGATTGCTGAAGAGCTGCCA
TCR18 SEQ ID NO: 636
AACACTGCTGCCACCCCCTCTGTTCCCTTATTGCTGCTTGTCACTGCCTGACATTCACGGCAGAGGC
AAGGCTGCTGCAGCCTCCCCTGGCTGTGCACATTCCCTCCTGCTCCCCAGAGA
TCR19 SEQ ID NO: 637
CTGCCTCCGCCATCCCACAGATGATGGATCTTCAGTGGGTTCTCTTGGGCTCTAGGTCCTGCAGAA
TGTTGTGAGGGGTTTATTTTTTTTTAATAGTGTTCATAAAGAAATACATAGTAT
TCR20 SEQ ID NO: 638
TCTTCTTCTCAAGACGTGGGGGGAAATTATCTCATTATCGAGGCCCTGCTATGCTGTGTATCTGGGC
GTGTTGTATGTCCTGCTGCCGATGCCTTCATTAAAATGATTTGGAAGAGCAGA
Blocking Oligonucleotides
Read1 and poly(T) SEQ ID NO: 639
CTACACGACGCTCTTCCGATCTNNNNNNNNNNNNNNNNNNNNNNNNNNNNTTTTTTTTTTTTTTTT
TTTTTTTTTTTTTTVN
Blocking Template Switching Oligonucleotide SEQ ID NO: 640
CCCATGTACTCTGCGTTGATACCACTGCTT

### Variable Region Primer

After PCR enrichment, the PCR product was purified before the PCR indexing reaction. Quant-iT (ThermoFisher Scientific) analysis measured the DNA concentration of each sample, which was also normalized to the input material in the PCR indexing reaction (1.25 ng/reaction). Indexing PCR was performed as previously described (Ståhl, P.L., et al., Visualization and analysis of gene expression in tissue sections by spatial transcriptomics, Science, 353(6294), 78-82, (2016)). In total, 24 unique indexes were used, with each cDNA library receiving a unique index (TRB, IGHG, and IGHM products from the same cDNA library received the same indexing since the TCR and BCR clonotypes can be distinguished from each other bioinformatically using the constant primer sequence). After purification, PCR reactions were pooled. The pooled PCR library was run on a gel and a large band at around 500 bp excised was gel-purified and sequenced (NovaSeq, 2× 150 bp). The resulting data were de-multiplexed and the FastQ files were analyzed using MiXCR (Bolotin, D.A., et al., MiXCR: software for comprehensive adaptive immune profiling, Nature Methods, 12, 380-381 (2015), which is incorporated herein by reference in its entirety).

After PCR variable region primer enrichment, a number of TRB and IGH clonotypes in all prepared libraries were detected. For TRB, about 10,000 unique clonotypes were detected in spatial libraries prepared from tonsil tissue (data not shown) and between about 12,000 and about 25,000 unique clonotypes were detected in spatial libraries prepared from lymph node (data not shown). The positive control (SmartSeq2 RNAseq after PCR enrichment) yielded about 35,000 unique clonotypes. Variable region primer enrichment of the Smartseq2 library increased the TRB unique clonotype count over 35-fold, however, the SmartSeq2 library contained RNA extracted from two tonsil sections, whereas only a single tissue section was used for the spatial samples.

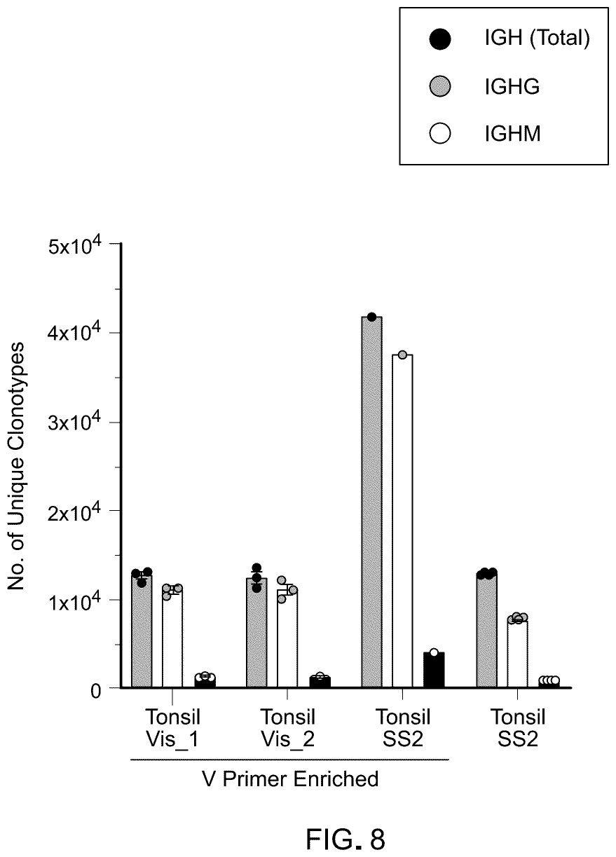

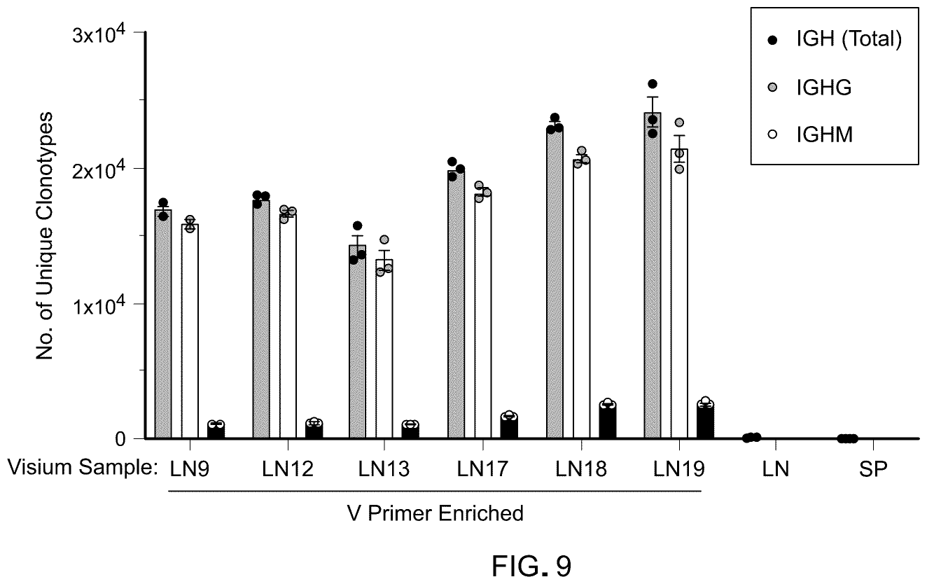

Similar results were observed for IGH detection with about 10,000 unique clonotypes detected from spatial libraries prepared from tonsil (FIG. 8) and between about 12,000 and about 25,000 unique clonotypes detected from spatial libraries prepared from the lymph node (FIG. 9). FIGS. 8 and 9 also shows the number of unique clonotypes found in non-enriched samples (far right) and the data show the number of unique clonotypes found is less than the V primer enriched samples. Approximately 10-fold higher IGHG clonotypes were detected relative to IGHM clonotypes similar to previous results for the SmartSeq2 RNAseq libraries (data not shown).

Variable region primer enrichment also resulted in a 4-fold increase in detected clonotypes for single-cell SmartSeq2 libraries. The clonotype increase observed after PCR variable primer enrichment of TRB relative to IGH is consistent with a known lower abundance of TRB transcripts in the cDNA library. For example, it is known that TRA/TRB transcript expression per T-cell is less relative to IGH/IGK/IGL expression per B-cell, and in particular, for plasma cells. Substantial, but not complete overlap, of IGH clonotypes between technical replicates detected in spatial libraries from tonsil tissue was observed. Similarly, substantial, but not complete, overlap of TRB clonotypes between technical replicates detected in spatial libraries from tonsil tissue was also observed. The data show that approximately half the clonotypes from a given technical replicate were detected in at least one or more of the other two technical replicates, which suggests many clonotypes were detected in a given technical replicate, but not all clonotypes were detected in each sample (data not shown). Approximately 10-20 fold increase in clonotype counts were detected with a poly(T) capture domain combined with PCR variable region primer enrichment relative to targeted capture, without variable region primer enrichment.

Collectively, these data show that PCR primer enrichment of analytes encoding immune cell receptors captured by poly(A) capture domains is possible. The use of a poly(A) capture domain allows for the simultaneous capture of analytes that do not encode for immune cell receptors and also does not require a custom array with targeted capture domains.

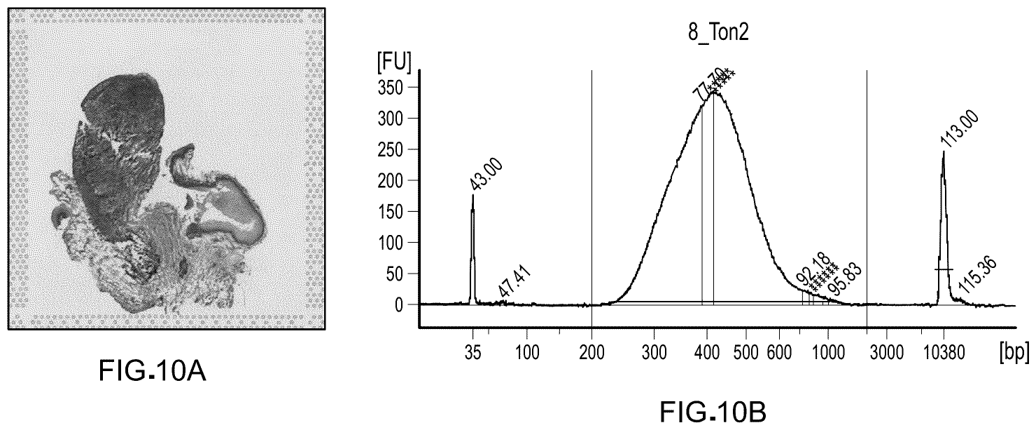

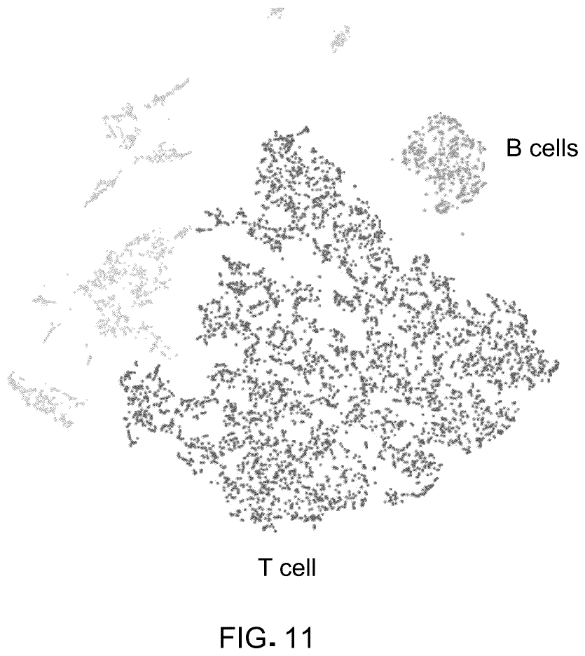

FIG. 10A shows H&E stained tonsil tissue on a spatial array and FIG. 10B shows the size distribution of spatial libraries prepared from tonsil tissue. The data shown in FIGS. 10A-B show the stained tonsil tissue and size distribution of the spatial libraries of the data included in this Example. Similar H&E staining was performed on breast tumor tissue on a spatial array and size distribution of spatial libraries were also prepared from breast tumor tissue (data not shown). FIG. 11 shows clustering of B-cells and T-cells from the single-cell analysis performed in this Example. FIG. 11 shows that while identifying populations of cells that include immune cell receptors, there is no connection to the spatial location of those cells within a biological sample. FIG. 11 shows a single-cell analysis which is not designed to be a spatial representation of immune cells within a biological sample.

### Example 3—Enrichment of Analytes Encoding Immune Cell Receptors

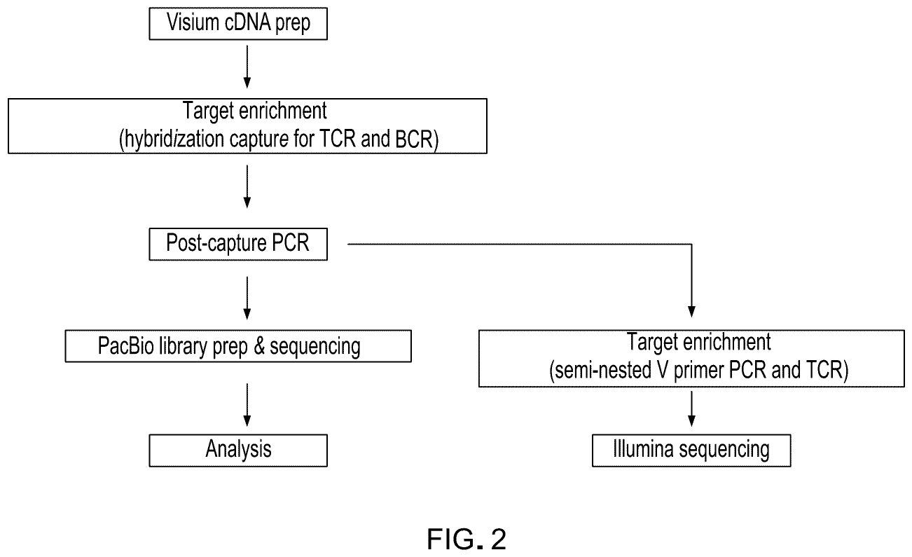

FIG. 2 shows an exemplary workflow for the enrichment of T-cell receptor (TCR) analytes and B-cell receptor (BCR) analytes after capture on a spatial array. After capture of analytes (e.g., TCR and BCR analytes) cDNA is synthesized, followed by target enrichment and either library preparation and sequencing or further target enrichment via a semi-nested PCR for TCR analytes followed by ILLUMINA® (sequencing technology) sequencing and finally analysis.

### Preparation of Visium Spatial Gene Expression Libraries

Sections of fresh-frozen breast tumor and tonsil tissue were sliced to 10 μm thickness and mounted onto slides from the Visium Spatial Gene Expression Slide & Reagent kit (10× Genomics®). Sequencing libraries were prepared following the manufacturer's protocol (Document number CG000239 Rev A, 10× Genomics®). Prior to imaging, coverslips were mounted on the slides according to the protocol's optional step “Coverslip Application & Removal”. Tissue images were taken at 20× magnification using a Metafer Slide Scanning platform (MetaSystems) and raw images were stitched with VSlide software (MetaSystems). Adaptions of the protocol were made in that the Hematoxylin and Eosin (H&E) staining time was reduced to 4 minutes and tissue permeabilization was performed for 12 minutes.

### Sequencing and Data Processing of Visium Spatial Gene Expression Libraries

Final sequencing libraries were sequenced on NextSeq™ 2000 (sequencer) (ILLUMINA® (sequencing technology)) with a 28-10-10-150 setup (tonsil), or NovaSeq6000 (ILLUMINA® (sequencing technology)) with a 28-10-10-120 setup (breast tumor). 172M and 93M raw read pairs were obtained from tonsil-1 and tonsil-2, respectively, and 215M and 244M from breast tumor 1 and breast tumor 2, respectively.

Following demultiplexing of the bel files, read 2 fastq files were trimmed using Cutadapt (Martin, M., Cutadapt removes adapter sequences from high-throughput sequencing reads, EMBnet Journal, 17 (1) (2011)) to remove full-length or truncated template switch oligo (TSO) sequences from the 5′ end (e.g., beginning of Read 2) and poly(A) homopolymers from the 3′ end (e.g., end of read 2). The TSO sequence (SEQ ID NO: 114) (AAGCAGTGGTATCAACGCAGAGTACATGGG) was used as a non-internal 5′ adapter with a minimum overlap of 5, meaning that partial matches (up to 5 base pairs) or intact TSO sequences were removed from the 5′ end. The error tolerance was set to 0.1 for the TSO trimming to allow for a maximum of 3 errors. For the 3′ end homopolymer trimming, a sequence of 10 As was used as a regular 3′ adapter to remove potential polyA tail products regardless of its position in the read, also with a minimum overlap of 5 base pairs. The trimmed data was processed with the SpaceRanger pipeline (10× Genomics®), version 1.2.1 (tonsil) and version 1.0.0 (BC) and mapped to the GRCH38 v93 genome assembly.

### Target Enrichment with Hybridization Capture

TCR and BCR target enrichment was performed using IDT xGen Hybridization and Wash Kit (#1080584) with one enrichment probe pool (IDT) each for BCR and TCR transcripts (IG and TCR pool, Table 3). Custom blocking oligos (IDT, Table 3) were designed to hybridize to adaptor sequences of the cDNA library and to prevent off-target fragments from binding to BCR/TCR transcripts and contaminating the enriched library. The IG and TCR enrichment probe pools were mixed at ratio 1:3 and 1:12, respectively and each sample was enriched using both settings.

The “xGen hybridization capture of DNA libraries”, version 4 (IDT) protocol was followed with an input of 10 μl Visium cDNA per reaction, corresponding to between about 45-130 ng and the hybridization enrichment reaction was performed overnight.

The enriched and purified libraries were amplified twice with an AMPure bead wash after each PCR reaction, using 25 μl 2xKAPA mix, 7.5 μl cDNA primers (10× Genomics®) and 17.5 μl sample in MQ water. The following settings were used for the PCRs: 1. 98° C. 3 min; 2. 98° C. 15 sec; 3. 63° C. 30 sec; 4. 72° C. 2 min; 5. Repeat steps 2-5 6× for a total of 7 cycles (1st PCR) and 4× for a total of 5 cycles (2nd PCR); 6. 72° C. 1 min

### Library Preparation and Sequencing

The resulting product from the hybridization enrichment capture method was used as input into the SMRTbell library preparation protocol (PacBio). The DNA was concentrated by AMPure Bead Purification (0.8×), eluting in 6 μl of Elution Buffer, using 1 μl for Qubit measurements. At least 1 μg of input was used for each library and multiplexed 8 samples in total per sequencing run. PacBio Barcoded Overhand Adapters was used for multiplexing and followed the manufacturer's instructions for the library preparations. The pooled library had a concentration of 11.4 ng/μl (50 μl total eluted volume). A SMRT Enzyme clean up kit was used to remove linear and single stranded DNA. The final libraries were sequenced at 2.7 million long read sequences (168-422K reads/sample) on a Sequel II at the National Genomics Infrastructure (NGI)/Uppsala Genome Center.

### Sequencing Data Analysis

The input for the analysis was de-multiplexed consensus reads obtained from PacBio sequencing and performed with Python programming language. The fastq files were parsed into a dataframe with readID, sequence and quality columns. Data was searched for the Truseq adapter sequence and the TSO sequence to anchor the ends of each of the reads, and reads that lacked these sequences were discarded. A portion of the Truseq adapter starting in the first seven bases of either the read or its reverse complement was identified. If any of the positions matched the sequence with hamming distance 1 or less they were tagged. The same was performed for a portion of the TSO sequence. The sequences were reverse complemented as needed so that all the reads had the Truseq adapter (SEQ ID NO: 115) at the beginning and the TSO (SEQ ID NO: 114) at the end. The spatial barcode and the UMI were identified. The first 16 bases were obtained following the TruSeq adapter to determine the spatial barcode and subsequent bases determined the unique molecular identifier (UMI). Additionally, following the sequence of the UMI at least 4 bases were identified as all thymines (e.g., the poly(dT) capture domain) and filtered out of the reads that had any other bases within that interval. Any read with a UMI identified as a poly(dT) sequence was removed. The end of poly(dT) region is defined as the first matching position for the pattern ‘[^T]T{0,2}[^T]T{0,2}[^T]’.

### Clonality Analysis and Visualization

To run MIXCR (version 3.0.3), poly(dT) and TSO sequences were trimmed and the reads were written to a new fastq file. The reads were analyzed with MIXCR and the following command:
‘mixer analyze shotgun -s hsa --align -OsaveOriginalReads=true --starting-material rna <TrimmedFastq><SampleName>’

The following MIXCR command was performed to report alignments for each read:
‘mixer exportAlignments -f -cloneIdWithMappingType -cloneId -readIds -descrsR1 <SampleName>.clna <ReportFile>’

The resulting tabular file was used to assign reads to the clonotypes in MIXCR output. Any reads that did not map to any clone were filtered out (cloneID==−1), then the reads were grouped in a table by the spatial barcode and UMI and counted how many reads were present and how many clones were associated with each UMI. UMIs that were assigned to more than one clonotype were filtered out, since they are likely due to PCR or sequencing errors.

The resulting clonotype count matrices were subsequently loaded into R (R Core Team, A language and environment for statistical computing, R Foundation for Statistical Computing, (2017)). Tissue images, spatial coordinates and total gene expression counts obtained through the Visium platform and SpaceRanger pipeline were also loaded, and one Seurat object (Stuart et al. Comprehensive Integration of Single-Cell Data, Cell, 177(7) (2019)) per sample type (tonsil and breast tumor tumor) was created using the STutility package (Bergenstråhle et al., Seamless integration of image and molecular analysis for spatial transcriptomics workflows, BMC Genomics, 21(1), (2020)). The clonotype count matrix was extended by adding any missing spatial barcodes that were present in the total gene expression count matrix, and filled with zero counts for all added barcodes. The new, extended matrix was loaded as a new assay into the Seurat object, where genes and clonotypes were visualized on the tissue images using built-in functions of the STUtility package.

### Cell Processing for Single-Cell RNA Sequencing

Single cell suspensions from five breast tumor regions (Tumor A-E) were prepared by enzymatic tissue dissociation using the human Tumor Dissociation Kit (Miltenyi Biotec, 130-095-929) and gentleMACS dissociator (Miltenyi Biotec). Cell suspensions were stained with the Zombie Aqua Fixable viability dye (Biolegend, 423101) at room temperature for 20 minutes, then washed with Phosphate Buffered Saline (PBS). The cells were incubated with Human TruStain Fc block (Biolegend, 422302) for 10 minutes to limit non-specific antibody binding, then stained for 20 minutes with anti-EPCAM (1:40, Biolegend, 324206) and anti-CD45 (1:40, Biolegend, 304021) in FACS buffer (PBS+0.5% Bovine Serum Albumin). The cells were subsequently washed and resuspended in FACS buffer. Fluorescence-activated cell sorting (FACS) using an influx flow cytometer (BD Biosciences) was performed to sort live EPCAM+CD45+ single cells an Eppendorf tube for 10× Genomics® Chromium Single Cell gene expression analysis. Single stain controls (e.g., cells and beads) and fluorescence minus one controls (FMO), containing all the fluorochromes in the panel except the one being measured, were used to set voltages and to define the proper gating strategy.

### 10× Genomics® Chromium Single-Cell Library Preparation and Sequencing

Single-cell gene expression and VDJ clonotype libraries were generated from EPCAM-CD45+ cells using the 10× Genomics® Chromium Single Cell 5′ assay following the manufacturer's instructions. Libraries were profiled and quantified using a Bioanalyzer High Sensitivity DNA kit (Agilent Technologies) and Qubit High sensitivity kit (Thermo Fischer Scientific). Final single-cell gene expression libraries were sequenced (aiming for at least 30,000 reads per cell) on a NovaSeq 6000 SP flowcell (ILLUMINA® (sequencing technology) 150-8-8-150 read set-up) by the National Genomics Infrastructure, SciLifeLab.

### Single-Cell Gene Expression and VDJ Data Processing

Sequencing outputs were processed by Cell Ranger (version 5.0, 10× Genomics®). Gene-barcode count matrices were analyzed with the Seurat package (version 4.0, Satija Lab). Two steps of filtering were introduced here. First, raw gene expression matrices were subset by the barcode list in VDJ output, including T cell subsets and B cell subsets. Based on the UMI count, gene count, and mitochondrial percentage of raw gene expression matrices and their subsets, each threshold was selected to keep the maximum count of high-quality cells and avoid losing T and B cells which have VDJ sequencing outputs. Second, doublets in each sample were detected and filtered out by HTODemux( ) function in Seurat. All samples were integrated and scaled into one count matrix by Seurat. Dimension reduction, UMAP generation, and clustering, were performed on the merged dataset by Seurat. The merged dataset was clustered by a gradient of the resolution, from 0.2 to 2. The final resolution was determined by the significance of top-listed differentially expressed genes in each cluster. Cell types were annotated by differentially expressed genes and their marker genes expression level. All dimension reduction and annotation results, along with the VDJ output files were imported into Loupe Browser (version 5.0, 10× Genomics®) and Loupe VDJ Browser (version 4.0, 10× Genomics®) for interactive analysis.

### Semi-Nested PCR

After hybridization capture and post-capture PCR amplification (14 cycles), semi-nested PCR reactions were performed with the following primers: V primers targeting either the TRAV or TRBV genes, 5′ of the CDR3 region (i.e. ‘Outer’ TRAV or TRBV primers, see Table 3 for sequences) and a primer (‘partRead1’, see Table 3) targeting the universal partial read 1 sequence present on the transcripts in Visium cDNA libraries. PartRead1 is also compatible with TruSeq indexes to allow multiplexing of samples for sequencing. For the semi-nested PCR experiments, the Visium cDNA was further pre-amplified prior to hybridization capture to generate more input needed for testing. The Outer V primer PCR input was 1-5 ng of hybridization captured cDNA from two breast tumor tissue Visium libraries (replicate, adjacent sections) and the reaction was run with KAPA HiFi HotStart ReadyMix (2×) (KAPA Biosystems). All primers were diluted 40× for a final concentration of 2.5 μM (Integrated DNA Technologies). The PCR was run for 15 cycles under the following conditions: 1. 98° C. 5 min; 2. 98° C. 20 sec; 3. 65° C. 30 sec; 4. 72° C. 1:30 min; 5. Repeat steps 2-5 14× for a total of 15 cycles; and 6. 72° C. 7 min.

Quantitative real-time PCR (qPCR) was performed to determine the appropriate number of cycles (to avoid exponential amplification). The Outer V primer PCR product was purified using AMPure beads (0.6×), followed by two 80% EtOH washes. The Outer V primer PCR product was eluted in EB buffer after incubation at 15 min at 37° C. The cleaned up PCR product was quantified using Qubit and BioAnalyzer (Agilent). 3-5 ng of each PCR product was used as input to the subsequent Inner V primer PCR.

The Inner V primer PCR was performed with the following primers: V primers targeting either the TRAV or the TRBV gene, close/adjacent to the CDR3 region (e.g., ‘Inner’ V primers) and the same universal partial read 1 primer as described for the Outer V primer PCR (‘partRead1’). These Inner V primers have a handle compatible with TruSeq indexing. The primer concentrations and reagents were as described for the OUTER V primer PCR. qPCR was used to determine the optimal number of cycles (7). The following conditions were used for the PCR reaction: 7. 98° C. 5 min; 8. 98° C. 20 sec; 9. 72° C. 30 sec; 10. 72° C. 1:30 min; 11. Repeat steps 2-5 14× for a total of 15 cycles; 12. 72° C. 7 min.

The same AMPure bead-clean up and ethanol washes were performed as described above. The final eluted PCR product was quantified using Qubit and BioAnalyzer (Agilent). The samples were PCR indexed using TruSeq Indexes (5 cycles) and sequenced on a Novaseq sequencing instrument using a short read 1 and a longer read 2 to capture the entire CDR3 region and part of the constant region from the 5′ end.

### Target enrichment with hybridization capture for TCR and BCR sequences

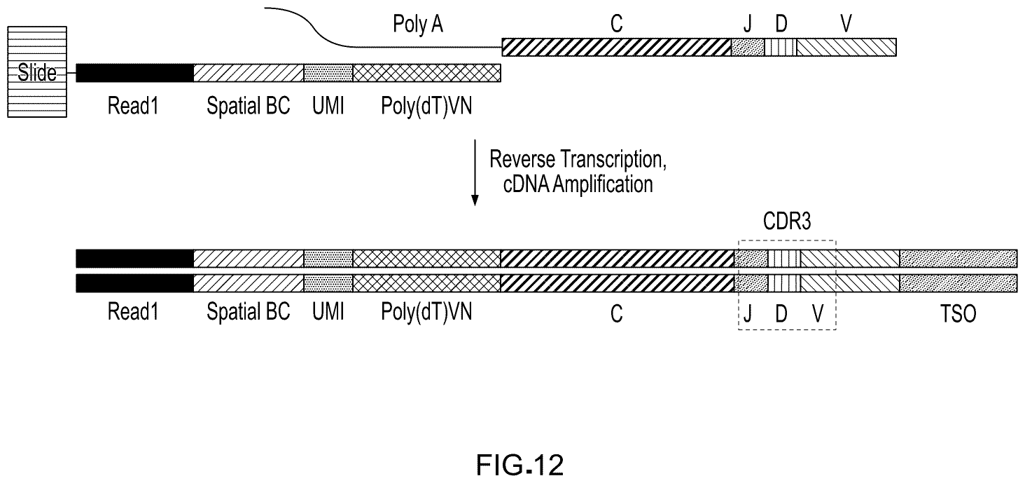

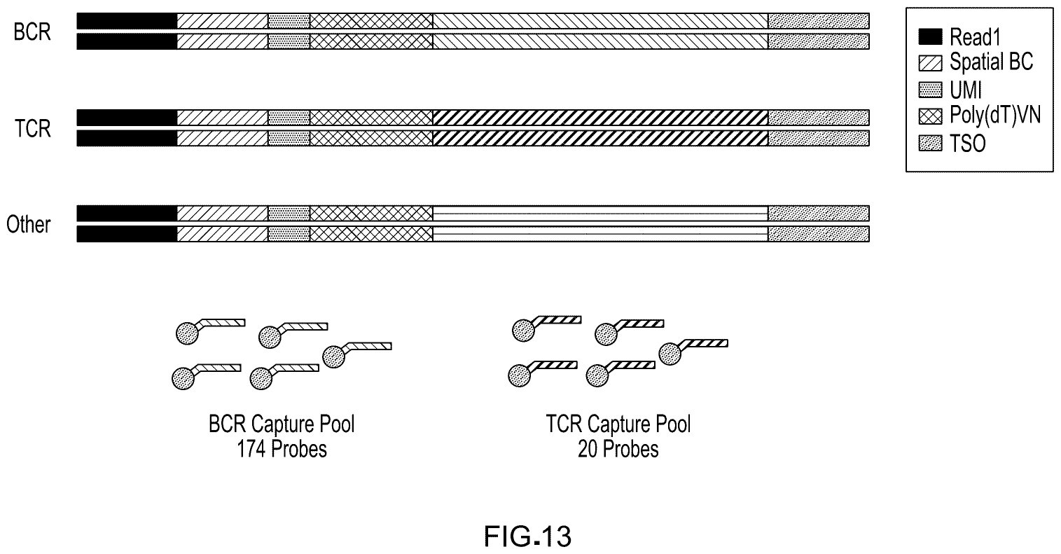

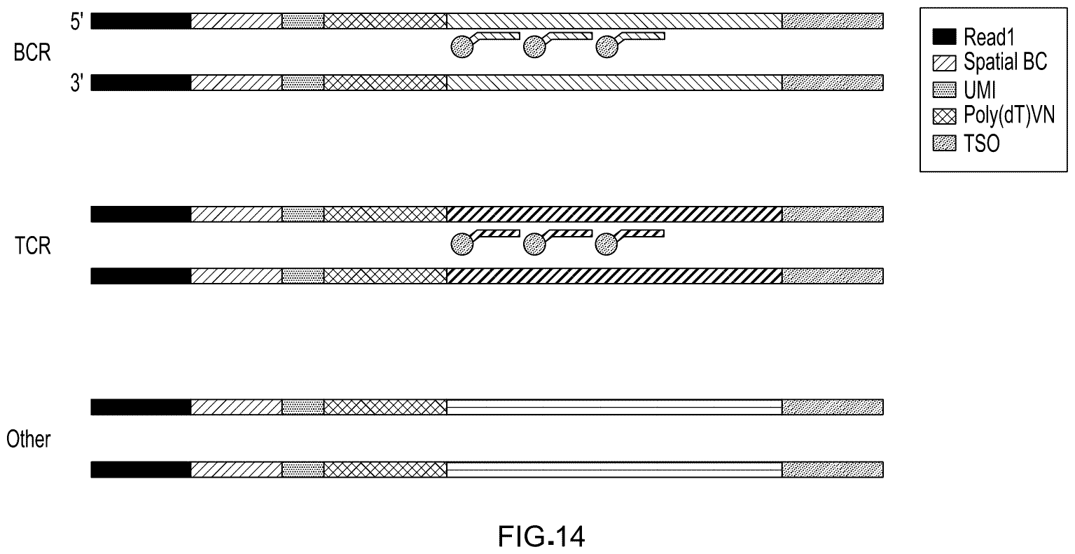

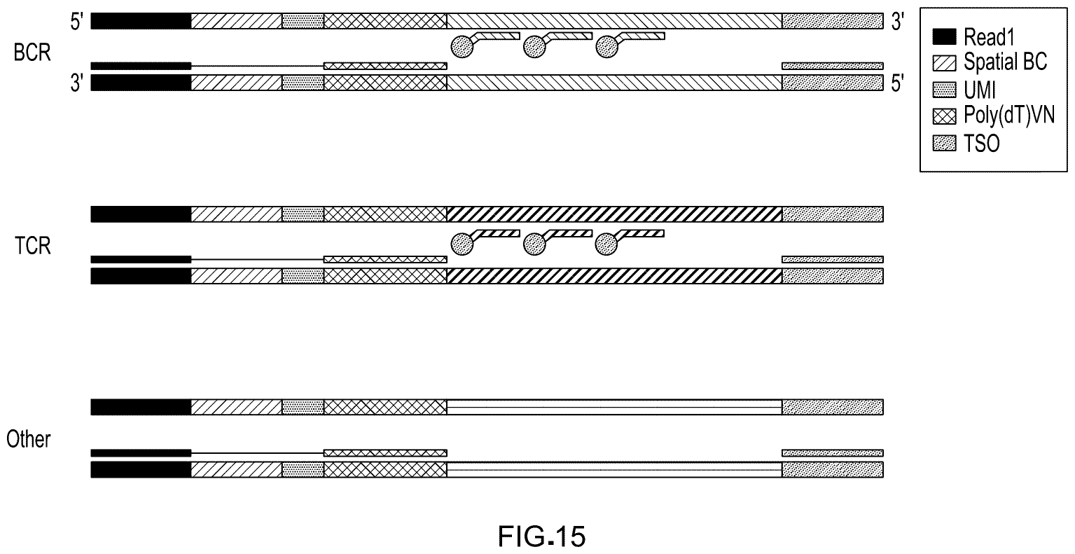

As discussed above BCR (IGH, IGK, IGL) and TCR (TRA, TRB) clones can be amplified using PCR from poly(dT) captured cDNA libraries, e.g., Visium (10× Genomics®). In some instances, the obtained amplicons lacked the spatial barcode. Therefore, to enrich for TCR and BCR sequences while preserving the spatial barcode and the CDR3 clonal information, a target enrichment strategy with hybridization probes (IDT technologies) was tested. Manufacturer's instructions were followed with some minor adaptations according to the methods described above. Visium cDNA from two tonsil sections (e.g., from the same tonsil, spaced 150 μM apart) were used as input material. FIG. 12 shows poly(A) capture with a poly(T) capture domain. A poly(T) capture domain can capture other mRNA analytes from a tissue, including mRNA analytes encoding immune cell receptors, however, immune cell analytes were enriched using a hybridization enrichment probe strategy. BCR hybridization probes (n=174) were designed to span all BCR constant genes (e.g., IGH, IGL, IGK), see Table 3. Similarly, TCR hybridization probes (n=20) were designed to target the TCR constant genes (e.g., TRA, TRB), see Table 3. FIG. 13 shows an exemplary cDNA library that would include BCR, TCR, other analytes and a pool of hybridization probes specific for BCR and TCR analytes. The Visium cDNA samples were hybridized with the hybridization enrichment capture probes and the hybridization reaction was performed overnight (FIG. 14), in the presence of blocking oligos as shown in FIG. 15 targeting Read 1, Poly(dT)VN, and TSO sequences present on the transcripts in the cDNA library. After a series of washes, a post-capture PCR reaction was performed, which amplifies all, or a portion of, the captured analyte pool. Indexed PacBio libraries were prepared for long read sequencing from the eluted PCR products. To avoid unnecessary PCR cycles, which can introduce artifacts, errors, and chimeric fragments, barcoded overhang adapter ligation was performed to add unique sample indexes to each sample. The enriched libraries were then sequenced, de-multiplexed, and analyzed.

### Clonotype Numbers

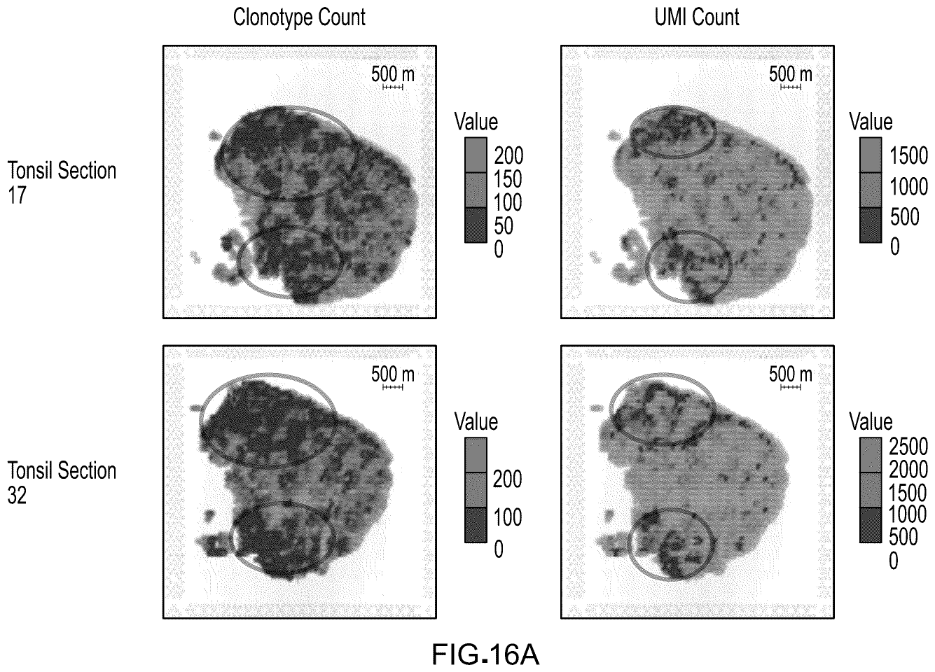

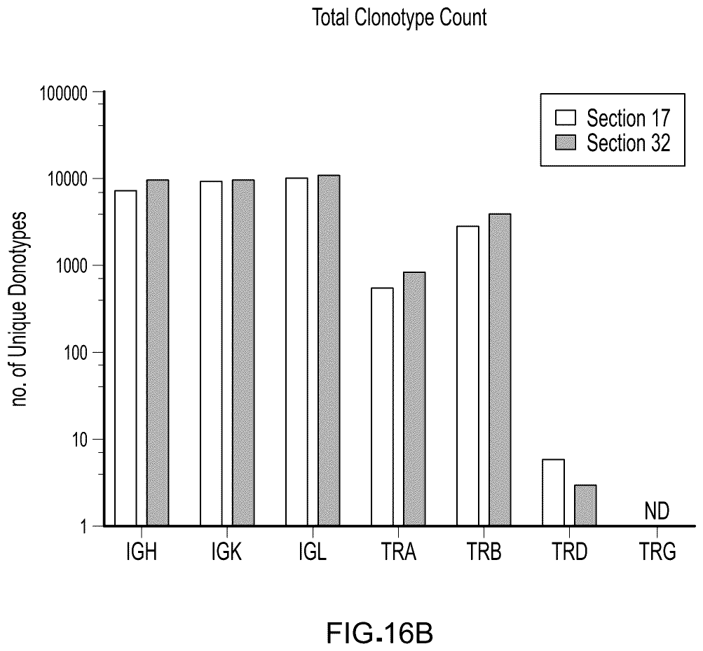

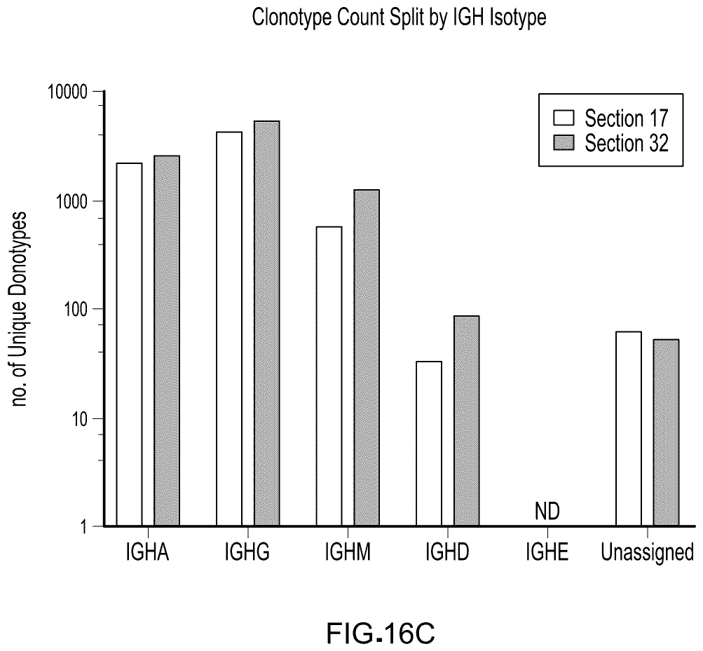

cDNA prepared from captured immune cell mRNA analytes were enriched via a hybridization capture approach as described above and combined with PacBio long read sequencing. The resulting data successfully identified spatially barcoded BCR and TCR clones from tonsil Visium libraries (FIGS. 16A-C). A clone was defined as a single-chain with a unique combination of VDJ gene segments and a CDR3 region, based on MIXCR analysis (previously described) (Bolotin et al., (2015)). FIG. 16A shows the distribution of the clonotype (left) and UMI (right) count for two tonsil sections, spaced 150 μm apart, from the same tonsil. The number of clonotypes per spot ranged between 0 and 300. For each tonsil sample, approximately 10,000 IGH, IGK and IGL clones (BCR) were identified (FIG. 16B). For TCRs, 3,437 TRB clonotypes and 687 TRA clonotypes were captured on average. The approximately five-fold lower capture of TRA clones was likely due to the lower expression of TRAC on a per cell basis consistent with previous results. The date demonstrate more successful capture of BCR clones (relative to TCR clones), which, without wishing to be bound by theory may be due to several reasons, including a higher receptor expression by B cell lineage cells (particularly plasma cells) and a higher number of cells per B cell clone. Furthermore, all IGH isotypes were found, except for IGHE, which is expressed by very rare IgE positive B cell lineage cells (FIG. 16C). A small number of IGH clones were not assigned a constant gene. As expected, IGHG and IGHA-expressing cells dominate, followed by IGHM. The BCR light chains (IGK and IGL) were expressed at comparable numbers.

Collectively, the data demonstrate that target enrichment with hybridization probes from Visium cDNA mRNA libraries successfully enrich BCR and TCR clones from lymphocyte rich tissue.

### B and T Cell Spatial Segregation in the Tonsil

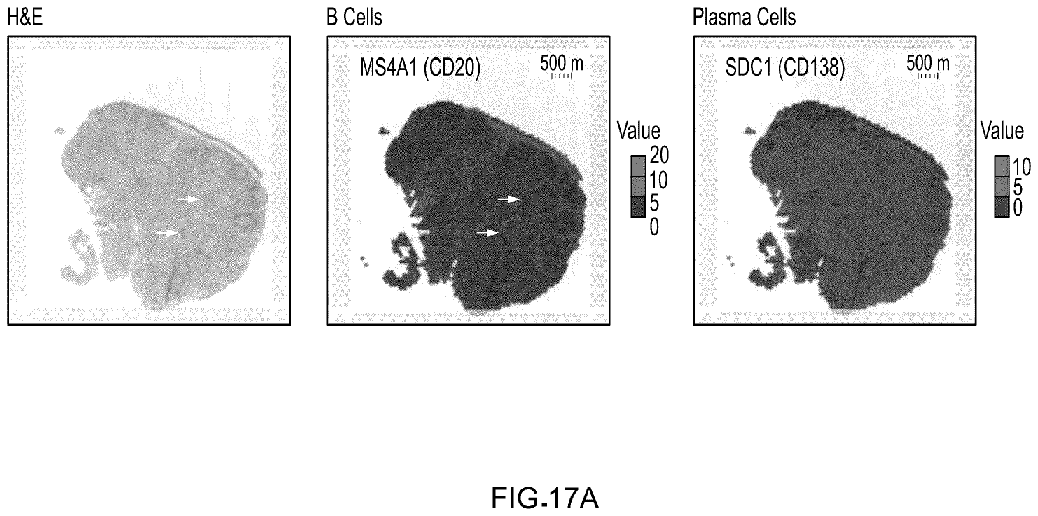

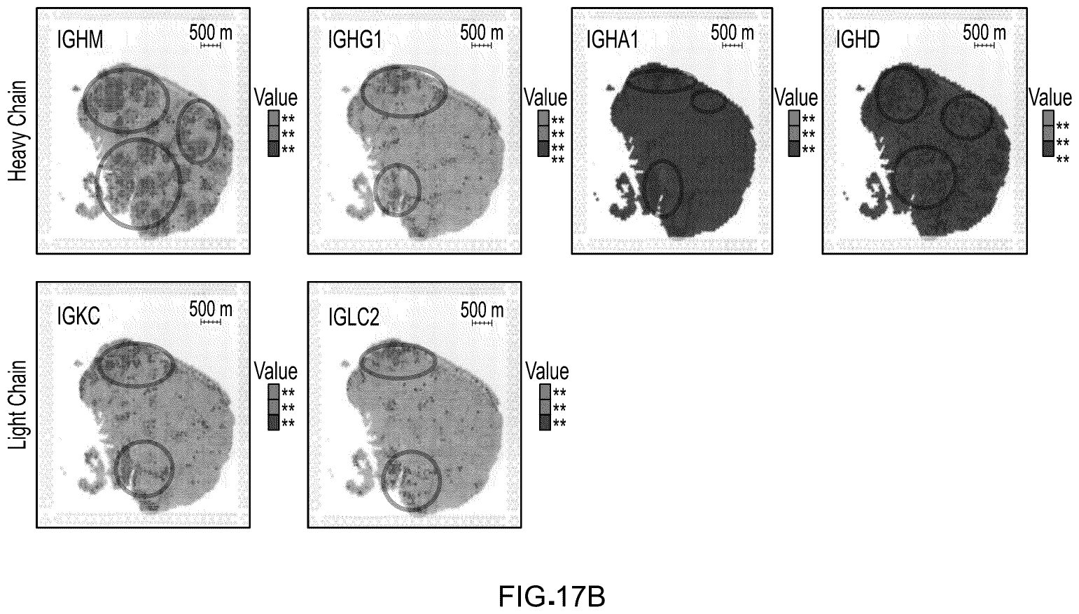

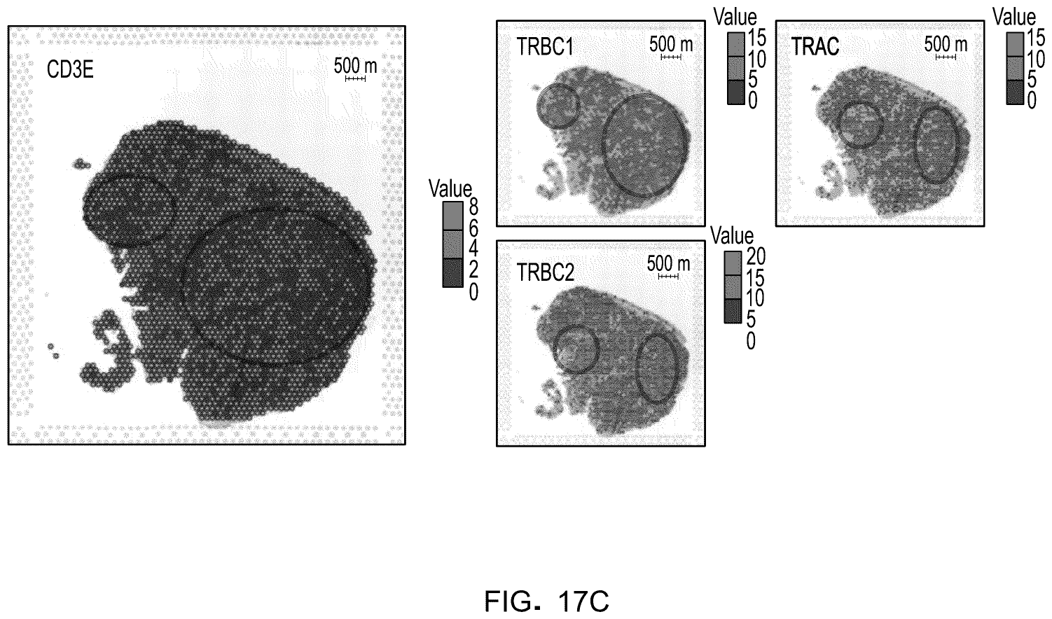

It was expected that with tonsil and similar tissues, e.g., lymph node, B cell clones would segregate mainly in follicles or germinal centers, in which B cell clonal selection and expansion occurs. In the Visium gene expression data, MS4A1, which encodes CD20 a B cell specific gene, was expressed in a cluster-like pattern that corresponded with increased cell density as visualized by the H&E staining, suggestive of B cell follicles (“B cell follicles”) (FIG. 17A, arrows). In contrast, SDC1, which encodes CD138 and is considered a reliable plasma cell enriched gene, was expressed mainly at the borders of the tissue and around B cell follicles, as expected from plasma cells (FIG. 17A). This cell type distribution was also supported by the spatial expression of the IGH constant gene (FIG. 17B, top), IGHM, which is expressed by B cells prior to class switching into other isotypes and was mostly enriched in the same B cell follicle-like areas as MS4A1. Similarly, IGHD, though more sparsely expressed, was also enriched in the B cell follicular areas. For other IGH isotypes (FIG. 17A, top) and the light chain (FIG. 17B, bottom), the highest gene expression was mainly outside B cell follicles, suggestive of increased expression by plasma cells. Based on CD3E and TCR constant gene (e.g., TRAC, TRBC1, TRBC2) expression, T cells were likely situated outside or around B cell follicles, which corresponds well with the presence of known, so-called “T cell zones” in lymphoid tissues (FIG. 17C).

### Clonotype Distribution in the Tonsil

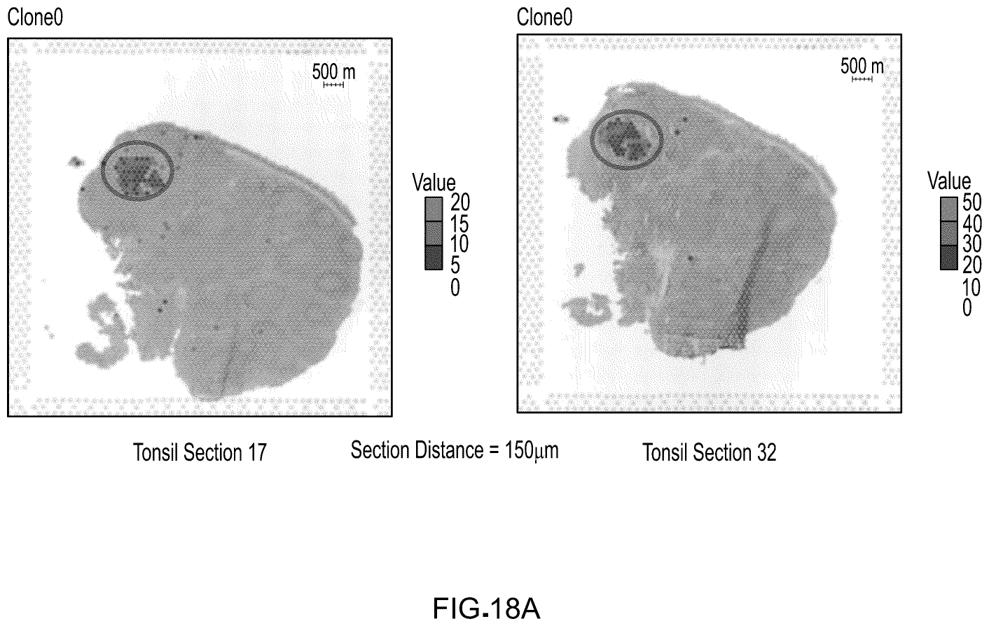

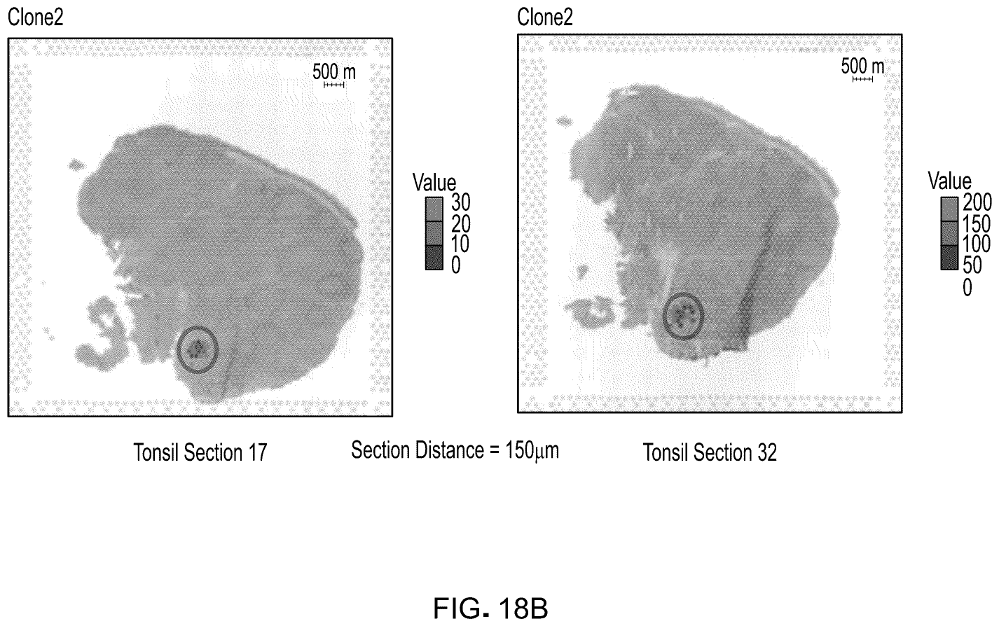

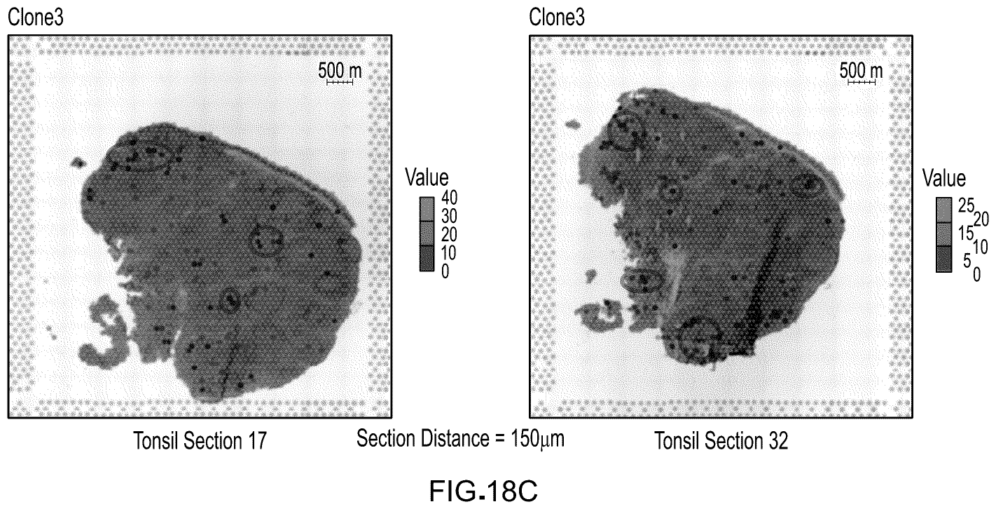

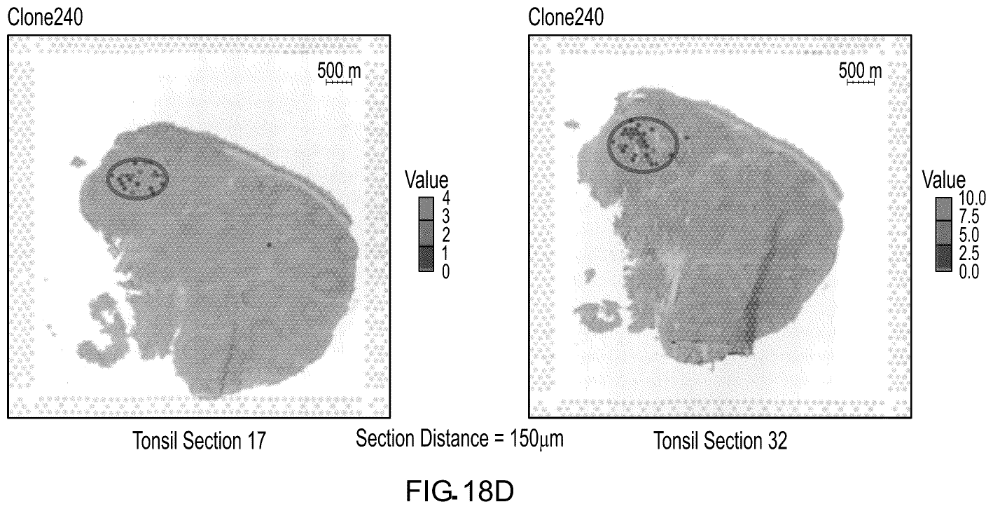

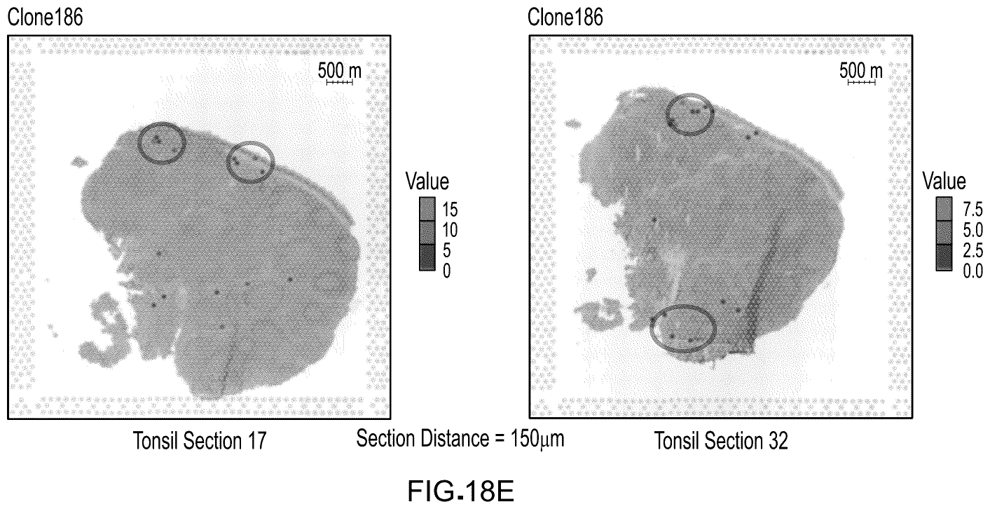

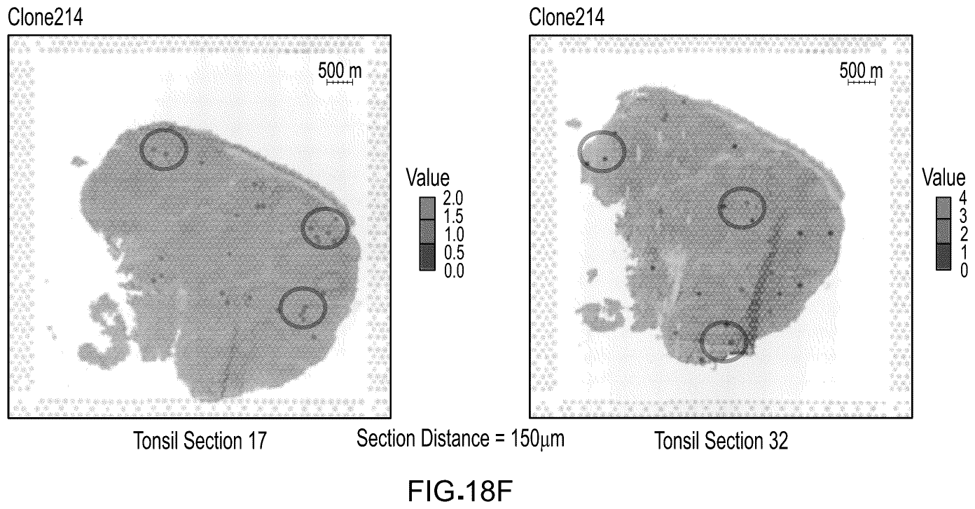

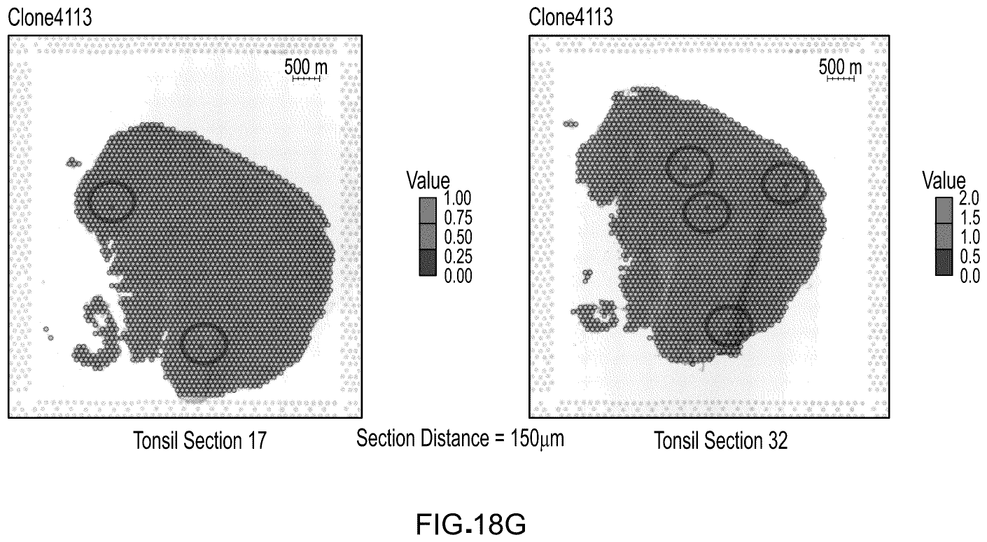

The data determined whether captured clones spatially segregate in tonsil tissue relative to the observed B and T cell segregation (FIGS. 17A-C and FIGS. 18A-G). The most abundant clone, IGKC, was highly expressed almost exclusively in a single B cell follicle, as captured by the two tonsil sections spaced 150 μM apart (FIG. 18A). Similar expression patterns were also observed for many clones; e.g., in FIG. 18B, a second representative clone, IGLC, was restricted to another B cell follicle. Without wishing to be bound by theory, these light chains may be expressed by B cells under-going selection and therefore are present in higher concentrations. Large clones, whose expression was not restricted to B cell follicles, were also found (see, e.g., FIG. 18C). These results indicate that clones can be captured with distinct spatial segregation within a tissue section. In accordance with IGHM gene expression, IGHM clones were also found in single follicles (sec, E.g., FIG. 18D for a representative example). In contrast, IGHA-expressing clones, tended to be expressed along the border of the tonsil tissue (FIG. 18E), consistent with the spatial IGHA gene expression (FIG. 18B). TCR clones tended to locate at the border of B cell follicles (see, e.g., FIG. 18F and FIG. 18G for representative examples of TRB and TRA clones, respectively). TCR clones also tended to have lower UMI counts per clone on average compared to the BCR clones, again, confirming that TCR transcripts are less abundant in tonsil Visium cDNA libraries and subsequently in the enriched libraries.

### Target Enrichment of Lymphocyte Receptors in Breast Tumor Tissue

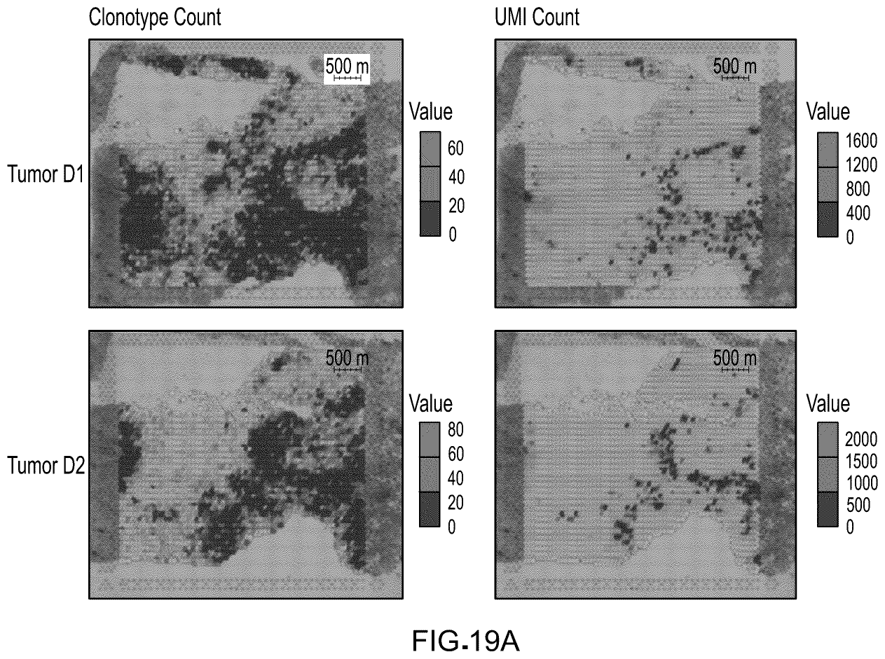

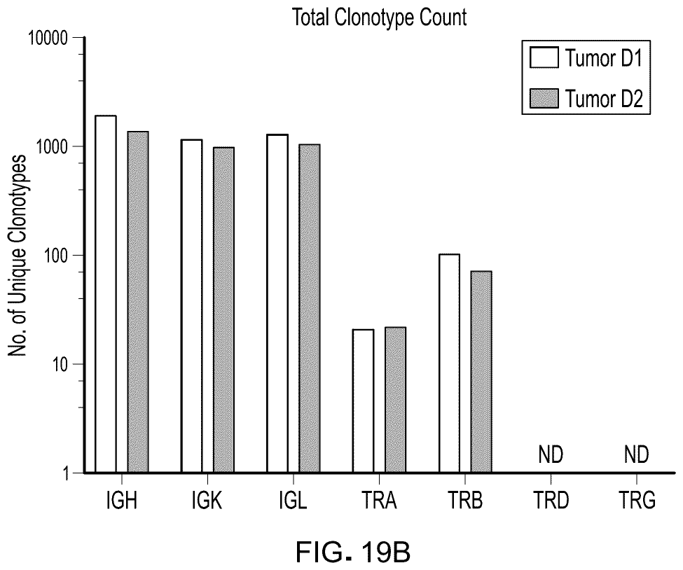

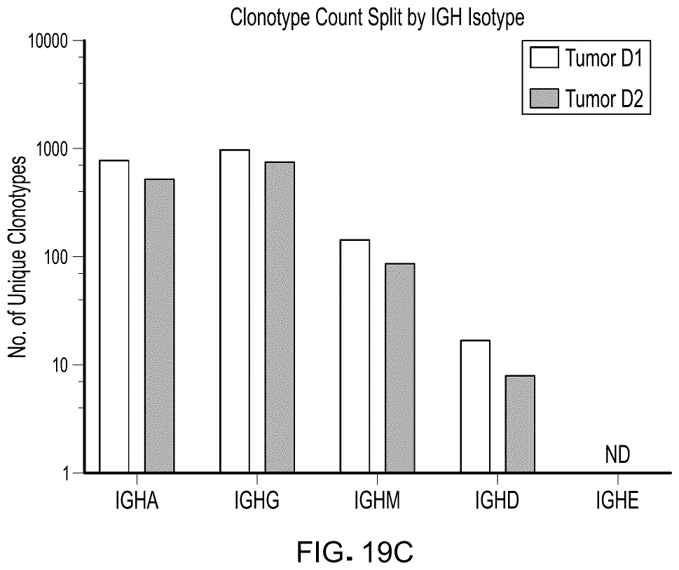

Target enrichment strategies as described herein were also tested on breast tumor tissue. Due to the high frequency of tumor cells and stromal cells in breast tumor tissue, it was expected that lymphocyte-associated transcripts would be less abundant, relative to tonsil tissue. Visium libraries were generated from two consecutive sections from breast tumor tissue, isolated from a HER2+ breast tumor patient. FIG. 19A shows the distribution of the clonotype (left) and UMI (right) count for two breast tumor sections. The number of clonotypes per spot ranged between 0 and 300. For each tonsil sample, we identified approximately 10,000 IGH, IGK and IGL clones (BCR) (FIG. 19B). Using the same approach, approximately 1000 IGH, IGK, and IGL clones and between 20-100 TCR clones from each breast tumor section were captured (FIGS. 19B and 19C). Fewer B and T cell clones were expected in the breast tumor samples relative to tonsil tissue, however, there were far fewer T cell clones relative to the B cell clones. Without wishing to be bound by theory, single-cell gene expression and VDJ libraries from the same tumor were prepared and 10-fold more T cells compared to B cell lineage cells (data not shown) were obtained from the single-cell data. Thus, the spatial methods described herein may be more efficient than single-cell approaches in capturing B cell expression and gene expression indicative of plasma, whereas single-cell techniques may be superior in capturing T cells, relative to spatial transcriptomics for antigen receptors.

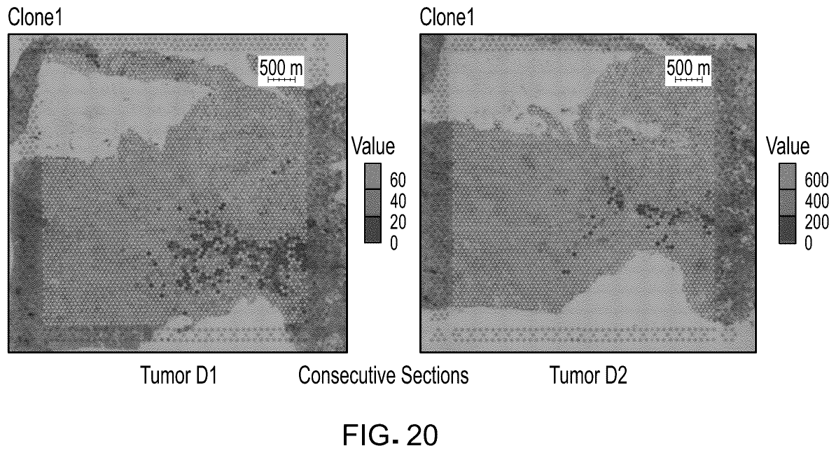

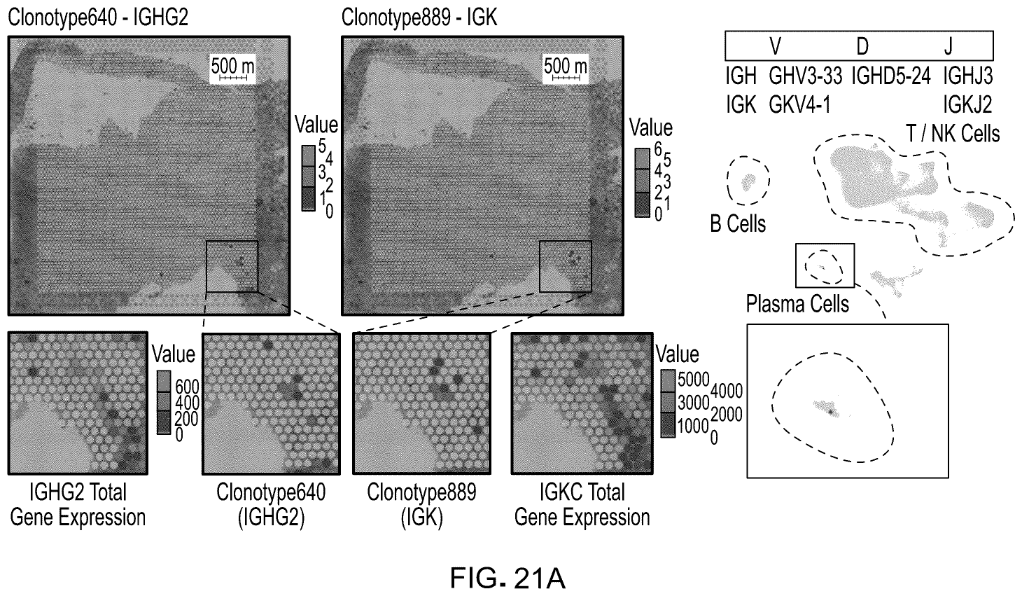

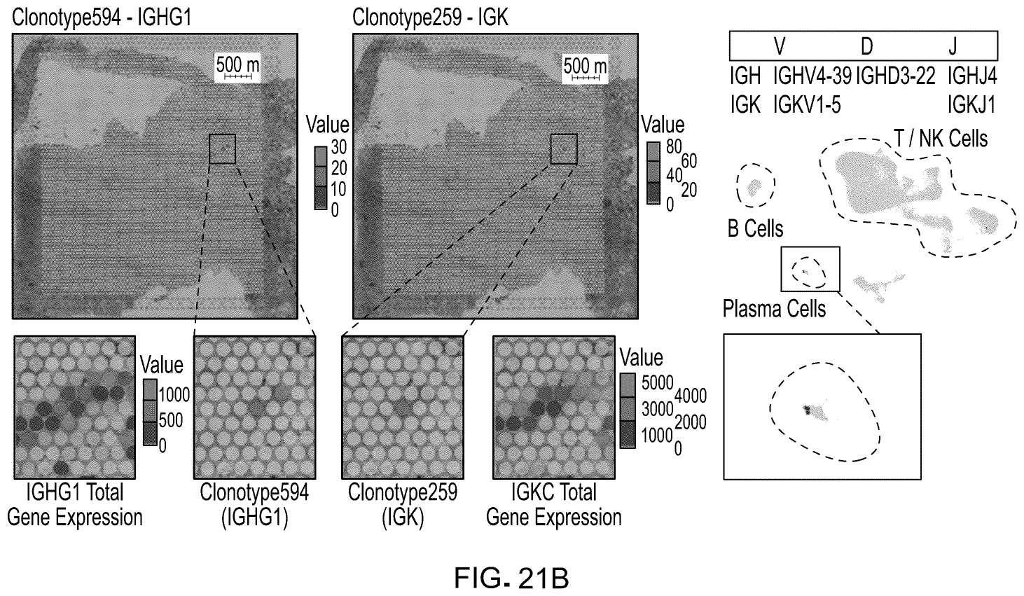

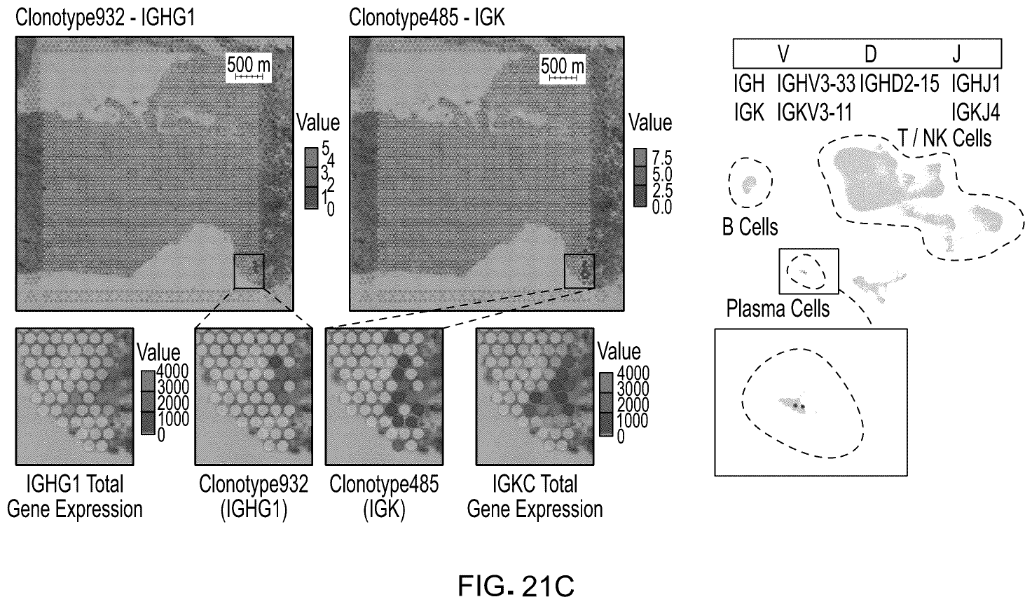

Spatial segregation of IGH clones (e.g., IGHV4-28, IGHD3-3, IGHD3-9, IGHJ4, IGHG1/IGHG3) within the breast tumor tissue was found consistent between two adjacent sections (See FIG. 20 for a representative example). Furthermore, since the linked single-cell VDJ data from the same tumor sample was available, detection of paired clones in the spatial clonotype data was also performed. While the single-cell data was processed from a much larger tissue section relative to the 10 μM tissue section used for spatial transcriptomic analysis, it was expected that large clones would be represented in both samples. By comparing the spatial transcriptomics for antigen receptor clonotype lists with the single-cell VDJ data, a total of 6 sets of paired BCR receptors were found in both datasets. The spatial gene expression of three such pairs are shown in FIGS. 21A-C. The similar spatial distribution for both chains for each clone and the concordance with the total respective IGH constant gene expression is demonstrated. For TCR clones, only two sets of paired receptors were found, with sparse UMI count (data not shown). The data show detection of paired receptors using spatial transcriptomics for antigen receptors and that these paired receptors are expressed in a spatially concordant manner.

### Target Enrichment for TCR Using Semi-Nested PCR

As described above, the hybridization probe approach was more efficient at capturing BCR clonotypes, most probably due to higher expression on a per cell basis than TCR clonotypes. In order to improve TCR capture, a second target enrichment step was introduced to increase the T cell clonotype yield and to prepare libraries compatible with ILLUMINA® (sequencing technology) sequencing. After hybridization probe capture and subsequent PCR amplification, TCR analytes were enriched using a semi-nested PCR approach as shown in FIG. 22. The PCR is a two-step PCR in which two sets of V (e.g., primer to the variable domain region “V”) primers (e.g., “Outer” and “Inner”) targeting the TRAV and TRBV genes, respectively, were combined with a universal primer targeting the partial Read1 present on transcripts in the Visium cDNA library. The “Outer” primer can be referred to as a second primer and the “Inner” primer can be referred to as a third primer. The outer primers target gene regions further away from the start of the CDR3 (e.g., between about 200-270 bp from the end of the coding V segment), whereas the inner primers target gene segments closer to the CDR3 (between about 20-25 bp from the end of the coding V segment).

The results show amplification of both TRA and TRB transcripts using the semi-nested PCR approach from breast tumor Visium libraries and that these libraries had the expected sizes (data not shown).

Collectively, the data demonstrate that spatial transcriptomics for antigen receptors can isolate high numbers of BCR and TCR clonotypes from tonsil and breast tumor tissue. These clones segregate in the tissue in characteristic ways concordant with their biology and cell type gene expression patterns.

### Embodiments

Embodiment 1 is a method for determining the presence and/or abundance of an immune cell clonotype at a location in a biological sample, the method comprising: (a) contacting a biological sample with an array comprising a plurality of capture probes, wherein a capture probe of the plurality of capture probes comprises (i) a spatial barcode and (ii) a capture domain that specifically binds to a nucleic acid encoding an immune cell receptor of the immune cell clonotype; and (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell clonotype at a location in the biological sample.

Embodiment 2 is the method of embodiment 1, wherein the immune cell clonotype is a T cell clonotype.

Embodiment 3. The method of embodiment 2, wherein the immune cell receptor is a T cell receptor alpha chain.

Embodiment 4 is the method of embodiment 3, wherein the capture domain binds specifically to a nucleic acid sequence encoding a constant region of the T cell receptor alpha chain.

Embodiment 5 is the method of embodiment 3 or 4, wherein step (b) comprises determining a sequence encoding CDR3 of the T cell receptor alpha chain.

Embodiment 6 is the method of embodiment 5, wherein step (b) further comprises determining a sequence encoding one or both of CDR1 and CDR2 of the T cell receptor alpha chain.

Embodiment 7 is the method of embodiment 5, wherein step (b) further comprises determining a sequence encoding a full-length variable domain of the T cell receptor alpha chain.

Embodiment 8 is the method of embodiment 2, wherein the immune cell receptor is a T cell receptor beta chain.

Embodiment 9 is the method of embodiment 8, wherein the capture domain binds specifically to a nucleic acid sequence encoding a constant region of the T cell receptor beta chain.

Embodiment 10 is the method of embodiment 8 or 9, wherein step (b) comprises determining a sequence encoding CDR3 of the T cell receptor beta chain.

Embodiment 11 is the method of embodiment 10, wherein step (b) further comprises determining a sequence encoding one or both of CDR1 and CDR2 of the T cell receptor beta chain.

Embodiment 12 is the method of embodiment 10, wherein step (b) further comprises determining a full-length variable domain of the T cell receptor beta chain.

Embodiment 13 is the method of embodiment 1, wherein the immune cell clonotype is a B cell clonotype.

Embodiment 14 is the method of embodiment 13, wherein the immune cell receptor is an immunoglobulin kappa light chain.

Embodiment 15 is the method of embodiment 14, wherein the capture domain binds specifically to a nucleic acid sequence encoding a constant region of the immunoglobulin kappa light chain.

Embodiment 16 is the method of embodiment 14 or 15, wherein step (b) comprises determining a sequence encoding CDR3 of the immunoglobulin kappa light chain.

Embodiment 17 is the method of embodiment 16, wherein step (b) further comprises determining a sequence encoding one or both of CDR1 and CDR2 of the immunoglobulin kappa light chain.

Embodiment 18 is the method of embodiment 16, wherein step (b) further comprises determining a sequence encoding a full-length variable domain of the immunoglobulin kappa light chain.

Embodiment 19 is the method of embodiment 13, wherein the immune cell receptor is an immunoglobulin lambda light chain.

Embodiment 20 is the method of embodiment 19, wherein the capture domain binds specifically to a nucleic acid sequence encoding a constant region of the immunoglobulin lambda light chain.

Embodiment 21 is the method of embodiment 19 or 20, wherein step (b) comprises determining a sequence encoding CDR3 of the immunoglobulin lambda light chain.

Embodiment 22 is the method of embodiment 21, wherein step (b) further comprises determining a sequence encoding one or both of CDR1 and CDR2 of the immunoglobulin lambda light chain.

Embodiment 23 is the method of embodiment 21, wherein step (b) further comprises determining a sequence encoding a full-length variable domain of the immunoglobulin lambda light chain.

Embodiment 24 is the method of embodiment 13, wherein the immune cell receptor is an immunoglobulin heavy chain.

Embodiment 25 is the method of embodiment 24, wherein the capture domain binds specifically to a nucleic acid sequence encoding a constant region of the immunoglobulin heavy chain.

Embodiment 26 is the method of embodiment 24 or 25, wherein step (b) comprises determining a sequence encoding CDR3 of the immunoglobulin heavy chain.

Embodiment 27 is the method of embodiment 26, wherein step (b) further comprises determining a sequence encoding one or both of CDR1 and CDR2 of the immunoglobulin heavy chain.

Embodiment 28 is the method of embodiment 26, wherein step (b) further comprises determining a sequence encoding a full-length variable domain of the immunoglobulin heavy chain.

Embodiment 29 is the method of any one of embodiments 1-28, wherein step (b) comprises the capture probe using the nucleic acid encoding the immune cell receptor as a template, thereby generating an extended capture probe.

Embodiment 30 is the method of embodiment 29, wherein step (b) comprises extending a 3′ end of the capture probe.

Embodiment 31 is the method of embodiment 29 or 30, wherein step (b) further comprises generating a second strand of nucleic acid that comprises (i) a sequence that is complementary to all or a portion of the spatial barcode, and (ii) a sequence that corresponds to all or a portion of the sequence of the nucleic acid encoding the immune cell receptor.

Embodiment 32 is the method of any one of embodiments 1-31, wherein the capture probe further comprises a cleavage domain, a functional domain, a unique molecular identifier, or any combination thereof.

Embodiment 33 is the method of any one of embodiments 1-30, wherein the capture probe further comprises a functional domain.

Embodiment 34 is the method of embodiment 33, wherein step (b) further comprises generating a second strand of nucleic acid that comprises (i) a sequence that is complementary to all or a portion of the functional domain, (ii) a sequence that is complementary to all or a portion of the spatial barcode, and (iii) a sequence that corresponds to all or a portion of the sequence of the nucleic acid encoding the immune cell receptor.

Embodiment 35 is the method of embodiment 34, wherein step (b) further comprises amplifying the second strand of nucleic acid using (i) a first primer comprising all or a portion of the functional domain, wherein the functional domain is 5′ to the spatial barcode in the second strand of nucleic acid, and (ii) a second primer comprising a sequence that is substantially complementary to a portion of a sequence encoding a variable region of the immune cell receptor.

Embodiment 36 is the method of any one of embodiments 1-35, wherein the biological sample comprises a tissue sample.

Embodiment 37 is the method of embodiment 36, wherein the tissue sample is a tissue section.

Embodiment 38 is the method of embodiment 37, wherein the tissue section is a fixed tissue section.

Embodiment 39 is the method of embodiment 38, wherein the fixed tissue section is a formalin-fixed paraffin-embedded tissue section.

Embodiment 40 is the method of any one of embodiments 37-39, wherein the tissue section comprises a tumor region.

Embodiment 41 is the method of any one of embodiments 1-40, wherein the nucleic acid encoding the immune cell receptor comprises RNA.

Embodiment 42 is the method of embodiment 41, wherein the RNA is mRNA.

Embodiment 43 is the method of any one of embodiments 1-40, wherein the nucleic acid encoding the immune cell receptor comprises DNA.

Embodiment 44 is the method of embodiment 43, wherein the DNA is genomic DNA.

Embodiment 45 is the method of any one of embodiments 1-44, wherein the method further comprises, prior to step (b), contacting the biological sample with ribosomal RNA depletion probes and mitochondrial RNA depletion probes.

Embodiment 46 is the method of any one of embodiments 1-45, wherein the method further comprises imaging the biological sample.

Embodiment 47 is the method of any one of embodiments 1-46, wherein the determining in step (b) comprises sequencing (i) all or a portion of the sequence of the spatial barcode or a complement thereof, and (ii) all or a portion of the sequence of the nucleic acid encoding the immune cell receptor or a complement thereof.

Embodiment 48 is the method of any one of embodiments 1-47, wherein step (b) comprises determining the presence of the immune cell clonotype at a location in the biological sample.

Embodiment 49 is the method of any one of embodiments 1-47, wherein step (b) comprises determining the abundance of the immune cell clonotype at a location in the biological sample.

Embodiment 50 is the method of any one of embodiments 1-47, wherein step (b) comprises determining the presence and abundance of the immune cell clonotype at a location in the biological sample.

Embodiment 51 is the method of any one of embodiments 1-47, wherein step (b) comprises determining the presence of two or more immune cell clonotypes at a location in the biological sample.

Embodiment 52 is the method of any one of embodiments 1-47, wherein step (b) comprises determining the abundance of two or more immune cell clonotypes at a location in the biological sample.

Embodiment 53 is the method of any one of embodiments 1-47, wherein step (b) comprises determining the presence and abundance of two or more immune cell clonotypes at a location in the biological sample.

Embodiment 54 is the method of any one of embodiments 51-53, wherein the method further comprises comparing the two or more immune cell clonotypes.

Embodiment 55 is the method of any one of embodiments 51-54, wherein the two or more immune cell clonotypes are each a B cell clonotype.

Embodiment 56 is the method of any one of embodiments 51-54, wherein the two or more immune cell clonotypes are each a T cell clonotype.

Embodiment 57 is the method of any one of embodiments 51-54, wherein the two or more immune cell clonotypes comprise at least one T cell clonotype and at least one B cell clonotype.

Embodiment 58 is a method for determining the presence and/or abundance of an immune cell receptor at a location in a biological sample, the method comprising: (a) contacting a biological sample with an array comprising a plurality of capture probes, wherein a capture probe of the plurality of capture probes comprises (i) a spatial barcode and (ii) a capture domain that specifically binds to a nucleic acid encoding an immune cell receptor; and (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell receptor at a location in the biological sample.

Embodiment 59 is the method of embodiment 58, wherein the immune cell receptor is a T cell receptor alpha chain.

Embodiment 60 is the method of embodiment 59, wherein the capture domain binds specifically to a nucleic acid sequence encoding a constant region of the T cell receptor alpha chain.

Embodiment 61 is the method of embodiment 59 or 60, wherein step (b) comprises determining a sequence encoding CDR3 of the T cell receptor alpha chain.

Embodiment 62 is the method of embodiment 61, wherein step (b) further comprises determining a sequence encoding one or both of CDR1 and CDR2 of the T cell receptor alpha chain.

Embodiment 63 is the method of embodiment 61, wherein step (b) further comprises determining a sequence encoding a full-length variable domain of the T cell receptor alpha chain.

Embodiment 64 is the method of embodiment 58, wherein the immune cell receptor is a T cell receptor beta chain.

Embodiment 65 is the method of embodiment 64, wherein the capture domain binds specifically to a nucleic acid sequence encoding a constant region of the T cell receptor beta chain.

Embodiment 66 is the method of embodiment 64 or 65, wherein step (b) comprises determining a sequence encoding CDR3 of the T cell receptor beta chain.

Embodiment 67 is the method of embodiment 66, wherein step (b) further comprises determining a sequence encoding one or both of CDR1 and CDR2 of the T cell receptor beta chain.

Embodiment 68 is the method of embodiment 66, wherein step (b) further comprises determining a full-length variable domain of the T cell receptor beta chain.

Embodiment 69 is the method of embodiment 58, wherein the immune cell receptor is an immunoglobulin kappa light chain.

Embodiment 70 is the method of embodiment 69, wherein the capture domain binds specifically to a nucleic acid sequence encoding a constant region of the immunoglobulin kappa light chain.

Embodiment 71 is the method of embodiment 69 or 70, wherein step (b) comprises determining a sequence encoding CDR3 of the immunoglobulin kappa light chain.

Embodiment 72 is the method of embodiment 71, wherein step (b) further comprises determining a sequence encoding one or both of CDR1 and CDR2 of the immunoglobulin kappa light chain.

Embodiment 73 is the method of embodiment 71, wherein step (b) further comprises determining a sequence encoding a full-length variable domain of the immunoglobulin kappa light chain.

Embodiment 74 is the method of embodiment 58, wherein the immune cell receptor is an immunoglobulin lambda light chain.

Embodiment 75 is the method of embodiment 74, wherein the capture domain binds specifically to a nucleic acid sequence encoding a constant region of the immunoglobulin lambda light chain.

Embodiment 76 is the method of embodiment 74 or 75, wherein step (b) comprises determining a sequence encoding CDR3 of the immunoglobulin lambda light chain.

Embodiment 77 is the method of embodiment 76, wherein step (b) further comprises determining a sequence encoding one or both of CDR1 and CDR2 of the immunoglobulin lambda light chain.

Embodiment 78 is the method of embodiment 76, wherein step (b) further comprises determining a sequence encoding a full-length variable domain of the immunoglobulin lambda light chain.

Embodiment 79 is the method of embodiment 58, wherein the immune cell receptor is an immunoglobulin heavy chain.

Embodiment 80 is the method of embodiment 79, wherein the capture domain binds specifically to a nucleic acid sequence encoding a constant region of the immunoglobulin heavy chain.

Embodiment 81 is the method of embodiment 79 or 80, wherein step (b) comprises determining a sequence encoding CDR3 of the immunoglobulin heavy chain.

Embodiment 82 is the method of embodiment 81, wherein step (b) further comprises determining a sequence encoding one or both of CDR1 and CDR2 of the immunoglobulin heavy chain.

Embodiment 83 is the method of embodiment 81, wherein step (b) further comprises determining a sequence encoding a full-length variable domain of the immunoglobulin heavy chain.

Embodiment 84 is the method of any one of embodiments 58-83, wherein step (b) comprises extending an end of the capture probe using the nucleic acid encoding the immune cell receptor as a template, thereby generating an extended capture probe.

Embodiment 85 is the method of embodiment 84, wherein step (b) comprises extending a 3′ end of the capture probe.

Embodiment 86 is the method of embodiment 84 or 85, wherein step (b) further comprises generating a second strand of nucleic acid that comprises (i) a sequence that is complementary to all or a portion of the spatial barcode, and (ii) a sequence that corresponds to all or a portion of the sequence of the nucleic acid encoding the immune cell receptor.

Embodiment 87 is the method of any one of embodiments 58-86, where the capture probe further comprises a cleavage domain, a functional domain, a unique molecular identifier, or any combination thereof.

Embodiment 88 is the method of any one of embodiments 58-85, wherein the capture probe further comprises a functional domain.

Embodiment 89 is the method of embodiment 88, wherein step (b) further comprises generating a second strand of nucleic acid that comprises (i) a sequence that is complementary to all or a portion of the functional domain, (ii) a sequence that is complementary to all or a portion of the spatial barcode, and (iii) a sequence that corresponds to all or a portion of the sequence of the nucleic acid encoding the immune cell receptor.

Embodiment 90 is the method of embodiment 89, wherein step (b) further comprises amplifying the second strand of nucleic acid using (i) a first primer comprising all or a portion of the functional domain, wherein the functional domain is 5′ to the spatial barcode in the second strand of nucleic acid, and (ii) a second primer comprising a sequence that is substantially complementary to a portion of a sequence encoding a variable region of the immune cell receptor.

Embodiment 91 is the method of any one of embodiments 58-90, wherein the biological sample comprises a tissue sample.

Embodiment 92 is the method of embodiment 91, wherein the tissue sample is a tissue section.

Embodiment 93 is the method of embodiment 92, wherein the tissue section is a fixed tissue section.

Embodiment 94 is the method of embodiment 93, wherein the fixed tissue section is a formalin-fixed paraffin-embedded tissue section.

Embodiment 95 is the method of any one of embodiments 92-94, wherein the tissue section comprises a tumor region.

Embodiment 96 is the method of any one of embodiments 58-95, wherein the nucleic acid encoding the immune cell receptor comprises RNA.

Embodiment 97 is the method of embodiment 96, wherein the RNA is mRNA.

Embodiment 98 is the method of any one of embodiments 58-95, wherein the nucleic acid encoding the immune cell receptor comprises DNA.

Embodiment 99 is the method of embodiment 98, wherein the DNA is genomic DNA. Embodiment 100 is the method of any one of embodiments 58-99, wherein the method further comprises, prior to step (b), contacting the biological sample with ribosomal RNA depletion probes and mitochondrial RNA depletion probes.

Embodiment 101 is the method of any one of embodiments 58-100, wherein the method further comprises imaging the biological sample.

Embodiment 102 is the method of any one of embodiments 58-101, wherein the determining in step (b) comprises sequencing (i) all or a portion of the sequence of the spatial barcode or a complement thereof, and (ii) all or a portion of the sequence of the nucleic acid encoding the immune cell receptor or a complement thereof.

Embodiment 103 is the method of any one of embodiments 58-102, wherein step (b) comprises determining the presence of the immune cell receptor at a location in the biological sample.

Embodiment 104 is the method of any one of embodiments 58-102, wherein step (b) comprises determining the abundance of the immune cell receptor at a location in the biological sample.

Embodiment 105 is the method of any one of embodiments 58-102, wherein step (b) comprises determining the presence and abundance of the immune cell receptor at a location in the biological sample.

Embodiment 106 is the method of any one of embodiments 58-102, wherein step (b) comprises determining the presence of two or more immune cell receptors at a location in the biological sample.

Embodiment 107 is the method of any one of embodiments 58-102, wherein step (b) comprises determining the abundance of two or more immune cell receptors at a location in the biological sample.

Embodiment 108 is the method of any one of embodiments 58-102, wherein step (b) comprises determining the presence and abundance of two or more immune cell receptors at a location in the biological sample.

Embodiment 109 is the method of any one of embodiments 106-108, wherein the method further comprises comparing the two or more immune cell receptors.

Embodiment 110 is the method of any one of embodiments 106-109, wherein the two or more immune cell clonotypes are each an immune cell receptor of a B cell.

Embodiment 111 is the method of any one of embodiments 106-109, wherein the two or more immune cell clonotypes are each an immune cell receptor of a T cell.

Embodiment 112 is the method of any one of embodiments 106-109, wherein the two or more immune cell clonotypes comprise at least one immune cell receptor of a T cell and at least one immune cell receptor from a B cell.

Embodiment 113 is an array comprising a plurality of capture probes, wherein a capture probe of the plurality of capture probes comprises (i) a spatial barcode and (ii) a capture domain that specifically binds to a nucleic acid encoding an immune cell receptor of an immune cell clonotype.

Embodiment 114 is the array of embodiment 113, wherein the immune cell clonotype is a T cell clonotype.

Embodiment 115 is the array of embodiment 114, wherein the immune cell receptor is a T cell receptor alpha chain.

Embodiment 116 is the array of embodiment 115, wherein the capture domain binds specifically to a nucleic acid sequence encoding a constant region of the T cell receptor alpha chain.

Embodiment 117 is the array of embodiment 114, wherein the immune cell receptor is a T cell receptor beta chain.

Embodiment 118 is the array of embodiment 117, wherein the capture domain binds specifically to a nucleic acid sequence encoding a constant region of the T cell receptor beta chain.

Embodiment 119 is the array of embodiment 113, wherein the immune cell clonotype is a B cell clonotype.

Embodiment 120 is the array of embodiment 119, wherein the immune cell receptor is an immunoglobulin kappa light chain.

Embodiment 121 is the array of embodiment 120, wherein the capture domain binds specifically to a nucleic acid sequence encoding a constant region of the immunoglobulin kappa light chain.

Embodiment 122 is the array of embodiment 119, wherein the immune cell receptor is an immunoglobulin lambda light chain.

Embodiment 123 is the array of embodiment 122, wherein the capture domain binds specifically to a nucleic acid sequence encoding a constant region of the immunoglobulin lambda light chain.

Embodiment 124 is the array of embodiment 119, wherein the immune cell receptor is an immunoglobulin heavy chain.

Embodiment 125 is the array of embodiment 124, wherein the capture domain binds specifically to a nucleic acid sequence encoding a constant region of the immunoglobulin heavy chain.

Embodiment 126 is the array of any one of embodiments 113-125, where the capture probe further comprises a cleavage domain, a functional domain, a unique molecular identifier, or any combination thereof.

Embodiment 127. A kit comprising: an array of any one of embodiments 113-126; and one or both of ribosomal RNA depletion probes and mitochondrial RNA depletion probes.

Embodiment 128 is a method for determining the presence and/or abundance of an immune cell clonotype at a location in a biological sample, the method comprising: (a) contacting a biological sample with an array comprising a plurality of capture probes, wherein a capture probe of the plurality of capture probes comprises (i) a spatial barcode and (ii) a capture domain that binds to a nucleic acid encoding an immune cell receptor of the immune cell clonotype; (b) determining (i) all or a portion of the sequence of the spatial barcode or a complement thereof, and (ii) all or a portion of the sequence of the nucleic acid encoding the immune cell receptor or a complement thereof, and using the determined sequences of (i) and (ii) to determine the presence and/or abundance of the immune cell clonotype at a location in the biological sample.

Embodiment 129 is the method of embodiment 1, wherein step (b) comprises extending the capture probe using the nucleic acid encoding the immune cell receptor as a template, thereby generating an extended capture probe.

Embodiment 130 is the method of embodiment 129, wherein step (b) comprises extending a 3′ end of the capture probe.

Embodiment 131 is the method of embodiment 129 or 130, wherein step (b) further comprises generating a second strand of nucleic acid that comprises (i) a sequence that is complementary to all or a portion of the spatial barcode, and (ii) a sequence that corresponds to all or a portion of the sequence of the nucleic acid encoding the immune cell receptor.

Embodiment 132 is the method of any one of embodiments 128-131, wherein the capture probe further comprises a cleavage domain, a functional domain, a unique molecular identifier, or any combination thereof.

Embodiment 133 is the method of any one embodiments 128-132, wherein the capture domain comprises a poly(T) sequence.

Embodiment 134 is the method of any one of embodiments 128-133, wherein the capture probe further comprises a functional domain.

Embodiment 135 is the method of embodiment 134, wherein step (b) further comprises generating a second strand of nucleic acid that comprises (i) a sequence that is complementary to all or a portion of the functional domain, (ii) a sequence that is complementary to all or a portion of the spatial barcode, and (iii) a sequence that corresponds to all or a portion of the sequence of the nucleic acid encoding the immune cell receptor.

Embodiment 136 is the method of embodiment 135, wherein step (b) further comprises amplifying the second strand of nucleic acid using (i) a first primer comprising all or a portion of the functional domain, wherein the functional domain is 5′ to the spatial barcode in the second strand of nucleic acid, and (ii) a second primer comprising a sequence that is substantially complementary to a portion of a sequence encoding a variable region of the immune cell receptor.

Embodiment 137 is the method of any one of embodiments 128-136, wherein the immune cell clonotype is a T cell clonotype.

Embodiment 138 is the method of embodiment 137, wherein the immune cell receptor is a T cell receptor alpha chain.

Embodiment 139 is the method of embodiment 138, wherein step (b) comprises determining a sequence encoding CDR3 of the T cell receptor alpha chain.

Embodiment 140 is the method of embodiment 139, wherein step (b) further comprises determining a sequence encoding one or both of CDR1 and CDR2 of the T cell receptor alpha chain.

Embodiment 141 is the method of embodiment 139, wherein step (b) further comprises determining a sequence encoding a full-length variable domain of the T cell receptor alpha chain.

Embodiment 142 is the method of embodiment 137, wherein the immune cell receptor is a T cell receptor beta chain.

Embodiment 143 is the method of embodiment 142, wherein step (b) comprises determining a sequence encoding CDR3 of the T cell receptor beta chain.

Embodiment 144 is the method of embodiment 143, wherein step (b) further comprises determining a sequence encoding one or both of CDR1 and CDR2 of the T cell receptor beta chain.

Embodiment 145 is the method of embodiment 143, wherein step (b) further comprises determining a full-length variable domain of the T cell receptor beta chain.

Embodiment 146 is the method of any one of embodiments 128-136, wherein the immune cell clonotype is a B cell clonotype.

Embodiment 147 is the method of embodiment 146, wherein the immune cell receptor is an immunoglobulin kappa light chain.

Embodiment 148 is the method of embodiment 147, wherein step (b) comprises determining a sequence encoding CDR3 of the immunoglobulin kappa light chain.

Embodiment 149 is the method of embodiment 148, wherein step (b) further comprises determining a sequence encoding one or both of CDR1 and CDR2 of the immunoglobulin kappa light chain.

Embodiment 150 is the method of embodiment 148, wherein step (b) further comprises determining a sequence encoding a full-length variable domain of the immunoglobulin kappa light chain.

Embodiment 151 is the method of embodiment 146, wherein the immune cell receptor is an immunoglobulin lambda light chain.

Embodiment 152 is the method of embodiment 151, wherein step (b) comprises determining a sequence encoding CDR3 of the immunoglobulin lambda light chain.

Embodiment 153 is the method of embodiment 152, wherein step (b) further comprises determining a sequence encoding one or both of CDR1 and CDR2 of the immunoglobulin lambda light chain.

Embodiment 154 is the method of embodiment 152, wherein step (b) further comprises determining a sequence encoding a full-length variable domain of the immunoglobulin lambda light chain.

Embodiment 155 is the method of embodiment 146, wherein the immune cell receptor is an immunoglobulin heavy chain.

Embodiment 156 is the method of embodiment 155, wherein step (b) comprises determining a sequence encoding CDR3 of the immunoglobulin heavy chain.

Embodiment 157 is the method of embodiment 156, wherein step (b) further comprises determining a sequence encoding one or both of CDR1 and CDR2 of the immunoglobulin heavy chain.

Embodiment 158 is the method of embodiment 156, wherein step (b) further comprises determining a sequence encoding a full-length variable domain of the immunoglobulin heavy chain.

Embodiment 159 is the method of any one of embodiments 128-158, wherein the biological sample comprises a tissue sample.

Embodiment 160 is the method of embodiment 159, wherein the tissue sample is a tissue section.

Embodiment 161 is the method of embodiment 160, wherein the tissue section is a fixed tissue section.

Embodiment 162 is the method of embodiment 161, wherein the fixed tissue section is a formalin-fixed paraffin-embedded tissue section.

Embodiment 163 is the method of any one of embodiments 160-162, wherein the tissue section comprises a tumor region.

Embodiment 164 is the method of any one of embodiments 128-163, wherein the nucleic acid encoding the immune cell receptor comprises RNA.

Embodiment 165 is the method of embodiment 164, wherein the RNA is mRNA.

Embodiment 166 is the method of any one of embodiments 128-163, wherein the nucleic acid encoding the immune cell receptor comprises DNA.

Embodiment 167 is the method of embodiment 166, wherein the DNA is genomic DNA.

Embodiment 168 is the method of any one of embodiments 128-167, wherein the method further comprises, prior to step (b), contacting the biological sample with ribosomal RNA depletion probes and mitochondrial RNA depletion probes.

Embodiment 169 is the method of any one of embodiments 128-168, wherein the method further comprises imaging the biological sample.

Embodiment 170 is the method of any one of embodiments 128-169, wherein the determining in step (b) comprises sequencing (i) all or a portion of the sequence of the spatial barcode or a complement thereof, and (ii) all or a portion of the sequence of the nucleic acid encoding the immune cell receptor or a complement thereof.

Embodiment 171 is the method of any one of embodiments 128-170, wherein step (b) comprises determining the presence of the immune cell clonotype at a location in the biological sample.

Embodiment 172 is the method of any one of embodiments 128-171, wherein step (b) comprises determining the abundance of the immune cell clonotype at a location in the biological sample.

Embodiment 173 is the method of any one of embodiments 128-172, wherein step (b) comprises determining the presence and abundance of the immune cell clonotype at a location in the biological sample.

Embodiment 174 is the method of any one of embodiments 128-173, wherein step (b) comprises determining the presence of two or more immune cell clonotypes at a location in the biological sample.

Embodiment 175 is the method of any one of embodiments 128-174, wherein step (b) comprises determining the abundance of two or more immune cell clonotypes at a location in the biological sample.

Embodiment 176 is the method of any one of embodiments 128-174, wherein step (b) comprises determining the presence and abundance of two or more immune cell clonotypes at a location in the biological sample.

Embodiment 177 is the method of any one of embodiments 174-176, wherein the method further comprises comparing the two or more immune cell clonotypes.

Embodiment 178 is the method of any one of embodiments 174-177, wherein the two or more immune cell clonotypes are each a B cell clonotype.

Embodiment 179 is the method of any one of embodiments 174-177, wherein the two or more immune cell clonotypes are each a T cell clonotype.

Embodiment 180 is the method of any one of embodiments 174-177, wherein the two or more immune cell clonotypes comprise at least one T cell clonotype and at least one B cell clonotype.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition section headings, the materials, methods, and examples are illustrative only and not intended to be limiting.

What is claimed is:

1. A method for determining a location of a T cell clonotype in a tissue sample, the method comprising:
(a) contacting the tissue sample with an array comprising a plurality of capture probes, wherein a capture probe of the plurality of capture probes comprises in a 5′ to 3′ direction: (i) an adaptor domain, (ii) a spatial barcode, and (iii) a capture domain that hybridizes to a nucleic acid encoding a T cell receptor of the T cell clonotype, wherein the array comprises a plurality of beads disposed on a substrate, the plurality of beads collectively comprising the plurality of capture probes;
(b) hybridizing the capture domain of the capture probe to a poly(A) sequence of the nucleic acid encoding the T cell receptor of the T cell clonotype;
(c) extending the capture probe using the nucleic acid encoding the T cell receptor as a template, thereby generating an extended capture probe;
(d) performing an enrichment polymerase chain reaction using (i) the extended capture probe, or a complement thereof, as a template; (ii) a first primer complementary to the adaptor domain of the capture probe, and (iii) a second primer complementary to a sequence 5′ to a sequence encoding CDR3 of the nucleic acid encoding the T cell receptor, thereby generating an enriched extended capture probe or a complement thereof; and
(e) determining (iv) the sequence of the spatial barcode of the enriched extended capture probe or a complement thereof, and (v) all or a portion of the sequence of the nucleic acid encoding the T cell receptor from the enriched extended capture probe or a complement thereof, and using the determined sequences of (iv) and (v) to determine the location of the T cell clonotype in the tissue sample.


SEQUENCE LISTING (640 sequences)

SEQ ID NO: 1 - DNA, 20 bp, synthetic construct
  source 1..20: mol_type = other DNA
tctgatggctcaaacacagc

SEQ ID NO: 2 - DNA, 21 bp, synthetic construct
  source 1..21: mol_type = other DNA
gccagggggaagaccgatggg

SEQ ID NO: 3 - DNA, 18 bp, synthetic construct
  source 1..18: mol_type = other DNA
cacgctgctcgtatccga

SEQ ID NO: 4 - DNA, 27 bp, synthetic construct
  source 1..27: mol_type = other DNA
caacagggagaagaggatcctcaggcc

SEQ ID NO: 5 - DNA, 29 bp, synthetic construct
  source 1..29: mol_type = other DNA
ggacaaaacattgaccagcccactgagat

SEQ ID NO: 6 - DNA, 30 bp, synthetic construct
  source 1..30: mol_type = other DNA
aaaaaccaagtggagcagagtcctcagtcc

SEQ ID NO: 7 - DNA, 25 bp, synthetic construct
  source 1..25: mol_type = other DNA
caacggaaggaggtggagcaggatc

SEQ ID NO: 8 - DNA, 29 bp, synthetic construct
  source 1..29: mol_type = other DNA
caacagaaggaggtggagcagaattctgg

SEQ ID NO: 9 - DNA, 27 bp, synthetic construct
  source 1..27: mol_type = other DNA
caacagaaggaggtggagcaggatcct

SEQ ID NO: 10 - DNA, 27 bp, synthetic construct
  source 1..27: mol_type = other DNA
gagaatgtggagcagcatccttcaacc

SEQ ID NO: 11 - DNA, 27 bp, synthetic construct
  source 1..27: mol_type = other DNA
gagagtgtggggctgcatcttcctacc

SEQ ID NO: 12 - DNA, 33 bp, synthetic construct
  source 1..33: mol_type = other DNA
cagaagataactcaaacccaaccaggaatgttc

SEQ ID NO: 13 - DNA, 27 bp, synthetic construct
  source 1..27: mol_type = other DNA
cagagagtgactcagcccgagaagctc

SEQ ID NO: 14 - DNA, 26 bp, synthetic construct
  source 1..26: mol_type = other DNA
gactcggttacccagacagaaggccc

SEQ ID NO: 15 - DNA, 33 bp, synthetic construct
  source 1..33: mol_type = other DNA
cagaaggtaactcaagcgcagactgaaatttct

SEQ ID NO: 16 - DNA, 30 bp, synthetic construct
  source 1..30: mol_type = other DNA
aaggaccaagtgtttcagccttccacagtg

SEQ ID NO: 17 - DNA, 25 bp, synthetic construct
  source 1..25: mol_type = other DNA
gaagaccaggtgacgcagagtcccg

SEQ ID NO: 18 - DNA, 26 bp, synthetic construct
  source 1..26: mol_type = other DNA
aaacaggaggtgacgcagattcctgc

SEQ ID NO: 19 - DNA, 31 bp, synthetic construct
  source 1..31: mol_type = other DNA
atacaagtggagcagagtcctccagacctga

SEQ ID NO: 20 - DNA, 29 bp, synthetic construct
  source 1..29: mol_type = other DNA
caacagaaggagaaaagtgaccagcagca

SEQ ID NO: 21 - DNA, 33 bp, synthetic construct
  source 1..33: mol_type = other DNA
atactgaacgtggaacaaagtcctcagtcactg

SEQ ID NO: 22 - DNA, 31 bp, synthetic construct
  source 1..31: mol_type = other DNA
caacaggtaatgcaaattcctcagtaccagc

SEQ ID NO: 23 - DNA, 21 bp, synthetic construct
  source 1..21: mol_type = other DNA
aagaccacccagcccccctcc

SEQ ID NO: 24 - DNA, 33 bp, synthetic construct
  source 1..33: mol_type = other DNA
aagaccacacagccaaattcaatggagagtaac

SEQ ID NO: 25 - DNA, 25 bp, synthetic construct
  source 1..25: mol_type = other DNA
cagctgctggagcagagccctcagt

SEQ ID NO: 26 - DNA, 33 bp, synthetic construct
  source 1..33: mol_type = other DNA
caacagaagaatgatgaccagcaagttaagcaa

SEQ ID NO: 27 - DNA, 25 bp, synthetic construct
  source 1..25: mol_type = other DNA
cagtcagtggctcagccggaagatc

SEQ ID NO: 28 - DNA, 27 bp, synthetic construct
  source 1..27: mol_type = other DNA
caacaaccagtgcagagtcctcaagcc

SEQ ID NO: 29 - DNA, 30 bp, synthetic construct
  source 1..30: mol_type = other DNA
caagaactggagcagagtcctcagtccttg

SEQ ID NO: 30 - DNA, 36 bp, synthetic construct
  source 1..36: mol_type = other DNA
caacagctgaatcagagtcctcaatctatgtttatc

SEQ ID NO: 31 - DNA, 33 bp, synthetic construct
  source 1..33: mol_type = other DNA
gaagacaaggtggtacaaagccctctatctctg

SEQ ID NO: 32 - DNA, 33 bp, synthetic construct
  source 1..33: mol_type = other DNA
cagacagtcactcagtctcaaccagagatgtct

SEQ ID NO: 33 - DNA, 30 bp, synthetic construct
  source 1..30: mol_type = other DNA
gagctgaaagtggaacaaaaccctctgttc

SEQ ID NO: 34 - DNA, 24 bp, synthetic construct
  source 1..24: mol_type = other DNA
aagaccacccagcccatctccatg

SEQ ID NO: 35 - DNA, 29 bp, synthetic construct
  source 1..29: mol_type = other DNA
aattcagtcaagcagacgggccaaataac

SEQ ID NO: 36 - DNA, 28 bp, synthetic construct
  source 1..28: mol_type = other DNA
gccaaaaatgaagtggagcagagtcctc

SEQ ID NO: 37 - DNA, 31 bp, synthetic construct
  source 1..31: mol_type = other DNA
gaggatgtggagcagagtcttttcctgagtg

SEQ ID NO: 38 - DNA, 30 bp, synthetic construct
  source 1..30: mol_type = other DNA
caaaagatagaacagaattccgaggccctg

SEQ ID NO: 39 - DNA, 25 bp, synthetic construct
  source 1..25: mol_type = other DNA
gaaaaccaggtggagcacagccctc

SEQ ID NO: 40 - DNA, 27 bp, synthetic construct
  source 1..27: mol_type = other DNA
cagtctgtgagccagcataaccaccac

SEQ ID NO: 41 - DNA, 24 bp, synthetic construct
  source 1..24: mol_type = other DNA
cagtcggtgacccagcttgacagc

SEQ ID NO: 42 - DNA, 27 bp, synthetic construct
  source 1..27: mol_type = other DNA
cagtcagtgacccagcctgacatccac

SEQ ID NO: 43 - DNA, 23 bp, synthetic construct
  source 1..23: mol_type = other DNA
cagtcggtgacccagcttggcag

SEQ ID NO: 44 - DNA, 26 bp, synthetic construct
  source 1..26: mol_type = other DNA
cagtctgtgacccagcttgacagcca

SEQ ID NO: 45 - DNA, 24 bp, synthetic construct
  source 1..24: mol_type = other DNA
cagtcggtgacccagcttgatggc

SEQ ID NO: 46 - DNA, 29 bp, synthetic construct
  source 1..29: mol_type = other DNA
gattcagtggtccagacagaaggccaagt

SEQ ID NO: 47 - DNA, 26 bp, synthetic construct
  source 1..26: mol_type = other DNA
aattcagtgacccagatggaagggcc

SEQ ID NO: 48 - DNA, 27 bp, synthetic construct
  source 1..27: mol_type = other DNA
gaacctgaagtcacccagactcccagc

SEQ ID NO: 49 - DNA, 30 bp, synthetic construct
  source 1..30: mol_type = other DNA
gctgtttcccagactccaaaatacctggtc

SEQ ID NO: 50 - DNA, 30 bp, synthetic construct
  source 1..30: mol_type = other DNA
gaagttacccagacaccaaaacacctggtc

SEQ ID NO: 51 - DNA, 35 bp, synthetic construct
  source 1..35: mol_type = other DNA
ggagtcactcaaactccaagatatctgatcaaaac

SEQ ID NO: 52 - DNA, 27 bp, synthetic construct
  source 1..27: mol_type = other DNA
ggtgtcactcagaccccaaaattccag

SEQ ID NO: 53 - DNA, 28 bp, synthetic construct
  source 1..28: mol_type = other DNA
ggagtctcccagtccctgagacacaagg

SEQ ID NO: 54 - DNA, 28 bp, synthetic construct
  source 1..28: mol_type = other DNA
ggagttacgcagacaccaagacacctgg

SEQ ID NO: 55 - DNA, 26 bp, synthetic construct
  source 1..26: mol_type = other DNA
ggtgtcactcagaccccaaaattccg

SEQ ID NO: 56 - DNA, 27 bp, synthetic construct
  source 1..27: mol_type = other DNA
ggagtctcccagtcccccagtaacaag

SEQ ID NO: 57 - DNA, 25 bp, synthetic construct
  source 1..25: mol_type = other DNA
gggatcacccaggcaccaacatctc

SEQ ID NO: 58 - DNA, 27 bp, synthetic construct
  source 1..27: mol_type = other DNA
ggagtctcccagacccccagtaacaag

SEQ ID NO: 59 - DNA, 26 bp, synthetic construct
  source 1..26: mol_type = other DNA
ggagtcacccaaagtcccacacacct

SEQ ID NO: 60 - DNA, 26 bp, synthetic construct
  source 1..26: mol_type = other DNA
ggagtcacacaaaccccaaagcacct

SEQ ID NO: 61 - DNA, 28 bp, synthetic construct
  source 1..28: mol_type = other DNA
gaaatcacccagagcccaagacacaaga

SEQ ID NO: 62 - DNA, 31 bp, synthetic construct
  source 1..31: mol_type = other DNA
gaagttgcccagtcccccagatataagatta

SEQ ID NO: 63 - DNA, 29 bp, synthetic construct
  source 1..29: mol_type = other DNA
ggaatcacccagagcccaagatacaagat

SEQ ID NO: 64 - DNA, 36 bp, synthetic construct
  source 1..36: mol_type = other DNA
ggagttgcccagtctcccagatataagattatagag

SEQ ID NO: 65 - DNA, 28 bp, synthetic construct
  source 1..28: mol_type = other DNA
ggagtctcccagtccccaaggtacaaag

SEQ ID NO: 66 - DNA, 26 bp, synthetic construct
  source 1..26: mol_type = other DNA
ggagtctcccagtccccaaggtacga

SEQ ID NO: 67 - DNA, 27 bp, synthetic construct
  source 1..27: mol_type = other DNA
ggtgtcactcagaccccaaaattccac

SEQ ID NO: 68 - DNA, 30 bp, synthetic construct
  source 1..30: mol_type = other DNA
ggagtctcccagtctcccaggtacaaagtc

SEQ ID NO: 69 - DNA, 29 bp, synthetic construct
  source 1..29: mol_type = other DNA
ggtgtcactcagaccccaaaattccacat

SEQ ID NO: 70 - DNA, 30 bp, synthetic construct
  source 1..30: mol_type = other DNA
ggagtctcccagtcccctaggtacaaagtc

SEQ ID NO: 71 - DNA, 28 bp, synthetic construct
  source 1..28: mol_type = other DNA
ggagtcacacaaagtcccacacacctga

SEQ ID NO: 72 - DNA, 27 bp, synthetic construct
  source 1..27: mol_type = other DNA
ggagtctcccagaaccccagacacaag

SEQ ID NO: 73 - DNA, 29 bp, synthetic construct
  source 1..29: mol_type = other DNA
ggagtcatccagtccccaagacatctgat

SEQ ID NO: 74 - DNA, 25 bp, synthetic construct
  source 1..25: mol_type = other DNA
ggagttatccagtcaccccgccatg

SEQ ID NO: 75 - DNA, 24 bp, synthetic construct
  source 1..24: mol_type = other DNA
ggagttatccagtcaccccggcac

SEQ ID NO: 76 - DNA, 27 bp, synthetic construct
  source 1..27: mol_type = other DNA
agagtcacccagacaccaaggcacaag

SEQ ID NO: 77 - DNA, 27 bp, synthetic construct
  source 1..27: mol_type = other DNA
ggagttactcagttccccagccacagc

SEQ ID NO: 78 - DNA, 30 bp, synthetic construct
  source 1..30: mol_type = other DNA
atggtcatccagaacccaagataccaggtt

SEQ ID NO: 79 - DNA, 23 bp, synthetic construct
  source 1..23: mol_type = other DNA
gagcctggagtcagccagacccc

SEQ ID NO: 80 - DNA, 24 bp, synthetic construct
  source 1..24: mol_type = other DNA
ggcgtcatgcagaacccaagacac

SEQ ID NO: 81 - DNA, 31 bp, synthetic construct
  source 1..31: mol_type = other DNA
ggaatcactcagtccccaaagtacctgttca

SEQ ID NO: 82 - DNA, 26 bp, synthetic construct
  source 1..26: mol_type = other DNA
gctgtcgtctctcaacatccgagctg

SEQ ID NO: 83 - DNA, 24 bp, synthetic construct
  source 1..24: mol_type = other DNA
attccagctcactggggctggatg

SEQ ID NO: 84 - DNA, 31 bp, synthetic construct
  source 1..31: mol_type = other DNA
aaagtcacacagactccaggacatttggtca

SEQ ID NO: 85 - DNA, 30 bp, synthetic construct
  source 1..30: mol_type = other DNA
gatgttacccagaccccaaggaataggatc

SEQ ID NO: 86 - DNA, 35 bp, synthetic construct
  source 1..35: mol_type = other DNA
gacatctaccagaccccaagataccttgttatagg

SEQ ID NO: 87 - DNA, 35 bp, synthetic construct
  source 1..35: mol_type = other DNA
gtagttacacaattcccaagacacagaatcattgg

SEQ ID NO: 88 - DNA, 30 bp, synthetic construct
  source 1..30: mol_type = other DNA
caagtgacccagaacccaagatacctcatc

SEQ ID NO: 89 - DNA, 27 bp, synthetic construct
  source 1..27: mol_type = other DNA
ggtggcagcagtcacagatgcctactc

SEQ ID NO: 90 - DNA, 27 bp, synthetic construct
  source 1..27: mol_type = other DNA
ggtggcagcagccacaggtgcccactc

SEQ ID NO: 91 - DNA, 27 bp, synthetic construct
  source 1..27: mol_type = other DNA
ggtggcagcagctacaggtgtccagtc

SEQ ID NO: 92 - DNA, 27 bp, synthetic construct
  source 1..27: mol_type = other DNA
ggtggsagcagcaacargwgcccactc

SEQ ID NO: 93 - DNA, 27 bp, synthetic construct
  source 1..27: mol_type = other DNA
gctggctgtagctccaggtgctcactc

SEQ ID NO: 94 - DNA, 33 bp, synthetic construct
  source 1..33: mol_type = other DNA
cctgctgctgaccaycccttcmtgggtcttgtc

SEQ ID NO: 95 - DNA, 33 bp, synthetic construct
  source 1..33: mol_type = other DNA
cctgctactgactgtcccgtcctgggtcttatc

SEQ ID NO: 96 - DNA, 37 bp, synthetic construct
  source 1..37: mol_type = other DNA
gggttttcctcgttgctcttttaagaggtgtccagtg

SEQ ID NO: 97 - DNA, 37 bp, synthetic construct
  source 1..37: mol_type = other DNA
gggttttccttgttgctattttaaaaggtgtccartg

SEQ ID NO: 98 - DNA, 37 bp, synthetic construct
  source 1..37: mol_type = other DNA
ggattttccttgctgctattttaaaaggtgtccagtg

SEQ ID NO: 99 - DNA, 37 bp, synthetic construct
  source 1..37: mol_type = other DNA
gggttttccttktkgctatwttagaaggtgtccagtg

SEQ ID NO: 100 - DNA, 27 bp, synthetic construct
  source 1..27: mol_type = other DNA
ggtggcrgctcccagatgggtcctgtc

SEQ ID NO: 101 - DNA, 25 bp, synthetic construct
  source 1..25: mol_type = other DNA
ctggctgttctccaaggagtctgtg

SEQ ID NO: 102 - DNA, 23 bp, synthetic construct
  source 1..23: mol_type = other DNA
ggcctcccatggggtgtcctgtc

SEQ ID NO: 103 - DNA, 26 bp, synthetic construct
  source 1..26: mol_type = other DNA
ggtggcagcagcaacaggtgcccact

SEQ ID NO: 104 - DNA, 28 bp, synthetic construct
  source 1..28: mol_type = other DNA
atggaactggggctccgctgggttttcc

SEQ ID NO: 105 - DNA, 27 bp, synthetic construct
  source 1..27: mol_type = other DNA
atggactgcacctggaggatcctcctc

SEQ ID NO: 106 - DNA, 26 bp, synthetic construct
  source 1..26: mol_type = other DNA
tggctgagctgggtttyccttgttgc

SEQ ID NO: 107 - DNA, 26 bp, synthetic construct
  source 1..26: mol_type = other DNA
ggagttkgggctgmgctgggttttcc

SEQ ID NO: 108 - DNA, 28 bp, synthetic construct
  source 1..28: mol_type = other DNA
gcacctgtggtttttcctcctgctggtg

SEQ ID NO: 109 - DNA, 25 bp, synthetic construct
  source 1..25: mol_type = other DNA
cacctgtggttcttcctcctsctgg

SEQ ID NO: 110 - DNA, 26 bp, synthetic construct
  source 1..26: mol_type = other DNA
ccaggatggggtcaaccgccatcctc

SEQ ID NO: 111 - DNA, 30 bp, synthetic construct
  source 1..30: mol_type = other DNA
cagaggactcaccatggagtttgggctgag

SEQ ID NO: 112 - DNA, 27 bp, synthetic construct
  source 1..27: mol_type = other DNA
ggactcaccatggagttgggactgagc

SEQ ID NO: 113 - DNA, 26 bp, synthetic construct
  source 1..26: mol_type = other DNA
gggctgagctggctttttcttgtggc

SEQ ID NO: 114 - DNA, 30 bp, synthetic construct
  source 1..30: mol_type = other DNA
aagcagtggtatcaacgcagagtacatggg

SEQ ID NO: 115 - DNA, 17 bp, synthetic construct
  source 1..17: mol_type = other DNA
tctgcgttgataccact

SEQ ID NO: 116 - DNA, 30 bp, Homo sapiens
  source 1..30: mol_type = genomic DNA
gaaggagctccagatgaaagactctgcctc

SEQ ID NO: 117 - DNA, 30 bp, Homo sapiens
  source 1..30: mol_type = genomic DNA
gttctcttcatcgctgctcatcctccaggt

SEQ ID NO: 118 - DNA, 30 bp, Homo sapiens
  source 1..30: mol_type = genomic DNA
cttgtgagcgactccgctttgtacttctgt

SEQ ID NO: 119 - DNA, 30 bp, Homo sapiens
  source 1..30: mol_type = genomic DNA
ttatccctgccgacagaaagtccagcactc

SEQ ID NO: 120 - DNA, 31 bp, Homo sapiens
  source 1..31: mol_type = genomic DNA
aaggataaacatctgtctctgcgcattgcag

SEQ ID NO: 121 - DNA, 30 bp, Homo sapiens
  source 1..30: mol_type = genomic DNA
ttgtttcatatcacagcctcccagcctgca

SEQ ID NO: 122 - DNA, 31 bp, Homo sapiens
  source 1..31: mol_type = genomic DNA
tacattacagccgtgcagcctgaagattcag

SEQ ID NO: 123 - DNA, 30 bp, Homo sapiens
  source 1..30: mol_type = genomic DNA
aatctgaggaaaccctctgtgcagtggagt

SEQ ID NO: 124 - DNA, 30 bp, Homo sapiens
  source 1..30: mol_type = genomic DNA
gaaacctccttccacctgacgaaaccctca

SEQ ID NO: 125 - DNA, 30 bp, Homo sapiens
  source 1..30: mol_type = genomic DNA
caatctgaggaaaccctctgtgcattggag

SEQ ID NO: 126 - DNA, 30 bp, Homo sapiens
  source 1..30: mol_type = genomic DNA
cacctgacgaaaccctcagcccatatgagc

SEQ ID NO: 127 - DNA, 30 bp, Homo sapiens
  source 1..30: mol_type = genomic DNA
ggaaaccctcagtccatataagcgacacgg

SEQ ID NO: 128 - DNA, 30 bp, Homo sapiens
  source 1..30: mol_type = genomic DNA
gaggaaaccatcaacccatgtgagtgatgc

SEQ ID NO: 129 - DNA, 32 bp, Homo sapiens
  source 1..32: mol_type = genomic DNA
ggaaggaacaaaggttttgaagccatgtaccg

SEQ ID NO: 130 - DNA, 30 bp, Homo sapiens
  source 1..30: mol_type = genomic DNA
tccacttggagaaaggctcagttcaagtgt

SEQ ID NO: 131 - DNA, 30 bp, Homo sapiens
  source 1..30: mol_type = genomic DNA
gcagacacaaagcaaagctctctgcacatc

SEQ ID NO: 132 - DNA, 30 bp, Homo sapiens
  source 1..30: mol_type = genomic DNA
gccagccagtatatttccctgctcatcaga

SEQ ID NO: 133 - DNA, 30 bp, Homo sapiens
  source 1..30: mol_type = genomic DNA
gccagccagtatgtttctctgctcatcaga

SEQ ID NO: 134 - DNA, 30 bp, Homo sapiens
  source 1..30: mol_type = genomic DNA
ggtttacagcacaggtcgataaatccagca

SEQ ID NO: 135 - DNA, 30 bp, Homo sapiens
  source 1..30: mol_type = genomic DNA
gccaaacatttctccctgcacatcacagag

SEQ ID NO: 136 - DNA, 30 bp, Homo sapiens
  source 1..30: mol_type = genomic DNA
tctgcaaattgcagctactcaacctggaga

SEQ ID NO: 137 - DNA, 30 bp, Homo sapiens
  source 1..30: mol_type = genomic DNA
gccaaccttgtcatctccgcttcacaactg

SEQ ID NO: 138 - DNA, 31 bp, Homo sapiens
  source 1..31: mol_type = genomic DNA
gaccttaacaaaggcgagacatctttccacc

SEQ ID NO: 139 - DNA, 30 bp, Homo sapiens
  source 1..30: mol_type = genomic DNA
gtcacgcttgacacttccaagaaaagcagt

SEQ ID NO: 140 - DNA, 30 bp, Homo sapiens
  source 1..30: mol_type = genomic DNA
cctatcaagagtgacagttccttccacctg

SEQ ID NO: 141 - DNA, 30 bp, Homo sapiens
  source 1..30: mol_type = genomic DNA
ggaacttccagaaatccaccagttccttca

SEQ ID NO: 142 - DNA, 29 bp, Homo sapiens
  source 1..29: mol_type = genomic DNA
agaaggaaagctttctgcacatcacagcc

SEQ ID NO: 143 - DNA, 30 bp, Homo sapiens
  source 1..30: mol_type = genomic DNA
caagtggaagacttaatgcctcgctggata

SEQ ID NO: 144 - DNA, 30 bp, Homo sapiens
  source 1..30: mol_type = genomic DNA
gactgtcgctacggaacgctacagcttatt

SEQ ID NO: 145 - DNA, 30 bp, Homo sapiens
  source 1..30: mol_type = genomic DNA
tgccaagcagttctcatcgcatatcatgga

SEQ ID NO: 146 - DNA, 30 bp, Homo sapiens
  source 1..30: mol_type = genomic DNA
gccactcttaataccaaggagggttacagc

SEQ ID NO: 147 - DNA, 30 bp, Homo sapiens
  source 1..30: mol_type = genomic DNA
cacatcacagccacccagactacagatgta

SEQ ID NO: 148 - DNA, 30 bp, Homo sapiens
  source 1..30: mol_type = genomic DNA
tcatcacagaagacagaaagtccagcacct

SEQ ID NO: 149 - DNA, 30 bp, Homo sapiens
  source 1..30: mol_type = genomic DNA
agaaagtccagtaccttgatcctgcaccgt

SEQ ID NO: 150 - DNA, 30 bp, Homo sapiens
  source 1..30: mol_type = genomic DNA
gttctctccacatcactgcagcccagactg

SEQ ID NO: 151 - DNA, 30 bp, Homo sapiens
  source 1..30: mol_type = genomic DNA
aaagtgccaagcacctctctctgcacattg

SEQ ID NO: 152 - DNA, 30 bp, Homo sapiens
  source 1..30: mol_type = genomic DNA
ctgtaccttacggcctcccagctcagttac

SEQ ID NO: 153 - DNA, 30 bp, Homo sapiens
  source 1..30: mol_type = genomic DNA
gccaagttggatgagaaaaagcagcaaagt

SEQ ID NO: 154 - DNA, 30 bp, Homo sapiens
  source 1..30: mol_type = genomic DNA
gacctcaaatggaagactgactgctcagtt

SEQ ID NO: 155 - DNA, 30 bp, Homo sapiens
  source 1..30: mol_type = genomic DNA
tttcagcatcctgaacatcacagccaccca

SEQ ID NO: 156 - DNA, 30 bp, Homo sapiens
  source 1..30: mol_type = genomic DNA
ccttcagtctcaagatctcagactcacagc

SEQ ID NO: 157 - DNA, 29 bp, Homo sapiens
  source 1..29: mol_type = genomic DNA
aatggcctcacttgataccaaagcccgtc

SEQ ID NO: 158 - DNA, 30 bp, Homo sapiens
  source 1..30: mol_type = genomic DNA
ctcccccattgtgaaatattcagtccaggt

SEQ ID NO: 159 - DNA, 30 bp, Homo sapiens
  source 1..30: mol_type = genomic DNA
catacaggaaaagcacagctccctgcacat

SEQ ID NO: 160 - DNA, 31 bp, Homo sapiens
  source 1..31: mol_type = genomic DNA
atatcgcagcctctcatctgggagattcagc

SEQ ID NO: 161 - DNA, 31 bp, Homo sapiens
  source 1..31: mol_type = genomic DNA
caggagctccagatgaaagactctgcctctt

SEQ ID NO: 162 - DNA, 32 bp, Homo sapiens
  source 1..32: mol_type = genomic DNA
acttccttccacttgaggaaaccctcagtcca

SEQ ID NO: 163 - DNA, 64 bp, synthetic construct
  source 1..64: mol_type = other DNA
gtgactggagttcagacgtgtgctcttccgatctgaaggagctccagatgaaagactctgcctc

SEQ ID NO: 164 - DNA, 64 bp, synthetic construct
  source 1..64: mol_type = other DNA
gtgactggagttcagacgtgtgctcttccgatctgttctcttcatcgctgctcatcctccaggt

SEQ ID NO: 165 - DNA, 64 bp, synthetic construct
  source 1..64: mol_type = other DNA
gtgactggagttcagacgtgtgctcttccgatctcttgtgagcgactccgctttgtacttctgt

SEQ ID NO: 166 - DNA, 64 bp, synthetic construct
  source 1..64: mol_type = other DNA
gtgactggagttcagacgtgtgctcttccgatctttatccctgccgacagaaagtccagcactc

SEQ ID NO: 167 - DNA, 65 bp, synthetic construct
  source 1..65: mol_type = other DNA
gtgactggagttcagacgtgtgctcttccgatctaaggataaacatctgtctctgcgcattgcag

SEQ ID NO: 168 - DNA, 64 bp, synthetic construct
  source 1..64: mol_type = other DNA
gtgactggagttcagacgtgtgctcttccgatctttgtttcatatcacagcctcccagcctgca

SEQ ID NO: 169 - DNA, 65 bp, synthetic construct
  source 1..65: mol_type = other DNA
gtgactggagttcagacgtgtgctcttccgatcttacattacagccgtgcagcctgaagattcag

SEQ ID NO: 170 - DNA, 64 bp, synthetic construct
  source 1..64: mol_type = other DNA
gtgactggagttcagacgtgtgctcttccgatctaatctgaggaaaccctctgtgcagtggagt

SEQ ID NO: 171 - DNA, 64 bp, synthetic construct
  source 1..64: mol_type = other DNA
gtgactggagttcagacgtgtgctcttccgatctgaaacctccttccacctgacgaaaccctca

SEQ ID NO: 172 - DNA, 64 bp, synthetic construct
  source 1..64: mol_type = other DNA
gtgactggagttcagacgtgtgctcttccgatctcaatctgaggaaaccctctgtgcattggag

SEQ ID NO: 173 - DNA, 64 bp, synthetic construct
  source 1..64: mol_type = other DNA
gtgactggagttcagacgtgtgctcttccgatctcacctgacgaaaccctcagcccatatgagc

SEQ ID NO: 174 - DNA, 64 bp, synthetic construct
  source 1..64: mol_type = other DNA
gtgactggagttcagacgtgtgctcttccgatctggaaaccctcagtccatataagcgacacgg

SEQ ID NO: 175 - DNA, 64 bp, synthetic construct
  source 1..64: mol_type = other DNA
gtgactggagttcagacgtgtgctcttccgatctgaggaaaccatcaacccatgtgagtgatgc

SEQ ID NO: 176 - DNA, 66 bp, synthetic construct
  source 1..66: mol_type = other DNA
gtgactggagttcagacgtgtgctcttccgatctggaaggaacaaaggttttgaagccatgtaccg

SEQ ID NO: 177 - DNA, 64 bp, synthetic construct
  source 1..64: mol_type = other DNA
gtgactggagttcagacgtgtgctcttccgatcttccacttggagaaaggctcagttcaagtgt

SEQ ID NO: 178 - DNA, 64 bp, synthetic construct
  source 1..64: mol_type = other DNA
gtgactggagttcagacgtgtgctcttccgatctgcagacacaaagcaaagctctctgcacatc

SEQ ID NO: 179 - DNA, 64 bp, synthetic construct
  source 1..64: mol_type = other DNA
gtgactggagttcagacgtgtgctcttccgatctgccagccagtatatttccctgctcatcaga

SEQ ID NO: 180 - DNA, 64 bp, synthetic construct
  source 1..64: mol_type = other DNA
gtgactggagttcagacgtgtgctcttccgatctgccagccagtatgtttctctgctcatcaga

SEQ ID NO: 181 - DNA, 64 bp, synthetic construct
  source 1..64: mol_type = other DNA
gtgactggagttcagacgtgtgctcttccgatctggtttacagcacaggtcgataaatccagca

SEQ ID NO: 182 - DNA, 64 bp, synthetic construct
  source 1..64: mol_type = other DNA
gtgactggagttcagacgtgtgctcttccgatctgccaaacatttctccctgcacatcacagag

SEQ ID NO: 183 - DNA, 64 bp, synthetic construct
  source 1..64: mol_type = other DNA
gtgactggagttcagacgtgtgctcttccgatcttctgcaaattgcagctactcaacctggaga

SEQ ID NO: 184 - DNA, 64 bp, synthetic construct
  source 1..64: mol_type = other DNA
gtgactggagttcagacgtgtgctcttccgatctgccaaccttgtcatctccgcttcacaactg

SEQ ID NO: 185 - DNA, 65 bp, synthetic construct
  source 1..65: mol_type = other DNA
gtgactggagttcagacgtgtgctcttccgatctgaccttaacaaaggcgagacatctttccacc

SEQ ID NO: 186 - DNA, 64 bp, synthetic construct
  source 1..64: mol_type = other DNA
gtgactggagttcagacgtgtgctcttccgatctgtcacgcttgacacttccaagaaaagcagt

SEQ ID NO: 187 - DNA, 64 bp, synthetic construct
  source 1..64: mol_type = other DNA
gtgactggagttcagacgtgtgctcttccgatctcctatcaagagtgacagttccttccacctg

SEQ ID NO: 188 - DNA, 64 bp, synthetic construct
  source 1..64: mol_type = other DNA
gtgactggagttcagacgtgtgctcttccgatctggaacttccagaaatccaccagttccttca

SEQ ID NO: 189 - DNA, 63 bp, synthetic construct
  source 1..63: mol_type = other DNA
gtgactggagttcagacgtgtgctcttccgatctagaaggaaagctttctgcacatcacagcc

SEQ ID NO: 190 - DNA, 64 bp, synthetic construct
  source 1..64: mol_type = other DNA
gtgactggagttcagacgtgtgctcttccgatctcaagtggaagacttaatgcctcgctggata

SEQ ID NO: 191 - DNA, 64 bp, synthetic construct
  source 1..64: mol_type = other DNA
gtgactggagttcagacgtgtgctcttccgatctgactgtcgctacggaacgctacagcttatt

SEQ ID NO: 192 - DNA, 64 bp, synthetic construct
  source 1..64: mol_type = other DNA
gtgactggagttcagacgtgtgctcttccgatcttgccaagcagttctcatcgcatatcatgga

SEQ ID NO: 193 - DNA, 64 bp, synthetic construct
  source 1..64: mol_type = other DNA
gtgactggagttcagacgtgtgctcttccgatctgccactcttaataccaaggagggttacagc

SEQ ID NO: 194 - DNA, 64 bp, synthetic construct
  source 1..64: mol_type = other DNA
gtgactggagttcagacgtgtgctcttccgatctcacatcacagccacccagactacagatgta

SEQ ID NO: 195 - DNA, 64 bp, synthetic construct
  source 1..64: mol_type = other DNA
gtgactggagttcagacgtgtgctcttccgatcttcatcacagaagacagaaagtccagcacct

SEQ ID NO: 196 - DNA, 64 bp, synthetic construct
  source 1..64: mol_type = other DNA
gtgactggagttcagacgtgtgctcttccgatctagaaagtccagtaccttgatcctgcaccgt

SEQ ID NO: 197 - DNA, 64 bp, synthetic construct
  source 1..64: mol_type = other DNA
gtgactggagttcagacgtgtgctcttccgatctgttctctccacatcactgcagcccagactg

SEQ ID NO: 198 - DNA, 64 bp, synthetic construct
  source 1..64: mol_type = other DNA
gtgactggagttcagacgtgtgctcttccgatctaaagtgccaagcacctctctctgcacattg

SEQ ID NO: 199 - DNA, 64 bp, synthetic construct
  source 1..64: mol_type = other DNA
gtgactggagttcagacgtgtgctcttccgatctctgtaccttacggcctcccagctcagttac

SEQ ID NO: 200 - DNA, 64 bp, synthetic construct
  source 1..64: mol_type = other DNA
gtgactggagttcagacgtgtgctcttccgatctgccaagttggatgagaaaaagcagcaaagt

SEQ ID NO: 201 - DNA, 64 bp, synthetic construct
  source 1..64: mol_type = other DNA
gtgactggagttcagacgtgtgctcttccgatctgacctcaaatggaagactgactgctcagtt

SEQ ID NO: 202 - DNA, 64 bp, synthetic construct
  source 1..64: mol_type = other DNA
gtgactggagttcagacgtgtgctcttccgatcttttcagcatcctgaacatcacagccaccca

SEQ ID NO: 203 - DNA, 64 bp, synthetic construct
  source 1..64: mol_type = other DNA
gtgactggagttcagacgtgtgctcttccgatctccttcagtctcaagatctcagactcacagc

SEQ ID NO: 204 - DNA, 63 bp, synthetic construct
  source 1..63: mol_type = other DNA
gtgactggagttcagacgtgtgctcttccgatctaatggcctcacttgataccaaagcccgtc

SEQ ID NO: 205 - DNA, 64 bp, synthetic construct
  source 1..64: mol_type = other DNA
gtgactggagttcagacgtgtgctcttccgatctctcccccattgtgaaatattcagtccaggt

SEQ ID NO: 206 - DNA, 64 bp, synthetic construct
  source 1..64: mol_type = other DNA
gtgactggagttcagacgtgtgctcttccgatctcatacaggaaaagcacagctccctgcacat

SEQ ID NO: 207 - DNA, 65 bp, synthetic construct
  source 1..65: mol_type = other DNA
gtgactggagttcagacgtgtgctcttccgatctatatcgcagcctctcatctgggagattcagc

SEQ ID NO: 208 - DNA, 65 bp, synthetic construct
  source 1..65: mol_type = other DNA
gtgactggagttcagacgtgtgctcttccgatctcaggagctccagatgaaagactctgcctctt

SEQ ID NO: 209 - DNA, 66 bp, synthetic construct
  source 1..66: mol_type = other DNA
gtgactggagttcagacgtgtgctcttccgatctacttccttccacttgaggaaaccctcagtcca

SEQ ID NO: 210 - DNA, 34 bp, synthetic construct
  source 1..34: mol_type = other DNA
gtgactggagttcagacgtgtgctcttccgatct

SEQ ID NO: 211 - DNA, 33 bp, synthetic construct
  source 1..33: mol_type = other DNA
aaaaaccaagtggagcagagtcctcagtccctg

SEQ ID NO: 212 - DNA, 31 bp, synthetic construct
  source 1..31: mol_type = other DNA
aaacaggaggtgacgcagattcctgcagctc

SEQ ID NO: 213 - DNA, 32 bp, synthetic construct
  source 1..32: mol_type = other DNA
aaggaccaagtgtttcagccttccacagtggc

SEQ ID NO: 214 - DNA, 28 bp, synthetic construct
  source 1..28: mol_type = other DNA
acccagcttgacagccaagtccctgtct

SEQ ID NO: 215 - DNA, 30 bp, synthetic construct
  source 1..30: mol_type = other DNA
acccagcttgatggccacatcactgtctct

SEQ ID NO: 216 - DNA, 25 bp, synthetic construct
  source 1..25: mol_type = other DNA
acccagcttggcagccacgtctctg

SEQ ID NO: 217 - DNA, 31 bp, synthetic construct
  source 1..31: mol_type = other DNA
actcaagcgcagactgaaatttctgtggtgg

SEQ ID NO: 218 - DNA, 29 bp, synthetic construct
  source 1..29: mol_type = other DNA
agaaggaggtggagcaggatcctggacca

SEQ ID NO: 219 - DNA, 34 bp, synthetic construct
  source 1..34: mol_type = other DNA
agaattccgaggctctgaacattcaggagggtaa

SEQ ID NO: 220 - DNA, 30 bp, synthetic construct
  source 1..30: mol_type = other DNA
agagagtgactcagcccgagaagctcctct

SEQ ID NO: 221 - DNA, 27 bp, synthetic construct
  source 1..27: mol_type = other DNA
agagcccagtcagtgacccagcctgac

SEQ ID NO: 222 - DNA, 27 bp, synthetic construct
  source 1..27: mol_type = other DNA
agagcccagtcagtgacccagcctgac

SEQ ID NO: 223 - DNA, 27 bp, synthetic construct
  source 1..27: mol_type = other DNA
agctgctggagcagagccctcagtttc

SEQ ID NO: 224 - DNA, 33 bp, synthetic construct
  source 1..33: mol_type = other DNA
agtcaacagggagaagaggatcctcaggccttg

SEQ ID NO: 225 - DNA, 31 bp, synthetic construct
  source 1..31: mol_type = other DNA
agtggagactcggttacccagacagaaggcc

SEQ ID NO: 226 - DNA, 31 bp, synthetic construct
  source 1..31: mol_type = other DNA
agtggagcagagtcctccagacctgattctc

SEQ ID NO: 227 - DNA, 28 bp, synthetic construct
  source 1..28: mol_type = other DNA
agtgtggggctgcatcttcctaccctga

SEQ ID NO: 228 - DNA, 37 bp, synthetic construct
  source 1..37: mol_type = other DNA
atactgaacgtggaacaaagtcctcagtcactgcatg

SEQ ID NO: 229 - DNA, 30 bp, synthetic construct
  source 1..30: mol_type = other DNA
attcagtgacccagatggaagggccagtga

SEQ ID NO: 230 - DNA, 29 bp, synthetic construct
  source 1..29: mol_type = other DNA
attgatgctaagaccacccagcccacctc

SEQ ID NO: 231 - DNA, 31 bp, synthetic construct
  source 1..31: mol_type = other DNA
cagaaggaggtggagcagaattctggacccc

SEQ ID NO: 232 - DNA, 28 bp, synthetic construct
  source 1..28: mol_type = other DNA
cagcaattcagtcaagcagacgggccaa

SEQ ID NO: 233 - DNA, 29 bp, synthetic construct
  source 1..29: mol_type = other DNA
ccaacaaccagtgcagagtcctcaagccg

SEQ ID NO: 234 - DNA, 27 bp, synthetic construct
  source 1..27: mol_type = other DNA
cggaaggaggtggagcaggatcctgga

SEQ ID NO: 235 - DNA, 35 bp, synthetic construct
  source 1..35: mol_type = other DNA
ctacatacgccggagcagagtccttcattcctgag

SEQ ID NO: 236 - DNA, 31 bp, synthetic construct
  source 1..31: mol_type = other DNA
ctcaaacccaaccaggaatgttcgtgcagga

SEQ ID NO: 237 - DNA, 35 bp, synthetic construct
  source 1..35: mol_type = other DNA
cttgctaagaccacccagcccatctccatggactc

SEQ ID NO: 238 - DNA, 33 bp, synthetic construct
  source 1..33: mol_type = other DNA
gaaaaccaggtggagcacagccctcattttctg

SEQ ID NO: 239 - DNA, 35 bp, synthetic construct
  source 1..35: mol_type = other DNA
gaagacaaggtggtacaaagccctctatctctggt

SEQ ID NO: 240 - DNA, 27 bp, synthetic construct
  source 1..27: mol_type = other DNA
gaagaccaggtgacgcagagtcccgag

SEQ ID NO: 241 - DNA, 31 bp, synthetic construct
  source 1..31: mol_type = other DNA
gaccagcagcaggtgaaacaaagtcctcaat

SEQ ID NO: 242 - DNA, 28 bp, synthetic construct
  source 1..28: mol_type = other DNA
gagcagagtcctcagaacctgactgccc

SEQ ID NO: 243 - DNA, 35 bp, synthetic construct
  source 1..35: mol_type = other DNA
gatgaccagcaagttaagcaaaattcaccatccct

SEQ ID NO: 244 - DNA, 29 bp, synthetic construct
  source 1..29: mol_type = other DNA
gccaagaactggagcagagtcctcagtcc

SEQ ID NO: 245 - DNA, 26 bp, synthetic construct
  source 1..26: mol_type = other DNA
gcccagtcggtgacccagcttgacag

SEQ ID NO: 246 - DNA, 30 bp, synthetic construct
  source 1..30: mol_type = other DNA
gcccagtctgtgagccagcataaccaccac

SEQ ID NO: 247 - DNA, 30 bp, synthetic construct
  source 1..30: mol_type = other DNA
gcctgttcacttgccttgtaaccactccac

SEQ ID NO: 248 - DNA, 31 bp, synthetic construct
  source 1..31: mol_type = other DNA
gctcagtcagtggctcagccggaagatcagg

SEQ ID NO: 249 - DNA, 35 bp, synthetic construct
  source 1..35: mol_type = other DNA
ggacaaaacattgaccagcccactgagatgacagc

SEQ ID NO: 250 - DNA, 32 bp, synthetic construct
  source 1..32: mol_type = other DNA
ggacaaagccttgagcagccctctgaagtgac

SEQ ID NO: 251 - DNA, 37 bp, synthetic construct
  source 1..37: mol_type = other DNA
ggacaacaggtaatgcaaattcctcagtaccagcatg

SEQ ID NO: 252 - DNA, 33 bp, synthetic construct
  source 1..33: mol_type = other DNA
ggagagaatgtggagcagcatccttcaaccctg

SEQ ID NO: 253 - DNA, 36 bp, synthetic construct
  source 1..36: mol_type = other DNA
ggagaggatgtggagcagagtcttttcctgagtgtc

SEQ ID NO: 254 - DNA, 33 bp, synthetic construct
  source 1..33: mol_type = other DNA
ggagattcagtggtccagacagaaggccaagtg

SEQ ID NO: 255 - DNA, 29 bp, synthetic construct
  source 1..29: mol_type = other DNA
gtctcaaccagagatgtctgtgcaggagg

SEQ ID NO: 256 - DNA, 31 bp, synthetic construct
  source 1..31: mol_type = other DNA
gtggaacaaaaccctctgttcctgagcatgc

SEQ ID NO: 257 - DNA, 33 bp, synthetic construct
  source 1..33: mol_type = other DNA
gtggtcaacagctgaatcagagtcctcaatcta

SEQ ID NO: 258 - DNA, 27 bp, synthetic construct
  source 1..27: mol_type = other DNA
gttccggcaggatccggggagaagact

SEQ ID NO: 259 - DNA, 31 bp, Homo sapiens
  source 1..31: mol_type = genomic DNA
gcctcctcccagacatctgtatatttctgcg

SEQ ID NO: 260 - DNA, 30 bp, Homo sapiens
  source 1..30: mol_type = genomic DNA
aatttccccctcactctggagtcagctacc

SEQ ID NO: 261 - DNA, 30 bp, Homo sapiens
  source 1..30: mol_type = genomic DNA
gatttcctcctcactctggagtccgctacc

SEQ ID NO: 262 - DNA, 31 bp, Homo sapiens
  source 1..31: mol_type = genomic DNA
aggctcaaaggagtagactccactctcaaga

SEQ ID NO: 263 - DNA, 29 bp, Homo sapiens
  source 1..29: mol_type = genomic DNA
caagatccagcctgcaaagcttgaggact

SEQ ID NO: 264 - DNA, 30 bp, Homo sapiens
  source 1..30: mol_type = genomic DNA
tagactccactctcaagatccagcctgcag

SEQ ID NO: 265 - DNA, 30 bp, Homo sapiens
  source 1..30: mol_type = genomic DNA
tggaacccagggacttgggcctatatttct

SEQ ID NO: 266 - DNA, 31 bp, Homo sapiens
  source 1..31: mol_type = genomic DNA
tcattctctactctgaagatccagcctgcag

SEQ ID NO: 267 - DNA, 30 bp, Homo sapiens
  source 1..30: mol_type = genomic DNA
cattctccactctgaagatccagccctcag

SEQ ID NO: 268 - DNA, 31 bp, Homo sapiens
  source 1..31: mol_type = genomic DNA
catcattctccactctgaagatccagccctc

SEQ ID NO: 269 - DNA, 30 bp, Homo sapiens
  source 1..30: mol_type = genomic DNA
cagcagagatgcctgatgcaactttagcca

SEQ ID NO: 270 - DNA, 29 bp, Homo sapiens
  source 1..29: mol_type = genomic DNA
gaactgaacatgagctccttggagctggg

SEQ ID NO: 271 - DNA, 31 bp, Homo sapiens
  source 1..31: mol_type = genomic DNA
ggaggattctggagtttatttctgtgccagc

SEQ ID NO: 272 - DNA, 30 bp, Homo sapiens
  source 1..30: mol_type = genomic DNA
ttctgctttcttgacatccgctcaccaggc

SEQ ID NO: 273 - DNA, 31 bp, Homo sapiens
  source 1..31: mol_type = genomic DNA
gagatccaggctacgaagcttgaggattcag

SEQ ID NO: 274 - DNA, 28 bp, Homo sapiens
  source 1..28: mol_type = genomic DNA
aacgtcttccacgctgaagatccatccc

SEQ ID NO: 275 - DNA, 30 bp, Homo sapiens
  source 1..30: mol_type = genomic DNA
aggatccagcaggtagtgcgaggagattcg

SEQ ID NO: 276 - DNA, 30 bp, Homo sapiens
  source 1..30: mol_type = genomic DNA
acccgacagctttctatctctgtgccagta

SEQ ID NO: 277 - DNA, 30 bp, Homo sapiens
  source 1..30: mol_type = genomic DNA
gtgcccatcctgaagacagcagcttctaca

SEQ ID NO: 278 - DNA, 28 bp, Homo sapiens
  source 1..28: mol_type = genomic DNA
cacaaagctggaggactcagccatgtac

SEQ ID NO: 279 - DNA, 30 bp, Homo sapiens
  source 1..30: mol_type = genomic DNA
tcaggggacacagcactgtatttctgtgcc

SEQ ID NO: 280 - DNA, 30 bp, Homo sapiens
  source 1..30: mol_type = genomic DNA
cacaccagccaaacagctttgtacttctgt

SEQ ID NO: 281 - DNA, 28 bp, Homo sapiens
  source 1..28: mol_type = genomic DNA
aatcctgtcctcagaaccgggagacacg

SEQ ID NO: 282 - DNA, 31 bp, Homo sapiens
  source 1..31: mol_type = genomic DNA
ccaaccagacagctctttacttctgtgccac

SEQ ID NO: 283 - DNA, 30 bp, Homo sapiens
  source 1..30: mol_type = genomic DNA
cacatacctctcagtacctctgtgccagca

SEQ ID NO: 284 - DNA, 32 bp, Homo sapiens
  source 1..32: mol_type = genomic DNA
ccaaccagacatctgtgtatctctatgccagc

SEQ ID NO: 285 - DNA, 30 bp, Homo sapiens
  source 1..30: mol_type = genomic DNA
accagacctctctgtacttctgtgccagca

SEQ ID NO: 286 - DNA, 30 bp, Homo sapiens
  source 1..30: mol_type = genomic DNA
aaccagacatctatgtacctctgtgccagc

SEQ ID NO: 287 - DNA, 32 bp, Homo sapiens
  source 1..32: mol_type = genomic DNA
acatgagccctgaagacagcagcatatatctc

SEQ ID NO: 288 - DNA, 30 bp, Homo sapiens
  source 1..30: mol_type = genomic DNA
agcttggtgactctgctgtgtatttctgtg

SEQ ID NO: 289 - DNA, 30 bp, Homo sapiens
  source 1..30: mol_type = genomic DNA
cttggtgactctgctgtgtatttctgtgcc

SEQ ID NO: 290 - DNA, 29 bp, Homo sapiens
  source 1..29: mol_type = genomic DNA
cagccagaagactcagccctgtatctctg

SEQ ID NO: 291 - DNA, 28 bp, Homo sapiens
  source 1..28: mol_type = genomic DNA
gccagaagactcggccctgtatctctgt

SEQ ID NO: 292 - DNA, 30 bp, Homo sapiens
  source 1..30: mol_type = genomic DNA
tattccttcacctacacaccctgcagccag

SEQ ID NO: 293 - DNA, 27 bp, Homo sapiens
  source 1..27: mol_type = genomic DNA
agatgaatgtgagcaccttggagctgg

SEQ ID NO: 294 - DNA, 29 bp, Homo sapiens
  source 1..29: mol_type = genomic DNA
tactgagtcaaacacggagctaggggact

SEQ ID NO: 295 - DNA, 30 bp, Homo sapiens
  source 1..30: mol_type = genomic DNA
gttgctctgagatgaatgtgagtgccttgg

SEQ ID NO: 296 - DNA, 30 bp, Homo sapiens
  source 1..30: mol_type = genomic DNA
atagctctgagctgaatgtgaacgccttgg

SEQ ID NO: 297 - DNA, 28 bp, Homo sapiens
  source 1..28: mol_type = genomic DNA
gagctgaatgtgaacgccttgttgctgg

SEQ ID NO: 298 - DNA, 31 bp, Homo sapiens
  source 1..31: mol_type = genomic DNA
aactatagctctgagctgaatgtgaacgcct

SEQ ID NO: 299 - DNA, 29 bp, Homo sapiens
  source 1..29: mol_type = genomic DNA
agctgaatgtgaacgccttgttgctaggg

SEQ ID NO: 300 - DNA, 29 bp, Homo sapiens
  source 1..29: mol_type = genomic DNA
ctgaatgtgaacgccttggagctggagga

SEQ ID NO: 301 - DNA, 28 bp, Homo sapiens
  source 1..28: mol_type = genomic DNA
gctccctcccagacatctgtgtacttct

SEQ ID NO: 302 - DNA, 28 bp, Homo sapiens
  source 1..28: mol_type = genomic DNA
gctgctccctcccaaacatctgtgtact

SEQ ID NO: 303 - DNA, 30 bp, Homo sapiens
  source 1..30: mol_type = genomic DNA
gctccctcccaaacatctgtgtacttctgt

SEQ ID NO: 304 - DNA, 29 bp, Homo sapiens
  source 1..29: mol_type = genomic DNA
aacacagatgatttccccctcacgttggc

SEQ ID NO: 305 - DNA, 29 bp, Homo sapiens
  source 1..29: mol_type = genomic DNA
gctgctccctcccagacatctgtgtactt

SEQ ID NO: 306 - DNA, 27 bp, Homo sapiens
  source 1..27: mol_type = genomic DNA
agttggctgctccctcccagacatctg

SEQ ID NO: 307 - DNA, 30 bp, Homo sapiens
  source 1..30: mol_type = genomic DNA
tcagctgctccctctcagacttctgtttac

SEQ ID NO: 308 - DNA, 31 bp, Homo sapiens
  source 1..31: mol_type = genomic DNA
taaacacagaggatttcccactcaggctggt

SEQ ID NO: 309 - DNA, 31 bp, Homo sapiens
  source 1..31: mol_type = genomic DNA
agtcagctgctccctcccagacatctgtata

SEQ ID NO: 310 - DNA, 27 bp, Homo sapiens
  source 1..27: mol_type = genomic DNA
cagcagggggacttggctgtgtatctc

SEQ ID NO: 311 - DNA, 25 bp, Homo sapiens
  source 1..25: mol_type = genomic DNA
gcaggaggactcggccgtgtatctc

SEQ ID NO: 312 - DNA, 29 bp, Homo sapiens
  source 1..29: mol_type = genomic DNA
tctactctgaagatccagcgcacagagcg

SEQ ID NO: 313 - DNA, 28 bp, Homo sapiens
  source 1..28: mol_type = genomic DNA
cacagagcagggggactcagctgtgtat

SEQ ID NO: 314 - DNA, 30 bp, Homo sapiens
  source 1..30: mol_type = genomic DNA
atctttctccacctgaagatccagcgcaca

SEQ ID NO: 315 - DNA, 29 bp, Homo sapiens
  source 1..29: mol_type = genomic DNA
ttctctgcagagaggcctgagggatccat

SEQ ID NO: 316 - DNA, 30 bp, Homo sapiens
  source 1..30: mol_type = genomic DNA
ctgagggatccgtctccactctgaagatcc

SEQ ID NO: 317 - DNA, 30 bp, Homo sapiens
  source 1..30: mol_type = genomic DNA
ggcctaagggatctttctccaccttggaga

SEQ ID NO: 318 - DNA, 30 bp, Homo sapiens
  source 1..30: mol_type = genomic DNA
ttccctcaaccctggagtctactagcacca

SEQ ID NO: 319 - DNA, 29 bp, Homo sapiens
  source 1..29: mol_type = genomic DNA
ttgagcatttccccaatcctggcatccac

SEQ ID NO: 320 - DNA, 30 bp, Homo sapiens
  source 1..30: mol_type = genomic DNA
gggactcagctttgtatttctgtgccagca

SEQ ID NO: 321 - DNA, 65 bp, synthetic construct
  source 1..65: mol_type = other DNA
gtgactggagttcagacgtgtgctcttccgatctgcctcctcccagacatctgtatatttctgcg

SEQ ID NO: 322 - DNA, 64 bp, synthetic construct
  source 1..64: mol_type = other DNA
gtgactggagttcagacgtgtgctcttccgatctaatttccccctcactctggagtcagctacc

SEQ ID NO: 323 - DNA, 64 bp, synthetic construct
  source 1..64: mol_type = other DNA
gtgactggagttcagacgtgtgctcttccgatctgatttcctcctcactctggagtccgctacc

SEQ ID NO: 324 - DNA, 65 bp, synthetic construct
  source 1..65: mol_type = other DNA
gtgactggagttcagacgtgtgctcttccgatctaggctcaaaggagtagactccactctcaaga

SEQ ID NO: 325 - DNA, 63 bp, synthetic construct
  source 1..63: mol_type = other DNA
gtgactggagttcagacgtgtgctcttccgatctcaagatccagcctgcaaagcttgaggact

SEQ ID NO: 326 - DNA, 64 bp, synthetic construct
  source 1..64: mol_type = other DNA
gtgactggagttcagacgtgtgctcttccgatcttagactccactctcaagatccagcctgcag

SEQ ID NO: 327 - DNA, 64 bp, synthetic construct
  source 1..64: mol_type = other DNA
gtgactggagttcagacgtgtgctcttccgatcttggaacccagggacttgggcctatatttct

SEQ ID NO: 328 - DNA, 65 bp, synthetic construct
  source 1..65: mol_type = other DNA
gtgactggagttcagacgtgtgctcttccgatcttcattctctactctgaagatccagcctgcag

SEQ ID NO: 329 - DNA, 64 bp, synthetic construct
  source 1..64: mol_type = other DNA
gtgactggagttcagacgtgtgctcttccgatctcattctccactctgaagatccagccctcag

SEQ ID NO: 330 - DNA, 65 bp, synthetic construct
  source 1..65: mol_type = other DNA
gtgactggagttcagacgtgtgctcttccgatctcatcattctccactctgaagatccagccctc

SEQ ID NO: 331 - DNA, 64 bp, synthetic construct
  source 1..64: mol_type = other DNA
gtgactggagttcagacgtgtgctcttccgatctcagcagagatgcctgatgcaactttagcca

SEQ ID NO: 332 - DNA, 63 bp, synthetic construct
  source 1..63: mol_type = other DNA
gtgactggagttcagacgtgtgctcttccgatctgaactgaacatgagctccttggagctggg

SEQ ID NO: 333 - DNA, 65 bp, synthetic construct
  source 1..65: mol_type = other DNA
gtgactggagttcagacgtgtgctcttccgatctggaggattctggagtttatttctgtgccagc

SEQ ID NO: 334 - DNA, 64 bp, synthetic construct
  source 1..64: mol_type = other DNA
gtgactggagttcagacgtgtgctcttccgatctttctgctttcttgacatccgctcaccaggc

SEQ ID NO: 335 - DNA, 65 bp, synthetic construct
  source 1..65: mol_type = other DNA
gtgactggagttcagacgtgtgctcttccgatctgagatccaggctacgaagcttgaggattcag

SEQ ID NO: 336 - DNA, 62 bp, synthetic construct
  source 1..62: mol_type = other DNA
gtgactggagttcagacgtgtgctcttccgatctaacgtcttccacgctgaagatccatccc

SEQ ID NO: 337 - DNA, 64 bp, synthetic construct
  source 1..64: mol_type = other DNA
gtgactggagttcagacgtgtgctcttccgatctaggatccagcaggtagtgcgaggagattcg

SEQ ID NO: 338 - DNA, 64 bp, synthetic construct
  source 1..64: mol_type = other DNA
gtgactggagttcagacgtgtgctcttccgatctacccgacagctttctatctctgtgccagta

SEQ ID NO: 339 - DNA, 64 bp, synthetic construct
  source 1..64: mol_type = other DNA
gtgactggagttcagacgtgtgctcttccgatctgtgcccatcctgaagacagcagcttctaca

SEQ ID NO: 340 - DNA, 62 bp, synthetic construct
  source 1..62: mol_type = other DNA
gtgactggagttcagacgtgtgctcttccgatctcacaaagctggaggactcagccatgtac

SEQ ID NO: 341 - DNA, 64 bp, synthetic construct
  source 1..64: mol_type = other DNA
gtgactggagttcagacgtgtgctcttccgatcttcaggggacacagcactgtatttctgtgcc

SEQ ID NO: 342 - DNA, 64 bp, synthetic construct
  source 1..64: mol_type = other DNA
gtgactggagttcagacgtgtgctcttccgatctcacaccagccaaacagctttgtacttctgt

SEQ ID NO: 343 - DNA, 62 bp, synthetic construct
  source 1..62: mol_type = other DNA
gtgactggagttcagacgtgtgctcttccgatctaatcctgtcctcagaaccgggagacacg

SEQ ID NO: 344 - DNA, 65 bp, synthetic construct
  source 1..65: mol_type = other DNA
gtgactggagttcagacgtgtgctcttccgatctccaaccagacagctctttacttctgtgccac

SEQ ID NO: 345 - DNA, 64 bp, synthetic construct
  source 1..64: mol_type = other DNA
gtgactggagttcagacgtgtgctcttccgatctcacatacctctcagtacctctgtgccagca

SEQ ID NO: 346 - DNA, 66 bp, synthetic construct
  source 1..66: mol_type = other DNA
gtgactggagttcagacgtgtgctcttccgatctccaaccagacatctgtgtatctctatgccagc

SEQ ID NO: 347 - DNA, 64 bp, synthetic construct
  source 1..64: mol_type = other DNA
gtgactggagttcagacgtgtgctcttccgatctaccagacctctctgtacttctgtgccagca

SEQ ID NO: 348 - DNA, 64 bp, synthetic construct
  source 1..64: mol_type = other DNA
gtgactggagttcagacgtgtgctcttccgatctaaccagacatctatgtacctctgtgccagc

SEQ ID NO: 349 - DNA, 66 bp, synthetic construct
  source 1..66: mol_type = other DNA
gtgactggagttcagacgtgtgctcttccgatctacatgagccctgaagacagcagcatatatctc

SEQ ID NO: 350 - DNA, 64 bp, synthetic construct
  source 1..64: mol_type = other DNA
gtgactggagttcagacgtgtgctcttccgatctagcttggtgactctgctgtgtatttctgtg

SEQ ID NO: 351 - DNA, 64 bp, synthetic construct
  source 1..64: mol_type = other DNA
gtgactggagttcagacgtgtgctcttccgatctcttggtgactctgctgtgtatttctgtgcc

SEQ ID NO: 352 - DNA, 63 bp, synthetic construct
  source 1..63: mol_type = other DNA
gtgactggagttcagacgtgtgctcttccgatctcagccagaagactcagccctgtatctctg

SEQ ID NO: 353 - DNA, 62 bp, synthetic construct
  source 1..62: mol_type = other DNA
gtgactggagttcagacgtgtgctcttccgatctgccagaagactcggccctgtatctctgt

SEQ ID NO: 354 - DNA, 64 bp, synthetic construct
  source 1..64: mol_type = other DNA
gtgactggagttcagacgtgtgctcttccgatcttattccttcacctacacaccctgcagccag

SEQ ID NO: 355 - DNA, 61 bp, synthetic construct
  source 1..61: mol_type = other DNA
gtgactggagttcagacgtgtgctcttccgatctagatgaatgtgagcaccttggagctgg

SEQ ID NO: 356 - DNA, 63 bp, synthetic construct
  source 1..63: mol_type = other DNA
gtgactggagttcagacgtgtgctcttccgatcttactgagtcaaacacggagctaggggact

SEQ ID NO: 357 - DNA, 64 bp, synthetic construct
  source 1..64: mol_type = other DNA
gtgactggagttcagacgtgtgctcttccgatctgttgctctgagatgaatgtgagtgccttgg

SEQ ID NO: 358 - DNA, 64 bp, synthetic construct
  source 1..64: mol_type = other DNA
gtgactggagttcagacgtgtgctcttccgatctatagctctgagctgaatgtgaacgccttgg

SEQ ID NO: 359 - DNA, 62 bp, synthetic construct
  source 1..62: mol_type = other DNA
gtgactggagttcagacgtgtgctcttccgatctgagctgaatgtgaacgccttgttgctgg

SEQ ID NO: 360 - DNA, 65 bp, synthetic construct
  source 1..65: mol_type = other DNA
gtgactggagttcagacgtgtgctcttccgatctaactatagctctgagctgaatgtgaacgcct

SEQ ID NO: 361 - DNA, 63 bp, synthetic construct
  source 1..63: mol_type = other DNA
gtgactggagttcagacgtgtgctcttccgatctagctgaatgtgaacgccttgttgctaggg

SEQ ID NO: 362 - DNA, 63 bp, synthetic construct
  source 1..63: mol_type = other DNA
gtgactggagttcagacgtgtgctcttccgatctctgaatgtgaacgccttggagctggagga

SEQ ID NO: 363 - DNA, 62 bp, synthetic construct
  source 1..62: mol_type = other DNA
gtgactggagttcagacgtgtgctcttccgatctgctccctcccagacatctgtgtacttct

SEQ ID NO: 364 - DNA, 62 bp, synthetic construct
  source 1..62: mol_type = other DNA
gtgactggagttcagacgtgtgctcttccgatctgctgctccctcccaaacatctgtgtact

SEQ ID NO: 365 - DNA, 64 bp, synthetic construct
  source 1..64: mol_type = other DNA
gtgactggagttcagacgtgtgctcttccgatctgctccctcccaaacatctgtgtacttctgt

SEQ ID NO: 366 - DNA, 63 bp, synthetic construct
  source 1..63: mol_type = other DNA
gtgactggagttcagacgtgtgctcttccgatctaacacagatgatttccccctcacgttggc

SEQ ID NO: 367 - DNA, 63 bp, synthetic construct
  source 1..63: mol_type = other DNA
gtgactggagttcagacgtgtgctcttccgatctgctgctccctcccagacatctgtgtactt

SEQ ID NO: 368 - DNA, 61 bp, synthetic construct
  source 1..61: mol_type = other DNA
gtgactggagttcagacgtgtgctcttccgatctagttggctgctccctcccagacatctg

SEQ ID NO: 369 - DNA, 64 bp, synthetic construct
  source 1..64: mol_type = other DNA
gtgactggagttcagacgtgtgctcttccgatcttcagctgctccctctcagacttctgtttac

SEQ ID NO: 370 - DNA, 65 bp, synthetic construct
  source 1..65: mol_type = other DNA
gtgactggagttcagacgtgtgctcttccgatcttaaacacagaggatttcccactcaggctggt

SEQ ID NO: 371 - DNA, 65 bp, synthetic construct
  source 1..65: mol_type = other DNA
gtgactggagttcagacgtgtgctcttccgatctagtcagctgctccctcccagacatctgtata

SEQ ID NO: 372 - DNA, 61 bp, synthetic construct
  source 1..61: mol_type = other DNA
gtgactggagttcagacgtgtgctcttccgatctcagcagggggacttggctgtgtatctc

SEQ ID NO: 373 - DNA, 59 bp, synthetic construct
  source 1..59: mol_type = other DNA
gtgactggagttcagacgtgtgctcttccgatctgcaggaggactcggccgtgtatctc

SEQ ID NO: 374 - DNA, 63 bp, synthetic construct
  source 1..63: mol_type = other DNA
gtgactggagttcagacgtgtgctcttccgatcttctactctgaagatccagcgcacagagcg

SEQ ID NO: 375 - DNA, 62 bp, synthetic construct
  source 1..62: mol_type = other DNA
gtgactggagttcagacgtgtgctcttccgatctcacagagcagggggactcagctgtgtat

SEQ ID NO: 376 - DNA, 64 bp, synthetic construct
  source 1..64: mol_type = other DNA
gtgactggagttcagacgtgtgctcttccgatctatctttctccacctgaagatccagcgcaca

SEQ ID NO: 377 - DNA, 63 bp, synthetic construct
  source 1..63: mol_type = other DNA
gtgactggagttcagacgtgtgctcttccgatctttctctgcagagaggcctgagggatccat

SEQ ID NO: 378 - DNA, 64 bp, synthetic construct
  source 1..64: mol_type = other DNA
gtgactggagttcagacgtgtgctcttccgatctctgagggatccgtctccactctgaagatcc

SEQ ID NO: 379 - DNA, 64 bp, synthetic construct
  source 1..64: mol_type = other DNA
gtgactggagttcagacgtgtgctcttccgatctggcctaagggatctttctccaccttggaga

SEQ ID NO: 380 - DNA, 64 bp, synthetic construct
  source 1..64: mol_type = other DNA
gtgactggagttcagacgtgtgctcttccgatctttccctcaaccctggagtctactagcacca

SEQ ID NO: 381 - DNA, 63 bp, synthetic construct
  source 1..63: mol_type = other DNA
gtgactggagttcagacgtgtgctcttccgatctttgagcatttccccaatcctggcatccac

SEQ ID NO: 382 - DNA, 64 bp, synthetic construct
  source 1..64: mol_type = other DNA
gtgactggagttcagacgtgtgctcttccgatctgggactcagctttgtatttctgtgccagca

SEQ ID NO: 383 - DNA, 30 bp, synthetic construct
  source 1..30: mol_type = other DNA
gctgaaatcacccagagcccaagacacaag

SEQ ID NO: 384 - DNA, 29 bp, synthetic construct
  source 1..29: mol_type = other DNA
cacagagacaggaaggcaggtgaccttga

SEQ ID NO: 385 - DNA, 30 bp, synthetic construct
  source 1..30: mol_type = other DNA
gatgctggaatcacccagagcccaagacac

SEQ ID NO: 386 - DNA, 30 bp, synthetic construct
  source 1..30: mol_type = other DNA
gccaggctgtggctttttggtgtgatccta

SEQ ID NO: 387 - DNA, 29 bp, synthetic construct
  source 1..29: mol_type = other DNA
ggcagagtgtggctttttggtgcaatcct

SEQ ID NO: 388 - DNA, 31 bp, synthetic construct
  source 1..31: mol_type = other DNA
ggctttttggtgcaatcctatttctggccac

SEQ ID NO: 389 - DNA, 30 bp, synthetic construct
  source 1..30: mol_type = other DNA
gatgctggtgttatccagtcacccaggcac

SEQ ID NO: 390 - DNA, 30 bp, synthetic construct
  source 1..30: mol_type = other DNA
gtcacccaagcatgaggtgacagaaatggg

SEQ ID NO: 391 - DNA, 27 bp, synthetic construct
  source 1..27: mol_type = other DNA
atgctggagttatccagtcaccccgcc

SEQ ID NO: 392 - DNA, 28 bp, synthetic construct
  source 1..28: mol_type = other DNA
gagttatccagtcaccccggcacgaggt

SEQ ID NO: 393 - DNA, 28 bp, synthetic construct
  source 1..28: mol_type = other DNA
gctagagtcacccagacaccaaggcaca

SEQ ID NO: 394 - DNA, 27 bp, synthetic construct
  source 1..27: mol_type = other DNA
gctgctggagtcatccagtccccaaga

SEQ ID NO: 395 - DNA, 29 bp, synthetic construct
  source 1..29: mol_type = other DNA
gttactcagttccccagccacagcgtaat

SEQ ID NO: 396 - DNA, 29 bp, synthetic construct
  source 1..29: mol_type = other DNA
gttacccagtttggaaagccagtgaccct

SEQ ID NO: 397 - DNA, 30 bp, synthetic construct
  source 1..30: mol_type = other DNA
gaagtcgcccagactccaaaacatcttgtc

SEQ ID NO: 398 - DNA, 29 bp, synthetic construct
  source 1..29: mol_type = other DNA
cagacacaaggtcaccaacatgggacagg

SEQ ID NO: 399 - DNA, 29 bp, synthetic construct
  source 1..29: mol_type = other DNA
gtcatgtttactggtatcggcagctccca

SEQ ID NO: 400 - DNA, 29 bp, synthetic construct
  source 1..29: mol_type = other DNA
atgccatgtactggtaccgacaggaccca

SEQ ID NO: 401 - DNA, 29 bp, synthetic construct
  source 1..29: mol_type = other DNA
gtcgtctctcaacatccgagctgggttat

SEQ ID NO: 402 - DNA, 29 bp, synthetic construct
  source 1..29: mol_type = other DNA
gaacctgaagtcacccagactcccagcca

SEQ ID NO: 403 - DNA, 28 bp, synthetic construct
  source 1..28: mol_type = other DNA
cacggacaccaaggtcacccagagacct

SEQ ID NO: 404 - DNA, 28 bp, synthetic construct
  source 1..28: mol_type = other DNA
agctcactggggctggatgggatgtgac

SEQ ID NO: 405 - DNA, 29 bp, synthetic construct
  source 1..29: mol_type = other DNA
gccaaagtcacacagactccaggacattt

SEQ ID NO: 406 - DNA, 29 bp, synthetic construct
  source 1..29: mol_type = other DNA
gtatcgacaagacccaggactgggcctac

SEQ ID NO: 407 - DNA, 29 bp, synthetic construct
  source 1..29: mol_type = other DNA
gctgacatctaccagaccccaagatacct

SEQ ID NO: 408 - DNA, 29 bp, synthetic construct
  source 1..29: mol_type = other DNA
gtatcgacaggacccaggacttggactga

SEQ ID NO: 409 - DNA, 28 bp, synthetic construct
  source 1..28: mol_type = other DNA
agcccaagtgacccagaacccaagatac

SEQ ID NO: 410 - DNA, 31 bp, synthetic construct
  source 1..31: mol_type = other DNA
ctcgtagatgtgaaagtaacccagagctcga

SEQ ID NO: 411 - DNA, 30 bp, synthetic construct
  source 1..30: mol_type = other DNA
gatatctgtcaacgtggaacctccctgacg

SEQ ID NO: 412 - DNA, 29 bp, synthetic construct
  source 1..29: mol_type = other DNA
ggtcacacagatgggaaacgacaagtcca

SEQ ID NO: 413 - DNA, 29 bp, synthetic construct
  source 1..29: mol_type = other DNA
ccgtttcccagactccaaaatacctggtc

SEQ ID NO: 414 - DNA, 30 bp, synthetic construct; identical to SEQ ID NO: 50
  source 1..30: mol_type = other DNA

SEQ ID NO: 415 - DNA, 29 bp, synthetic construct
  source 1..29: mol_type = other DNA
gagttacgcagacaccaagacacctggtc

SEQ ID NO: 416 - DNA, 28 bp, synthetic construct
  source 1..28: mol_type = other DNA
ggagttacgcagacaccaagacacctgg

SEQ ID NO: 417 - DNA, 29 bp, synthetic construct
  source 1..29: mol_type = other DNA
gtgacactgagctgctcccctatctctgg

SEQ ID NO: 418 - DNA, 29 bp, synthetic construct
  source 1..29: mol_type = other DNA
gaatcacccaagctccaagacacctgatc

SEQ ID NO: 419 - DNA, 27 bp, synthetic construct
  source 1..27: mol_type = other DNA
ctggagtcacccaaagtcccacacacc

SEQ ID NO: 420 - DNA, 28 bp, synthetic construct
  source 1..28: mol_type = other DNA
gactggagtcacccaaagtcccacacac

SEQ ID NO: 421 - DNA, 29 bp, synthetic construct
  source 1..29: mol_type = other DNA
gtcccacacacctgatcaaaacgagagga

SEQ ID NO: 422 - DNA, 28 bp, synthetic construct
  source 1..28: mol_type = other DNA
tagtggacgctggagtcacccaaagtcc

SEQ ID NO: 423 - DNA, 29 bp, synthetic construct
  source 1..29: mol_type = other DNA
ctgatcaaaacgagaggacagcacgtgac

SEQ ID NO: 424 - DNA, 29 bp, synthetic construct
  source 1..29: mol_type = other DNA
gagtcacacaaagtcccacacacctgatc

SEQ ID NO: 425 - DNA, 29 bp, synthetic construct
  source 1..29: mol_type = other DNA
gtgaatgctggtgtcactcagaccccaaa

SEQ ID NO: 426 - DNA, 29 bp, synthetic construct
  source 1..29: mol_type = other DNA
gaatgctggtgtcactcagaccccaaaat

SEQ ID NO: 427 - DNA, 29 bp, synthetic construct
  source 1..29: mol_type = other DNA
gctggtgtcactcagaccccaaaattccg

SEQ ID NO: 428 - DNA, 29 bp, synthetic construct
  source 1..29: mol_type = other DNA
gatcacccaggcaccaacatctcagatcc

SEQ ID NO: 429 - DNA, 29 bp, synthetic construct
  source 1..29: mol_type = other DNA
gctggtgtcactcagaccccaaaattcca

SEQ ID NO: 430 - DNA, 29 bp, synthetic construct
  source 1..29: mol_type = other DNA
gctggtgtcactcagaccccaaaattccg

SEQ ID NO: 431 - DNA, 29 bp, synthetic construct
  source 1..29: mol_type = other DNA
gaatgctggtgtcactcagaccccaaaat

SEQ ID NO: 432 - DNA, 29 bp, synthetic construct
  source 1..29: mol_type = other DNA
gctggtgtcactcagaccccaaaattcca

SEQ ID NO: 433 - DNA, 29 bp, synthetic construct
  source 1..29: mol_type = other DNA
gaatgctggtgtcactcagaccccaaaat

SEQ ID NO: 434 - DNA, 28 bp, synthetic construct
  source 1..28: mol_type = other DNA
gtgctggagtctcccagtccctgagaca

SEQ ID NO: 435 - DNA, 29 bp, synthetic construct
  source 1..29: mol_type = other DNA
gtcccccagtaacaaggtcacagagaagg

SEQ ID NO: 436 - DNA, 29 bp, synthetic construct
  source 1..29: mol_type = other DNA
gacccccagtaacaaggtcacagagaagg

SEQ ID NO: 437 - DNA, 29 bp, synthetic construct
  source 1..29: mol_type = other DNA
cagtccccaaggtacaaagtcgcaaagag

SEQ ID NO: 438 - DNA, 27 bp, synthetic construct
  source 1..27: mol_type = other DNA
gtctcccagtccccaaggtacgaagtc

SEQ ID NO: 439 - DNA, 26 bp, synthetic construct
  source 1..26: mol_type = other DNA
cacaggtgctggagtctcccagtctc

SEQ ID NO: 440 - DNA, 26 bp, synthetic construct
  source 1..26: mol_type = other DNA
gtgctggagtctcccagtcccctagg

SEQ ID NO: 441 - DNA, 27 bp, synthetic construct
  source 1..27: mol_type = other DNA
ctggagtctcccagaaccccagacaca

SEQ ID NO: 442 - DNA, 28 bp, synthetic construct
  source 1..28: mol_type = other DNA
gaggcagggatcagccagataccaagat

SEQ ID NO: 443 - DNA, 27 bp, synthetic construct
  source 1..27: mol_type = other DNA
gatgctgggatcacccagatgccaaga

SEQ ID NO: 444 - DNA, 28 bp, synthetic construct
  source 1..28: mol_type = other DNA
tggagtcacacaaaccccaaagcacctg

SEQ ID NO: 445 - DNA, 120 bp, synthetic construct
  source 1..120: mol_type = other DNA
gaactgtggctgcaccatctgtcttcatcttcccgccatctgatgagcagttgaaatctggaactgcctctgttgtgtgcctgctgaataacttctatcccagagaggccaaagtacagt

SEQ ID NO: 446 - DNA, 120 bp, synthetic construct
  source 1..120: mol_type = other DNA
ggaaggtggataacgccctccaatcgggtaactcccaggagagtgtcacagagcaggacagcaaggacagcacctacagcctcagcagcaccctgacgctgagcaaagcagactacgaga

SEQ ID NO: 447 - DNA, 120 bp, synthetic construct
  source 1..120: mol_type = other DNA
ctcgcccgtcacaaagagcttcaacaggggagagtgttagagggagaagtgcccccacctgctcctcagttccagcctgaccccctcccatcctttggcctctgaccctttttccacagg

SEQ ID NO: 448 - DNA, 120 bp, synthetic construct
  source 1..120: mol_type = other DNA
ggacctacccctattgcggtcctccagctcatctttcacctcacccccctcctcctccttggctttaattatgctaatgttggaggagaatgaataaataaagtgaatctttgcacctgt

SEQ ID NO: 449 - DNA, 120 bp, synthetic construct
  source 1..120: mol_type = other DNA
gtcagcccaaggccaaccccactgtcactctgttcccgccctcctctgaggagctccaagccaacaaggccacactagtgtgtctgatcagtgacttctacccgggagctgtgacagtgg

SEQ ID NO: 450 - DNA, 120 bp, synthetic construct
  source 1..120: mol_type = other DNA
gacgcccgagcagtggaagtcccacagaagctacagctgccaggtcacgcatgaagggagcaccgtggagaagacagtggcccctacagaatgttcataggttcccaactctaaccccac

SEQ ID NO: 451 - DNA, 120 bp, synthetic construct
  source 1..120: mol_type = other DNA
ccacgggagcctggagctgcaggatcccaggggaggggtctctctccccatcccaagtcatccagcccttctccctgcactcatgaaaccccaataaatatcctcattgacaaccagaaa

SEQ ID NO: 452 - DNA, 120 bp, synthetic construct
  source 1..120: mol_type = other DNA
gtcagcccaaggctgccccctcggtcactctgttcccgccctcctctgaggagcttcaagccaacaaggccacactggtgtgtctcataagtgacttctacccgggagccgtgacagtgg

SEQ ID NO: 453 - DNA, 120 bp, synthetic construct
  source 1..120: mol_type = other DNA
cgcctgagcagtggaagtcccacagaagctacagctgccaggtcacgcatgaagggagcaccgtggagaagacagtggcccctacagaatgttcataggttctcaaccctcaccccccac

SEQ ID NO: 454 - DNA, 120 bp, synthetic construct
  source 1..120: mol_type = other DNA
cacgggagactagagctgcaggatcccaggggaggggtctctcctcccaccccaaggcatcaagcccttctccctgcactcaataaaccctcaataaatattctcattgtcaatcagaaa

SEQ ID NO: 455 - DNA, 120 bp, synthetic construct
  source 1..120: mol_type = other DNA
gtcagcccaaggctgccccctcggtcactctgttcccaccctcctctgaggagcttcaagccaacaaggccacactggtgtgtctcataagtgacttctacccgggagccgtgacagtgg

SEQ ID NO: 456 - DNA, 120 bp, synthetic construct
  source 1..120: mol_type = other DNA
cgcctgagcagtggaagtcccacaaaagctacagctgccaggtcacgcatgaagggagcaccgtggagaagacagtggcccctacagaatgttcataggttctcatccctcaccccccac

SEQ ID NO: 457 - DNA, 120 bp, synthetic construct; identical to SEQ ID NO: 454
  source 1..120: mol_type = other DNA

SEQ ID NO: 458 - DNA, 120 bp, synthetic construct
  source 1..120: mol_type = other DNA
gtcagcccaaggctgccccctcggtcactctgttcccaccctcctctgaggagcttcaagccaacaaggccacactggtgtgtctcgtaagtgacttcaacccgggagccgtgacagtgg

SEQ ID NO: 459 - DNA, 120 bp, synthetic construct
  source 1..120: mol_type = other DNA
ccagcagctacctgagcctgacgcccgagcagtggaagtcccacagaagctacagctgccgggtcacgcatgaagggagcaccgtggagaagacagtggcccctgcagaatgctcttagg

SEQ ID NO: 460 - DNA, 120 bp, synthetic construct
  source 1..120: mol_type = other DNA
cccccgaccctcaccccacccacaggggcctggagctgcaggttcccaggggaggggtctctgcccccatcccaagtcatccagcccttctcaataaatatcctcatcgtcaacgagaaa

SEQ ID NO: 461 - DNA, 120 bp, synthetic construct
  source 1..120: mol_type = other DNA
gcatccccgaccagccccaaggtcttcccgctgagcctcgacagcaccccccaagatgggaacgtggtcgtcgcatgcctggtccagggcttcttcccccaggagccactcagtgtgacc

SEQ ID NO: 462 - DNA, 120 bp, synthetic construct
  source 1..120: mol_type = other DNA
tggagcgaaagcggacagaacgtgaccgccagaaacttcccacctagccaggatgcctccggggacctgtacaccacgagcagccagctgaccctgccggccacacagtgcccagacggc

SEQ ID NO: 463 - DNA, 120 bp, synthetic construct
  source 1..120: mol_type = other DNA
aagtccgtgacatgccacgtgaagcactacacgaattccagccaggatgtgactgtgccctgccgagttcccccacctcccccatgctgccacccccgactgtcgctgcaccgaccggcc

SEQ ID NO: 464 - DNA, 120 bp, synthetic construct
  source 1..120: mol_type = other DNA
ctcgaggacctgctcttaggttcagaagcgaacctcacgtgcacactgaccggcctgagagatgcctctggtgccaccttcacctggacgccctcaagtgggaagagcgctgttcaagga

SEQ ID NO: 465 - DNA, 120 bp, synthetic construct
  source 1..120: mol_type = other DNA
ccacctgagcgtgacctctgtggctgctacagcgtgtccagtgtcctgcctggctgtgcccagccatggaaccatggggagaccttcacctgcactgctgcccaccccgagttgaagacc

SEQ ID NO: 466 - DNA, 120 bp, synthetic construct
  source 1..120: mol_type = other DNA
ccactaaccgccaacatcacaaaatccggaaacacattccggcccgaggtccacctgctgccgccgccgtcggaggagctggccctgaacgagctggtgacgctgacgtgcctggcacgt

SEQ ID NO: 467 - DNA, 120 bp, synthetic construct
  source 1..120: mol_type = other DNA
ggcttcagccccaaggatgtgctggttcgctggctgcaggggtcacaggagctgccccgcgagaagtacctgacttgggcatcccggcaggagcccagccagggcaccaccacctacgct

SEQ ID NO: 468 - DNA, 120 bp, synthetic construct
  source 1..120: mol_type = other DNA
gtaaccagcatactgcgcgtggcagctgaggactggaagaagggggagaccttctcctgcatggtgggccacgaggccctgccgctggccttcacacagaagaccatcgaccgcatggcg

SEQ ID NO: 469 - DNA, 120 bp, synthetic construct
  source 1..120: mol_type = other DNA
ggctcttgctgtgttgcagattggcagatgccgcctccctatgtggtgctggacttgccgcaggagaccctggaggaggagacccccggcgccaacctgtggcccaccaccatcaccttc

SEQ ID NO: 470 - DNA, 120 bp, synthetic construct
  source 1..120: mol_type = other DNA
ctcaccctcttcctgctgagcctgttctatagcacagcactgaccgtgaccagcgtccggggcccatctggcaagagggagggcccccagtactgagcgggagccggcaaggcacaggga

SEQ ID NO: 471 - DNA, 120 bp, synthetic construct
  source 1..120: mol_type = other DNA
ggaagtgtggaggaacctcttggagaagccagctatgcttgccagaactcagccctttcagacatcaccgacccgcccttactcacgtggcttccaggtgcaataaagtggccccaagga

SEQ ID NO: 472 - DNA, 120 bp, synthetic construct
  source 1..120: mol_type = other DNA
gcctccacacagagcccatccgtcttccccttgacccgctgctgcaaaaacattccctccaatgccacctccgtgactctgggctgcctggccacgggctacttcccggagccggtgatg

SEQ ID NO: 473 - DNA, 120 bp, synthetic construct
  source 1..120: mol_type = other DNA
gtgacctgggacacaggctccctcaacgggacaactatgaccttaccagccaccaccctcacgctctctggtcactatgccaccatcagcttgctgaccgtctcgggtgcgtgggccaag

SEQ ID NO: 474 - DNA, 120 bp, synthetic construct
  source 1..120: mol_type = other DNA
cagatgttcacctgccgtgtggcacacactccatcgtccacagactgggtcgacaacaaaaccttcagcgtctgctccagggacttcaccccgcccaccgtgaagatcttacagtcgtcc

SEQ ID NO: 475 - DNA, 120 bp, synthetic construct
  source 1..120: mol_type = other DNA
tgcgacggcggcgggcacttccccccgaccatccagctcctgtgcctcgtctctgggtacaccccagggactatcaacatcacctggctggaggacgggcaggtcatggacgtggacttg

SEQ ID NO: 476 - DNA, 120 bp, synthetic construct
  source 1..120: mol_type = other DNA
tccaccgcctctaccacgcaggagggtgagctggcctccacacaaagcgagctcaccctcagccagaagcactggctgtcagaccgcacctacacctgccaggtcacctatcaaggtcac

SEQ ID NO: 477 - DNA, 120 bp, synthetic construct
  source 1..120: mol_type = other DNA
acctttgaggacagcaccaagaagtgtgcagattccaacccgagaggggtgagcgcctacctaagccggcccagcccgttcgacctgttcatccgcaagtcgcccacgatcacctgtctg

SEQ ID NO: 478 - DNA, 120 bp, synthetic construct
  source 1..120: mol_type = other DNA
tcccgggccagtgggaagcctgtgaaccactccaccagaaaggaggagaagcagcgcaatggcacgttaaccgtcacgtccaccctgccggtgggcacccgagactggatcgagggggag

SEQ ID NO: 479 - DNA, 120 bp, synthetic construct
  source 1..120: mol_type = other DNA
acctaccagtgcagggtgacccacccccacctgcccagggccctcatgcggtccacgaccaagaccagcggcccgcgtgctgccccggaagtctatgcgtttgcgacgccggagtggccg

SEQ ID NO: 480 - DNA, 120 bp, synthetic construct
  source 1..120: mol_type = other DNA
gggagccgggacaagcgcaccctcgcctgcctgatccagaacttcatgcctgaggacatctcggtgcagtggctgcacaacgaggtgcagctcccggacgcccggcacagcacgacgcag

SEQ ID NO: 481 - DNA, 120 bp, synthetic construct
  source 1..120: mol_type = other DNA
ccccgcaagaccaagggctccggcttcttcgtcttcagccgcctggaggtgaccagggccgaatgggagcagaaagatgagttcatctgccgtgcagtccatgaggcagcaagcccctca

SEQ ID NO: 482 - DNA, 120 bp, synthetic construct
  source 1..120: mol_type = other DNA
cagaccgtccagcgagcggtgtctgtaaatcccgagctggacgtgtgcgtggaggaggccgagggcgaggcgccgtggacgtggaccggcctctgcatcttcgccgcactcttcctgctc

SEQ ID NO: 483 - DNA, 120 bp, synthetic construct
  source 1..120: mol_type = other DNA
agcgtgagctacagcgccgccatcacgctcctcatggtgcagcggttcctctcagccacgcggcaggggaggccccagacctccctcgactacaccaacgtcctccagccccacgcctag

SEQ ID NO: 484 - DNA, 120 bp, synthetic construct
  source 1..120: mol_type = other DNA
tcctgcctccctccctcccagggctccatccagctgtgcagtggggaggactggccagaccttctgtccactgttgcaatgaccccaggaagctacccccaataaactgtgcctgctcag

SEQ ID NO: 485 - DNA, 120 bp, synthetic construct
  source 1..120: mol_type = other DNA
gcttccaccaagggcccatcggtcttccccctggcgccctgctccaggagcacctccgagagcacagccgccctgggctgcctggtcaaggactacttccccgaaccggtgacggtgtcg

SEQ ID NO: 486 - DNA, 120 bp, synthetic construct
  source 1..120: mol_type = other DNA
tggaactcaggcgccctgaccagcggcgtgcacaccttcccggctgtcctacagtcctcaggactctactccctcagcagcgtggtgaccgtgccctccagcagcttgggcacgaagacc

SEQ ID NO: 487 - DNA, 120 bp, synthetic construct
  source 1..120: mol_type = other DNA
tacacctgcaacgtagatcacaagcccagcaacaccaaggtggacaagagagttgagtccaaatatggtcccccatgcccatcatgcccagcacctgagttcctggggggaccatcagtc

SEQ ID NO: 488 - DNA, 120 bp, synthetic construct
  source 1..120: mol_type = other DNA
ttcctgttccccccaaaacccaaggacactctcatgatctcccggacccctgaggtcacgtgcgtggtggtggacgtgagccaggaagaccccgaggtccagttcaactggtacgtggat

SEQ ID NO: 489 - DNA, 120 bp, synthetic construct
  source 1..120: mol_type = other DNA
ggcgtggaggtgcataatgccaagacaaagccgcgggaggagcagttcaacagcacgtaccgtgtggtcagcgtcctcaccgtcctgcaccaggactggctgaacggcaaggagtacaag

SEQ ID NO: 490 - DNA, 120 bp, synthetic construct
  source 1..120: mol_type = other DNA
tgcaaggtctccaacaaaggcctcccgtcctccatcgagaaaaccatctccaaagccaaagggcagccccgagagccacaggtgtacaccctgcccccatcccaggaggagatgaccaag

SEQ ID NO: 491 - DNA, 120 bp, synthetic construct
  source 1..120: mol_type = other DNA
aaccaggtcagcctgacctgcctggtcaaaggcttctaccccagcgacatcgccgtggagtgggagagcaatgggcagccggagaacaactacaagaccacgcctcccgtgctggactcc

SEQ ID NO: 492 - DNA, 120 bp, synthetic construct
  source 1..120: mol_type = other DNA
gacggctccttcttcctctacagcaggctcaccgtggacaagagcaggtggcaggaggggaatgtcttctcatgctccgtgatgcatgaggctctgcacaaccactacacacagaagagc

SEQ ID NO: 493 - DNA, 120 bp, synthetic construct
  source 1..120: mol_type = other DNA
ctctccctgtctctggagctgcaactggaggagagctgtgcggaggcgcaggacggggagctggacgggctgtggacgaccatcaccatcttcatcacactcttcctgctaagcgtgtgc

SEQ ID NO: 494 - DNA, 120 bp, synthetic construct
  source 1..120: mol_type = other DNA
tacagtgccaccgtcaccttcttcaaggtgaagtggatcttctcctcagtggtggacctgaagcagaccatcgtccccgactacaggaacatgataaggcagggggcctagggccaccct

SEQ ID NO: 495 - DNA, 120 bp, synthetic construct
  source 1..120: mol_type = other DNA
ccccctgacctcaccgccctcaaccccatggctctctggcttcgcagtcgccctctgagccctgaaacgccccccttccagaccctgtgcatagcaggtctaccccagacctccgctgct

SEQ ID NO: 496 - DNA, 120 bp, synthetic construct
  source 1..120: mol_type = other DNA
tggtgcatgcagggcgctgagggccaggtgtcccctcagcaggacgtccctgccctctggaccaccaggtgctcacacaaaaggaggtaaccggcatcccaggcccccactcaggcagga

SEQ ID NO: 497 - DNA, 120 bp, synthetic construct
  source 1..120: mol_type = other DNA
cctcgccctggagccaaccccgtccacgccagcctcctgaacacaggcatggtttccagatggtgagtgggagcatcagtcgccaaggtagggaagccacagcaccatcaggccctgttg

SEQ ID NO: 498 - DNA, 120 bp, synthetic construct
  source 1..120: mol_type = other DNA
gggaggcttccgagagctgcgaaggctcactcagacggccttcctcccagcccgcagccagccagcctccattccgggcactcccgtgaactcctgacatgaggaatgaggttgttctga

SEQ ID NO: 499 - DNA, 120 bp, synthetic construct
  source 1..120: mol_type = other DNA
tttcaagcaaagaacgctgctctctggctcctgggaacagtctcggtgccagcaccaccccttggctgcctgcccacactgctggattctcgggtggaactggacccgcagggacagcca

SEQ ID NO: 500 - DNA, 120 bp, synthetic construct
  source 1..120: mol_type = other DNA
gccccagagtccgcactggggagagaaagggccaggcccaggacactgccacctaccacccactccagtccaccgagatcactcggagaagagcctgggccatgtggccgctgcaggagc

SEQ ID NO: 501 - DNA, 120 bp, synthetic construct
  source 1..120: mol_type = other DNA
cccacagtgcaagggtgaggatagcccaaggaagggctgggcatctgcccagacaggcctcccacagaaggctggtgaccaggtcccaggcgggcaagactcagccttggtggggcctga

SEQ ID NO: 502 - DNA, 120 bp, synthetic construct
  source 1..120: mol_type = other DNA
ggacagaggaggcccaggagcatcggggagagaggtggagggacaccgggagagccaggagcgtggacacagccagaactcatcacagaggctggcgtccagtcccgggtcacgtgcagc

SEQ ID NO: 503 - DNA, 120 bp, synthetic construct
  source 1..120: mol_type = other DNA
aggaacaagcagccactctgggggcaccaggtggagaggcaagacgacaaagagggtgcccgtgttcttgcgaaagcggggctgctggccacgagtgctggacagaggcccccacgctct

SEQ ID NO: 504 - DNA, 120 bp, synthetic construct
  source 1..120: mol_type = other DNA
gctgcccccatcacaccgttccgtgactgtcacgcagaatccacagacaggaagggaggctcgagcgggactgcggccagcgcctgcctcggccgtcagggaggactcccgggctcactc

SEQ ID NO: 505 - DNA, 120 bp, synthetic construct
  source 1..120: mol_type = other DNA
gaaggaggtgtcaccatttcagctttggcttttcttcttcttttaaattttctaaagctcattaattgtctttgatgtttcttttgtgatgacaataaaatatcctttttaagtcttgta

SEQ ID NO: 506 - DNA, 120 bp, synthetic construct
  source 1..120: mol_type = other DNA
agcccccgctccccgggctctcggggtcgcgcgaggatgcttggcacgtaccccgtgtacatacttcccgggcgcccagcatggaaataaagcacccagcgctgccctgggcccctgcga

SEQ ID NO: 507 - DNA, 120 bp, synthetic construct
  source 1..120: mol_type = other DNA
gcctccaccaagggcccatcggtcttccccctggcgccctgctccaggagcacctccgagagcacagcggccctgggctgcctggtcaaggactacttccccgaaccggtgacggtgtcg

SEQ ID NO: 508 - DNA, 120 bp, synthetic construct
  source 1..120: mol_type = other DNA
tggaactcaggcgctctgaccagcggcgtgcacaccttcccggctgtcctacagtcctcaggactctactccctcagcagcgtggtgaccgtgccctccagcaacttcggcacccagacc

SEQ ID NO: 509 - DNA, 120 bp, synthetic construct
  source 1..120: mol_type = other DNA
tacacctgcaacgtagatcacaagcccagcaacaccaaggtggacaagacagttgagcgcaaatgttgtgtcgagtgcccaccgtgcccagcaccacctgtggcaggaccgtcagtcttc

SEQ ID NO: 510 - DNA, 120 bp, synthetic construct
  source 1..120: mol_type = other DNA
ctcttccccccaaaacccaaggacaccctcatgatctcccggacccctgaggtcacgtgcgtggtggtggacgtgagccacgaagaccccgaggtccagttcaactggtacgtggacggc

SEQ ID NO: 511 - DNA, 120 bp, synthetic construct
  source 1..120: mol_type = other DNA
gtggaggtgcataatgccaagacaaagccacgggaggagcagttcaacagcacgttccgtgtggtcagcgtcctcaccgtcgtgcaccaggactggctgaacggcaaggagtacaagtgc

SEQ ID NO: 512 - DNA, 120 bp, synthetic construct
  source 1..120: mol_type = other DNA
aaggtctccaacaaaggcctcccagcccccatcgagaaaaccatctccaaaaccaaagggcagccccgagaaccacaggtgtacaccctgcccccatcccgggaggagatgaccaagaac

SEQ ID NO: 513 - DNA, 120 bp, synthetic construct
  source 1..120: mol_type = other DNA
caggtcagcctgacctgcctggtcaaaggcttctaccccagcgacatctccgtggagtgggagagcaatgggcagccggagaacaactacaagaccacacctcccatgctggactccgac

SEQ ID NO: 514 - DNA, 120 bp, synthetic construct
  source 1..120: mol_type = other DNA
ggctccttcttcctctacagcaagctcaccgtggacaagagcaggtggcagcaggggaacgtcttctcatgctccgtgatgcatgaggctctgcacaaccactacacacagaagagcctc

SEQ ID NO: 515 - DNA, 120 bp, synthetic construct
  source 1..120: mol_type = other DNA
tccctgtctccggagctgcaactggaggagagctgtgcggaggcgcaggacggggagctggacgggctgtggaccaccatcaccatcttcatcacactcttcctgctaagcgtgtgctac

SEQ ID NO: 516 - DNA, 120 bp, synthetic construct
  source 1..120: mol_type = other DNA
agtgccaccatcaccttcttcaaggtgaagtggatcttctcctcagtggtggacctgaagcagaccatcgtccccgactacaggaacatgatcaggcagggggcctagggccaccctctg

SEQ ID NO: 517 - DNA, 120 bp, synthetic construct
  source 1..120: mol_type = other DNA
cccccgacctcaccgccctcaaccccatggctctctggcctcgcagtcgccctctgaccctgacacgccccccttccagaccctgtgcatagcaggtctaccccagacctccgctgcttg

SEQ ID NO: 518 - DNA, 120 bp, synthetic construct
  source 1..120: mol_type = other DNA
gtgcatgcagggcgctgggggccaagtgtcccctcagcaggacgtccctgccctccggcccgccaggtgctcacacaaaaggaggtagtgaccagcatcccaggcccccactcaggcagg

SEQ ID NO: 519 - DNA, 120 bp, synthetic construct
  source 1..120: mol_type = other DNA
acctcgccctggagccaaccctgtccacgccagcctcctgaacacaggcgtggtttccagatggtgagtgggagcatcagtcgccaaggtagggaagtcacagcaccatcaggccctgtt

SEQ ID NO: 520 - DNA, 120 bp, synthetic construct
  source 1..120: mol_type = other DNA
ggggaggcttccgagagctgcgaaggctcactcagacggccttcctcccagcccgcagccagccagcctccattccaggcactcccgtgaactcctgacatgaggaatgaggttgttctg

SEQ ID NO: 521 - DNA, 120 bp, synthetic construct
  source 1..120: mol_type = other DNA
atttcaagcaaagaacgctgctctctggctcctgggaacagtctcagtgccagcaccaccccttggctgcctgcccacactgctggattctcgggtggaactcgacccgcagggacagcc

SEQ ID NO: 522 - DNA, 120 bp, synthetic construct
  source 1..120: mol_type = other DNA
agccccagagtccgcactggggagagaaggggccaggcccaggacactgccacctaccacccactccagtccaccgagatcactcggagaagagcctgggccatgtggccgctgcaggag

SEQ ID NO: 523 - DNA, 120 bp, synthetic construct
  source 1..120: mol_type = other DNA
ccccacggtgcaagggtgaggatagcccaaggaagggctgggcatctgcccagacaggcctcccagagaaggctggtgaccaggtcccaggcgggcaagactcagccttggtggggcctg

SEQ ID NO: 524 - DNA, 120 bp, synthetic construct
  source 1..120: mol_type = other DNA
aggacagaggaggcccaggagcatcggggagagaggtggagggacaccgggagagccaggagcgtggacacagccagaactcatcacagaggctggcgtccagccccgggtcacgtgcag

SEQ ID NO: 525 - DNA, 120 bp, synthetic construct
  source 1..120: mol_type = other DNA
caggaacaagcagccactctgggggcaccaggtggagaggcaagacgacaaagagggtgcccgtgttcttgtgaaagcggggctgctggccacgagtgctggacagaggcccccacgctc

SEQ ID NO: 526 - DNA, 120 bp, synthetic construct
  source 1..120: mol_type = other DNA
tgctgcccccatcacgccgttccgtgactgtcacgcagaatccgcagacagggagactcgagcgggagtgcggccagcgcctgcctcagctgtcagggaggactcccgggctcactcgaa

SEQ ID NO: 527 - DNA, 120 bp, synthetic construct
  source 1..120: mol_type = other DNA
ggaggtgccaccatttcagctttggtagcttttcttcttcttttaaattttctaaagctcattaattgtctttgatgtttcttttgtgatgacaataaaatatcctttttaagtcttgta

SEQ ID NO: 528 - DNA, 120 bp, synthetic construct
  source 1..120: mol_type = other DNA
agcccccgctccccaggctctcggggtcgcgcgaggatgcttggcacgtaccccgtctacatacttcccgggcacccagcatggaaataaagcacccagcgctgccctgggcccctgcga

SEQ ID NO: 529 - DNA, 120 bp, synthetic construct
  source 1..120: mol_type = other DNA
gcatccccgaccagccccaaggtcttcccgctgagcctctgcagcacccagccagatgggaacgtggtcatcgcctgcctggtccagggcttcttcccccaggagccactcagtgtgacc

SEQ ID NO: 530 - DNA, 120 bp, synthetic construct
  source 1..120: mol_type = other DNA
tggagcgaaagcggacagggcgtgaccgccagaaacttcccacccagccaggatgcctccggggacctgtacaccacgagcagccagctgaccctgccggccacacagtgcctagccggc

SEQ ID NO: 531 - DNA, 120 bp, synthetic construct
  source 1..120: mol_type = other DNA
aagtccgtgacatgccacgtgaagcactacacgaatcccagccaggatgtgactgtgccctgcccagttccctcaactccacctaccccatctccctcaactccacctaccccatctccc

SEQ ID NO: 532 - DNA, 120 bp, synthetic construct
  source 1..120: mol_type = other DNA
tcatgctgccacccccgactgtcactgcaccgaccggccctcgaggacctgctcttaggttcagaagcgaacctcacgtgcacactgaccggcctgagagatgcctcaggtgtcaccttc

SEQ ID NO: 533 - DNA, 120 bp, synthetic construct
  source 1..120: mol_type = other DNA
acctggacgccctcaagtgggaagagcgctgttcaaggaccacctgagcgtgacctctgtggctgctacagcgtgtccagtgtcctgccgggctgtgccgagccatggaaccatgggaag

SEQ ID NO: 534 - DNA, 120 bp, synthetic construct
  source 1..120: mol_type = other DNA
ggaggagctggccctgaacgagctggtgacgctgacgtgcctggcacgcggcttcagccccaaggatgtgctggttcgctggctgcaggggtcacaggagctgccccgcgagaagtacct

SEQ ID NO: 535 - DNA, 120 bp, synthetic construct
  source 1..120: mol_type = other DNA
gacttgggcatcccggcaggagcccagccagggcaccaccaccttcgctgtgaccagcatactgcgcgtggcagccgaggactggaagaagggggacaccttctcctgcatggtgggcca

SEQ ID NO: 536 - DNA, 120 bp, synthetic construct
  source 1..120: mol_type = other DNA
cgaggccctgccgctggccttcacacagaagaccatcgaccgcttggcggattggcagatgccgcctccctatgtggtgctggacttgccgcaggagaccctggaggaggagacccccgg

SEQ ID NO: 537 - DNA, 120 bp, synthetic construct
  source 1..120: mol_type = other DNA
cgccaacctgtggcccaccaccatcaccttcctcaccctcttcctgctgagcctgttctatagcacagcactgaccgtgaccagcgtccggggcccatctggcaacagggagggccccca

SEQ ID NO: 538 - DNA, 120 bp, synthetic construct
  source 1..120: mol_type = other DNA
gtactgagcaggagccggcaaggcacagggaggaagtgtggaggaacctcttggagaagccagctatgcttgccagaactcagccctttcagacatcaccgacccgcccttactcacatg

SEQ ID NO: 539 - DNA, 120 bp, synthetic construct
  source 1..120: mol_type = other DNA
cttggcgggtaaacccacccatgtcaatgtgtctgttgtcatggcggaggtggacggcacctgctactgagccgcccgcctgtccccacccctgaataaactccatgctcccccaagcag

SEQ ID NO: 540 - DNA, 120 bp, synthetic construct
  source 1..120: mol_type = other DNA
gcctccaccaagggcccatcggtcttccccctggcaccctcctccaagagcacctctgggggcacagcagccctgggctgcctggtcaaggactacttccccgaaccggtgacggtgtcg

SEQ ID NO: 541 - DNA, 120 bp, synthetic construct
  source 1..120: mol_type = other DNA
tggaactcaggcgccctgaccagcggcgtgcacaccttcccggctgtcctacagtcctcaggactctactccctcagcagcgtggtgaccgtgccctccagcagcttgggcacccagacc

SEQ ID NO: 542 - DNA, 120 bp, synthetic construct
  source 1..120: mol_type = other DNA
tacatctgcaacgtgaatcacaagcccagcaacaccaaggtggacaagaaagttgagcccaaatcttgtgacaaaactcacacatgcccaccgtgcccagcacctgaactcctgggggga

SEQ ID NO: 543 - DNA, 120 bp, synthetic construct
  source 1..120: mol_type = other DNA
ccgtcagtcttcctcttccccccaaaacccaaggacaccctcatgatctcccggacccctgaggtcacatgcgtggtggtggacgtgagccacgaagaccctgaggtcaagttcaactgg

SEQ ID NO: 544 - DNA, 120 bp, synthetic construct
  source 1..120: mol_type = other DNA
tacgtggacggcgtggaggtgcataatgccaagacaaagccgcgggaggagcagtacaacagcacgtaccgtgtggtcagcgtcctcaccgtcctgcaccaggactggctgaatggcaag

SEQ ID NO: 545 - DNA, 120 bp, synthetic construct
  source 1..120: mol_type = other DNA
gagtacaagtgcaaggtctccaacaaagccctcccagcccccatcgagaaaaccatctccaaagccaaagggcagccccgagaaccacaggtgtacaccctgcccccatcccgggatgag

SEQ ID NO: 546 - DNA, 120 bp, synthetic construct
  source 1..120: mol_type = other DNA
ctgaccaagaaccaggtcagcctgacctgcctggtcaaaggcttctatcccagcgacatcgccgtggagtgggagagcaatgggcagccggagaacaactacaagaccacgcctcccgtg

SEQ ID NO: 547 - DNA, 120 bp, synthetic construct
  source 1..120: mol_type = other DNA
ctggactccgacggctccttcttcctctacagcaagctcaccgtggacaagagcaggtggcagcaggggaacgtcttctcatgctccgtgatgcatgaggctctgcacaaccactacaca

SEQ ID NO: 548 - DNA, 120 bp, synthetic construct
  source 1..120: mol_type = other DNA
cagaagagcctctccctgtctccggagctgcaactggaggagagctgtgcggaggcgcaggacggggagctggacgggctgtggacgaccatcaccatcttcatcacactcttcctgtta

SEQ ID NO: 549 - DNA, 120 bp, synthetic construct
  source 1..120: mol_type = other DNA
agcgtgtgctacagtgccaccgtcaccttcttcaaggtgaagtggatcttctcctcggtggtggacctgaagcagaccatcatccccgactacaggaacatgatcggacagggggcctag

SEQ ID NO: 550 - DNA, 120 bp, synthetic construct
  source 1..120: mol_type = other DNA
cgccctcaaccccatgactctctggcctcgcagttgccctctgaccctgacacacctgacacgccccccttccagaccctgtgcatagcaggtctaccccagacctccgctgcttggtgc

SEQ ID NO: 551 - DNA, 120 bp, synthetic construct
  source 1..120: mol_type = other DNA
atgcagggcactgggggccaggtgtcccctcagcaggacgtccttgccctccggaccacaaggtgctcacacaaaaggaggcagtgaccggtatcccaggcccccacccaggcaggacct

SEQ ID NO: 552 - DNA, 120 bp, synthetic construct
  source 1..120: mol_type = other DNA
cgccctggagccaaccccgtccacgccagcctcctgaacacaggcgtggtttccagatggtgagtgggagcgtcagccgccaaggtagggaagccacagcaccatcaggccctgttgggg

SEQ ID NO: 553 - DNA, 120 bp, synthetic construct
  source 1..120: mol_type = other DNA
aggcttccgagagctgcgaaggctcactcagacggccttcctcccagcccgcagccagccagcctccattccgggcactcccgtgaactcctgacatgaggaatgaggttgttctgattt

SEQ ID NO: 554 - DNA, 120 bp, synthetic construct
  source 1..120: mol_type = other DNA
caagcaaagaacgctgctctctggctcctgggaacagtctcagtgccagcaccaccccttggctgcctgcccacactgctggattctcgggtggaactggacccgcagggacagccagcc

SEQ ID NO: 555 - DNA, 120 bp, synthetic construct
  source 1..120: mol_type = other DNA
ccagagtccgcactggggagagaaggggccaggcccaggacactgccacctcccacccactccagtccaccgagatcactcagagaagagcctgggccatgtggccgctgcaggagcccc

SEQ ID NO: 556 - DNA, 120 bp, synthetic construct
  source 1..120: mol_type = other DNA
acagtgcaagggtgaggatagcccaaggaagggctgggcatctgcccagacaggcctcccagagaaggctggtgaccaggtcccaggcgggcaagactcagccttggtggggcctgagga

SEQ ID NO: 557 - DNA, 120 bp, synthetic construct
  source 1..120: mol_type = other DNA
cagaggaggcccaggagcatcggggagagaggtggagggacaccgggagagccaggagcgtggacacagccagaactcatcacagaggctggcgtccagccccgggtcacgtgcagcagg

SEQ ID NO: 558 - DNA, 120 bp, synthetic construct
  source 1..120: mol_type = other DNA
aacaagcagccactctgggggcaccaggtggagaggcaagacgacaaagagggtgcccgtgttcttgcgaaagcagggctgctggccacgagtgctggacagaggcccccacgctctgct

SEQ ID NO: 559 - DNA, 120 bp, synthetic construct
  source 1..120: mol_type = other DNA
gcccccatcacgccgttccgtgactgtcacgcagaatctgcagacaggaagggagactcgagcgggagtgcggccagcgcctgcctcggccgtcagggaggactcctgggctcactcgaa

SEQ ID NO: 560 - DNA, 120 bp, synthetic construct; identical to SEQ ID NO: 527
  source 1..120: mol_type = other DNA

SEQ ID NO: 561 - DNA, 120 bp, synthetic construct
  source 1..120: mol_type = other DNA
aagcccccgctccccaggctctcggggtcgcgcgaggatgcttggcacgtaccccgtgtacatacttcccaggcacccagcatggaaataaagcacccagcgcttccctgggcccctgcg

SEQ ID NO: 562 - DNA, 120 bp, synthetic construct
  source 1..120: mol_type = other DNA
cttccaccaagggcccatcggtcttccccctggcgccctgctccaggagcacctctgggggcacagcggccctgggctgcctggtcaaggactacttcccagaaccggtgacggtgtcgt

SEQ ID NO: 563 - DNA, 120 bp, synthetic construct
  source 1..120: mol_type = other DNA
ggaactcaggcgccctgaccagcggcgtgcacaccttcccggctgtcctacagtcctcaggactctactccctcagcagcgtggtgaccgtgccctccagcagcttgggcacccagacct

SEQ ID NO: 564 - DNA, 120 bp, synthetic construct
  source 1..120: mol_type = other DNA
acacctgcaacgtgaatcacaagcccagcaacaccaaggtggacaagagagttgagctcaaaaccccacttggtgacacaactcacacatgcccacggtgcccagagcccaaatcttgtg

SEQ ID NO: 565 - DNA, 120 bp, synthetic construct
  source 1..120: mol_type = other DNA
acacacctcccccgtgcccacggtgcccagagcccaaatcttgtgacacacctcccccatgcccacggtgcccagagcccaaatcttgtgacacacctcccccgtgcccaaggtgcccag

SEQ ID NO: 566 - DNA, 120 bp, synthetic construct
  source 1..120: mol_type = other DNA
cacctgaactcctgggaggaccgtcagtcttcctcttccccccaaaacccaaggatacccttatgatttcccggacccctgaggtcacgtgcgtggtggtggacgtgagccacgaagacc

SEQ ID NO: 567 - DNA, 120 bp, synthetic construct
  source 1..120: mol_type = other DNA
ccgaggtccagttcaagtggtacgtggacggcgtggaggtgcataatgccaagacaaagccgcgggaggagcagtacaacagcacgttccgtgtggtcagcgtcctcaccgtcctgcacc

SEQ ID NO: 568 - DNA, 120 bp, synthetic construct
  source 1..120: mol_type = other DNA
aggactggctgaacggcaaggagtacaagtgcaaggtctccaacaaagccctcccagcccccatcgagaaaaccatctccaaaaccaaaggacagccccgagaaccacaggtgtacaccc

SEQ ID NO: 569 - DNA, 120 bp, synthetic construct
  source 1..120: mol_type = other DNA
tgcccccatcccgggaggagatgaccaagaaccaggtcagcctgacctgcctggtcaaaggcttctaccccagcgacatcgccgtggagtgggagagcagcgggcagccggagaacaact

SEQ ID NO: 570 - DNA, 120 bp, synthetic construct
  source 1..120: mol_type = other DNA
acaacaccacgcctcccatgctggactccgacggctccttcttcctctacagcaagctcaccgtggacaagagcaggtggcagcaggggaacatcttctcatgctccgtgatgcatgagg

SEQ ID NO: 571 - DNA, 120 bp, synthetic construct
  source 1..120: mol_type = other DNA
ctctgcacaaccgcttcacgcagaagagcctctccctgtctccggagctgcaactggaggagagctgtgcggaggcgcaggacggggagctggacgggctgtggacgaccatcaccatct

SEQ ID NO: 572 - DNA, 120 bp, synthetic construct
  source 1..120: mol_type = other DNA
tcatcacactcttcctgttaagcgtgtgctacagtgccaccgtcaccttcttcaaggtgaagtggatcttctcctcggtggtggacctgaagcagaccatcatccccgactataggaaca

SEQ ID NO: 573 - DNA, 120 bp, synthetic construct
  source 1..120: mol_type = other DNA
gacctcaccgccctcaaccccatggctctctgtctttgcagtcgccctctgagccctgacacgccccccttccagaccctgtgcatagcaggtctaccccagacctccgctgcttggtgc

SEQ ID NO: 574 - DNA, 120 bp, synthetic construct
  source 1..120: mol_type = other DNA
atgcagggagctggggaccaggtgtcccctcagcaggatgtccctgccctccagaccgccagatgctcacacaaaaggaggcagtgaccagcatccgaggcccccacccaggcaggagct

SEQ ID NO: 575 - DNA, 120 bp, synthetic construct
  source 1..120: mol_type = other DNA
ggccctggagccaaccccgtccacgccagcctcctgaacacaggcgtggtttccagatggtgagtgggagcatcagccgccaaggtagggaagccacagcaccatcaggccctgttgggg

SEQ ID NO: 576 - DNA, 120 bp, synthetic construct; identical to SEQ ID NO: 553
  source 1..120: mol_type = other DNA

SEQ ID NO: 577 - DNA, 120 bp, synthetic construct
  source 1..120: mol_type = other DNA
caagcaaagaacgctgctctctggctcctgggaacagtctcggtgccagcaccaccccttggctgcctgcctacactgctggattctcgggtggaactggacccgcagggacagccagcc

SEQ ID NO: 578 - DNA, 120 bp, synthetic construct
  source 1..120: mol_type = other DNA
ccagagtccgcactggggagagaaggggccaggcccaggacactgccacctcccacccactccagtccaccgagatcactcagagaagagcctgggccatgtggccactgcaggagcccc

SEQ ID NO: 579 - DNA, 120 bp, synthetic construct
  source 1..120: mol_type = other DNA
acagtgcaagagtgaggatagcccaaggaagggctgggcatctgcccagacaggcctcccagagaaggctggtgaccaggtcccaggcgggcaagactcagccttggtggggcctgagga

SEQ ID NO: 580 - DNA, 120 bp, synthetic construct; identical to SEQ ID NO: 557
  source 1..120: mol_type = other DNA

SEQ ID NO: 581 - DNA, 120 bp, synthetic construct
  source 1..120: mol_type = other DNA
aacaagcagccactctgggggcaccaggtggagaggcaagatgccaaagagggtgcccgtgttcttgcgaaagcggggctgctggccacgagtgctggacagaggcccccacgctctgct

SEQ ID NO: 582 - DNA, 120 bp, synthetic construct
  source 1..120: mol_type = other DNA
gcccccatcacgccgttccgtgactgtcacgcagaatccgcagacaggaagggaggctcgagcgggactgcggccagcgcctgcctcggccgtcagggaggactcccgggctcactcgaa

SEQ ID NO: 583 - DNA, 120 bp, synthetic construct; identical to SEQ ID NO: 527
  source 1..120: mol_type = other DNA

SEQ ID NO: 584 - DNA, 120 bp, synthetic construct
  source 1..120: mol_type = other DNA
agcccccgctccccgggctctcggggtcgcgcgaggatgcttggcacgtaccccgtgtacatacttcccgggcacccagcatggaaataaagcacccagcgctgccctgggcccctgcga

SEQ ID NO: 585 - DNA, 120 bp, synthetic construct
  source 1..120: mol_type = other DNA
cacccaccaaggctccggatgtgttccccatcatatcagggtgcagacacccaaaggataacagccctgtggtcctggcatgcttgataactgggtaccacccaacgtccgtgactgtca

SEQ ID NO: 586 - DNA, 120 bp, synthetic construct
  source 1..120: mol_type = other DNA
cctggtacatggggacacagagccagccccagagaaccttccctgagatacaaagacgggacagctactacatgacaagcagccagctctccacccccctccagcagtggcgccaaggcg

SEQ ID NO: 587 - DNA, 120 bp, synthetic construct
  source 1..120: mol_type = other DNA
agtacaaatgcgtggtccagcacaccgccagcaagagtaagaaggagatcttccgctggccagagtctccaaaggcacaggcctcctcagtgcccactgcacaaccccaagcagagggca

SEQ ID NO: 588 - DNA, 120 bp, synthetic construct
  source 1..120: mol_type = other DNA
gcctcgccaaggcaaccacagccccagccaccacccgtaacacaggaagaggaggagaagagaagaagaaggagaaggagaaagaggaacaagaagagagagagacaaagacaccagagt

SEQ ID NO: 589 - DNA, 120 bp, synthetic construct
  source 1..120: mol_type = other DNA
gtccgagccacacccagcctcttggcgtctacctgctaacccctgcagtgcaggacctgtggctccgggacaaagccaccttcacctgcttcgtggtgggcagtgacctgaaggatgctc

SEQ ID NO: 590 - DNA, 120 bp, synthetic construct
  source 1..120: mol_type = other DNA
acctgacctgggaggtggctgggaaggtccccacagggggcgtggaggaagggctgctggagcggcacagcaacggctcccagagccagcacagccgtctgaccctgcccaggtccttgt

SEQ ID NO: 591 - DNA, 120 bp, synthetic construct
  source 1..120: mol_type = other DNA
ggcctcgtctgaccctcccgaggcggcctcgtggctcctgtgtgaggtgtctggcttctcgccccccaacatcctcctgatgtggctggaggaccagcgtgaggtgaacacttctgggtt

SEQ ID NO: 592 - DNA, 120 bp, synthetic construct
  source 1..120: mol_type = other DNA
tgcccccgcacgcccccctccacagcccaggagcaccacgttctgggcctggagtgtgctgcgtgtcccagccccgcccagccctcagccagccacctacacgtgtgtggtcagccacga

SEQ ID NO: 593 - DNA, 120 bp, synthetic construct
  source 1..120: mol_type = other DNA
ggactcccggactctgctcaacgccagccggagcctagaagtcagctacctggccatgacccccctgatccctcagagcaaggatgagaacagcgatgactacacgacctttgatgatgt

SEQ ID NO: 594 - DNA, 120 bp, synthetic construct
  source 1..120: mol_type = other DNA
gggcagcctgtggaccaccctgtccacgtttgtggccctcttcatcctcaccctcctctacagcggcattgtcactttcatcaaggtgaagtagccccagaagagcaggacgccctgtac

SEQ ID NO: 595 - DNA, 120 bp, synthetic construct
  source 1..120: mol_type = other DNA
ctgcagagaagggaagcagcctctgtacctcatctgtggctaccagagagcagaaaggacccaccctggactcttctgtgtgcaggaagatgcgccagcccctgcccccggctcccctct

SEQ ID NO: 596 - DNA, 120 bp, synthetic construct
  source 1..120: mol_type = other DNA
gtccgccacagaacccagtcttctagaccagggggacgggcacccatcactccgcaggcgaatcagagcccccctgccccggccctaacccctgtgcctccttcccatgcttccccgaga

SEQ ID NO: 597 - DNA, 120 bp, synthetic construct
  source 1..120: mol_type = other DNA
gccagctacacccctgccccggccctaacccccatgcctccttcctgtgcttcccccagagccagctagtcccacctgcagcccgctggcctccccataaacacactttggttcatttca

SEQ ID NO: 598 - DNA, 120 bp, synthetic construct
  source 1..120: mol_type = other DNA
gggagtgcatccgccccaacccttttccccctcgtctcctgtgagaattccccgtcggatacgagcagcgtggccgttggctgcctcgcacaggacttccttcccgactccatcactttc

SEQ ID NO: 599 - DNA, 120 bp, synthetic construct
  source 1..120: mol_type = other DNA
tcctggaaatacaagaacaactctgacatcagcagcacccggggcttcccatcagtcctgagagggggcaagtacgcagccacctcacaggtgctgctgccttccaaggacgtcatgcag

SEQ ID NO: 600 - DNA, 120 bp, synthetic construct
  source 1..120: mol_type = other DNA
ggcacagacgaacacgtggtgtgcaaagtccagcaccccaacggcaacaaagaaaagaacgtgcctcttccagtgattgctgagctgcctcccaaagtgagcgtcttcgtcccaccccgc

SEQ ID NO: 601 - DNA, 120 bp, synthetic construct
  source 1..120: mol_type = other DNA
gacggcttcttcggcaacccccgcaagtccaagctcatctgccaggccacgggtttcagtccccggcagattcaggtgtcctggctgcgcgaggggaagcaggtggggtctggcgtcacc

SEQ ID NO: 602 - DNA, 120 bp, synthetic construct
  source 1..120: mol_type = other DNA
acggaccaggtgcaggctgaggccaaagagtctgggcccacgacctacaaggtgaccagcacactgaccatcaaagagagcgactggctcggccagagcatgttcacctgccgcgtggat

SEQ ID NO: 603 - DNA, 120 bp, synthetic construct
  source 1..120: mol_type = other DNA
cacaggggcctgaccttccagcagaatgcgtcctccatgtgtgtccccgatcaagacacagccatccgggtcttcgccatccccccatcctttgccagcatcttcctcaccaagtccacc

SEQ ID NO: 604 - DNA, 120 bp, synthetic construct
  source 1..120: mol_type = other DNA
aagttgacctgcctggtcacagacctgaccacctatgacagcgtgaccatctcctggacccgccagaatggcgaagctgtgaaaacccacaccaacatctccgagagccaccccaatgcc

SEQ ID NO: 605 - DNA, 120 bp, synthetic construct
  source 1..120: mol_type = other DNA
agcgccgtgggtgaggccagcatctgcgaggatgactggaattccggggagaggttcacgtgcaccgtgacccacacagacctgccctcgccactgaagcagaccatctcccggcccaag

SEQ ID NO: 606 - DNA, 120 bp, synthetic construct
  source 1..120: mol_type = other DNA
ggggtggccctgcacaggcccgatgtctacttgctgccaccagcccgggagcagctgaacctgcgggagtcggccaccatcacgtgcctggtgacgggcttctctcccgcggacgtcttc

SEQ ID NO: 607 - DNA, 120 bp, synthetic construct
  source 1..120: mol_type = other DNA
gtgcagtggatgcagagggggcagcccttgtccccggagaagtatgtgaccagcgccccaatgcctgagccccaggccccaggccggtacttcgcccacagcatcctgaccgtgtccgaa

SEQ ID NO: 608 - DNA, 120 bp, synthetic construct
  source 1..120: mol_type = other DNA
gaggaatggaacacgggggagacctacacctgcgtggtggcccatgaggccctgcccaacagggtcaccgagaggaccgtggacaagtccaccgagggggaggtgagcgccgacgaggag

SEQ ID NO: 609 - DNA, 120 bp, synthetic construct
  source 1..120: mol_type = other DNA
ggctttgagaacctgtgggccaccgcctccaccttcatcgtcctcttcctcctgagcctcttctacagtaccaccgtcaccttgttcaaggtgaaatgatcccaacagaagaacatcgga

SEQ ID NO: 610 - DNA, 120 bp, synthetic construct
  source 1..120: mol_type = other DNA
gaccagagagaggaactcaaaggggcgctgcctccgggtctggggtcctggcctgcgtggcctgttggcacgtgtttctcttccccgcccggcctccagttgtgtgctctcacacaggct

SEQ ID NO: 611 - DNA, 120 bp, synthetic construct
  source 1..120: mol_type = other DNA
tccttctcgaccggcaggggctggctggcttgcaggccacgaggtgggctctaccccacactgctttgctgtgtatacgcttgttgccctgaaataaatatgcacattttatccatgaaa

SEQ ID NO: 612 - DNA, 120 bp, synthetic construct
  source 1..120: mol_type = other DNA
tgctggcctgcccacaggctcggggcggctggccgctctgtgtgtgcatgcaaactaaccgtgtcaacggggtgagatgttgcatcttataaaattagaaataaaaagatccattcaaaa

SEQ ID NO: 613 - DNA, 120 bp, synthetic construct
  source 1..120: mol_type = other DNA
gccacccccttggtcactctgttcccgccctcctctgaggagctccaagccaacaaggccatgctggtgtgtctcataaatgacttctacccaggagccatagaaggaaaatggcaccct

SEQ ID NO: 614 - DNA, 120 bp, synthetic construct
  source 1..120: mol_type = other DNA
atgcggccagcagctacctgagcctgacgcccgagcagtggaagtcccacagaagctacagctgccaggtcacgcacaaagaaagtaccatggagaagacaatggcccatgcagaatgtt

SEQ ID NO: 615 - DNA, 120 bp, synthetic construct
  source 1..120: mol_type = other DNA
acaaggccacactggtgtgtctcatgagtgacttctacccgagagccatgacagtggcctggaagatagatggcatcaccatcacccagggtgtggagaccaccacaccctccaaacaga

SEQ ID NO: 616 - DNA, 120 bp, synthetic construct
  source 1..120: mol_type = other DNA
tatgcggccagcagctacctaagactggcacccgacagtggaagtcccacaacctctacagctgccaggtcacgcatgaaaggaacactgtggagaagacagtggcccctgcagaatgtt

SEQ ID NO: 617 - DNA, 120 bp, synthetic construct
  source 1..120: mol_type = other DNA
gtcagcccaaggctgccccatcggtcactctgttcccgccctcctctgaggagcttcaagccaacaaggccacactggtgtgcctgatcagtgacttctacccgggagctgtgaaagtgg

SEQ ID NO: 618 - DNA, 120 bp, synthetic construct
  source 1..120: mol_type = other DNA
gcggccagcagctagctacctgagcctgacgcctgagcagtggaagtcccacagaagctacagttgccaggtcacgcatgaagggagcaccgtggagaagacagtggcccctgcagaatg

SEQ ID NO: 619 - DNA, 120 bp, synthetic construct
  source 1..120: mol_type = other DNA
aggacctgaacaaggtgttcccacccgaggtcgctgtgtttgagccatcagaagcagagatctcccacacccaaaaggccacactggtgtgcctggccacaggcttcttccctgaccacg

SEQ ID NO: 620 - DNA, 120 bp, synthetic construct
  source 1..120: mol_type = other DNA
tggagctgagctggtgggtgaatgggaaggaggtgcacagtggggtcagcacggacccgcagcccctcaaggagcagcccgccctcaatgactccagatactgcctgagcagccgcctga

SEQ ID NO: 621 - DNA, 120 bp, synthetic construct
  source 1..120: mol_type = other DNA
gggtctcggccaccttctggcagaacccccgcaaccacttccgctgtcaagtccagttctacgggctctcggagaatgacgagtggacccaggatagggccaaacccgtcacccagatcg

SEQ ID NO: 622 - DNA, 120 bp, synthetic construct
  source 1..120: mol_type = other DNA
ctcggtgtcctaccagcaaggggtcctgtctgccaccatcctctatgagatcctgctagggaaggccaccctgtatgctgtgctggtcagcgcccttgtgttgatggccatggtcaagag

SEQ ID NO: 623 - DNA, 120 bp, synthetic construct
  source 1..120: mol_type = other DNA
aaaggatttctgaaggcagccctggaagtggagttaggagcttctaacccgtcatggtttcaatacacattcttcttttgccagcgcttctgaagagctgctctcacctctctgcatccc

SEQ ID NO: 624 - DNA, 120 bp, synthetic construct
  source 1..120: mol_type = other DNA
aatagatatccccctatgtgcatgcacacctgcacactcacggctgaaatctccctaacccagggggaccttagcatgcctaagtgactaaaccaataaaaatgttctggtctggcctga

SEQ ID NO: 625 - DNA, 120 bp, synthetic construct
  source 1..120: mol_type = other DNA
aggacctgaaaaacgtgttcccacccaaggtcgctgtgtttgagccatcagaagcagagatctcccacacccaaaaggccacactggtgtgcctggccacaggcttctaccccgaccacg

SEQ ID NO: 626 - DNA, 120 bp, synthetic construct
  source 1..120: mol_type = other DNA
tggagctgagctggtgggtgaatgggaaggaggtgcacagtggggtcagcacagacccgcagcccctcaaggagcagcccgccctcaatgactccagatactgcctgagcagccgcctga

SEQ ID NO: 627 - DNA, 120 bp, synthetic construct
  source 1..120: mol_type = other DNA
gggtctcggccaccttctggcagaacccccgcaaccacttccgctgtcaagtccagttctacgggctctcggagaatgacgagtggacccaggatagggccaaacctgtcacccagatcg

SEQ ID NO: 628 - DNA, 120 bp, synthetic construct
  source 1..120: mol_type = other DNA
acctccgagtcttaccagcaaggggtcctgtctgccaccatcctctatgagatcttgctagggaaggccaccttgtatgccgtgctggtcagtgccctcgtgctgatggccatggtcaag

SEQ ID NO: 629 - DNA, 120 bp, synthetic construct
  source 1..120: mol_type = other DNA
agaaaggattccagaggctagctccaaaaccatcccaggtcattcttcatcctcacccaggattctcctgtacctgctcccaatctgtgttcctaaaagtgattctcactctgcttctca

SEQ ID NO: 630 - DNA, 120 bp, synthetic construct
  source 1..120: mol_type = other DNA
tctcctacttacatgaatacttctctcttttttctgtttccctgaagattgagctcccaacccccaagtacgaaataggctaaaccaataaaaaattgtgtgttgggcctggttgcattt

SEQ ID NO: 631 - DNA, 120 bp, synthetic construct
  source 1..120: mol_type = other DNA
atatccagaaccctgaccctgccgtgtaccagctgagagactctaaatccagtgacaagtctgtctgcctattcaccgattttgattctcaaacaaatgtgtcacaaagtaaggattctg

SEQ ID NO: 632 - DNA, 120 bp, synthetic construct
  source 1..120: mol_type = other DNA
atgtgtatatcacagacaaaactgtgctagacatgaggtctatggacttcaagagcaacagtgctgtggcctggagcaacaaatctgactttgcatgtgcaaacgccttcaacaacagca

SEQ ID NO: 633 - DNA, 120 bp, synthetic construct
  source 1..120: mol_type = other DNA
ttattccagaagacaccttcttccccagcccagaaagttcctgtgatgtcaagctggtcgagaaaagctttgaaacagatacgaacctaaactttcaaaacctgtcagtgattgggttcc

SEQ ID NO: 634 - DNA, 120 bp, synthetic construct
  source 1..120: mol_type = other DNA
gaatcctcctcctgaaagtggccgggtttaatctgctcatgacgctgcggctgtggtccagctgagatctgcaagattgtaagacagcctgtgctccctcgctccttcctctgcattgcc

SEQ ID NO: 635 - DNA, 120 bp, synthetic construct
  source 1..120: mol_type = other DNA
acagagggaactctcctacccccaaggaggtgaaagctgctaccacctctgtgcccccccggcaatgccaccaactggatcctacccgaatttatgattaagattgctgaagagctgcca

SEQ ID NO: 636 - DNA, 120 bp, synthetic construct
  source 1..120: mol_type = other DNA
aacactgctgccaccccctctgttcccttattgctgcttgtcactgcctgacattcacggcagaggcaaggctgctgcagcctcccctggctgtgcacattccctcctgctccccagaga

SEQ ID NO: 637 - DNA, 120 bp, synthetic construct
  source 1..120: mol_type = other DNA
ctgcctccgccatcccacagatgatggatcttcagtgggttctcttgggctctaggtcctgcagaatgttgtgaggggtttatttttttttaatagtgttcataaagaaatacatagtat

SEQ ID NO: 638 - DNA, 120 bp, synthetic construct
  source 1..120: mol_type = other DNA
tcttcttctcaagacgtggggggaaattatctcattatcgaggccctgctatgctgtgtatctgggcgtgttgtatgtcctgctgccgatgccttcattaaaatgatttggaagagcaga

SEQ ID NO: 639 - DNA, 82 bp, synthetic construct
  source 1..82: mol_type = other DNA
ctacacgacgctcttccgatctnnnnnnnnnnnnnnnnnnnnnnnnnnnnttttttttttttttttttttttttttttttvn

SEQ ID NO: 640 - DNA, 30 bp, synthetic construct
  source 1..30: mol_type = other DNA
cccatgtactctgcgttgataccactgctt